SEQUENCE LISTING (40 sequences)

SEQ ID NO: 1 - protein, 460 aa, Artificial Sequence
  Synthetic Polypeptide
MDWTWRILFLVAAATGAHSEVQLVQSGAEVKKPGATVKISCKASGFSIKDDYIHWVQQAPGKGLEWMGWIDAMNDDSQYSSKFQGRVTITVDTSTNTAYMKLSSLRSEDTAVYYCTYAFGGYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 241 aa, Artificial Sequence
  Synthetic Polypeptide
MDMRVPAQLLGLLLLWLRGARCDIVMTQTPLSLSVTPGQPASISCRSSQSLVHSDSNTYLHWYLQKPGQSPQLLIYLVSNRFSGVPDRFSGSGSGTDFTLKISRVEAEDVGVYFCGQSTHVPYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 114 aa, Artificial Sequence
  Synthetic Polypeptide
EVQLVQSGAEVKKPGATVKISCKASGFSIKDDYIHWVQQAPGKGLEWMGWIDAMNDDSQYSSKFQGRVTITVDTSTNTAYMKLSSLRSEDTAVYYCTYAFGGYWGQGTTVTVSS

SEQ ID NO: 4 - protein, 112 aa, Artificial Sequence
  Synthetic Polypeptide
DIVMTQTPLSLSVTPGQPASISCRSSQSLVHSDSNTYLHWYLQKPGQSPQLLIYLVSNRFSGVPDRFSGSGSGTDFTLKISRVEAEDVGVYFCGQSTHVPYAFGGGTKVEIK

SEQ ID NO: 5 - protein, 5 aa, Artificial Sequence
  Synthetic Oligopeptide
DDYIH

SEQ ID NO: 6 - protein, 17 aa, Artificial Sequence
  Synthetic Oligopeptide
WIDAMNDDSQYSSKFQG

SEQ ID NO: 7 - protein, 5 aa, Artificial Sequence
  Synthetic Oligopeptide
AFGGY

SEQ ID NO: 8 - protein, 16 aa, Artificial Sequence
  Synthetic Oligopeptide
RSSQSLVHSDSNTYLH

SEQ ID NO: 9 - protein, 7 aa, Artificial Sequence
  Synthetic Oligopeptide
LVSNRFS

SEQ ID NO: 10 - protein, 9 aa, Artificial Sequence
  Synthetic Oligopeptide
GQSTHVPYA

SEQ ID NO: 11 - DNA, 1383 bp, Artificial Sequence
  Synthetic Polynucleotide
atggactggacctggaggatcctcttcttggtggcagcagccacaggtgcccactccgaggtgcagctggtgcagtccggcgctgaggtgaagaagcctggcgccaccgtgaagatctcctgcaaggcctccggcttcagcatcaaggacgactacatccactgggtgcagcaggcccccggaaaaggcctggagtggatgggctggatcgacgccatgaacgacgactcccagtactccagcaagttccagggcagggtgacaatcaccgtggacacctccaccaacaccgcctacatgaagctgtcctccctgcggtccgaggataccgccgtgtactactgcacctacgccttcggcggatactggggccagggcaccacagtgaccgtgtcctccgctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 12 - DNA, 726 bp, Artificial Sequence
  Synthetic Polynucleotide
atggacatgagggtccccgctcagctcctggggctcctgctactctggctccgaggtgccagatgtgacatcgtgatgacccagacacccctgtccctgtccgtgacacctggacagcccgcctccatctcctgcaggtcctcccagtccctggtgcactccgactccaacacctacctccactggtacctgcagaagcctggccagtccccccagctgctgatctacctggtgtccaaccggttcagcggcgtgcctgacaggttcagcggaagcggctccggcaccgacttcaccctgaagatctccagggtggaggccgaggatgtgggcgtgtacttctgcggccagtccacccacgtgccctatgctttcggcggcggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 13 - protein, 342 aa, Artificial Sequence
  Synthetic Polynucleotide
GAGGTGCAGCTGGTGCAGTCCGGCGCTGAGGTGAAGAAGCCTGGCGCCACCGTGAAGATCTCCTGCAAGGCCTCCGGCTTCAGCATCAAGGACGACTACATCCACTGGGTGCAGCAGGCCCCCGGAAAAGGCCTGGAGTGGATGGGCTGGATCGACGCCATGAACGACGACTCCCAGTACTCCAGCAAGTTCCAGGGCAGGGTGACAATCACCGTGGACACCTCCACCAACACCGCCTACATGAAGCTGTCCTCCCTGCGGTCCGAGGATACCGCCGTGTACTACTGCACCTACGCCTTCGGCGGATACTGGGGCCAGGGCACCACAGTGACCGTGTCCTCC

SEQ ID NO: 14 - DNA, 336 bp, Artificial Sequence
  Synthetic Polynucleotide
gacatcgtgatgacccagacacccctgtccctgtccgtgacacctggacagcccgcctccatctcctgcaggtcctcccagtccctggtgcactccgactccaacacctacctccactggtacctgcagaagcctggccagtccccccagctgctgatctacctggtgtccaaccggttcagcggcgtgcctgacaggttcagcggaagcggctccggcaccgacttcaccctgaagatctccagggtggaggccgaggatgtgggcgtgtacttctgcggccagtccacccacgtgccctatgctttcggcggcggcaccaaggtggagatcaag

SEQ ID NO: 15 - DNA, 15 bp, Artificial Sequence
  Synthetic Oligonucleotide
gacgactacatccac

SEQ ID NO: 16 - DNA, 51 bp, Artificial Sequence
  Synthetic Oligonucleotide
tggatcgacgccatgaacgacgactcccagtactccagcaagttccagggc

SEQ ID NO: 17 - DNA, 15 bp, Artificial Sequence
  Synthetic Oligonucleotide
gccttcggcggatac

SEQ ID NO: 18 - DNA, 48 bp, Artificial Sequence
  Synthetic Oligonucleotide
aggtcctcccagtccctggtgcactccgactccaacacctacctccac

SEQ ID NO: 19 - DNA, 21 bp, Artificial Sequence
  Synthetic Oligonucleotide
ctggtgtccaaccggttcagc

SEQ ID NO: 20 - DNA, 27 bp, Artificial Sequence
  Synthetic Oligonucleotide
ggccagtccacccacgtgccctatgct

SEQ ID NO: 21 - protein, 441 aa, Artificial Sequence
  Synthetic Polypeptide
EVQLVQSGAEVKKPGATVKISCKASGFSIKDDYIHWVQQAPGKGLEWMGWIDAMNDDSQYSSKFQGRVTITVDTSTNTAYMKLSSLRSEDTAVYYCTYAFGGYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 219 aa, Artificial Sequence
  Synthetic Polypeptide
DIVMTQTPLSLSVTPGQPASISCRSSQSLVHSDSNTYLHWYLQKPGQSPQLLIYLVSNRFSGVPDRFSGSGSGTDFTLKISRVEAEDVGVYFCGQSTHVPYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 117 aa, Artificial Sequence
  Synthetic Polypeptide
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSA

SEQ ID NO: 24 - protein, 108 aa, Artificial Sequence
  Synthetic Polypeptide
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKR

SEQ ID NO: 25 - protein, 10 aa, Artificial Sequence
  Synthetic Oligopeptide
GFTFSSYDMS

SEQ ID NO: 26 - protein, 10 aa, Artificial Sequence
  Synthetic Oligopeptide
TISGGGSYTY

SEQ ID NO: 27 - protein, 7 aa, Artificial Sequence
  Synthetic Oligopeptide
PYYAMDY

SEQ ID NO: 28 - protein, 11 aa, Artificial Sequence
  Synthetic Oligopeptide
KASQDVGTAVA

SEQ ID NO: 29 - protein, 7 aa, Artificial Sequence
  Synthetic Oligopeptide
WASTLHT

SEQ ID NO: 30 - protein, 9 aa, Artificial Sequence
  Synthetic Oligopeptide
QHYSSYPWT

SEQ ID NO: 31 - protein, 440 aa, Artificial Sequence
  Synthetic Polypeptide
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 213 aa, Artificial Sequence
  Synthetic Polypeptide
DIQMQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 10 aa, Artificial Sequence
  Synthetic Oligopeptide
GFTFSSYDMS

SEQ ID NO: 34 - protein, 17 aa, Artificial Sequence
  Synthetic oligopeptide
TISGGGTYTYYQDSVKG

SEQ ID NO: 35 - protein, 3 aa, Artificial Sequence
  Synthetic Oligopeptide
MDY

SEQ ID NO: 36 - protein, 11 aa, Artificial Sequence
  Synthetic Oligopeptide
RASQSIRRYLN

SEQ ID NO: 37 - protein, 7 aa, Artificial Sequence
  Synthetic Oligopeptide
GASTLQS

SEQ ID NO: 38 - protein, 9 aa, Artificial Sequence
  Synthetic Oligopeptide
QQSHSAPLT

SEQ ID NO: 39 - protein, 443 aa, Artificial Sequence
  Synthetic Polypeptide
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 214 aa, Artificial Sequence
  Synthetic Polypeptide
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0135670 A1
(43) Pub. Date: May 5, 2022

(54) ANTIBODY AGENTS DIRECTED AGAINST LYMPHOCYTE ACTIVATION GENE-3 (LAG-3) AND USES THEREOF

(71) Applicants: TESARO, INC., Waltham, MA (US); ANAPTYSBIO, INC., San Diego, CA (US)

(72) Inventors: Helen Toni Jun, San Diego, CA (US); Marilyn Kehry, San Diego, CA (US); Peter Bowers, San Diego, CA (US); David J. King, San Diego, CA (US); Dmitri Bobilev, Waltham, MA (US); Srimoyee Ghosh, Waltham, MA (US); Baochuan Huang, Waltham, MA (US); David Jenkins, Waltham, MA (US)

(21) Appl. No.: 16/608,111

(22) PCT Filed: Apr. 27, 2018
(86) PCT No.: PCT/US2018/030027

§ 371 Date: Oct. 24, 2019

Related U.S. Application Data
(60) Provisional application No. 62/491,221, filed on Apr. 27, 2017.
(60) Provisional application No. 62/578,215, filed on Oct. 27, 2017.
(60) Provisional application No. 62/614,998, filed on Jan. 8, 2018.
(60) Provisional application No. 62/625,276, filed on Feb. 1, 2018.
(60) Provisional application No. 62/657,384, filed on Apr. 13, 2018.

(51) Int. Cl. C07K 16/28; A61K 45/06; A61K 39/395; A61P 35/00

(52) U.S. Cl. CPC C07K 16/2803; A61K 45/06; A61K 2039/545; A61P 35/00; A61K 39/3955

(57) ABSTRACT

The disclosure provides antibody agents that bind to a Lymphocyte Activation Gene-3 (LAG-3) protein. Particular immunoglobulin heavy chain polypeptide and immunoglobulin light chain polypeptide sequences are explicitly provided. Also provided are related nucleic acids, vectors, compositions, and methods of using anti-LAG-3 antibody agents to treat a disorder or disease that is responsive to LAG-3 inhibition, such as, for example, cancer or an infectious disease.

### CROSS-REFERENCE TO RELATED APPLICATIONS

The present application claims benefit of U.S. Provisional Application No. 62/491,221, filed Apr. 27, 2017; U.S. Provisional Application No. 62/578,215, filed Oct. 27, 2017; U.S. Provisional Application No. 62/614,998, filed Jan. 8, 2018; U.S. Provisional Application No. 62/625,276, filed Feb. 1, 2018; and U.S. Provisional Application No. 62/657,384, filed Apr. 13, 2018, each of which is incorporated by reference in its entirety.

### SEQUENCE LISTING

The present specification makes reference to a Sequence Listing provided in electronic form as an ASCII.txt file named “TSR-007WO_ST25.txt” that was generated on Apr. 23, 2018, and is 39,964 bytes in size.

### FIELD OF THE INVENTION

This invention relates to antibody agents that bind to a Lymphocyte Activation Gene-3 (LAG-3) polypeptide.

### BACKGROUND

Cancer is a serious public health problem, with about 600,920 people in the United States of America expected to die of cancer in 2017 alone according to the American Cancer Society, Cancer Facts & FIGS. 2017 (hftps://www.cancer.org/researchlcancer-facts-statistics/all-cancer-facts-figures/cancer-facts-figures-20 17 html). Accordingly, there continues to be a need for effective therapies to treat cancer patients.

### SUMMARY OF THE INVENTION

The present disclosure provides, among other things, antibody agents that bind to an epitope of Lymphocyte Activation Gene-3 (LAG-3) polypeptide and various compositions and methods relating thereto including, for example, polypeptides, nucleic acids, cells, and various methodologies, etc.

In some embodiments, a polypeptide that is capable of binding Lymphocyte Activation Gene-3 (LAG-3) comprises one, two or three amino acid sequences selected from: (a) an amino acid sequence of SEQ ID NO: 5, (b) an amino acid sequence of SEQ ID NO: 6, and (c) an amino acid sequence of SEQ ID NO: 7.

In some embodiments, a polypeptide is or comprises a heavy chain variable domain comprising one, two or three CDRs selected from: (a) CDR-H1 comprising an amino acid sequence of SEQ ID NO: 5, (b) CDR-H2 comprising an amino acid sequence of SEQ ID NO: 6, and (c) CDR-H3 comprising an amino acid sequence of SEQ ID NO: 7.

In some embodiments, a polypeptide that is capable of binding Lymphocyte Activation Gene-3 (LAG-3), wherein said polypeptide comprises a heavy chain variable region comprising: a CDR-H1 defined by an amino acid sequence at least 80%, 85% or 90% identical to SEQ ID NO: 5, and/or a CDR-H2 defined by an amino acid sequence at least 80%, 85% or 90% identical to SEQ ID NO: 6; and/or a CDR-H3 defined by an amino acid sequence at least 80%, 85% or 90% identical to SEQ ID NO: 7. In some embodiments, a polypeptide that is capable of binding LAG-3, wherein said polypeptide comprises a heavy chain variable region comprising: a CDR-H1 defined by an amino acid sequence at least 80%, 85% or 90% identical to SEQ ID NO: 5; a CDR-H2 defined by an amino acid sequence at least 80%, 85% or 90% identical to SEQ ID NO: 6; and a CDR-H3 defined by an amino acid sequence at least 80%, 85% or 90% identical to SEQ ID NO: 7.

In some embodiments, a polypeptide that is capable of binding Lymphocyte Activation Gene-3 (LAG-3) comprises one, two or three amino acid sequences selected from: (a) an amino acid sequence of SEQ ID NO: 8, (b) an amino acid sequence of SEQ ID NO: 9, and (c) an amino acid sequence of SEQ ID NO: 10.

In some embodiments, a polypeptide is or comprises a light chain variable region comprising one, two or three CDRs selected from: (a) CDR-L1 comprising an amino acid sequence of SEQ ID NO: 8, (b) CDR-L2 comprising an amino acid sequence of SEQ ID NO: 9, and (c) CDR-L3 comprising an amino acid sequence of SEQ ID NO: 10.

In some embodiments, a polypeptide that is capable of binding Lymphocyte Activation Gene-3 (LAG-3) comprises a CDR-L1 defined by an amino acid sequence at least 80%, 85% or 90% identical to SEQ ID NO: 8; and/or a CDR-L2 defined by an amino acid sequence at least 80%, 85% or 90% identical to SEQ ID NO: 9; and/or a CDR-L3 defined by an amino acid sequence at least 80%, 85% or 90% identical to SEQ ID NO: 10. In some embodiments, a polypeptide that is capable of binding LAG-3 comprises a CDR-L1 defined by an amino acid sequence at least 80%, 85% or 90% identical to SEQ ID NO: 8; a CDR-L2 defined by an amino acid sequence at least 80%, 85% or 90% identical to SEQ ID NO: 9; and a CDR-L3 defined by an amino acid sequence at least 80%, 85% or 90% identical to SEQ ID NO: 10.

The present disclosure provides, among other things, polypeptides that are capable of binding Lymphocyte Activation Gene-3 (LAG-3), wherein the polypeptide comprises an amino acid sequence having at least 80%, 85%, 90%, 95%, or 98% sequence identity to SEQ ID NO: 1, SEQ ID NO:3, or SEQ ID NO:21. In some embodiments, a polypeptide is or comprises a heavy chain polypeptide comprising an amino acid sequence as set forth in SEQ ID NO: 1, SEQ ID NO:3, or SEQ ID NO: 21.

In some embodiments, a polypeptide (e.g., antibody agent) that is capable of binding Lymphocyte Activation Gene-3 (LAG-3), wherein the polypeptide comprises an amino acid sequence having at least 80%, 85%, 90%, 95%, or 98% sequence identity to SEQ ID NO: 2, SEQ ID NO: 4, or SEQ ID NO: 22. In some embodiments, is or comprises a light chain polypeptide comprising an amino acid sequence having at least 80%, 85%, 90%, 95%, or 98% sequence identity to SEQ ID NO: 2, SEQ ID NO: 4, or SEQ ID NO: 22.

In embodiments, a polypeptide that is capable of binding Lymphocyte Activation Gene-3 (LAG-3) comprises a heavy chain variable region amino acid sequence having at least 80%, 85%, 90%, 95%, or 98% sequence identity to SEQ ID NO: 3. In embodiments, a polypeptide that is capable of binding LAG-3 comprises a heavy chain variable region amino acid sequence defined by SEQ ID NO: 3.

In embodiments, a polypeptide that is capable of binding Lymphocyte Activation Gene-3 (LAG-3) comprises a light chain variable region amino acid sequence having at least 80%, 85%, 90%, 95%, or 98% sequence identity to SEQ ID NO: 4. In embodiments, a polypeptide that is capable of binding LAG-3 comprises a light chain variable region amino acid sequence defined by SEQ ID NO: 4.

In embodiments, a polypeptide that is capable of binding Lymphocyte Activation Gene-3 (LAG-3) comprises a heavy chain polypeptide sequence having at least 80%, 85%, 90%, 95%, or 98% sequence identity to SEQ ID NO: 1. In embodiments, a polypeptide that is capable of binding LAG-3 comprises a heavy chain polypeptide sequence defined by SEQ ID NO: 1.

In embodiments, a polypeptide that is capable of binding Lymphocyte Activation Gene-3 (LAG-3) comprises a heavy chain polypeptide sequence having at least 80%, 85%, 90%, 95%, or 98% sequence identity to SEQ ID NO: 21. In embodiments, a polypeptide that is capable of binding LAG-3 comprises a heavy chain polypeptide sequence defined by SEQ ID NO: 21.

In embodiments, a polypeptide that is capable of binding Lymphocyte Activation Gene-3 (LAG-3) comprises a light chain polypeptide sequence having at least 80%, 85%, 90%, 95%, or 98% sequence identity to SEQ ID NO: 2. In embodiments, a polypeptide that is capable of binding LAG-3 comprises a light chain polypeptide sequence defined by SEQ ID NO: 2.

In embodiments, a polypeptide that is capable of binding Lymphocyte Activation Gene-3 (LAG-3) comprises a light chain polypeptide sequence having at least 80%, 85%, 90%, 95%, or 98% sequence identity to SEQ ID NO: 22. In embodiments, a polypeptide that is capable of binding LAG-3 comprises a light chain polypeptide sequence defined by SEQ ID NO: 22.

In some embodiments, a polypeptide that is capable of binding Lymphocyte Activation Gene-3 (LAG-3) comprises (i) an amino acid having at least 80%, 85%, 90%, 95%, 98%, 99% or 100% sequence identity to SEQ ID NO: 1, SEQ ID NO:3, or SEQ ID NO: 21; and (ii) an amino acid having at least 80%, 85%, 90%, 95%, 98%, 99% or 100% sequence identity to SEQ ID NO: 2, SEQ ID NO: 4, or SEQ ID NO: 22.

In some embodiments, a polypeptide that is capable of binding Lymphocyte Activation Gene-3 (LAG-3) comprises
i) one, two or three amino acid sequences selected from:
   (a) an amino acid sequence that is identical in sequence or contains between 1-5 amino acid substitutions as compared to SEQ ID NO: 5,
   (b) an amino acid sequence that is identical in sequence or contains between 1-5 amino acid substitutions as compared to SEQ ID NO: 6, and
   (c) an amino acid sequence that is identical in sequence or contains between 1-5 amino acid substitutions as compared to SEQ ID NO: 7;
      and
ii) one, two or three amino acid sequences selected from:
   (a) an amino acid sequence that is identical in sequence or contains between 1-5 amino acid substitutions as compared to SEQ ID NO: 8,
   (b) an amino acid sequence that is identical in sequence or contains between 1-5 amino acid substitutions as compared to SEQ ID NO: 9, and
   (c) an amino acid sequence that is identical in sequence or contains between 1-5 amino acid substitutions as compared to SEQ ID NO: 10.

In some embodiments, a polypeptide that is capable of binding Lymphocyte Activation Gene-3 (LAG-3) comprises (i) one, two or three amino acid sequences selected from: (a) an amino acid sequence of SEQ ID NO: 5, (b) an amino acid sequence of SEQ ID NO: 6, and (c) an amino acid sequence of SEQ ID NO: 7; and (ii) one, two or three amino acid sequences selected from: (a) an amino acid sequence of SEQ ID NO: 8, (b) an amino acid sequence of SEQ ID NO: 9, and (c) an amino acid sequence of SEQ ID NO: 10.

In some embodiments, a polypeptide that is capable of binding LAG-3 is isolated. In some embodiments, a polypeptide that is capable of binding LAG-3 can be purified to greater than 95% or 99% purity. In some embodiments, an anti-LAG-3 antibody agent is isolated. In some embodiments, an antibody agent can be purified to greater than 95% or 99% purity.

In some embodiments, a polypeptide that is capable of binding LAG-3 comprises at least one disulfide bond formed by a first cysteine and a second cysteine; wherein the first cysteine is selected from residue 41, 115, 147, 160, 216, 239, 242, 274, 334, 380 and 438 of SEQ ID NO: 1, and the second cysteine is selected from residue 41, 115, 147, 160, 216, 239, 242, 274, 334, 380 and 438 of SEQ ID NO: 1. In some embodiments, a polypeptide that is capable of binding LAG-3 comprises at least one disulfide bond formed by a first cysteine and a second cysteine; wherein the first cysteine is selected from residue 41, 115, 147, 160, 216, 239, 242, 274, 334, 380 and 438 of SEQ ID NO: 1, and the second cysteine is selected from residue 45, 115, 161, 221 and 241 of SEQ ID NO: 2. In some embodiments, a polypeptide that is capable of binding LAG-3 comprises at least one disulfide bond formed by a first cysteine and a second cysteine; wherein the first cysteine is selected from residue 45, 115, 161, 221 and 241 of SEQ ID NO: 2, and the second cysteine is selected from residue 45, 115, 161, 221 and 241 of SEQ ID NO: 2.

In some embodiments, a polypeptide comprises at least one disulfide bond formed by a first cysteine and a second cysteine; wherein the first residue is residue 45 of SEQ ID NO: 2, and the second residue is residue 115 of SEQ ID NO: 2. In some embodiments, a polypeptide comprises at least one disulfide bond formed by a first cysteine and a second cysteine; wherein the first residue is residue 161 of SEQ ID NO: 2, and the second residue is residue 221 of SEQ ID NO: 2. In some embodiments, a polypeptide comprises at least one disulfide bond formed by a first cysteine and a second cysteine; wherein the first residue is residue 147 of SEQ ID NO: 1, and the second residue is residue 241 of SEQ ID NO: 2. In some embodiments, a polypeptide comprises at least one disulfide bond formed by a first cysteine and a second cysteine; wherein the first residue is residue 41 of SEQ ID NO: 1, and the second residue is residue 115 of SEQ ID NO: 1. In some embodiments, a polypeptide comprises at least one disulfide bond formed by a first cysteine and a second cysteine; wherein the first residue is residue 160 of SEQ ID NO: 1, and the second residue is residue 216 of SEQ ID NO: 1. In some embodiments, a polypeptide of the present disclosure comprises at least one disulfide bond formed by a first cysteine and a second cysteine; wherein the first residue is residue 239 of SEQ ID NO: 1, and the second residue is residue 242 of SEQ ID NO: 1. In some embodiments, a polypeptide comprises at least one disulfide bond formed by a first cysteine and a second cysteine; wherein the first residue is residue 274 of SEQ ID NO: 1, and the second residue is residue 334 of SEQ ID NO: 1. In some embodiments, a polypeptide comprises at least one disulfide bond formed by a first cysteine and a second cysteine; wherein the first residue is residue 380 of SEQ ID NO: 1, and the second residue is residue 438 of SEQ ID NO: 1.

In some embodiments, a polypeptide of the present disclosure contains at least one asparagine that is glycosylated. In some embodiments, a polypeptide that is capable of binding Lymphocyte Activation Gene-3 (LAG-3) comprises at least one asparagine that is glycosylated.

In some embodiments, a polypeptide that is capable of binding Lymphocyte Activation Gene-3 (LAG-3) contains (i) a heavy chain variable region comprising a CDR-H1 comprising an amino acid sequence of SEQ ID NO: 5, a CDR-H2 comprising an amino acid sequence of SEQ ID NO: 6, and a CDR-H3 comprising an amino acid sequence of SEQ ID NO: 7; and (ii) a light chain variable region comprising a CDR-L1 comprising an amino acid sequence of SEQ ID NO: 8, a CDR-L2 comprising an amino acid sequence of SEQ ID NO: 9, and a CDR-L3 comprising an amino acid sequence of SEQ ID NO: 10. In some embodiments, a polypeptide that is capable of binding LAG-3 contains at least one disulfide bond formed by a first cysteine and a second cysteine; wherein the first cysteine is selected from residue 41, 115, 147, 160, 216, 239, 242, 274, 334, 380 and 438 of SEQ ID NO: 1, and the second cysteine is selected from residue 45, 115, 161, 221 and 241 of SEQ ID NO: 2. In some embodiments, a polypeptide that is capable of binding LAG-3 contains at least one disulfide bond formed by a first cysteine and a second cysteine; wherein the first cysteine is selected from residue 41, 115, 147, 160, 216, 239, 242, 274, 334, 380 and 438 of SEQ ID NO: 1, and the second cysteine is selected from residue 45, 115, 161, 221 and 241 of SEQ ID NO: 2. In some embodiments, a polypeptide that is capable of binding LAG-3 contains at least one disulfide bond formed by a first cysteine and a second cysteine; wherein the first cysteine is selected from residue 45, 115, 161, 221 and 241 of SEQ ID NO: 2, and the second cysteine is selected from residue 45, 115, 161, 221 and 241 of SEQ ID NO: 2.

In some embodiments, a polypeptide that is capable of binding Lymphocyte Activation Gene-3 (LAG-3) comprises a heavy chain variable region amino acid sequence having at least 80%, 85%, 90%, 95%, 98%, 99%, or 100% sequence identity to SEQ ID NO: 3 and/or a light chain variable region amino acid sequence having at least 80%, 85%, 90%, 95%, 98%, 99% or 100% sequence identity to SEQ ID NO: 4. In some embodiments, a polypeptide that is capable of binding LAG-3 comprises a heavy chain having at least 80%, 85%, 90%, 95%, 98%, 99%, or 100% sequence identity to SEQ ID NO: 1 and/or a light chain having at least 80%, 85%, 90%, 95%, 98%, 99% or 100% sequence identity to SEQ ID NO: 2. In some embodiments, a polypeptide that is capable of binding LAG-3 contains at least one disulfide bond formed by a first cysteine and a second cysteine; wherein the first cysteine is selected from residue 41, 115, 147, 160, 216, 239, 242, 274, 334, 380 and 438 of SEQ ID NO: 1, and the second cysteine is selected from residue 45, 115, 161, 221 and 241 of SEQ ID NO: 2. In some embodiments, a polypeptide that is capable of binding LAG-3 contains at least one disulfide bond formed by a first cysteine and a second cysteine; wherein the first cysteine is selected from residue 41, 115, 147, 160, 216, 239, 242, 274, 334, 380 and 438 of SEQ ID NO: 1, and the second cysteine is selected from residue 45, 115, 161, 221 and 241 of SEQ ID NO: 2. In some embodiments, a polypeptide that is capable of binding LAG-3 contains at least one disulfide bond formed by a first cysteine and a second cysteine; wherein the first cysteine is selected from residue 45, 115, 161, 221 and 241 of SEQ ID NO: 2, and the second cysteine is selected from residue 45, 115, 161, 221 and 241 of SEQ ID NO: 2.

In embodiments, a polypeptide comprises a glycosylated asparagine on the heavy chain. In embodiments, a glycosylated asparagine is N291 of the heavy chain. In embodiments, total N-linked oligosaccharides comprise G0F. In embodiments, total N-linked oligosaccharides comprise G1F. In embodiments, total N-linked oligosaccharides comprise G2F. In embodiments, total N-linked oligosaccharides comprise Man-5. In embodiments, total N-linked oligosaccharides comprise G0F and G1F. In embodiments, total N-linked oligosaccharides comprise G0F, G1F, G2F, and Man-5.

In some embodiments, a polypeptide (of the present disclosure binds lymphocyte activation gene-3 (LAG-3) and/or inhibits the interaction between LAG-3 and MHC II.

In some embodiments, a polypeptide that is capable of binding Lymphocyte Activation Gene-3 (LAG-3) is human or humanized. In some embodiments, a polypeptide that is capable of binding LAG-3 is an antibody agent that is or comprises a human antibody variable domain. In some embodiments, a polypeptide that is capable of binding LAG-3 is an antibody agent that is or comprises a humanized antibody variable domain.

In some embodiments, an amino acid sequence is substantially identical to a reference amino acid sequence in that it is either identical in sequence or contains between 1-5 amino acid substitutions as compared with the reference sequence. In embodiments, an amino acid sequence is substantially identical to a provided reference sequence (e.g., any of SEQ ID NOs: 1-10 and 21-40). In embodiments, an amino acid sequence is identical to a provided reference sequence (e.g., any of SEQ ID NOs: 1-10 and 21-40). In embodiments, an amino acid sequence contains between 1-5 amino acid substitutions as compared to a provided reference sequence (e.g., between 1-5 amino acid substitutions as compared to any of SEQ ID NOs: 1-10 and 21-40).

In embodiments, an amino acid sequence is substantially identical to SEQ ID NO: 1. In embodiments, an amino acid sequence is identical to SEQ ID NO: 1. In embodiments, an amino acid sequence contains between 1-5 amino acid substitutions as compared to SEQ ID NO: 1.

In embodiments, an amino acid sequence is substantially identical to SEQ ID NO: 2. In embodiments, an amino acid sequence is identical to SEQ ID NO: 2. In embodiments, an amino acid sequence contains between 1-5 amino acid substitutions as compared to SEQ ID NO: 2.

In embodiments, an amino acid sequence is substantially identical to SEQ ID NO: 3. In embodiments, an amino acid sequence is identical to SEQ ID NO: 3. In embodiments, an amino acid sequence contains between 1-5 amino acid substitutions as compared to SEQ ID NO: 3.

In embodiments, an amino acid sequence is substantially identical to SEQ ID NO: 4. In embodiments, an amino acid sequence is identical to SEQ ID NO: 4. In embodiments, an amino acid sequence contains between 1-5 amino acid substitutions as compared to SEQ ID NO: 4.

In embodiments, an amino acid sequence is substantially identical to SEQ ID NO: 5. In embodiments, an amino acid sequence is identical to SEQ ID NO: 5. In embodiments, an amino acid sequence contains between 1-5 amino acid substitutions as compared to SEQ ID NO: 5.

In embodiments, an amino acid sequence is substantially identical to SEQ ID NO: 6. In embodiments, an amino acid sequence is identical to SEQ ID NO: 6. In embodiments, an amino acid sequence contains between 1-5 amino acid substitutions as compared to SEQ ID NO: 6.

In embodiments, an amino acid sequence is substantially identical to SEQ ID NO: 7. In embodiments, an amino acid sequence is identical to SEQ ID NO: 7. In embodiments, an amino acid sequence contains between 1-5 amino acid substitutions as compared to SEQ ID NO: 7.

In embodiments, an amino acid sequence is substantially identical to SEQ ID NO: 8. In embodiments, an amino acid sequence is identical to SEQ ID NO: 8. In embodiments, an amino acid sequence contains between 1-5 amino acid substitutions as compared to SEQ ID NO: 8.

In embodiments, an amino acid sequence is substantially identical to SEQ ID NO: 9. In embodiments, an amino acid sequence is identical to SEQ ID NO: 9. In embodiments, an amino acid sequence contains between 1-5 amino acid substitutions as compared to SEQ ID NO: 9.

In embodiments, an amino acid sequence is substantially identical to SEQ ID NO: 10. In embodiments, an amino acid sequence is identical to SEQ ID NO: 10. In embodiments, an amino acid sequence contains between 1-5 amino acid substitutions as compared to SEQ ID NO:10.

In embodiments, an amino acid sequence is substantially identical to SEQ ID NO: 21. In embodiments, an amino acid sequence is identical to SEQ ID NO: 21. In embodiments, an amino acid sequence contains between 1-5 amino acid substitutions as compared to SEQ ID NO: 21.

In embodiments, an amino acid sequence is substantially identical to SEQ ID NO: 22. In embodiments, an amino acid sequence is identical to SEQ ID NO: 22. In embodiments, an amino acid sequence contains between 1-5 amino acid substitutions as compared to SEQ ID NO: 22.

In embodiments, an amino acid sequence is substantially identical to SEQ ID NO: 23. In embodiments, an amino acid sequence is identical to SEQ ID NO: 23. In embodiments, an amino acid sequence contains between 1-5 amino acid substitutions as compared to SEQ ID NO: 23.

In embodiments, an amino acid sequence is substantially identical to SEQ ID NO: 24. In embodiments, an amino acid sequence is identical to SEQ ID NO: 24. In embodiments, an amino acid sequence contains between 1-5 amino acid substitutions as compared to SEQ ID NO: 24.

In embodiments, an amino acid sequence is substantially identical to SEQ ID NO: 25. In embodiments, an amino acid sequence is identical to SEQ ID NO: 25. In embodiments, an amino acid sequence contains between 1-5 amino acid substitutions as compared to SEQ ID NO: 25.

In embodiments, an amino acid sequence is substantially identical to SEQ ID NO: 26. In embodiments, an amino acid sequence is identical to SEQ ID NO: 26. In embodiments, an amino acid sequence contains between 1-5 amino acid substitutions as compared to SEQ ID NO: 26.

In embodiments, an amino acid sequence is substantially identical to SEQ ID NO: 27. In embodiments, an amino acid sequence is identical to SEQ ID NO: 27. In embodiments, an amino acid sequence contains between 1-5 amino acid substitutions as compared to SEQ ID NO: 27.

In embodiments, an amino acid sequence is substantially identical to SEQ ID NO: 28. In embodiments, an amino acid sequence is identical to SEQ ID NO: 28. In embodiments, an amino acid sequence contains between 1-5 amino acid substitutions as compared to SEQ ID NO: 28.

In embodiments, an amino acid sequence is substantially identical to SEQ ID NO: 29. In embodiments, an amino acid sequence is identical to SEQ ID NO: 29. In embodiments, an amino acid sequence contains between 1-5 amino acid substitutions as compared to SEQ ID NO: 29.

In embodiments, an amino acid sequence is substantially identical to SEQ ID NO: 30. In embodiments, an amino acid sequence is identical to SEQ ID NO: 30. In embodiments, an amino acid sequence contains between 1-5 amino acid substitutions as compared to SEQ ID NO: 30.

In embodiments, an amino acid sequence is substantially identical to SEQ ID NO: 31. In embodiments, an amino acid sequence is identical to SEQ ID NO: 31. In embodiments, an amino acid sequence contains between 1-5 amino acid substitutions as compared to SEQ ID NO: 31.

In embodiments, an amino acid sequence is substantially identical to SEQ ID NO: 32. In embodiments, an amino acid sequence is identical to SEQ ID NO: 32. In embodiments, an amino acid sequence contains between 1-5 amino acid substitutions as compared to SEQ ID NO: 32.

In embodiments, an amino acid sequence is substantially identical to SEQ ID NO: 33. In embodiments, an amino acid sequence is identical to SEQ ID NO: 33. In embodiments, an amino acid sequence contains between 1-5 amino acid substitutions as compared to SEQ ID NO: 33.

In embodiments, an amino acid sequence is substantially identical to SEQ ID NO: 34. In embodiments, an amino acid sequence is identical to SEQ ID NO: 34. In embodiments, an amino acid sequence contains between 1-5 amino acid substitutions as compared to SEQ ID NO: 34.

In embodiments, an amino acid sequence is substantially identical to SEQ ID NO: 35. In embodiments, an amino acid sequence is identical to SEQ ID NO: 35. In embodiments, an amino acid sequence contains between 1-5 amino acid substitutions as compared to SEQ ID NO: 35.

In embodiments, an amino acid sequence is substantially identical to SEQ ID NO: 36. In embodiments, an amino acid sequence is identical to SEQ ID NO: 36. In embodiments, an amino acid sequence contains between 1-5 amino acid substitutions as compared to SEQ ID NO: 36.

In embodiments, an amino acid sequence is substantially identical to SEQ ID NO: 37. In embodiments, an amino acid sequence is identical to SEQ ID NO: 37. In embodiments, an amino acid sequence contains between 1-5 amino acid substitutions as compared to SEQ ID NO: 37.

In embodiments, an amino acid sequence is substantially identical to SEQ ID NO: 38. In embodiments, an amino acid sequence is identical to SEQ ID NO: 38. In embodiments, an amino acid sequence contains between 1-5 amino acid substitutions as compared to SEQ ID NO: 38.

In embodiments, an amino acid sequence is substantially identical to SEQ ID NO: 39. In embodiments, an amino acid sequence is identical to SEQ ID NO: 39. In embodiments, an amino acid sequence contains between 1-5 amino acid substitutions as compared to SEQ ID NO: 39.

In embodiments, an amino acid sequence is substantially identical to SEQ ID NO: 40. In embodiments, an amino acid sequence is identical to SEQ ID NO: 40. In embodiments, an amino acid sequence contains between 1-5 amino acid substitutions as compared to SEQ ID NO: 40.

Also provided are isolated nucleic acid sequences encoding a polypeptide that is capable of binding Lymphocyte Activation Gene-3 (LAG-3). In some embodiments, an isolated nucleic acid encoding a polypeptide that is capable of binding LAG-3 comprises a nucleic acid of SEQ ID NO: 11, SEQ ID NO: 12, SEQ ID NO: 13, SEQ ID NO: 14, SEQ ID NO: 21, or SEQ ID NO: 22. In some embodiments, an isolated nucleic acid encoding a polypeptide that is capable of binding LAG-3 comprises one, two or three nucleic acid sequences selected from: (a) a nucleic acid sequence of SEQ ID NO: 15, (b) a nucleic acid sequence of SEQ ID NO: 16, and (c) a nucleic acid sequence of SEQ ID NO: 17. In some embodiments, an isolated nucleic acid encoding a polypeptide that is capable of binding LAG-3 comprises one, two or three nucleic acid sequences selected from: (a) a nucleic acid sequence of SEQ ID NO: 18, (b) a nucleic acid sequence of SEQ ID NO: 19, and (c) a nucleic acid sequence of SEQ ID NO: 20.

Provided are vectors comprising an isolated nucleic acid sequence encoding a polypeptide that is capable of binding LAG-3 and isolated cells comprising said vectors.

In some embodiments are provided compositions comprising a polypeptide that is capable of binding Lymphocyte Activation Gene-3 (LAG-3). In some embodiments are provided compositions comprising isolated nucleic acids and/or vectors a polypeptide that is capable of binding LAG-3. In some embodiments, an anti-LAG-3 antibody agent (e.g., a polypeptide, nucleic acid and/or vector agent) is isolated. In some embodiments, an antibody agent (e.g., a polypeptide, nucleic acid and/or vector agent) can be purified to greater than 95% or 99% purity. In some embodiments, the composition further comprises a pharmaceutically acceptable carrier.

In some embodiments are provided, isolated cells comprising a nucleic acid and/or vector encoding a polypeptide that is capable of binding LAG-3. In some embodiments, the composition further comprises a pharmaceutically acceptable carrier.

Also provided are antibody agents comprising a polypeptide that is capable of binding Lymphocyte Activation Gene-3 (LAG-3). In some embodiments, an antibody agent binds to LAG-3 with a K_{D} between about 1 picomolar (pM) and about 100 micromolar (μM). In some embodiments, an antibody agent binds to LAG-3 with a K_{D} in the range of about 5 pM to about 5 μM. In some embodiments, an antibody agent binds to LAG-3 with a K_{D} in the range of about 10 pM to about 100 nanomolar (nM). In some embodiments, an antibody agent binds to LAG-3 with a K_{D} in the range of about 50 pM to about 50 nanomolar (nM). In some embodiments, an antibody agent binds to LAG-3 with a K_{D} in the range of about 100 pM to about 10 nanomolar (nM).

Also provided are methods of inducing an immune response in a mammal having a disorder that is responsive to Lymphocyte Activation Gene-3 (LAG-3) inhibition. In some embodiments, such a method comprises administering an effective amount of an agent that is capable of inhibiting Lymphocyte Activation Gene-3 (LAG-3) signaling (LAG-3 agent). In some embodiments, such a method comprises administering an effective amount of an agent that is capable of inhibiting Lymphocyte Activation Gene-3 (LAG-3) signaling (LAG-3 agent), an effective amount of an agent that is capable of inhibiting programmed death-1 protein (PD-1) signaling (PD-1 agent), and an effective amount of an agent that is capable of inhibiting T cell immunoglobulin and mucin protein 3 (TIM-3) signaling (TIM-3 agent). In some embodiments, such a method comprises administering an effective amount of a polypeptide that is capable of binding LAG-3. In some embodiments, such a method comprises administering an effective amount of an isolated nucleic acid encoding polypeptide that is capable of binding LAG-3. In some embodiments, such a method comprises administering an effective amount of a vector that encodes a polypeptide that is capable of binding LAG-3. In some embodiments, such a method comprises administering an effective amount of an isolated cell comprising a nucleic acid or a vector encoding polypeptide that is capable of binding LAG-3. In some embodiments, such a method comprises administering an effective amount of a composition comprising a polypeptide, nucleic acid, vector or cell as described herein. In some embodiments, upon administration of a polypeptide, nucleic acid, vector, cell or composition of the present disclosure, an immune response is induced in the mammal. In embodiments, a PD-1 agent is TSR-042. In embodiments, a TIM-3 agent is TSR-033. In embodiments, a PD-1 agent is TSR-042, and a TIM-3 agent is TSR-033.

Also provided are methods of enhancing an immune response or increasing the activity of an immune cell in a mammal having a disorder that is responsive to Lymphocyte Activation Gene-3 (LAG-3) inhibition. In some embodiments, such a method comprises administering an effective amount of an agent that is capable of inhibiting Lymphocyte Activation Gene-3 (LAG-3) signaling (LAG-3 agent). In some embodiments, such a method comprises administering an effective amount of an agent that is capable of inhibiting Lymphocyte Activation Gene-3 (LAG-3) signaling (LAG-3 agent), an effective amount of an agent that is capable of inhibiting programmed death-1 protein (PD-1) signaling (PD-1 agent), and an effective amount of an agent that is capable of inhibiting T cell immunoglobulin and mucin protein 3 (TIM-3) signaling (TIM-3 agent). In some embodiments, such a method comprises administering an effective amount of a polypeptide capable of binding LAG-3, or an isolated nucleic acid encoding such a polypeptide, or a vector comprising such a nucleic acid, or an isolated cell comprising a vector, or a composition comprising any of the preceding, whereupon the an immune response is induced in the mammal. In some embodiments, the immune response is a humoral or cell mediated immune response. In some embodiments, the immune response is a CD4 or CD8 T cell response. In some embodiments, the immune response is a B cell response. In embodiments, a PD-1 agent is TSR-042. In embodiments, a TIM-3 agent is TSR-033. In embodiments, a PD-1 agent is TSR-042, and a TIM-3 agent is TSR-033.

Also provided are methods of treating a disorder in a mammal that is responsive to Lymphocyte Activation Gene-3 (LAG-3) inhibition. In some embodiments, such a method comprises administering an effective amount of an agent that is capable of inhibiting Lymphocyte Activation Gene-3 (LAG-3) signaling (LAG-3 agent). In some embodiments, such a method comprises administering an effective amount of an agent that is capable of inhibiting Lymphocyte Activation Gene-3 (LAG-3) signaling (LAG-3 agent), an effective amount of an agent that is capable of inhibiting programmed death-1 protein (PD-1) signaling (PD-1 agent), and an effective amount of an agent that is capable of inhibiting T cell immunoglobulin and mucin protein 3 (TIM-3) signaling (TIM-3 agent). In some embodiments, such a method comprises administering an effective amount of a polypeptide capable of binding LAG-3, or an isolated nucleic acid encoding such a polypeptide, or a vector comprising such a nucleic acid, or an isolated cell comprising a vector, or a composition comprising any of the preceding, to a mammal having a disorder that is responsive to LAG-3 inhibition, whereupon the disorder is treated in the mammal. In embodiments, a PD-1 agent is TSR-042. In embodiments, a TIM-3 agent is TSR-033. In embodiments, a PD-1 agent is TSR-042, and a TIM-3 agent is TSR-033.

In embodiments, a cancer is adenocarcinoma, endometrial cancer, breast cancer, ovarian cancer, cervical cancer, fallopian tube cancer, testicular cancer, primary peritoneal cancer, colon cancer, colorectal cancer, small intestine cancer, squamous cell carcinoma of the anus, squamous cell carcinoma of the penis, squamous cell carcinoma of the cervix, squamous cell carcinoma of the vagina, squamous cell carcinoma of the vulva, soft tissue sarcoma, melanoma, renal cell carcinoma, lung cancer, non-small cell lung cancer, adenocarcinoma of the lung, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, a hematological cancer, multiple myeloma, B-cell lymphoma, T-cell lymphoma, Hodgkin's lymphoma/primary mediastinal B-cell lymphoma, chronic myelogenous leukemia, acute myeloid leukemia, acute lymphoblastic leukemia, non-Hodgkin's lymphoma, neuroblastoma, a CNS tumor, diffuse intrinsic pontine glioma (DIPG), Ewing's sarcoma, embryonal rhabdomyosarcoma, osteosarcoma, or Wilms tumor. In embodiments, a cancer is MSS or MSI-L, is characterized by microsatellite instability, is MSI-H, has high TMB, has high TMB and is MSS or MSI-L, has high TMB and is MSI-H, has a defective DNA mismatch repair system, has a defect in a DNA mismatch repair gene, is a hypermutated cancer, is an HRD or HRR cancer, comprises a mutation in polymerase delta (POLD) or comprises a mutation in polymerase epsilon (POLE).

In embodiments, a cancer is large B-cell lymphoma, thymoma, acute myeloid leukemia, testicular tumor, lung adenocarcinoma, non-small cell lung cancer, kidney clear cell cancer, breast cancer, triple negative breast cancer (TNBC), non-triple negative breast cancer (non-TNBC), gastric cancer, lung squamous cell cancer, mesothelioma, pancreatic cancer, cervical cancer, head and neck cancer, melanoma, hepatocellular carcinoma, nasopharyngeal cancer, esophageal cancer, colon adenocarcinoma, colorectal cancer, rectum carcinoma, cholangiocarcinoma, uterine endometrial cancer, sarcoma, bladder cancer, thyroid carcinoma, kidney papillary cancer, glioblastoma multiforme, liver cancer, uterine carcinosarcoma, pheocromocytoma, lower grade glioma, kidney chromophobe, adrenocortical cancer, or uveal melanoma. In embodiments, a cancer is MSS or MSI-L, is characterized by microsatellite instability, is MSI-H, has high TMB, has high TMB and is MSS or MSI-L, has high TMB and is MSI-H, has a defective DNA mismatch repair system, has a defect in a DNA mismatch repair gene, is a hypermutated cancer, is an HRD or HRR cancer, comprises a mutation in polymerase delta (POLD) or comprises a mutation in polymerase epsilon (POLE).

In embodiments, a cancer is melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, endometrial cancer, ovarian cancer, or Merkel cell carcinoma.

In embodiments, a cancer is non-small cell lung cancer, endometrial cancer, renal cell carcinoma, cervical cancer, stomach cancer, colorectal cancer, or triple negative breast cancer (TNBC).

In embodiments, a cancer has homologous recombination repair deficiency/homologous repair deficiency (“HRD”) or is characterized by a homologous recombination repair (HRR) gene mutation or deletion. In embodiments, a cancer is endometrial cancer, optionally MSI-H or MSS/MSI-L endometrial cancer. In embodiments, a cancer is endometrial cancer (e.g., MSI-H or MSS/MSI-L endometrial cancer). In embodiments, a cancer is a MSI-H cancer comprising a mutation in POLE or POLD (e.g., a MSI-H non-endometrial cancer comprising a mutation in POLE or POLD).

In embodiments, a cancer is breast cancer (e.g., triple negative breast cancer). In embodiments, a cancer is ovarian cancer (e.g., epithelial ovarian cancer). In embodiments, a cancer is lung cancer (e.g., non-small cell lung cancer). In embodiments, a cancer is a melanoma. In embodiments, a cancer is acute myeloid leukemia. In embodiments, a cancer is acute lymphoblastic leukemia. In embodiments, a cancer is non-Hodgkin's lymphoma. In embodiments, a cancer is Hodgkin's lymphoma. In embodiments, a cancer is neuroblastoma. In embodiments, a cancer is a CNS tumor. In embodiments, a cancer is diffuse intrinsic pontine glioma (DIPG). In embodiments, a cancer is Ewing's sarcoma. In embodiments, a cancer is embryonal rhabdomyosarcoma. In embodiments, a cancer is osteosarcoma. In embodiments, a cancer is Wilm's tumor. In embodiments, a cancer is a soft tissue sarcoma (e.g., leiomyosarcoma).

In some embodiments, a patient has cancer, such as: a non-small cell lung cancer (NSCLC), a hepatocellular cancer, a renal cancer, a melanoma, a cervical cancer, a colorectal cancer, a squamous cell carcinoma of the anogenital region, a head and neck cancer, a triple negative breast cancer, an ovarian cancer or an endometrial cancer. In some embodiments, a patient has a cancer with microsatellite instability. In some embodiments, the microsatellite instability is considered high, wherein the instability is significantly higher than that observed in a control cell (e.g., MSI-H status). In some embodiments, the patient has a solid tumor. In some embodiments, the patient has an advanced stage solid tumor. In some embodiments, a patient has an advanced stage solid tumor, such as a non-small cell lung cancer (NSCLC), a hepatocellular cancer, a renal cancer, a melanoma, a cervical cancer, a colorectal cancer, a squamous cell carcinoma of the anogenital region, a head and neck cancer, a triple negative breast cancer, an ovarian cancer or a endometrial cancer. In some embodiments, a patient has an advanced stage solid tumor with microsatellite instability.

In some embodiments, the patient has a hematological cancer. In some embodiments, the patient has a hematological cancer such as diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), acute lymphoblastic leukemia (“ALL”), or Multiple myeloma (“MM”). In some embodiments, a patient has a hematological cancer with microsatellite instability.

In some embodiments, a patient has a cancer characterized by PD-1 and/or PD-L1 expression. In some embodiments, a cancer has high PD-1 and/or PD-L1 expression (e.g., by high PD-1 and/or high PD-L1 expression). In some embodiment, a cancer characterized by PD-1 and/or PD-L1 expression is a head and neck cancer, a lung cancer (e.g., a non-small cell lung cancer (NSCLC)), a renal cancer, a bladder cancer, a melanoma, Merkel cell carcinoma, a cervical cancer, a vaginal cancer, a vulvar cancer, a uterine cancer, a endometrial cancer, an ovarian cancer, a fallopian tube cancer, a breast cancer, a prostate cancer, a salivary gland tumor, a thymoma, a adrenocortical carcinoma, a esophageal cancer, a gastric cancer, a colorectal cancer, an appendiceal cancer, a urothelial cell carcinoma, or a squamous cell carcinoma (e.g., of the lung; of the anogenital region including anus, penis, cervix, vagina, or vulva; or of the esophagus). In some certain embodiments, a cancer characterized by PD-1 and/or PD-L1 expression is an anal cancer, a fallopian tube cancer, an ovarian cancer, or a lung cancer.

In some embodiments, the patient has a head and neck cancer, a lung cancer (e.g., a non-small cell lung cancer (NSCLC)), a renal cancer, a bladder cancer, a melanoma, Merkel cell carcinoma, a cervical cancer, a vaginal cancer, a vulvar cancer, a uterine cancer, an endometrial cancer, an ovarian cancer, a fallopian tube cancer, a breast cancer, a prostate cancer, a salivary gland tumor, a thymoma, an adrenocortical carcinoma, an esophageal cancer, a gastric cancer, a colorectal cancer, an appendiceal cancer, a urothelial cell carcinoma, or a squamous cell carcinoma.

In embodiments, a cancer is an advanced cancer. In embodiments, a cancer is a metastatic cancer. In embodiments, a cancer is a MSI-H cancer. In embodiments, a cancer is a MSS cancer. In embodiments, a cancer is a POLE-mutant cancer. In embodiments, a cancer is a POLD-mutant cancer. In embodiments, a cancer is a high TMB cancer. In embodiments, a cancer is associated with homologous recombination repair deficiency/homologous repair deficiency (“HRD”) or is characterized by a homologous recombination repair (HRR) gene mutation or deletion.

In embodiments, a cancer is a solid tumor. In embodiments, a solid tumor is advanced. In embodiments, a solid tumor is a metastatic solid tumor. In embodiments, a solid tumor is a MSI-H solid tumor. In embodiments, a solid tumor is a MSS solid tumor. In embodiments, a solid tumor is a POLE-mutant solid tumor. In embodiments, a solid tumor is a POLD-mutant solid tumor. In embodiments, a solid tumor is a high TMB solid tumor. In embodiments, a solid tumor is associated with homologous recombination repair deficiency/homologous repair deficiency (“HRD”) or is characterized by a homologous recombination repair (HRR) gene mutation or deletion.

In embodiments, a cancer is a non-endometrial cancer (e.g., a non-endometrial solid tumor). In embodiments, a non-endometrial cancer is an advanced cancer. In embodiments, a non-endometrial cancer is a metastatic cancer. In embodiments, a non-endometrial cancer is a MSI-H cancer. In embodiments, a non-endometrial cancer is a MSS cancer. In embodiments, a non-endometrial cancer is a POLE-mutant cancer. In embodiments, a non-endometrial cancer is a solid tumor (e.g., a MSS solid tumor, a MSI-H solid tumor, a POLD mutant solid tumor, or a POLE-mutant solid tumor). In embodiments, a non-endometrial cancer is a high TMB cancer. In embodiments, a non-endometrial cancer is associated with homologous recombination repair deficiency/homologous repair deficiency (“HRD”) or is characterized by a homologous recombination repair (HRR) gene mutation or deletion.

In embodiments, a cancer is endometrial cancer (e.g., a solid tumor). In embodiments, an endometrial cancer is an advanced cancer. In embodiments, an endometrial cancer is a metastatic cancer. In embodiments, an endometrial cancer is a MSI-H endometrial cancer. In embodiments, an endometrial cancer is a MSS endometrial cancer. In embodiments, an endometrial cancer is a POLE-mutant endometrial cancer. In embodiments, an endometrial cancer is a POLD-mutant endometrial cancer. In embodiments, an endometrial cancer is a high TMB endometrial cancer. In embodiments, an endometrial cancer is associated with homologous recombination repair deficiency/homologous repair deficiency (“HRD”) or is characterized by a homologous recombination repair (HRR) gene mutation or deletion.

In embodiments, a cancer is a lung cancer (e.g., a solid tumor). In embodiments, a lung cancer is an advanced lung cancer. In embodiments, a lung cancer is a metastatic lung cancer. In embodiments, a lung cancer is squamous cell carcinoma of the lung. In embodiments, a lung cancer is small cell lung cancer (SCLC). In embodiments, a lung cancer is non-small cell lung cancer (NSCLC). In embodiments, a lung cancer is an ALK-translocated lung cancer (e.g., a lung cancer with a known ALK-translocation). In embodiments, a lung cancer is an EGFR-mutant lung cancer (e.g., a lung cancer with a known EGFR mutation). In embodiments, a lung cancer is a MSI-H lung cancer. In embodiments, a lung cancer is a MSS lung cancer. In embodiments, a lung cancer is a POLE-mutant lung cancer. In embodiments, a lung cancer is a POLD-mutant lung cancer. In embodiments, a lung cancer is a high TMB lung cancer. In embodiments, a lung cancer is associated with homologous recombination repair deficiency/homologous repair deficiency (“HRD”) or is characterized by a homologous recombination repair (HRR) gene mutation or deletion.

In embodiments, a cancer is a colorectal (CRC) cancer (e.g., a solid tumor). In embodiments, a colorectal cancer is an advanced colorectal cancer. In embodiments, a colorectal cancer is a metastatic colorectal cancer. In embodiments, a colorectal cancer is a MSI-H colorectal cancer. In embodiments, a colorectal cancer is a MSS colorectal cancer. In embodiments, a colorectal cancer is a POLE-mutant colorectal cancer. In embodiments, a colorectal cancer is a POLD-mutant colorectal cancer. In embodiments, a colorectal cancer is a high TMB colorectal cancer. In embodiments, a colorectal cancer is associated with homologous recombination repair deficiency/homologous repair deficiency (“HRD”) or is characterized by a homologous recombination repair (HRR) gene mutation or deletion.

In embodiments, a cancer is a melanoma. In embodiments, a melanoma is an advanced melanoma. In embodiments, a melanoma is a metastatic melanoma. In embodiments, a melanoma is a MSI-H melanoma. In embodiments, a melanoma is a MSS melanoma. In embodiments, a melanoma is a POLE-mutant melanoma. In embodiments, a melanoma is a POLD-mutant melanoma. In embodiments, a melanoma is a high TMB melanoma. In embodiments, a melanoma is associated with homologous recombination repair deficiency/homologous repair deficiency (“HRD”) or is characterized by a homologous recombination repair (HRR) gene mutation or deletion.

In embodiments, a cancer is squamous cell carcinoma of the anogenital region (e.g., of the anus, penis, cervix, vagina, or vulva). In embodiments, a squamous cell carcinoma of the anogenital region (e.g., of the anus, penis, cervix, vagina, or vulva) is an advanced cancer. In embodiments, a squamous cell carcinoma of the anogenital region (e.g., of the anus, penis, cervix, vagina, or vulva) is a metastatic cancer. In embodiments, a squamous cell carcinoma of the anogenital region (e.g., of the anus, penis, cervix, vagina, or vulva) is MSI-H. In embodiments, a squamous cell carcinoma of the anogenital region (e.g., of the anus, penis, cervix, vagina, or vulva) is MSS. In embodiments, a lung cancer is a POLE-mutant cancer. In embodiments, a squamous cell carcinoma of the anogenital region (e.g., of the anus, penis, cervix, vagina, or vulva) is associated with homologous recombination repair deficiency/homologous repair deficiency (“HRD”) or is characterized by a homologous recombination repair (HRR) gene mutation or deletion.

In embodiments, a cancer is an ovarian cancer. In embodiments, an ovarian cancer is an advanced ovarian cancer. In embodiments, an ovarian cancer is a metastatic ovarian cancer. In embodiments, an ovarian cancer is a MSI-H ovarian cancer. In embodiments, an ovarian cancer is a MSS ovarian cancer. In embodiments, an ovarian cancer is a POLE-mutant ovarian cancer. In embodiments, an ovarian cancer is a POLD-mutant ovarian cancer. In embodiments, an ovarian cancer is a high TMB ovarian cancer. In embodiments, an ovarian cancer is associated with homologous recombination repair deficiency/homologous repair deficiency (“HRD”) or is characterized by a homologous recombination repair (HRR) gene mutation or deletion. In embodiments, an ovarian cancer is a serous cell ovarian cancer. In embodiments, an ovarian cancer is a clear cell ovarian cancer.

In embodiments, a cancer is a fallopian cancer. In embodiments, a fallopian cancer is an advanced fallopian cancer. In embodiments, a fallopian cancer is a metastatic fallopian cancer. In embodiments, a fallopian cancer is a MSI-H fallopian cancer. In embodiments, a fallopian cancer is a MSS fallopian cancer. In embodiments, a fallopian cancer is a POLE-mutant fallopian cancer. In embodiments, a fallopian cancer is a POLD-mutant fallopian cancer. In embodiments, a fallopian cancer is a high TMB fallopian cancer. In embodiments, a fallopian cancer is associated with homologous recombination repair deficiency/homologous repair deficiency (“HRD”) or is characterized by a homologous recombination repair (HRR) gene mutation or deletion. In embodiments, a fallopian cancer is a serous cell fallopian cancer. In embodiments, a fallopian cancer is a clear cell fallopian cancer.

In embodiments, a cancer is a primary peritoneal cancer. In embodiments, a primary peritoneal cancer is an advanced primary peritoneal cancer. In embodiments, a primary peritoneal cancer is a metastatic primary peritoneal cancer. In embodiments, a primary peritoneal cancer is a MSI-H primary peritoneal cancer. In embodiments, a primary peritoneal cancer is a MSS primary peritoneal cancer. In embodiments, a primary peritoneal cancer is a POLE-mutant primary peritoneal cancer. In embodiments, a primary peritoneal cancer is a POLD-mutant primary peritoneal cancer. In embodiments, a primary peritoneal cancer is a high TMB primary peritoneal cancer. In embodiments, a primary peritoneal cancer is associated with homologous recombination repair deficiency/homologous repair deficiency (“HRD”) or is characterized by a homologous recombination repair (HRR) gene mutation or deletion. In embodiments, a primary peritoneal cancer is a serous cell primary peritoneal cancer. In embodiments, a primary peritoneal cancer is a clear cell primary peritoneal cancer.

In embodiments, a cancer is acute lymphoblastic leukemia (“ALL”). In embodiments, acute lymphoblastic leukemia is advanced acute lymphoblastic leukemia. In embodiments, acute lymphoblastic leukemia is metastatic acute lymphoblastic leukemia. In embodiments, acute lymphoblastic leukemia is MSI-H acute lymphoblastic leukemia. In embodiments, acute lymphoblastic leukemia is MSS acute lymphoblastic leukemia. In embodiments, acute lymphoblastic leukemia is POLE-mutant acute lymphoblastic leukemia. In embodiments, acute lymphoblastic leukemia is POLD-mutant acute lymphoblastic leukemia. In embodiments, an acute lymphoblastic leukemia is associated with homologous recombination repair deficiency/homologous repair deficiency (“HRD”) or is characterized by a homologous recombination repair (HRR) gene mutation or deletion.

In embodiments, a cancer is acute myeloid leukemia (“AML”). In embodiments, acute myeloid leukemia is advanced acute myeloid leukemia. In embodiments, acute myeloid leukemia is metastatic acute myeloid leukemia. In embodiments, acute myeloid leukemia is MSI-H acute myeloid leukemia. In embodiments, acute myeloid leukemia is MSS acute myeloid leukemia. In embodiments, acute myeloid leukemia is POLE-mutant acute myeloid leukemia. In embodiments, acute myeloid leukemia is POLD-mutant acute myeloid leukemia. In embodiments, an acute myeloid leukemia is associated with homologous recombination repair deficiency/homologous repair deficiency (“HRD”) or is characterized by a homologous recombination repair (HRR) gene mutation or deletion.

In embodiments, a cancer is non-Hodgkin's lymphoma (NHL). In embodiments, non-Hodgkin's lymphoma is advanced non-Hodgkin's lymphoma. In embodiments, non-Hodgkin's lymphoma is metastatic non-Hodgkin's lymphoma. In embodiments, non-Hodgkin's lymphoma is MSI-H non-Hodgkin's lymphoma. In embodiments, non-Hodgkin's lymphoma is MSS non-Hodgkin's lymphoma In embodiments, non-Hodgkin's lymphoma is POLE-mutant non-Hodgkin's lymphoma. In embodiments, non-Hodgkin's lymphoma is POLD-mutant non-Hodgkin's lymphoma. In embodiments, non-Hodgkin's lymphoma is associated with homologous recombination repair deficiency/homologous repair deficiency (“HRD”) or is characterized by a homologous recombination repair (HRR) gene mutation or deletion.

In embodiments, a cancer is Hodgkin's lymphoma (HL). In embodiments, Hodgkin's lymphoma is advanced Hodgkin's lymphoma. In embodiments, Hodgkin's lymphoma is metastatic Hodgkin's lymphoma. In embodiments, Hodgkin's lymphoma is MSI-H Hodgkin's lymphoma. In embodiments, Hodgkin's lymphoma is MSS Hodgkin's lymphoma In embodiments, Hodgkin's lymphoma is POLE-mutant Hodgkin's lymphoma. In embodiments, Hodgkin's lymphoma is POLD-mutant Hodgkin's lymphoma. In embodiments, Hodgkin's lymphoma is associated with homologous recombination repair deficiency/homologous repair deficiency (“HRD”) or is characterized by a homologous recombination repair (HRR) gene mutation or deletion.

In embodiments, a cancer is a neuroblastoma (NB). In embodiments, a neuroblastoma is an advanced neuroblastoma. In embodiments, a neuroblastoma is a metastatic neuroblastoma. In embodiments, neuroblastoma is a MSI-H neuroblastoma. In embodiments, a neuroblastoma is a MSS neuroblastoma. In embodiments, a neuroblastoma is a POLE-mutant neuroblastoma. In embodiments, a neuroblastoma is a POLD-mutant neuroblastoma. In embodiments, a neuroblastoma is a high TMB neuroblastoma. In embodiments, a neuroblastoma is associated with homologous recombination repair deficiency/homologous repair deficiency (“HRD”) or is characterized by a homologous recombination repair (HRR) gene mutation or deletion.

In embodiments, a cancer is a CNS tumor. In embodiments, a CNS tumor is advanced. In embodiments, a CNS tumor is a metastatic CNS tumor. In embodiments, a CNS tumor is a MSI-H CNS tumor. In embodiments, a CNS tumor is a MSS CNS tumor. In embodiments, a CNS tumor is a POLE-mutant CNS tumor. In embodiments, a CNS tumor is a POLD-mutant CNS tumor. In embodiments, a CNS tumor is a high TMB CNS tumor. In embodiments, a CNS tumor is associated with homologous recombination repair deficiency/homologous repair deficiency (“HRD”) or is characterized by a homologous recombination repair (HRR) gene mutation or deletion.

In embodiments, a cancer is diffuse intrinsic pontine glioma (DIPG). In embodiments, a DIPG is an advanced DIPG. In embodiments, a DIPG is a metastatic DIPG. In embodiments, DIPG is a MSI-H DIPG. In embodiments, a DIPG is a MSS DIPG. In embodiments, a DIPG is a POLE-mutant DIPG. In embodiments, a DIPG is a POLD-mutant DIPG. In embodiments, a DIPG is a high TMB DIPG. In embodiments, a DIPG is associated with homologous recombination repair deficiency/homologous repair deficiency (“HRD”) or is characterized by a homologous recombination repair (HRR) gene mutation or deletion.

In embodiments, a cancer is Ewing's sarcoma. In embodiments, Ewing's sarcoma is an advanced Ewing's sarcoma. In embodiments, Ewing's sarcoma is a metastatic Ewing's sarcoma. In embodiments, Ewing's sarcoma is a MSI-H Ewing's sarcoma. In embodiments, Ewing's sarcoma is a MSS Ewing's sarcoma. In embodiments, Ewing's sarcoma is a POLE-mutant Ewing's sarcoma. In embodiments, Ewing's sarcoma is a POLD-mutant Ewing's sarcoma. In embodiments, Ewing's sarcoma is a high TMB Ewing's sarcoma. In embodiments, Ewing's sarcoma is associated with homologous recombination repair deficiency/homologous repair deficiency (“HRD”) or is characterized by a homologous recombination repair (HRR) gene mutation or deletion.

In embodiments, a cancer is an embryonal rhabdomyosarcoma (ERS). In embodiments, an embryonal rhabdomyosarcoma is an advanced embryonal rhabdomyosarcoma. In embodiments, an embryonal rhabdomyosarcoma is a metastatic embryonal rhabdomyosarcoma. In embodiments, an embryonal rhabdomyosarcoma is a MSI-H embryonal rhabdomyosarcoma. In embodiments, an embryonal rhabdomyosarcoma is a MSS embryonal rhabdomyosarcoma. In embodiments, an embryonal rhabdomyosarcoma is a POLE-mutant embryonal rhabdomyosarcoma. In embodiments, an embryonal rhabdomyosarcoma is a POLD-mutant embryonal rhabdomyosarcoma. In embodiments, an embryonal rhabdomyosarcoma is a high TMB embryonal rhabdomyosarcoma. In embodiments, an embryonal rhabdomyosarcoma is associated with homologous recombination repair deficiency/homologous repair deficiency (“HRD”) or is characterized by a homologous recombination repair (HRR) gene mutation or deletion.

In embodiments, a cancer is an osteosarcoma (OS). In embodiments, an osteosarcoma is an advanced osteosarcoma. In embodiments, an osteosarcoma is a metastatic osteosarcoma. In embodiments, an osteosarcoma is a MSI-H osteosarcoma. In embodiments, an osteosarcoma is a MSS osteosarcoma. In embodiments, an osteosarcoma is a POLE-mutant osteosarcoma. In embodiments, an osteosarcoma is a POLD-mutant osteosarcoma. In embodiments, an osteosarcoma is a high TMB osteosarcoma. In embodiments, an osteosarcoma is associated with homologous recombination repair deficiency/homologous repair deficiency (“HRD”) or is characterized by a homologous recombination repair (HRR) gene mutation or deletion.

In embodiments, a cancer is a soft tissue sarcoma. In embodiments, a soft tissue sarcoma is an advanced soft tissue sarcoma. In embodiments, a soft tissue sarcoma is a metastatic soft tissue sarcoma. In embodiments, a soft tissue sarcoma is a MSI-H soft tissue sarcoma. In embodiments, a soft tissue sarcoma is a MSS soft tissue sarcoma. In embodiments, a soft tissue sarcoma is a POLE-mutant soft tissue sarcoma. In embodiments, a soft tissue sarcoma is a POLD-mutant soft tissue sarcoma. In embodiments, a soft tissue sarcoma is a high TMB soft tissue sarcoma. In embodiments, a soft tissue sarcoma is associated with homologous recombination repair deficiency/homologous repair deficiency (“HRD”) or is characterized by a homologous recombination repair (HRR) gene mutation or deletion. In embodiments, a soft tissue sarcoma is leiomyosarcoma.

In embodiments, a cancer is Wilms tumor. In embodiments, Wilms tumor is an advanced Wilms tumor. In embodiments, Wilms tumor is a metastatic Wilms tumor. In embodiments, Wilms tumor is a MSI-H Wilms tumor. In embodiments, Wilms tumor is a MSS Wilms tumor. In embodiments, Wilms tumor is a POLE-mutant Wilms tumor. In embodiments, Wilms tumor is a POLD-mutant Wilms tumor. In embodiments, Wilms tumor is a high TMB Wilms tumor. In embodiments, Wilms tumor is associated with homologous recombination repair deficiency/homologous repair deficiency (“HRD”) or is characterized by a homologous recombination repair (HRR) gene mutation or deletion.

In embodiments, a subject has previously been treated with one or more different cancer treatment modalities (e.g., one or more of surgery, radiotherapy, chemotherapy, or immunotherapy).

In embodiments, a subject has previously been treated with one different cancer treatment modalities (e.g., one or more of surgery, radiotherapy, chemotherapy, or immunotherapy). In embodiments, a subject has previously been treated with two or more different cancer treatment modalities (e.g., one or more of surgery, radiotherapy, chemotherapy, or immunotherapy). In embodiments, a subject has been previously treated with a cytotoxic therapy. In embodiments, a subject has been previously treated with chemotherapy. In embodiments, a subject has previously been treated with two different cancer treatment modalities (e.g., one or more of surgery, radiotherapy, chemotherapy, or immunotherapy). In embodiments, a subject has previously been treated with three different cancer treatment modalities (e.g., one or more of surgery, radiotherapy, chemotherapy, or immunotherapy).

In embodiments of methods described herein, a method further comprises administering one or more of surgery, a radiotherapy, a chemotherapy, an immunotherapy, an anti-angiogenic agent, or an anti-inflammatory. In embodiments, a method further comprises administering a chemotherapy.

In some embodiments, at least some of the patients in the cancer patient population have previously been treated with chemotherapy (e.g., platinum-based chemotherapy). For example, a patient who has received two lines of cancer treatment can be identified as a 2L cancer patient (e.g., a 2L NSCLC patient). In embodiments, a patient has received two lines or more lines of cancer treatment (e.g., a 2L+ cancer patient such as a 2L+ endometrial cancer patient). In embodiments, a patient has not been previously treated with an anti-PD-1 therapy. In embodiments, a patient previously received at least one line of cancer treatment (e.g., a patient previously received at least one line or at least two lines of cancer treatment). In embodiments, a patient previously received at least one line of treatment for metastatic cancer (e.g., a patient previously received one or two lines of treatment for metastatic cancer).

In embodiments, a subject is resistant to treatment with an agent that inhibits PD-1.

In embodiments, a subject is refractory to treatment with an agent that inhibits PD-1.

In embodiments, a method described herein sensitizes a subject to treatment with an agent that inhibits PD-1.

In embodiments, a subject comprises an exhausted immune cell (e.g., an exhausted immune cell that is an exhausted T cell).

In some embodiments, a disorder to be treated by the methods of the present disclosure is an infectious disease. In some embodiments, the infectious disease is caused by a virus or a bacterium. In some embodiments, the virus is human immunodeficiency virus (HIV), respiratory syncytial virus (RSV), influenza virus, dengue virus, Epstein-Barr virus (EBV) human papillomavirus (HPV), hepatitis B virus (HBV), or hepatitis C virus (HCV), optionally wherein the cancer is virally infected head and neck cancer, cervical cancer, hepatocellular carcinoma, or nasopharyngeal cancer.

In some embodiments, a disorder to be treated by the methods of the present disclosure is an autoimmune disease. In some embodiments, the autoimmune disease is multiple sclerosis, type 1 diabetes mellitus, rheumatoid arthritis, scleroderma, Crohn's disease, psoriasis, systemic lupus erythematosus (SLE), or ulcerative colitis.

In embodiments, methods of administering a LAG-3 agent as described herein further comprise administering another therapeutic agent or treatment. In embodiments, a method further comprises administering one or more of surgery, a radiotherapy, a chemotherapy, an immunotherapy, an anti-angiogenic agent, or an anti-inflammatory.

In embodiments, a subject has been further administered or will be administered an immune checkpoint inhibitor, such that the mammal receives the LAG-3 agent and the immune checkpoint inhibitor (e.g., an additional one, two, or three immune checkpoint inhibitors).

In embodiments, an immune checkpoint inhibitor is an inhibitor of PD-1, TIM-3, CTLA-4, TIGIT, CEACAM, VISTA, BTLA, LAIR1, CD160, 2B4, CD80, CD86, B7-H3 (CD276), B7-H4 (VTCN1), HVEM, KIR, A2aR, MHC class I, MHC class II, GALS, adenosine, TGFR, B7-H1, B7-H4 (VTCN1), OX-40, CD137, CD40, IDO, or CSF1R.

In embodiments, an immune checkpoint inhibitor is an agent that inhibits programmed death-1 protein (PD-1) signaling, T cell immunoglobulin and mucin protein 3 (TIM-3), cytotoxic T-lymphocyte-associated protein 4 (CTLA-4), T cell immunoglobulin and ITIM domain (TIGIT), indoleamine 2,3-dioxygenase (IDO), or colony-stimulating factor 1 receptor (CSF1R).

The present disclosure also encompasses the recognition, among other things, that any of the methods described herein may further comprise administering an agent that inhibits PD-1 signaling to a mammal. Agents that inhibit PD-1 signaling include those that bind to and block PD-1 receptors on T cells without triggering inhibitory signal transduction, agents that bind to PD-1 ligands to prevent their binding to PD-1, agents that do both and agents that prevent expression of genes that encode either PD-1 or natural ligands of PD-1.

In embodiments, an agent that inhibits PD-1 is a small molecule, a nucleic acid, a polypeptide (e.g. an antibody, a carbohydrate, a lipid, a metal, a toxin, or a PD-1 binding agent. In embodiments, an agent that inhibits PD-1 is a PD-1 binding agent (e.g., an antibody, an antibody conjugate, or an antigen-binding fragment thereof). In embodiments, a PD-1 binding agent is selected from the group consisting of: BGB-A317, BI 754091, IBI308, INCSHR-1210, JNJ-63723283, JS-001, MEDI-0680, MGA-012, nivolumab, PDR001, pembrolizumab, PF-06801591, REGN-2810, TSR-042, and derivatives thereof.

In some embodiments, an agent that inhibits PD-1 signaling is an antibody agent. Anti-PD-1 antibody agents can include any polypeptide or polypeptide complex that includes immunoglobulin structural elements sufficient to confer specific binding. Exemplary antibody agents include, but are not limited to, monoclonal antibodies, polyclonal antibodies, antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody agent that inhibits PD-1 signaling is a monoclonal antibody or a derivative thereof. In some embodiments, an antibody agent that inhibits PD-1 signaling is a PD-1 antibody, a PD-L1 antibody, or a derivative thereof. PD-1 and PD-L1 antibodies include, for example, atezolizumab, avelumab, BGB-A317, BI 754091, CX-072, durvalumab, FAZ053, IBI308, INCSHR-1210, JNJ-63723283, JS-001, LY3300054, MEDI-0680, MGA-012, nivolumab, PD-L1 millamolecule, PDR001, pembrolizumab, PF-06801591, REGN-2810, TSR-042, any of the antibodies disclosed in WO2014/179664, and any derivatives thereof. In some embodiments, an agent that inhibits PD-1 signaling is TSR-042. In some certain embodiments, an agent includes combinations of agents that inhibit PD-1 signaling.

In embodiments, an agent that inhibits PD-1 signaling is an anti-PD-L1/L2 agent. In embodiments, an anti-PD-L1/L2 agent is an anti-PD-L1 antibody. In embodiments, an anti-PD-L1 antibody agent is atezolizumab, avelumab, CX-072, durvalumab, FAZ053, LY3300054, PD-L1 millamolecule, or derivatives thereof.

In embodiments, an agent that inhibits PD-1 is administered at a dose of about 500 mg/patient to about 1000 mg/patient. In embodiments, an agent that inhibits PD-1 is administered at a dose of about 500 mg/patient. In embodiments, an agent that inhibits PD-1 is administered at a dose of about 1000 mg/patient. In embodiments, an agent that inhibits PD-1 is administered to the patient once every three weeks. In embodiments, an agent that inhibits PD-1 is administered for multiple cycles. In embodiments, an agent that inhibits PD-1 is administered for 2, 3, 4, 5, 6, or more cycles. In embodiments, an agent that inhibits PD-1 is administered for three, four, or five cycles. In embodiments, an agent that inhibits PD-1 is administered for four cycles. In embodiments, after the third, fourth, or fifth cycle, the agent that inhibits PD-1 is administered at a higher dose once every 6 weeks or more. In embodiments, an agent that inhibits PD-1 is administered at a higher dose once every 6 weeks. In embodiments, an agent that inhibits PD-1 is administered at a first dose of about 500 mg/patient. In embodiments, an agent that inhibits PD-1 is administered at a higher dose of about 1000 mg. In embodiments, an agent that inhibits PD-1 is administered at a first dose of about 500 mg once every 3 weeks for 3, 4, or 5 cycles followed by a second dose of about 1000 mg once every 6 weeks or more. In embodiments, an agent that inhibits PD-1 is administered at a first dose of about 500 mg once every 3 weeks for 3 cycles followed by a second dose of about 1000 mg once every 6 weeks or more. In embodiments, an agent that inhibits PD-1 is administered at a first dose of about 500 mg once every 3 weeks for 4 cycles followed by a second dose of about 1000 mg once every 6 weeks or more. In embodiments, an agent that inhibits PD-1 is administered at a first dose of about 500 mg once every 3 weeks for 5 cycles followed by a second dose of about 1000 mg once every 6 weeks or more. In embodiments, a second dose of 1000 mg is administered once every 6 weeks. In some embodiments, an agent that inhibits PD-1 signaling is administered intravenously.

In some embodiments, an agent that inhibits PD-1 signaling is administered to a subject who is receiving, has received or will receive treatment with an anti-LAG-3 antibody agent (e.g., as described herein). In some embodiments, an anti-LAG-3 antibody agent is administered to a subject who is receiving, has received or will receive treatment with an agent that inhibits PD-1 signaling.

In some related embodiments, administration of the anti-LAG-3 antibody agent improves the subject's response to an agent that inhibits PD-1 signaling. In some related embodiments, the subject is resistant for an agent that inhibits PD-1 signaling. In some related embodiments, the subject is refractory for an agent that inhibits PD-1 signaling. In some related embodiments, the subject overcomes resistance to an agent that inhibits PD-1 signaling after treatment with the anti-LAG-3 antibody agent reagent. In some related embodiments, the administration of the anti-LAG-3 antibody agent sensitizes the subject to an agent that inhibits PD-1 signaling.

The present disclosure also encompasses the recognition, among other things, that any of the above methods may further comprise administering an agent that inhibits TIM-3 signaling to a mammal.

In embodiments, an agent that inhibits TIM-3 is a small molecule, a nucleic acid, a polypeptide (e.g. an antibody), a carbohydrate, a lipid, a metal, a toxin or a TIM-3 binding agent.

In some embodiments, an agent that inhibits TIM-3 signaling is a TIM-3-binding agent (e.g., an antibody, an antibody conjugate, or an antigen-binding fragment thereof). In some embodiments, a TIM-3-binding agent is an antibody agent. Anti-TIM-3 antibody agents can include any polypeptide or polypeptide complex that includes immunoglobulin structural elements sufficient to confer specific binding. Exemplary antibody agents include, but are not limited to, monoclonal antibodies, polyclonal antibodies, antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody agent that inhibits TIM-3 signaling is a monoclonal antibody or a derivative thereof. In some embodiments, an antibody agent that inhibits TIM-3 signaling is a TIM-3 antibody or a derivative thereof. TIM-3 antibodies include, for example, MBG453, LY3321367, Sym023, any of the antibodies disclosed in WO2016/161270, and any derivatives thereof. In some embodiments, an antibody agent that inhibits TIM-3 signaling is TSR-022. In some certain embodiments, an agent includes combinations of agents that inhibit TIM-3 signaling.

In embodiments, an agent that inhibits TIM-3 signaling is administered at a dose of about 1, 3 or 10 mg/kg. In embodiments, an agent that inhibits TIM-3 signaling is administered at a dose of about 100-1500 mg. In embodiments, an agent that inhibits TIM-3 signaling is administered at a dose of about 100-500 mg. In embodiments, an agent that inhibits TIM-3 signaling is administered at a dose of about 1000-1500 mg.

In embodiments, an agent that inhibits TIM-3 signaling is administered at a dose of about 100 mg; a flat dose about 200 mg; a flat dose about 300 mg; a flat dose about 400 mg; a flat dose about 500 mg; a flat dose about 600 mg; a flat dose about 700 mg; a flat dose about 800 mg; a flat dose about 900 mg; a flat dose about 1000 mg; a flat dose about 1100 mg; a flat dose about 1200 mg; a flat dose about 1300 mg; a flat dose about 1400 mg; or a flat dose about 1500 mg. In embodiments, an agent that inhibits TIM-3 signaling is administered at a dose of about 100 mg. In embodiments, an agent that inhibits TIM-3 signaling is administered at a dose of about 200 mg. In embodiments, an agent that inhibits TIM-3 signaling is administered at a dose of about 300 mg. In embodiments, an agent that inhibits TIM-3 signaling is administered at a dose of about 400 mg. In embodiments, an agent that inhibits TIM-3 signaling is administered at a dose of about 500 mg. In embodiments, an agent that inhibits TIM-3 signaling is administered at a dose of about 600 mg. In embodiments, an agent that inhibits TIM-3 signaling is administered at a dose of about 700 mg. In embodiments, an agent that inhibits TIM-3 signaling is administered at a dose of about 800 mg. In embodiments, an agent that inhibits TIM-3 signaling is administered at a dose of about 900 mg. In embodiments, an agent that inhibits TIM-3 signaling is administered at a dose of about 1000 mg. In embodiments, an agent that inhibits TIM-3 signaling is administered at a dose of about 1100 mg. In embodiments, an agent that inhibits TIM-3 signaling is administered at a dose of about 1200 mg. In embodiments, an agent that inhibits TIM-3 signaling is administered at a dose of about 1300 mg. In embodiments, an agent that inhibits TIM-3 signaling is administered at a dose of about 1400 mg. In embodiments, an agent that inhibits TIM-3 signaling is administered at a dose of about 1500 mg. In embodiments, an agent that inhibits TIM-3 is administered at the administration interval of once a week, once every 2 weeks, once every 3 weeks, once every 4 weeks, once every 5 weeks, or once every 6 weeks. In embodiments, an agent that inhibits TIM-3 is administered at the administration interval of once every 2 weeks. In embodiments, an agent that inhibits TIM-3 is administered at the administration interval of once every 3 weeks. In embodiments, an agent that inhibits TIM-3 is administered for the period of at least 2, 4, 6, 8, 10, 12, 14, 16, 18, or 20 weeks. In embodiments, an agent that inhibits TIM-3 is administered intravenously.

In some embodiments, an agent that inhibits TIM-3 signaling is administered to a subject who is receiving, has received or will receive treatment with an anti-LAG-3 antibody agent (e.g., as described herein). In some embodiments, an anti-LAG-3 antibody agent is administered to a subject who is receiving, has received or will receive treatment with an agent that inhibits TIM-3 signaling.

In some embodiments, an anti-LAG-3 antibody agent is administered to a subject who is receiving, has received or will receive treatment with an agent that inhibits TIM-3 signaling and treatment with an agent that inhibits PD-1 signaling. In some embodiments, an agent that inhibits TIM-3 signaling is administered to a subject who is receiving, has received or will receive treatment with an anti-LAG-3 antibody agent and treatment with an agent that inhibits PD-1 signaling. In some embodiments, an agent that inhibits PD-1 signaling is administered to a subject who is receiving, has received or will receive treatment with an agent that inhibits TIM-3 signaling and/or treatment with an anti-LAG-3 antibody agent. In embodiments, an agent that inhibits PD-1 and/or an agent that inhibits TIM-3 is administered at a reduced dose.

In some related embodiments, administration of the anti-LAG-3 antibody agent and the agent that inhibits TIM-3 signaling improves the subject's response to an agent that inhibits PD-1 signaling. In some related embodiments, the subject is resistant for an agent that inhibits PD-1 signaling. In some related embodiments, the subject is refractory for an agent that inhibits PD-1 signaling. In some related embodiments, the subject overcomes resistance to an agent that inhibits PD-1 signaling after treatment with the anti-LAG-3 antibody agent and the agent that inhibits TIM-3 signaling. In some related embodiments, the administration of the anti-LAG-3 antibody agent and the agent that inhibits TIM-3 signaling sensitizes the subject to an agent that inhibits PD-1 signaling.

In some embodiments, the immune checkpoint inhibitor is a CTLA-4 inhibitor. In some embodiments, the CTLA-4 inhibitor is a small molecule, a nucleic acid, a polypeptide (e.g. an antibody), a carbohydrate, a lipid, a metal, a toxin, or a CTLA-4 binding agent. In some embodiments, the CTLA-4 binding agent is an antibody, an antibody conjugate, or an antigen-binding fragment thereof.

In some embodiments, the immune checkpoint inhibitor is a TIGIT inhibitor. In some embodiments, the TIGIT inhibitor is a small molecule, a nucleic acid, a polypeptide (e.g. an antibody), a carbohydrate, a lipid, a metal, a toxin, or a TIGIT binding agent. In some embodiments, the TIGIT binding agent is an antibody, an antibody conjugate, or an antigen-binding fragment thereof.

In some embodiments, the immune checkpoint inhibitor is an IDO inhibitor. In some embodiments, the IDO inhibitor is a small molecule, a nucleic acid, a polypeptide (e.g. an antibody, a carbohydrate, a lipid, a metal, a toxin, or an IDO binding agent. In some embodiments, the IDO binding agent is an antibody, an antibody conjugate, or an antigen-binding fragment thereof.

In some embodiments, the immune checkpoint inhibitor is a CSF1R inhibitor. In some embodiments, the CSF1R inhibitor is a small molecule, a nucleic acid, a polypeptide (e.g. an antibody, a carbohydrate, a lipid, a metal, a toxin, or a CSF1R binding agent. In some embodiments, the CSF1R binding agent is an antibody, an antibody conjugate, or an antigen-binding fragment thereof.

In embodiments of methods described herein, a subject has further been administered or will be administered an agent that inhibits poly (ADP-ribose) polymerase (PARP). In embodiments, an agent that inhibits PARP is a small molecule, a nucleic acid, a polypeptide (e.g., an antibody), a carbohydrate, a lipid, a metal, or a toxin. In embodiments, an agent that inhibits PARP is selected from the group consisting of: ABT-767, AZD 2461, BGB-290, BGP 15, CEP 8983, CEP 9722, DR 2313, E7016, E7449, fluzoparib (SHR 3162), IMP 4297, IN01001, JPI 289, JPI 547, monoclonal antibody B3-LysPE40 conjugate, MP 124, niraparib (ZEJULA) (MK-4827), NU 1025, NU 1064, NU 1076, NU1085, olaparib (AZD2281), ON02231, PD 128763, R 503, R554, rucaparib (RUBRACA) (AG-014699, PF-01367338), SBP 101, SC 101914, simmiparib, talazoparib (BMN-673), veliparib (ABT-888), WW 46, 2-(4-(trifluoromethyl)phenyl)-7,8-dihydro-5H-thiopyrano[4,3-d]pyrimidin-4-ol, and salts or derivatives thereof.

In embodiments, an agent that inhibits PARP is niraparib. In embodiments, niraparib is administered at a dose equivalent to about 100 mg of niraparib free base (e.g., a pharmaceutically acceptable salt of niraparib such as niraparib tosylate monohydrate is administered at a dose equivalent to about 100 mg of niraparib free base). In embodiments, niraparib is administered at a dose equivalent to about 200 mg of niraparib free base (e.g., a pharmaceutically acceptable salt of niraparib such as niraparib tosylate monohydrate is administered at a dose equivalent to about 200 mg of niraparib free base In embodiments, niraparib is administered at a dose equivalent to about 300 mg of niraparib free base (e.g., a pharmaceutically acceptable salt of niraparib such as niraparib tosylate monohydrate is administered at a dose equivalent to about 300 mg of niraparib free base).

In embodiments of the methods described herein, a subject (e.g., a mammal) has been administered or will be administered an agent that inhibits TIM-3 and an agent that inhibits PD-1 such that the mammal receives all three.

In embodiments, an agent that inhibits PD-1 is BGB-A317, BI 754091, IBI308, INCSHR-1210, JNJ-63723283, JS-001, MEDI-0680, MGA-012, nivolumab, PDR001, pembrolizumab, PF-06801591, REGN-2810, TSR-042, atezolizumab, avelumab, CX-072, durvalumab, FAZ053, LY3300054, PD-L1 millamolecule, or derivatives thereof.

In embodiments, an agent that inhibits TIM-3 is MBG453, LY3321367, Sym023, TSR-022 or a derivative thereof.

In embodiments, a subject (e.g., a mammal) has been administered or will be administered an agent that inhibits TIM-3 that is TSR-022 and an agent that inhibits PD-1 that is TSR-042.

In embodiments, an agent that inhibits PD-1 is administered at a dose of about 500 mg/patient to about 1000 mg/patient. In embodiments, an agent that inhibits PD-1 is administered at a dose of about 500 mg/patient. In embodiments, an agent that inhibits PD-1 is administered at a dose of about 1000 mg/patient. In embodiments, an agent that inhibits PD-1 is administered to the patient once every three weeks. In embodiments, an agent that inhibits PD-1 is administered for multiple cycles. In embodiments, an agent that inhibits PD-1 is administered for 2, 3, 4, 5, 6, or more cycles. In embodiments, an agent that inhibits PD-1 is administered for three, four, or five cycles. In embodiments, after the third, fourth, or fifth cycle, the agent that inhibits PD-1 is administered at a higher dose once every 6 weeks or more. In embodiments, an agent that inhibits PD-1 is administered at a higher dose once every 6 weeks. In embodiments, an agent that inhibits PD-1 is administered at a first dose of about 500 mg/patient. In embodiments, an agent that inhibits PD-1 is administered at a higher dose of about 1000 mg. In embodiments, an agent that inhibits PD-1 is administered at a first dose of about 500 mg once every 3 weeks for 3, 4, or 5 cycles followed by a second dose of about 1000 mg once every 6 weeks or more. In embodiments, an agent that inhibits PD-1 is administered at a first dose of about 500 mg once every 3 weeks for 3 cycles followed by a second dose of about 1000 mg once every 6 weeks or more. In embodiments, an agent that inhibits PD-1 is administered at a first dose of about 500 mg once every 3 weeks for 4 cycles followed by a second dose of about 1000 mg once every 6 weeks or more. In embodiments, an agent that inhibits PD-1 is administered at a first dose of about 500 mg once every 3 weeks for 5 cycles followed by a second dose of about 1000 mg once every 6 weeks or more. In embodiments, a second dose of 1000 mg is administered once every 6 weeks.

In embodiments, an agent that inhibits TIM-3 is administered at a dose of about 1, 3 or 10 mg/kg. In embodiments, an agent that inhibits TIM-3 is administered at a dose of about 100-1500 mg. In embodiments, an agent that inhibits TIM-3 is administered at a flat dose about 100 mg; a flat dose about 200 mg; a flat dose about 300 mg; a flat dose about 400 mg; a flat dose about 500 mg; a flat dose about 600 mg; a flat dose about 700 mg; a flat dose about 800 mg; a flat dose about 900 mg; a flat dose about 1000 mg; a flat dose about 1100 mg; a flat dose about 1200 mg; a flat dose about 1300 mg; a flat dose about 1400 mg; or a flat dose about 1500 mg. In embodiments, a dose is a flat dose of no more than about 1200 mg. In embodiments, a dose is a flat dose of no more than about 900 mg. In embodiments, a dose is a flat dose between about 100-500 mg. In embodiments, a dose is a flat dose between about 1000-1500 mg. In embodiments, an agent that inhibits TIM-3 is administered at the administration interval of once a week, once every 2 weeks, once every 3 weeks, once every 4 weeks, once every 5 weeks, or once every 6 weeks. In embodiments, an agent that inhibits TIM-3 is administered at the administration interval of once every 2 weeks. In embodiments, an agent that inhibits TIM-3 is administered at the administration interval of once every 3 weeks. In embodiments, an agent that inhibits TIM-3 is administered for the period of at least 2, 4, 6, 8, 10, 12, 14, 16, 18, or 20 weeks.

In embodiments, an agent that inhibits PD-1 is TSR-042 and is administered in an amount of about 500 mg every three weeks; and an agent that inhibits TIM-3 is TSR-022 and is administered in an amount of no more than about 1200 mg every three weeks. In embodiments, TSR-022 is administered in an amount of no more than about 900 mg every three weeks.

In embodiments, an agent that inhibits PD-1 and/or an agent that inhibits TIM-3 is administered intravenously.

In embodiments, an agent that inhibits LAG-3, an agent that inhibits PD-1 and/or an agent that inhibits TIM-3 is administered at a reduced dose.

In embodiments, a suitable dose of an anti-LAG-3 antibody agent is within a range of about 240 mg/patient to about 720 mg/patient. In embodiments, a suitable dose is about 240 mg/patient, about 320 mg/patient, about 400 mg/patient about 480 mg/patient, about 560 mg/patient, about 640 mg/patient, or about 720/mg patient. In embodiments, a suitable dose is about 200 mg/patient, about 300 mg/patient, about 400 mg/patient, about 500 mg/patient, about 600 mg/patient, or about 700 mg/patient. In other embodiments, a suitable dose is about 250 mg/patient, about 300 mg/patient, about 350 mg/patient, about 400 mg/patient, about 450 mg/patient, about 500 mg/patient, about 550 mg/patient, about 600 mg/patient, about 650 mg/patient, or about 700 mg/patient.

In some embodiments, the methods of the present disclosure include administering the LAG-3 agent at a dose of about 1 to about 5000 mg, about 1 mg, about 2 mg, about 2.5 mg, about 3 mg, about 4 mg, about 5 mg, about 6 mg, about 7 mg, about 8 mg, about 9 mg, about 10 mg, about 50 mg, about 100 mg, about 200 mg, about 250 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 2000 mg, about 3000 mg, about 4000 mg, or about 5000 mg. In embodiments, the methods of the present disclosure include administering the LAG-3 agent at a dose of about 20 mg, about 80 mg, about 240 mg, about 500 mg, about 720 mg, about 900 mg, about 1000 mg, about 1200 mg, about 1500 mg, about 1800 mg, about 2100 mg, about 2200 mg, or about 2500 mg.

In some embodiments, the methods of the present disclosure include administering the LAG-3 agent at a dose of about 0.01 mg/kg to about 100 mg/kg, about 0.1 mg/kg, about 0.5 mg/kg, about 1 mg/kg, about 2 mg/kg, about 2.5 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg of the mammal. In some embodiments, the methods of the present disclosure include administering the LAG-3 agent at a dose of about 1 mg/kg, about 3 mg/kg, about 10 mg/kg, about 12 mg/kg, about 15 mg/kg, about 20 mg/kg, or about 25 mg/kg.

In some embodiments, a method comprises administering the LAG-3 agent at a dose of about 1 mg/kg to about 10 mg/kg. In some embodiments, the methods of the present disclosure include administering the LAG-3 agent at a dose of about 1 mg/kg to about 30 mg/kg. In some embodiments, the methods of the present disclosure include administering the LAG-3 agent at a dose of about 1 mg/kg to about 10 mg/kg, about 1 mg/kg to about 25 mg/kg, or about 1 mg/kg to about 15 mg/kg.

In some embodiments, a method comprises administering the LAG-3 agent at a dose of about 20 mg, about 80 mg, about 240 mg, about 500 mg, about 720 mg, about 900 mg, or about 1000 mg, about 240-720 mg, about 240-1000 mg, or no more than about 1000 mg.

In some embodiments, the methods of the present disclosure include administering the LAG-3 agent at a dose of about 20 mg/patient, about 80 mg/patient, about 240 mg/patient, about 500 mg, or about 720 mg/patient. In some embodiments, the method includes administering the LAG-3 agent at a dose of 20 mg/patient. In some embodiments, the method includes administering the LAG-3 agent at a dose of about 80 mg/patient. In some embodiments, the method includes administering the LAG-3 agent at a dose of about 240 mg/patient. In some embodiments, the method includes administering the LAG-3 agent at a dose of about 500 mg/patient. In some embodiments, the method includes administering the LAG-3 agent at a dose of about 720 mg/patient.

In embodiments, a method comprises administering the LAG-3 agent every week, every two weeks, every three weeks, every four weeks, every five weeks, every six weeks, every seven weeks, or every eight weeks. In embodiments, a method comprises administering the LAG-3 agent every two weeks. In embodiments, a method comprises administering the LAG-3 agent every three weeks.

In embodiments, a method comprises administering the LAG-3 agent (e.g., a dose of about 20 mg, about 80 mg, about 240 mg, about 500 mg, about 720 mg every two weeks, or about 240-720 mg) every two weeks. In embodiments, a method comprises administering the LAG-3 agent (e.g., a dose of about 20 mg, about 80 mg, about 240 mg, about 500 mg, about 720 mg every three weeks, or about 240-720 mg) every three weeks.

In embodiments, a method comprises administering a LAG-3 agent at a dose of about 20 mg, about 80 mg, about 240 mg, about 500 mg, about 720 mg, about 900 mg, about 1000 mg, or about 1500 mg every two weeks, or about 240-720 mg or about 240-1500 mg every two weeks.

In embodiments, a method comprises administering a LAG-3 agent at a dose of about 3 mg/kg, about 10 mg/kg, about 12 mg/kg, or about 15 mg/kg every two weeks.

In embodiments, a method comprises administering a LAG-3 agent at a dose of about 20 mg, about 80 mg, about 240 mg, about 720 mg, about 500 mg about 900 mg, about 1000 mg, about 1500 mg, about 1800 mg, about 2100 mg, about 2200 mg, or about 2500 mg every three weeks, or about 240-720 mg or about 240-2500 mg every three weeks.

In embodiments, a method comprises administering a LAG-3 agent at a dose of about 10 mg/kg, about 12 mg/kg, about 15 mg/kg, about 20 mg/kg, or about 25 mg/kg every three weeks.

In embodiments, a method comprises administering a LAG-3 agent at a dose of about 240-720 mg/patient. In embodiments, a dose is administered once every two weeks. In embodiments, a dose is administered once every three weeks.

In embodiments, a method comprises administering a LAG-3 agent at a dose of about 20 mg/patient. In embodiments, a dose is administered once every two weeks. In embodiments, a dose is administered once every three weeks.

In embodiments, a method comprises administering a LAG-3 agent at a dose of about 80 mg/patient. In embodiments, a dose is administered once every two weeks. In embodiments, a dose is administered once every three weeks.

In embodiments, a method comprises administering a LAG-3 agent at a dose of about 240 mg/patient. In embodiments, a dose is administered once every two weeks. In embodiments, a dose is administered once every three weeks.

In embodiments, a method comprises administering a LAG-3 agent at a dose of about 500 mg/patient. In embodiments, a dose is administered once every two weeks. In embodiments, a dose is administered once every three weeks.

In embodiments, a method comprises administering a LAG-3 agent at a dose of about 720 mg/patient. In embodiments, a dose is administered once every two weeks. In embodiments, a dose is administered once every three weeks.

In embodiments, a method comprises administering a LAG-3 agent at a dose of about 900 mg/patient. In embodiments, a dose is administered once every two weeks. In embodiments, a dose is administered once every three weeks.

In embodiments, a method comprises administering a LAG-3 agent at a dose of about 1000 mg/patient. In embodiments, a dose is administered once every two weeks. In embodiments, a dose is administered once every three weeks.

In embodiments, a method comprises administering a LAG-3 agent at a dose of about 1500 mg/patient. In embodiments, a dose is administered once every two weeks. In embodiments, a dose is administered once every three weeks.

In embodiments, a method comprises administering a LAG-3 agent at a dose of about 1800 mg/patient. In embodiments, a dose is administered once every two weeks. In embodiments, a dose is administered once every three weeks.

In embodiments, a method comprises administering a LAG-3 agent at a dose of about 2100 mg/patient. In embodiments, a dose is administered once every two weeks. In embodiments, a dose is administered once every three weeks.

In embodiments, a method comprises administering a LAG-3 agent at a dose of about 2200 mg/patient. In embodiments, a dose is administered once every two weeks. In embodiments, a dose is administered once every three weeks.

In embodiments, a method comprises administering a LAG-3 agent at a dose of about 2500 mg/patient. In embodiments, a dose is administered once every two weeks. In embodiments, a dose is administered once every three weeks.

In embodiments, a method comprises administering a LAG-3 agent at a dose of about 3 mg/kg. In embodiments, a dose is administered once every two weeks. In embodiments, a dose is administered once every three weeks.

In embodiments, a method comprises administering a LAG-3 agent at a dose of about 10 mg/kg. In embodiments, a dose is administered once every two weeks. In embodiments, a dose is administered once every three weeks.

In embodiments, a method comprises administering a LAG-3 agent at a dose of about 12 mg/kg. In embodiments, a dose is administered once every two weeks. In embodiments, a dose is administered once every three weeks.

In embodiments, a method comprises administering a LAG-3 agent at a dose of about 15 mg/kg. In embodiments, a dose is administered once every two weeks. In embodiments, a dose is administered once every three weeks.

In embodiments, a method comprises administering a LAG-3 agent at a dose of about 20 mg/kg. In embodiments, a dose is administered once every two weeks. In embodiments, a dose is administered once every three weeks.

In embodiments, a method comprises administering a LAG-3 agent at a dose of about 25 mg/kg. In embodiments, a dose is administered once every two weeks. In embodiments, a dose is administered once every three weeks.

In embodiments, a LAG-3 agent is administered ocularly, oral, parenterally, topically, bronchially, buccally, intradermally, interdermally, transdermally, enterally, intra-arterially, intradermally, intragastricly, intramedullarily, intramuscular, intranasally, intraperitoneally, intrathecally, intravenously, intraventricular, within a specific organ (e.g., intrahepaticly), mucosally, nasally, orally, rectally, subcutaneously, sublingually, topically, tracheally, vaginally, vitreally, or any combination thereof. In embodiments, an anti-LAG-3 antibody agent is administered intravenously (e.g., by intravenous infusion).

In embodiments, a LAG-3 agent is IMP321, relatlimab (BMS-986016), BI 754111, GSK2831781 (IMP-731), Novartis LAG525 (IMP701), REGN3767, MK-4280, MGD-013, GSK-2831781, FS-118, XmAb22841, INCAGN-2385, FS-18, ENUM-006, AVA-017, AM-0003, Avacta PD-L1/LAG-3 bispecific affamer, iOnctura anti-LAG-3 antibody, Arcus anti-LAG-3 antibody, or Sym022.

In embodiments, a LAG-3 agent is a polypeptide as described herein; an isolated nucleic acid as described herein; a vector as described herein; an isolated cell as described herein; any composition as described herein; or any antibody agent as described herein.

In embodiments, a LAG-3 agent is a polypeptide comprising:
a CDR-H1 defined by SEQ ID NO: 5,
a CDR-H2 defined by SEQ ID NO: 6,
a CDR-H3 defined by SEQ ID NO: 7;
a CDR-L1 defined by SEQ ID NO: 8;
a CDR-L2 defined by SEQ ID NO: 9; and
a CDR-L3 defined by SEQ ID NO: 10.

In embodiments, a LAG-3 agent is a polypeptide comprising:
a heavy chain variable region amino acid sequence having at least 80%, 85%, 90%, 95%, or 98% sequence identity to SEQ ID NO: 3; and
a light chain variable region amino acid sequence having at least 80%, 85%, 90%, 95%, or 98% sequence identity to SEQ ID NO: 4.

In embodiments, a LAG-3 agent is a polypeptide comprising:
a heavy chain polypeptide sequence having at least 80%, 85%, 90%, 95%, or 98% sequence identity to SEQ ID NO: 1 or SEQ ID NO: 21; and
a light chain polypeptide sequence having at least 80%, 85%, 90%, 95%, or 98% sequence identity to SEQ ID NO: 2 or SEQ ID NO: 22.

In embodiments, a LAG-3 agent is TSR-033.

In embodiments of methods described herein, a subject is an animal (e.g., a mammal). In embodiments, a subject is a human. In embodiments, a subject is a non-human mammal (e.g., mice, rats, rabbits, or non-human primates). Accordingly, methods described herein can be useful in both treatment of humans and in veterinary medicine.

In embodiments of methods described herein, a subject (e.g., a mammal) has previously been treated with one or more different cancer treatment modalities (e.g., one or more of surgery, radiotherapy, chemotherapy, or immunotherapy). In embodiments, the mammal has been treated with one, two, three, four, or five lines of prior therapy. In embodiments, a line of prior therapy is cytotoxic therapy.

In some embodiments, a method described herein provides a clinical benefit to a subject. In embodiments, a clinical benefit is a complete response (“CR”), a partial response (“PR”) or a stable disease (“SD”). In some embodiments, a clinical benefit corresponds to at least SD. In some embodiments, a clinical benefit corresponds to at least a PR. In some embodiments, a clinical benefit corresponds to a CR. In some embodiments, at least 1%, 2%, 3%, 4%, 5%, 6%, 7%, 8%, 9%, 10%, 15%, 20%, 25%, 30%, 35%, 40%, 50%, 55%, 60%, 65%, 70%, 75%, 80%, 85%, 90% or 95% of patients achieve a clinical benefit. In some embodiments, at least 5% of patients achieve a clinical benefit. In some embodiments, at least 5% of patients achieve SD. In some embodiments, at least 5% of patients achieve at least a PR. In some embodiments, at least 5% of patients achieve CR. In some embodiments, at least 20% of patients achieve a clinical benefit. In some embodiments, at least 20% of patients achieve SD.

In some embodiments, the clinical benefit (e.g., SD, PR and/or CR) is determined in accordance with Response Evaluation Criteria in Solid Tumors (RECIST). In some embodiments, the clinical benefit (e.g., SD, PR and/or CR) is determined in accordance RECIST guidelines. In some embodiments, the clinical benefit (e.g., SD, PR and/or CR) is determined in accordance RECIST guidelines (version 1.1). In some embodiments, the clinical benefit (e.g., SD, PR and/or CR) is determined in accordance immune-related RECIST (irRECIST) guidelines. In some embodiments, tumor response can be assessed by either irRECIST or RECIST version 1.1. In some embodiments, tumor response can be assessed by both irRECIST and RECIST version 1.1. When used herein, the term “RECIST guidelines” can refer to RECIST 1.0, RECIST 1.1 or it RECIST interchangeably.

Also provided are methods of manufacturing polypeptides capable of binding LAG-3, by expressing a nucleic acid encoding the polypeptide in a host cell culture. In some embodiments, an anti-LAG-3 antibody agent (e.g., a polypeptide agent) is isolated. In some embodiments, an antibody agent (e.g., a polypeptide agent) can be purified to greater than 95% or 99% purity. In some embodiments, a method of manufacturing is a composition comprising a polypeptide capable of binding LAG-3, by combining the polypeptide (e.g., an isolated polypeptide) with the pharmaceutically acceptable carrier and formulating for administration to a subject. In some embodiments, the step of formulating for administration comprises formulating for parenteral delivery.

### BRIEF DESCRIPTION OF THE DRAWING

The Drawing included herein, which is composed of the following Figures, is for illustration purposes only and not for limitation.

FIG. 1 depicts a schematic illustration, not to scale, of immune checkpoint signaling and T-cell exhaustion.

FIG. 2 shows a graph depicting receptor occupancy of an exemplary anti-LAG-3 antibody agent on human PBMCs.

FIG. 3A shows a graph depicting receptor-ligand competition by an exemplary anti-LAG-3 antibody agent. An exemplary anti-LAG-3 antibody agent can block binding of a DyLight 650 (DyL650)-labeled LAG-3 fusion protein to MHC class II on Daudi cells. Diamonds represent isotype control and circles represent an exemplary anti-LAG-3 antibody agent. FIG. 3B is a schematic of a LAG-3 reporter gene assay schematic. FIG. 3C shows that an exemplary anti-LAG-3 antibody agent TSR-033 is a potent antagonist of LAG-3/MHC-II binding.

FIG. 4 depicts a mixed lymphocyte reaction (MLR) assay. Incubation of CD4+ T cells with an exemplary anti-LAG-3 antibody agent dose-dependently increased IL-2 production, and this effect was enhanced by combination with an exemplary anti-PD-1 antibody agent. Open diamonds represent isotype control, solid circles represent treatment with an exemplary anti-LAG-3 antibody agent, open squares represent an exemplary anti-LAG-3 antibody agent in combination with 2 ng/mL of an anti-PD-1 antibody agent, and open hexagons represent an exemplary anti-LAG-3 antibody agent in combination with 20 ng/mL of an anti-PD-1 antibody agent.

FIG. 5 shows a graph depicting IL-2 production from human PBMCs (from 5 donors) stimulated with 100 ng/mL SEB for 3 days. An exemplary anti-LAG-3 antibody agent dose-dependently increased IL-2 production, and this effect was enhanced by combination with an exemplary anti-PD-1 antibody agent. Small circles represent isotype control, squares represent anti-LAG-3 antibody agent, large circles represent an anti-PD-1 antibody agent, and triangles represent an exemplary anti-LAG-3 antibody agent in combination with an anti-PD-1 antibody agent.

FIG. 6 shows exemplary tumor quantification data from a mouse xenograft study where Balb/c mice were implanted with A20 lymphoma cells and treated with isotype control, an exemplary anti-PD-1 antibody agent, an anti-LAG-3 antibody agent, and a combination of an anti-PD-1 antibody agent and an anti-LAG-3 antibody agent. Mice treated with a combination of an anti-PD-1 antibody agent and an anti-LAG-3 antibody agent strongly inhibited tumor growth.

FIG. 7 shows exemplary splenic T cell activation data from a mouse xenograft study where Balb/c mice were implanted with A20 lymphoma cells and treated with isotype control, an exemplary anti-PD-1 antibody agent, an anti-LAG-3 antibody agent, and a combination of an anti-PD-1 antibody agent and an anti-LAG-3 antibody agent. Mice treated with a combination of an anti-PD-1 antibody agent and an anti-LAG-3 antibody agent had significant increases of both splenic proliferating T cells and CD8 T cells.

FIG. 8 shows exemplary tumor and splenic T cell quantification data from xenograft mice that were re-challenged with A20 lymphoma cells for naïve mice and mice treated with an exemplary anti-PD-1 antibody agent, and a combination of an anti-PD-1 antibody agent and an anti-LAG-3 antibody agent. The left panel depicts a graph of tumor volume and the right panel depicts T cell quantification (% CD4 T cells and % CD8 T cells, samples correspond to naïve mice, left column; anti-PD-1 treated animals, middle column; anti-PD-1 and anti-LAG-3 treated animals, right column).

FIGS. 9A-9B depict results from an exemplary in vitro T cell exhaustion model. (9A) Target expression of PD-1 and LAG-3 in responsive (pre-stimulated) cells and exhausted (post-stimulated cells). (9B) Quantification of IFNγ production in exhausted (post-stimulated) cells treated with a combination of an anti-PD-1 antibody agent and an anti-LAG-3 antibody agent (black bars) and isotype control (gray bars).

FIG. 10A depicts a dosing schematic with anti-LAG-3 as a monotherapy and as a combination therapy with anti-PD-1. FIG. 10B provides a schematic of a receptor occupancy (RO) assay that permits detection of both bound TSR-033 and total LAG-3 on the surface of T cells in patient peripheral blood. FIG. 10C depicts receptor occupany (RO) as measured using patient T cells, where receptor occupancy approaches saturation at a 240 mg dose (top data set) and approximately 50% at an 80 mg dose (middle data set).

FIG. 11 depicts the twelve (12) intrachain disulfide bonds and four (4) interchain disulfide bonds of an anti-LAG-3 antibody agent.

FIGS. 12A-12F depict appreciable co-expression of PD-1, TIM-3, and LAG-3 was detected on tumor infiltrating cells—particularly CD8+ T cells—in non-small cell lung cancer (NSCLC) (FIG. 12A), endometrial (FIG. 12B), renal (RCC) (FIG. 12C), cervical (FIG. 12D), gastric (FIG. 12E), and colorectal cancer (CRC) (FIG. 12F).

FIG. 13A shows the immune composition of tumor-infiltrating leukocytes as determined by flow cytometry using tumor samples from NSCLC and RCC patients. As read clockwise from the 12′ position, the sections of the graph depict the amount of CD8+, Th, Treg, other CD3+/NKT, NK, monocytes, DC, granulocytes, and other CD45+ cells. FIG. 13B depicts studies using granzyme B as a functional marker for T and NK cells from NSCLC and RCC patients. FIG. 13C depicts analysis of TILs from primary EGFR+ NSCLC and found that PD-1⁺TIM-3⁺LAG-3⁺ cytotoxic T cells were highly dysfunctional, as assessed by Granzyme B status: dual or triple checkpoint expression marks dysfunctional CD8+ T cells.

FIGS. 14A-14G depict studies in humanized NOG-EXL mice that were first inoculated subcutaneously with A549 non-small cell lung cancer (NSCLC) and then treated with test antibodies that were administered intraperitoneally twice weekly, with dosing at 10 mg/kg. Tumor volume (mm³) was measured from 0-40 days following treatment with a human IgG4 isotype control (FIG. 14A); anti-PD-1 antibody TSR-042 (FIG. 14B); anti-TIM-3 antibody TSR-022 (FIG. 14C); a combination of TSR-042 and TSR-022 (FIG. 14D); anti-LAG-3 antibody TSR-033 (FIG. 14E); a combination of TSR-042 and TSR-033 (FIG. 14F); and a combination of TSR-042, TSR-022, and TSR-033 (FIG. 14G).

FIGS. 15A-15C depict studies of NSCLC tumors in animals that remained following termination of the study described in Example 9. FIG. 15A shows the fold change in tumor-infiltrating lymphocytes (CD45). FIG. 15B shows the fold change in regulatory T cells (Tregs), with Tregs being identified as CD4+FOXP3+. FIG. 15C shows the fold change in proliferating Tregs, and Ki-67 was used as a marker for proliferating cells. An asterisk is used to identify a p<0.05 in unpaired Student's T-test, comparing anti-PD-1 monotherapy to ether dual or triple checkpoint combinations.

FIG. 16A depicts a reduction in tumor associated macrophages (TAMs) upon dual or triple checkpoint blockade. FIG. 16B depicts increased M1/M2 ratios observed upon dual or triple checkpoint blockade. FIGS. 16C-D shows that dual blockade of LAG-3 and PD-1 using TSR-033 and TSR-042 improves therapeutic efficacy and immune activation in a humanized NSCLC tumor mouse model. FIG. 16C shows that the combination of TSR-033 with TSR-042 has an additive effect (coefficient of drug interaction, CDI=1.001) on restricting tumor growth in HuNOG-EXL mice inoculated with A549 cells. Mice were randomized at tumor volumes of 80-120 mm³, followed by administration of the indicated regimens (Methods). Tumor growth inhibition at termination for each treatment arm is indicated in parentheses. FIG. 16D shows that, relative to TSR-042 monotherapy, the combination group had increased tumor infiltrating lymphocytes, intra-tumoral proliferating T cells, CD8/Treg ratios and reduced TAMs (unpaired Student's T-test). Data represent two independent experiments (n=10 per treatment arm) and have been normalized to fold change over isotype control for each treatment arm. FIGS. 16E-16F show increased effector memory T cells and ex vivo cytokine production by splenic T cells from the combination group relative to TSR-042 alone. FIG. 16E shows increased effector memory CD4 and CD8 T cells in the combination group compared to TSR-042 alone. FIG. 16F shows a significant increase in IFNγ and TNFα production by CD4 T cells in the combination group over TSR-042 alone upon ex vivo stimulation of mouse splenocytes.

FIGS. 17A-17G depict studies in humanized NOG-EXL mice that were first inoculated subcutaneously with MDA-MB436 triple negative breast cancer (TNBC) and then treated with test antibodies that were administered intraperitoneally twice weekly, with dosing at 10 mg/kg. Tumor volume (mm³) was measured from 0-40 days following treatment with a human IgG4 isotype control (FIG. 17A); anti-PD-1 antibody TSR-042 (FIG. 17B); anti-TIM-3 antibody TSR-022 (FIG. 17C); a combination of TSR-042 and TSR-022 (FIG. 17D); anti-LAG-3 antibody TSR-033 (FIG. 17E); a combination of TSR-042 and TSR-033 (FIG. 17F); and a combination of TSR-042, TSR-022, and TSR-033 (FIG. 17G).

FIGS. 18A-18G depict studies in a syngeneic tumor mouse model wherein BALB/c mice were first inoculated subcutaneously with EMT-6 breast carcinoma cell lines and then treated with test antibodies that were administered intraperitoneally twice weekly, with dosing at 10 mg/kg. Tumor volume (mm³) was measured from 0-20 days following treatment with: a human IgG4 isotype control (FIG. 18A); anti-PD-1 antibody TSR-042 (FIG. 18B); anti-TIM-3 antibody TSR-022 (FIG. 18C); a combination of anti-PD-1 TSR-042 and anti-TIM-3 TSR-022 (FIG. 18D); anti-LAG-3 antibody TSR-033 (FIG. 18E); a combination of anti-PD-1 TSR-042 and anti-LAG-3 TSR-033 (FIG. 18F); and a combination of anti-PD-1 TSR-042, anti-TIM-3 antibody TSR-022, and anti-LAG-3 TSR-033 (FIG. 18G).

FIG. 19 relates to a framework developed to identify cancers that can benefit from triple blockade therapy and depicts a summary of the signatures used.

### CERTAIN DEFINITIONS

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. Examples of routes of administration include parenteral, e.g., intravenous, intradermal, subcutaneous, oral (e.g., inhalation), transdermal (i.e., topical), transmucosal, and rectal administration. For example, in some embodiments, administration may be ocular, oral, parenteral, topical, etc. In embodiments, administration is parenteral (e.g., intravenous administration). In embodiments, intravenous administration is intravenous infusion. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

Affinity: As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Those skilled in the art are well familiar with antibody structure and sequence elements, recognize “variable” and “constant” regions in provided sequences, and understand that there may be some flexibility in definition of a “boundary” between such domains such that different presentations of the same antibody chain sequence may, for example, indicate such a boundary at a location that is shifted one or a few residues relative to a different presentation of the same antibody chain sequence. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc), or other pendant group (e.g., poly-ethylene glycol, etc).

Antibodies include antibody fragments. Antibodies also include, but are not limited to, polyclonal monoclonal, chimeric dAb (domain antibody), single chain, F_{ab}, F_{ab′}, F_{(ab′)2} fragments, scFvs, and F_{ab} expression libraries. An antibody may be a whole antibody, or immunoglobulin, or an antibody fragment.

As detailed above, whole antibodies consist of two pairs of a “light chain” (LC) and a “heavy chain” (HC) (such light chain (LC)/heavy chain pairs are abbreviated herein as LC/HC). The light chains and heavy chains of such antibodies are polypeptides consisting of several domains. In a whole antibody, each heavy chain comprises a heavy chain variable region (abbreviated herein as HCVR or VH) and a heavy chain constant region. The heavy chain constant region comprises the heavy chain constant domains CH1, CH2 and CH3 (antibody classes IgA, IgD, and IgG) and optionally the heavy chain constant domain CH4 (antibody classes IgE and IgM). Each light chain comprises a light chain variable domain VL and a light chain constant domain CL. The variable domains VH and VL can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (Janeway, C. A., Jr, et al, (2001). Immunobiology., 5th ed., Garland Publishing; and Woof, J., Burton, D., Nat Rev Immunol 4 (2004) 89-99). The two pairs of heavy chain and light chain (HC/LC) are capable of specifically binding to the same antigen. Thus said whole antibody is a bivalent, monospecific antibody. Such “antibodies” include e.g., mouse antibodies, human antibodies, chimeric antibodies, humanized antibodies and genetically engineered antibodies (variant or mutant antibodies) as long as their characteristic properties are retained. In some embodiments, antibodies or binding agents are humanized antibodies, especially as recombinant human or humanized antibodies.

In some embodiments, the antibody or binding agent can be “symmetrical.” By “symmetrical” is meant that the antibody or binding agent has the same kind of Fv regions (e.g., the antibody has two Fab regions). In some embodiments, the antibody or binding agent can be “asymmetrical.” By “asymmetrical” is meant that the antibody or binding agent has at least two different kinds of Fv regions (e.g., the antibody has: Fab and scFv regions, Fab and scFv2 regions, or Fab-VHH regions). Various asymmetrical antibody or binding agent architectures are known in the art (Brinkman and Kontermann et al. 2017 Mabs (9)(2):182-212).

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc, as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®; Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments, an antibody agent is or comprises a polypeptide whose amino acid sequence includes at least one CDR (e.g., at least one heavy chain CDR and/or at least one light chain CDR) that is substantially identical to one found in a reference antibody. In some embodiments, an included CDR is substantially identical to a reference CDR in that it is either identical in sequence or contains between 1-5 amino acid substitutions as compared with the reference CDR. In some embodiments, an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments, an included CDR is substantially identical to a reference CDR in that it shows at least 90%, 91%, 92%, 93%, 94%, 95%, 96%, 97%, 98%, 99%, or 100% sequence identity with the reference CDR. In some embodiments, an included CDR is substantially identical to a reference CDR in that it shows at least 95%, 96%, 97%, 98%, 99%, or 100% sequence identity with the reference CDR. In some embodiments, an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an included CDR is substantially identical to a reference CDR in that at least one amino acid within the included CDR is substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89.

For example, in some instances the following six groups each contain amino acids that are conservative substitutions for one another: 1) Serine, Threonine; 2) Aspartic Acid, Glutamic Acid; 3) Asparagine, Glutamine; 4) Arginine, Lysine; 5) Isoleucine, Leucine, Methionine, Alanine, Valine, and 6) Phenylalanine, Tyrosine, Tryptophan. Other appropriate substitutions are known to the person of ordinary skill in the art in addition to the non-limiting examples described herein.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell). In some embodiments, “binding” refers to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473).

Binding agent: In general, the term “binding agent” is used herein to refer to any entity that binds to a target of interest as described herein. In many embodiments, a binding agent of interest is one that binds specifically with its target in that it discriminates its target from other potential binding partners in a particular interaction context. In general, a binding agent may be or comprise an entity of any chemical class (e.g., polymer, non-polymer, small molecule, polypeptide, carbohydrate, lipid, nucleic acid, etc.). In some embodiments, a binding agent is a single chemical entity. In some embodiments, a binding agent is a complex of two or more discrete chemical entities associated with one another under relevant conditions by non-covalent interactions. For example, those skilled in the art will appreciate that in some embodiments, a binding agent may comprise a “generic” binding moiety (e.g., one of biotin/avidin/streptavidin and/or a class-specific antibody) and a “specific” binding moiety (e.g., an antibody or aptamers with a particular molecular target) that is linked to the partner of the generic biding moiety. In some embodiments, such an approach can permit modular assembly of multiple binding agents through linkage of different specific binding moieties with the same generic binding moiety partner. In some embodiments, binding agents are or comprise polypeptides (including, e.g., antibodies or antibody fragments). In some embodiments, binding agents are or comprise small molecules. In some embodiments, binding agents are or comprise nucleic acids. In some embodiments, binding agents are aptamers. In some embodiments, binding agents are polymers; in some embodiments, binding agents are not polymers. In some embodiments, binding agents are non-polymeric in that they lack polymeric moieties. In some embodiments, binding agents are or comprise carbohydrates. In some embodiments, binding agents are or comprise lectins. In some embodiments, binding agents are or comprise peptidomimetics. In some embodiments, binding agents are or comprise scaffold proteins. In some embodiments, binding agents are or comprise mimeotopes. In some embodiments, binding agents are or comprise nucleic acids, such as DNA or RNA. In embodiments, a binding agent is an isolated polypeptide as described herein. In embodiments, a binding agent is an antibody, an antibody conjugate, or an antigen-binding fragment thereof. In embodiments, a binding agent is an antibody.

Cancer: The terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure identifies certain cancers to which its teachings may be relevant. In some embodiments, a relevant cancer may be characterized as a solid tumor. In some embodiments, a relevant cancer may be characterized as a hematologic tumor. In embodiments, a cancer is adenocarcinoma, adenocarcinoma of the lung, acute myeloid leukemia (“AML”), acute lymphoblastic leukemia (“ALL”), adrenocortical carcinoma, anal cancer (e.g,. squamous cell carcinoma of the anus), appendiceal cancer, B-cell derived leukemia, B-cell derived lymphoma, bladder cancer, brain cancer, breast cancer (e.g., triple negative breast cancer (TNBC)), cancer of the fallopian tube(s), cancer of the testes, cerebral cancer, cervical cancer (e.g., squamous cell carcinoma of the cervix), cholagiocarcinoma, choriocarcinoma, chronic myelogenous leukemia, a CNS tumor, colon cancer or colorectal cancer (e.g., colon adenocarcinoma), diffuse intrinsic pontine glioma (DIPG), diffuse large B cell lymphoma (“DLBCL”), embryonal rhabdomyosarcoma (ERMS), endometrial cancer, epithelial cancer, esophageal cancer (e.g., squamous cell carcinoma of the esophagus), Ewing's sarcoma, eye cancer (e.g., uveal melanoma), follicular lymphoma (“FL”), gallbladder cancer, gastric cancer, gastrointestinal cancer, glioma, head and neck cancer (e.g., squamous cell carcinoma of the head and neck (SCHNC)), a hematological cancer, hepatocellular cancer, Hodgkin's lymphoma (HL)/primary mediastinal B-cell lymphoma, kidney cancer, kidney clear cell cancer, laryngeal cancer, leukemia, liver cancer, lung cancer (e.g., non-small cell lung cancer (NSCLC), small cell lung cancer, lung adenocarcinoma, or squamous cell carcinoma of the lung), lymphoma, melanoma, Merkel cell carcinoma, mesothelioma, monocytic leukemia, multiple myeloma, myeloma, a neuroblastic-derived CNS tumor (e.g., neuroblastoma (NB)), non-Hodgkin's lymphoma (NHL), oral cancer, osteosarcoma, ovarian cancer, ovarian carcinoma, pancreatic cancer, peritoneal cancer, primary peritoneal cancer, prostate cancer, relapsed or refractory classic Hodgkin's Lymphoma (cHL), renal cancer (e.g., renal cell carcinoma), rectal cancer, salivary gland cancer (e.g., a salivary gland tumor), sarcoma, skin cancer, small intestine cancer, stomach cancer, squamous cell carcinoma, squamous cell carcinoma of the penis, stomach cancer, T-cell derived leukemia, T-cell derived lymphoma, thymic cancer, a thymoma, thyroid cancer, uveal melanoma, urothelial cell carcinoma, uterine cancer (e.g., uterine endometrial cancer or uterine sarcoma), vaginal cancer (e.g., squamous cell carcinoma of the vagina), vulvar cancer (e.g., squamous cell carcinoma of the vulva), or Wilms tumor.

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components. In some embodiments, the carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some cases, it may be desirable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

CDR: The term “CDR” as used herein, refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Boundaries of CDRs have been defined differently depending on the system, of which several are known in the art (e.g., Kabat, Chothia, etc.).

Combination therapy: As used herein, the term “combination therapy” refers to a clinical intervention in which a subject is simultaneously exposed to two or more therapeutic regimens (e.g. two or more therapeutic agents). In some embodiments, the two or more therapeutic regimens may be administered simultaneously. In some embodiments, the two or more therapeutic regimens may be administered sequentially (e.g., a first regimen administered prior to administration of any doses of a second regimen). In some embodiments, the two or more therapeutic regimens are administered in overlapping dosing regimens. In some embodiments, administration of combination therapy may involve administration of one or more therapeutic agents or modalities to a subject receiving the other agent(s) or modality. In some embodiments, combination therapy does not necessarily require that individual agents be administered together in a single composition (or even necessarily at the same time). In some embodiments, two or more therapeutic agents or modalities of a combination therapy are administered to a subject separately, e.g., in separate compositions, via separate administration routes (e.g., one agent orally and another agent intravenously), and/or at different time points. In some embodiments, two or more therapeutic agents may be administered together in a combination composition, or even in a combination compound (e.g., as part of a single chemical complex or covalent entity), via the same administration route, and/or at the same time.

Compound and Agent: The terms “compound” and “agent” are used herein interchangeably. They refer to any naturally occurring or non-naturally occurring (i.e., synthetic or recombinant) molecule, such as a biological macromolecule (e.g., nucleic acid, polypeptide or protein), organic or inorganic molecule, or an extract made from biological materials such as bacteria, plants, fungi, or animal (e.g., mammalian, including human) cells or tissues. The compound may be a single molecule or a mixture or complex of at least two molecules.

Comparable: The term “comparable” as used herein, refers to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Control: As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

Epitope: As used herein, the term “epitope” includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Framework or framework region: As used herein, refers to the sequences of a variable region minus the CDRs. Because a CDR sequence can be determined by different systems, likewise a framework sequence is subject to correspondingly different interpretations. The six CDRs divide the framework regions on the heavy and light chains into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, FR1, for example, represents the first framework region closest to the amino terminal end of the variable region and 5′ with respect to CDR1, and FRs represents two or more of the sub-regions constituting a framework region.

Glycan: as used herein, “glycan” refers to a sugar polymer (moiety) component (e.g., such as of a glycoprotein). The term “glycan” can encompass free glycans, including glycans that have been cleaved or otherwise released from a glycoprotein. The term “glycoform” used herein can refer to a particular form of a glycoprotein. That is, when a glycoprotein includes a particular polypeptide that has the potential to be linked to different glycans or sets of glycans, then each different version of the glycoprotein (i.e., where the polypeptide is linked to a particular glycan or set of glycans) can be referred to as a “glycoform.”

Homology: As used herein, the term “homology” refers to the overall relatedness between polymeric molecules, e.g., between nucleic acid molecules (e. g. , DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution. As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences. Representative algorithms and computer programs useful in determining the percent homology between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent homology between two nucleotide sequences can, alternatively, be determined for example using the GAP program in the GCG software package using an NWSgapdna.CMP matrix.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α,α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Human antibody: As used herein, is intended to include antibodies having variable and constant regions generated (or assembled) from human immunoglobulin sequences. In some embodiments, antibodies (or antibody components) may be considered to be “human” even though their amino acid sequences include residues or elements not encoded by human germline immunoglobulin sequences (e.g., include sequence variations, for example that may (originally) have been introduced by random or site-specific mutagenesis in vitro or by somatic mutation in vivo), for example in one or more CDRs and in particular CDR3.

Humanized: As is known in the art, the term “humanized” is commonly used to refer to antibodies (or antibody components) whose amino acid sequence includes V_{H} and V_{L} region sequences from a reference antibody raised in a non-human species (e.g., a mouse), but also includes modifications in those sequences relative to the reference antibody intended to render them more “human-like”, i.e., more similar to human germline sequences. In some embodiments, a “humanized” antibody (or antibody component) is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a CH₁, hinge, CH₂, CH₃, and, optionally, a CH₄ region of a heavy chain constant region. In some embodiments, a humanized antibody only contains a humanized VL region. In some embodiments, a humanized antibody only contains a humanized VH region. In some certain embodiments, a humanized antibody contains humanized VH and VL regions.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical or at least 80%, 85%, 90%, 95%, or 99% identical. In some embodiments, a nucleic acid sequence or amino acid sequence is substantially identical to a reference sequence in that it is either identical in sequence or contains between 1-5 substitutions as compared with the reference sequence. For example, in some embodiments, an amino acid sequence is substantially identical to a reference amino acid sequence in that it is either identical in sequence or contains between 1-5 amino acid substitutions as compared with the reference sequence. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control individual (or multiple control individuals) in the absence of the treatment described herein. A “control individual” is an individual afflicted with the same type and approximately the same severity of a disease, disorder, or condition as the individual being treated, who is about the same age as the individual being treated (to ensure that the stages of the disease in the treated individual and the control individual(s) are comparable).

Isolated: as used herein, refers to a substance and/or entity (e.g. a nucleic acid or a polypeptide) that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

K_{D}: as used herein, refers to the dissociation constant of a binding agent (e.g., an antibody or binding component thereof) from a complex with its partner (e.g., the epitope to which the antibody or binding component thereof binds).

K_{off}: as used herein, refers to the off rate constant for dissociation of a binding agent (e.g., an antibody or binding component thereof) from a complex with its partner (e.g., the epitope to which the antibody or binding component thereof binds).

Kₒₙ: as used herein, refers to the on rate constant for association of a binding agent (e.g., an antibody or binding component thereof) with its partner (e.g., the epitope to which the antibody or binding component thereof binds).

Kit: As used herein, the term “kit” refers to any delivery system for delivering materials. Such delivery systems may include systems that allow for the storage, transport, or delivery of various diagnostic or therapeutic reagents (e.g., oligonucleotides, enzymes, etc. in the appropriate containers) and/or supporting materials (e.g., buffers, written instructions for performing the assay, etc.) from one location to another. For example, kits include one or more enclosures (e.g., boxes, cartridges, bottles, ampoules, etc.) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte Specific Reagents (ASR's) regulated under section 520(e) of the Federal Food, Drug, and Cosmetic Act, but are not limited thereto. Indeed, any delivery system comprising two or more separate containers that each contain a subportion of the total kit components are included in the term “fragmented kit.” In contrast, a “combined kit” refers to a delivery system containing all of the components in a single container (e.g., in a single box housing each of the desired components). The term “kit” includes both fragmented and combined kits.

Normal: As used herein, the term “normal,” when used to modify the term “individual” or “subject” refers to an individual or group of individuals who does not have a particular disease or condition and is also not a carrier of the disease or condition. The term “normal” is also used herein to qualify a biological specimen or sample isolated from a normal or wild-type individual or subject, for example, a “normal biological sample.”

Nucleic acid: as used herein, the term “nucleic acid” refers to a polymer of at least three nucleotides. In some embodiments, a nucleic acid comprises DNA. In some embodiments comprises RNA. In some embodiments, a nucleic acid is single stranded. In some embodiments, a nucleic acid is double stranded. In some embodiments, a nucleic acid can contain non-natural or altered nucleotides. The terms “nucleic acid” and “polynucleotide” as used herein can refer to a polymeric form of nucleotides of any length, either ribonucleotides (RNA) or deoxyribonucleotides (DNA). These terms can refer to the primary structure of the molecule, and thus include double- and single-stranded DNA, and double- and single-stranded RNA. The terms can include, as equivalents, analogs of either RNA or DNA made from nucleotide analogs and modified polynucleotides such as, though not limited to, methylated and/or capped polynucleotides. Nucleic acids can be linked via phosphate bonds to form nucleic acid sequences or polynucleotides, though many other linkages are known in the art (e.g., phosphorothioates, boranophosphates, and the like).

Patient or subject: As used herein, the term “patient” or “subject” refers to any organism to which provided compound or compounds described herein are administered in accordance with the present invention e.g., for experimental, diagnostic, prophylactic, and/or therapeutic purposes. Typical subjects include animals. The term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone. In embodiments, animals are mammals such as mice, rats, rabbits, non-human primates, and humans; insects; worms; etc. In embodiments, a subject is a human. In some embodiments, a subject may be suffering from, and/or susceptible to a disease, disorder, and/or condition (e.g., cancer). As used herein, a “patient population” or “population of subjects” refers to a plurality of patients or subjects.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

Polypeptide: As used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Sample: As used herein, the term “sample” encompasses any sample obtained from a biological source. The terms “biological sample” and “sample” are used interchangeably. A biological sample can, by way of non-limiting example, include skin tissue, liver tissue, kidney tissue, lung tissue, cerebrospinal fluid (CSF), blood, amniotic fluid, sera, urine, feces, epidermal sample, skin sample, cheek swab, sperm, amniotic fluid, cultured cells, bone marrow sample and/or chorionic villi. Cell cultures of any biological samples can also be used as biological samples. A biological sample can also be, e.g., a sample obtained from any organ or tissue (including a biopsy or autopsy specimen), can comprise cells (whether primary cells or cultured cells), medium conditioned by any cell, tissue or organ, tissue culture. In some embodiments, biological samples suitable for the invention are samples which have been processed to release or otherwise make available a nucleic acid for detection as described herein. Fixed or frozen tissues also may be used.

Solid Tumor: As used herein, the term “solid tumor” refers to an abnormal mass of tissue that usually does not contain cysts or liquid areas. In some embodiments, a solid tumor may be benign; in some embodiments, a solid tumor may be malignant. Those skilled in the art will appreciate that different types of solid tumors are typically named for the type of cells that form them. Examples of solid tumors are carcinomas, lymphomas, and sarcomas. In some embodiments, solid tumors may be or comprise adrenal, bile duct, bladder, bone, brain, breast, cervix, colon, endometrium, esophagum, eye, gall bladder, gastrointestinal tract, kidney, larynx, liver, lung, nasal cavity, nasopharynx, oral cavity, ovary, penis, pituitary, prostate, retina, salivary gland, skin, small intestine, stomach, testis, thymus, thyroid, uterine, vaginal, and/or vulval tumors.

Suffering from: An individual who is “suffering from” a disease, disorder, and/or condition (e.g., any cancer described herein) has been diagnosed with or displays one or more symptoms of the disease, disorder, and/or condition.

Susceptible to: An individual who is “susceptible to” a disease, disorder, and/or condition has not been diagnosed with and/or may not exhibit symptoms of the disease, disorder, and/or condition. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein associated with the disease, disorder, and/or condition; (4) habits and/or lifestyles associated with development of the disease, disorder, and/or condition; (5) a family history of the disease, disorder, and/or condition; (6) reaction to certain bacteria or viruses; (7) exposure to certain chemicals. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutically Effective Amount: As used herein, a “therapeutically effective amount” or “effective amount” is meant an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, and/or delays progression of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”) refers to any administration of a therapeutic molecule (e.g., any compound described herein) that partially or completely alleviates, ameliorates, relieves, inhibits, delays onset of, delays progression of, reduces severity of and/or reduces incidence of one or more symptoms or features of a particular disease, disorder, and/or condition (e.g., cancer). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In embodiments, treatment comprises administration of a LAG-3 agent as described herein.

### DETAILED DESCRIPTION OF CERTAIN EMBODIMENTS

Lymphocyte Activation Gene-3 (LAG-3), which is also known as cluster of differentiation 223 (CD223), is a member of the immunoglobulin supergene family and is structurally and genetically related to CD4. LAG-3 is expressed on T-cells, B cells, natural killer (NK) cells and plasmacytoid dendritic cells (pDCs). Like CD4, LAG-3 ectodomain is composed of four Ig-like domains (D1-D4) and LAG-3 has been demonstrated to interact with MHC Class II molecules (Baixeras et al., J. Exp. Med., 176: 327-337 (1992)), but binds at a distinct site (Huard et al., Proc. Nail Acad. Sci. USA, 94(11): 5744-5749 (1997)). For example, a soluble LAG-3 immunoglobulin fusion protein (sLAG-3Ig) directly and specifically binds via LAG-3 to MHC class II on the cell surface (Huard et al., Eur. J. Immunol., 26: 1180-1186 (1996)).

LAG-3 is upregulated following T-cell activation, and modulates T-cell function as well as T-cell homeostasis (Sierro et al., Expert Opin. Ther. Targets, 15(1): 91-101(2011)). The LAG-3/MHC class II interaction may play a role in down-regulating antigen-dependent stimulation of CD4+ T lymphocytes, as demonstrated in in vitro studies of antigen-specific T-cell proliferation, higher expression of activation antigens such as CD25, and higher concentrations of cytokines such as interferon-gamma and interleukin-4 (Huard et al., Eur. J. Immunol., 24: 3216-3221 (1994)). CD4+CD25+ regulatory T-cells (Treg) also have been shown to express LAG-3 upon activation and antibodies to LAG-3 inhibit suppression by induced Treg cells, both in vitro and in vivo, suggesting that LAG-3 contributes to the suppressor activity of Treg cells (Huang et al., Immunity, 21: 503-513 (2004)). Furthermore, LAG-3 has been shown to negatively regulate T-cell homeostasis by regulatory T cell-dependent and -independent mechanisms (Workman, C. J. and Vignali, D. A., J. Immunol, 174: 688-695 (2005)).

Subsets of conventional T-cells that are anergic or display impaired functions express LAG-3, and LAG-3+ T-cells are enriched at tumor sites and during chronic viral infections. However, while LAG-3 knockout mice have been shown to mount normal virus-specific CD4+ and CD8 T-cell responses, blockade of the PD-1/PD-L1 pathway combined with LAG-3 blockade improved viral control as compared with PD-L1 blockade alone (Blackburn et al., Nat. Immunol., 10: 29-37 (2009); and Riehter et al., Int. Immunol., 22: 13-2 (2010)). In a self-tolerance/tumor mouse model where transgenic CD8+ T-cells were rendered unresponsive/anergic in vivo, LAG-3 blockade or deficiency in CD8+ T-cells enhanced T-cell proliferation, T-cell recruitment and effector functions at the tumor site (Grosso et al., J. Clin. Invest., 117: 3383-92 (2007)).

In addition, the interaction between LAG-3 and its major ligand, MHC class II, may play a role in modulating dendritic cell function (Andreae et al., J Immunol., 168:3874-3880, 2002). Recent preclinical studies have documented a role for LAG-3 in CD8+ T cell exhaustion (Blackburn et al., Nat Immunol., 10: 29-37, 2009), and blockade of the LAG-3/MHC class II interaction using a LAG-3Ig fusion protein may be useful for cancer therapy.

There is a need for antagonists of LAG-3 (e.g., anti-LAG-3 antibody agents) that bind LAG-3 with high affinity and/or effectively neutralizes LAG-3 activity. The present disclosure provides such LAG-3 binding agents.

### LAG-3 Agents

LAG-3 Antibody Agents

The present disclosure provides, among other things, anti-LAG-3 antibody agents that bind to an epitope of a protein encoded by the lymphocyte activation gene 3 (LAG-3) and various compositions and methods relating thereto. For example, the disclosure provides amino acid sequences of anti-LAG-3 antibody agents, corresponding nucleic acid sequences encoding such amino acid sequences, as well as variants of such anti-LAG-3 antibody agents. The disclosure also provides related vectors, compositions, and methods of using anti-LAG-3 antibody agents to treat a disorder or disease that is responsive to LAG-3 inhibition, such as cancer or an infectious disease.

In some embodiments, anti-LAG-3 antibody agents (e.g., anti-LAG-3 antibodies or fragments thereof) can bind to an epitope of LAG-3 which blocks the binding of LAG-3 to MHC class II molecules and inhibit LAG-3-mediated signaling. For example, an anti-LAG-3 antibody agent can bind to one or more of the four Ig-like extracellular domains (D1-D4) of the LAG-3 protein (see, e.g. Triebel et al., J Exp. Med., 171(5): 1393-1405 (1990); and Bruniquei et al., Immunogenetics, 47: 96-98 (1997)). In some embodiments, anti-LAG-3 antibody agents can bind to domain 1 (D1) and/or domain 2 (D2) of the LAG-3 protein. In some embodiments, an anti-LAG-3 antibody agent can bind an epitope of LAG-3 which blocks the binding of LAG-3 to any one or more of its putative ligands. In some embodiments, an anti-LAG-3 antibody agent can bind an epitope of LAG-3 which blocks the binding of LAG-3 to two or more of its putative ligands.

In some embodiments, an anti-LAG-3 antibody agent can inhibit or neutralize the activity of LAG-3. The terms “inhibit” or “neutralize,” as used herein with respect to the activity of an antibody agent, can refer to the ability to substantially antagonize, prohibit, prevent, restrain, slow, disrupt, alter, eliminate, stop, or reverse the progression or severity of, for example, the biological activity of a target, or a disease or condition associated with a target. In some embodiments, a target is LAG-3. In some embodiments, a disease or condition is associated with LAG-3. In some embodiments, an anti-LAG-3 antibody agent can inhibit or neutralize the activity of LAG-3 by at least about 20%, about 30%, about 40%, about 50%, about 60%, about 70%, about 80%, about 90%, about 95%, about 100%, or a range defined by any two of the foregoing values (e.g., 20% to 100%, 40% to 100% or 60% to 95%, etc.).

In some embodiments, an anti-LAG-3 antibody agent is isolated. In some embodiments, an antibody agent can be purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) (See, e.g., Flatman et al., J. Chromatogr., B 848:79-87 (2007)).

In some embodiments, an anti-LAG-3 antibody agent comprises an Fc. Fc domains can interact with cell surface receptors which can allow antibodies to activate the immune system. In IgG, IgA and IgD antibody isotypes, a Fc region is composed of two identical protein fragments, derived from the second and third constant domains of the antibody's two heavy chains; IgM and IgE Fc regions contain three heavy chain constant domains (C_{H} domains 2-4) in each polypeptide chain. The Fc regions of IgGs bear a highly conserved N-glycosylation site (N297). Glycosylation of the Fc fragment can be essential for Fc receptor-mediated activity. The N-glycans attached to this site can predominantly be core-fucosylated diantennary structures of the complex type.

While the constant regions of the light and heavy chains may not be directly involved in binding of the antibody to an antigen, the constant regions can influence the orientation of the variable regions. The constant regions can also exhibit various effector functions, such as participation in antibody-dependent complement-mediated lysis or antibody-dependent cellular toxicity via interactions with effector molecules and cells.

The disclosed anti-LAG-3 antibody agents can be antibodies of any isotype, including isotype IgA, isotype IgD, isotype IgE, isotype IgG, or isotype IgM. In some embodiments, an anti-LAG-3 antibody contains a IgG γ1, γ2, γ3, or γ4 constant domain. In an exemplary embodiment, an anti-LAG-3 antibody contains IgG γ4 constant domain.

In some embodiments, an anti-LAG-3 antibody agent can contain an immunoglobulin heavy chain which comprises the amino acid sequence of SEQ ID NO: 1. In some cases, an anti-LAG-3 antibody can contain an immunoglobulin heavy chain having at least 60%, 65%, 70%, 75%, 80%, 85%, 90%, 91%, 92%, 93%, 94%, 95%, 96%, 97%, 98%, 99%, or 100% sequence identity to the amino acid sequence of SEQ ID NO: 1. In some embodiments, an anti-LAG-3 antibody agent can contain an immunoglobulin light chain which comprises the amino acid sequence of SEQ ID NO: 2. In some cases, an anti-LAG-3 antibody can contain an immunoglobulin light chain having at least 60%, 65%, 70%, 75%, 80%, 85%, 90%, 91%, 92%, 93%, 94%, 95%, 96%, 97%, 98%, 99%, or 100% sequence identity to the amino acid sequence of SEQ ID NO: 2. In some embodiments, an anti-LAG-3 antibody agent can contain an immunoglobulin heavy chain which includes a signal peptide (underlined in SEQ ID NO: 1). In some embodiments, an anti-LAG-3 antibody agent can contain an immunoglobulin heavy chain which does not include the signal peptide (SEQ ID NO: 21). In some embodiments, an anti-LAG-3 antibody agent can contain an immunoglobulin light chain which includes a single peptide (underlined in SEQ ID NO: 2). In some embodiments, an anti-LAG-3 antibody agent can contain an immunoglobulin light chain which does not include the signal peptide (SEQ ID NO: 22).

In some embodiments, an anti-LAG-3 antibody agent (e.g., an anti-LAG-3 antibody) can comprise a V_{H} sequence having at least 80%, 85%, 90%, 91%, 92%, 93%, 94%, 95%, 96%, 97%, 98%, 99%, or 100% sequence identity to the amino acid sequence of SEQ ID NO: 3. In some embodiments, a V_{H} sequence having at least 80%, 85%, 90%, 91%, 92%, 93%, 94%, 95%, 96%, 97%, 98%, or 99% sequence identity can contain substitutions (e.g., conservative substitutions), insertions, or deletions relative to the reference sequence, but an anti-LAG-3 antibody comprising that sequence retains the ability to bind to LAG-3. In some embodiments, a total of 1 to 10 amino acids have been substituted, inserted and/or deleted in the amino acid sequence of SEQ ID NO: 3. In some embodiments, substitutions, insertions, or deletions occur in regions outside the CDRs (e.g., in the FRs). In some embodiments, an anti-LAG-3 antibody agent can comprise the V_{H} sequence of the amino acid sequence of SEQ ID NO: 3, including post-translational modifications of that sequence. In some embodiments, the V_{H} can comprise one, two or three CDRs selected from: (a) CDR-H1 comprising the amino acid sequence of SEQ ID NO: 5, (b) CDR-H2 comprising the amino acid sequence of SEQ ID NO: 6, and (c) CDR-H3 comprising the amino acid sequence of SEQ ID NO: 7.

In some embodiments, an anti-LAG-3 antibody agent (e.g., an anti-LAG-3 antibody) is provided, wherein the antibody agent comprises a light chain variable domain (V_{L}) having at least 80%, 85%, 90%, 91%, 92%, 93%, 94%, 95%, 96%, 97%, 98%, 99%, or 100% sequence identity to the amino acid sequence of SEQ ID NO: 4. In some embodiments, a V_{L} sequence having at least 80%, 85%, 90%, 91%, 92%, 93%, 94%, 95%, 96%, 97%, 98%, or 99% identity can contain substitutions (e.g., conservative substitutions), insertions, or deletions relative to the reference sequence, but an anti-LAG-3 antibody agent comprising that sequence retains the ability to bind to LAG-3. In some embodiments, a total of 1 to 10 amino acids have been substituted, inserted and/or deleted in any one of the amino acid sequence of SEQ ID NO: 4. In some embodiments, the substitutions, insertions, or deletions occur in regions outside the CDRs (e.g., in the FRs). Optionally, the anti-LAG-3 antibody agent comprises the V_{L} sequence of SEQ ID NO: 4, including post-translational modifications of that sequence. In embodiments, the V_{L} comprises one, two or three CDRs selected from (a) CDR-L1 comprising the amino acid sequence of SEQ ID NO: 8; (b) CDR-L2 comprising the amino acid sequence of SEQ ID NO: 9; and (c) CDR-L3 comprising the amino acid sequence of SEQ ID NO: 10.

In some embodiments, an anti-LAG-3 antibody agent (e.g., an anti-LAG-3 antibody) is provided, wherein the antibody agent comprises a V_{H} as in any of the embodiments provided above, and a V_{L} as in any of the embodiments provided above. In some embodiments, the antibody agent comprises a V_{H} comprising the amino acid sequence of SEQ ID NO: 3, and a V_{L} sequence in SEQ ID NO: 4, including post-translational modifications of those sequences.

In addition, the present disclosure provides isolated or purified nucleic acid sequences encoding the foregoing immunoglobulin polypeptides. The disclosed anti-LAG-3 antibody agents can contain an immunoglobulin heavy chain which is encoded by the nucleic acid sequence of SEQ ID NO: 11. In some embodiments, an anti-LAG-3 antibody agent can contain an immunoglobulin heavy chain which is encoded by a nucleic acid sequence having at least 60%, 65%, 70%, 75%, 80%, 85%, 90%, 91%, 92%, 93%, 94%, 95%, 96%, 97%, 98%, 99%, or 100% sequence identity to the nucleic acid sequence of SEQ ID NO: 11. The disclosed anti-LAG-3 antibody agents can contain an immunoglobulin light chain which is encoded by the nucleic acid sequence of SEQ ID NO: 12. In some cases, an anti-LAG-3 antibody agent can contain an immunoglobulin light chain which is encoded by a nucleic acid sequence having at least 60%, 65%, 70%, 75%, 80%, 85%, 90%, 91%, 92%, 93%, 94%, 95%, 96%, 97%, 98%, 99%, or 100% sequence identity to the nucleic acid sequence of SEQ ID NO: 12.

In some embodiments, an anti-LAG-3 antibody agent (e.g., an anti-LAG-3 antibody) comprises a V_{H} sequence which is encoded by a nucleic acid sequence having at least 80%, 85%, 90%, 91%, 92%, 93%, 94%, 95%, 96%, 97%, 98%, 99%, or 100% sequence identity to the nucleic acid sequence of SEQ ID NO: 13. In some embodiments, a V_{H} sequence which is encoded by a nucleic acid sequence having at least 80%, 85%, 90%, 91%, 92%, 93%, 94%, 95%, 96%, 97%, 98%, or 99% sequence identity to the nucleic acid sequence of SEQ ID NO: 13 can contain nucleotide substitutions, insertions, or deletions relative to the reference sequence, but an anti-LAG-3 antibody agent (e.g., an anti-LAG-3 antibody) comprising V_{H} encoded by that sequence retains the ability to bind to LAG-3. In some embodiments, a total of 1 to 10 nucleotides have been substituted, inserted and/or deleted in the nucleic acid sequence of SEQ ID NO: 13. In some embodiments, substitutions, insertions, or deletions occur in regions outside the CDRs (e.g., in the FRs). In some embodiments, the V_{H} can comprise one, two or three CDRs selected from: (a) CDR-H1 encoded by the nucleic acid sequence of SEQ ID NO: 15, (b) CDR-H2 encoded by the nucleic acid sequence of SEQ ID NO: 16, and (c) CDR-H3 encoded by the nucleic acid sequence of SEQ ID NO: 17. In some embodiments, an anti-LAG-3 antibody agent (e.g., an anti-LAG-3 antibody) comprises a light chain variable domain (V_{L}) which is encoded by a nucleic acid sequence having at least 80%, 85%, 90%, 91%, 92%, 93%, 94%, 95%, 96%, 97%, 98%, 99%, or 100% sequence identity to the nucleic acid sequence of SEQ ID NO: 14. In some embodiments, a V_{L} sequence which is encoded by a nucleic acid sequence having at least 80%, 85%, 90%, 91%, 92%, 93%, 94%, 95%, 96%, 97%, 98%, or 99% identity to the nucleic acid sequence of SEQ ID NO: 14 can contain nucleotide substitutions, insertions, or deletions relative to the reference sequence, but an anti-LAG-3 antibody agent (e.g., an anti-LAG-3 antibody) comprising a V_{L} encoded by that sequence retains the ability to bind to LAG-3. In some embodiments, a total of 1 to 10 nucleotides have been substituted, inserted and/or deleted in the nucleic acid sequence of SEQ ID NO: 14. In some embodiments, the substitutions, insertions, or deletions occur in regions outside the CDRs (e.g., in the FRs). In certain embodiments, a V_{L} comprises one, two or three CDRs selected from (a) CDR-L1 encoded by nucleic acid sequence of SEQ ID NO: 18; (b) CDR-L2 encoded by nucleic acid sequence of SEQ ID NO: 19; and (c) CDR-L3 encoded by nucleic acid sequence of SEQ ID NO: 20.

In some embodiments, an anti-LAG-3 antibody agent can comprise a deletion at an end of a light chain. In some embodiments, an anti-LAG-3 antibody agent can comprise a deletion of 3 or more amino acids at an end of the light chain. In some embodiments, an anti-LAG-3 antibody agent can comprise a deletion of 7 or less amino acids at an end of the light chain. In some embodiments, an anti-LAG-3 antibody agent can comprise a deletion of 3, 4, 5, 6, or 7 amino acids at an end of the light chain. In some embodiments, an anti-LAG-3 antibody agent can comprise an insertion in a light chain. In some embodiments, an anti-LAG-3 antibody agent can comprise an insertion of 1, 2, 3, 4, 5, 6, 7, 8, 9, or 10, or more amino acids in the light chain.

In some embodiments, a provided anti-LAG-3 antibody agent has a structure that includes one or more disulfide bonds. In some embodiments, the one or more disulfide bonds are or include a disulfide bond at the expected position for an IgG4 immunoglobulin. In some embodiments, a disulfide bond is present at one or more residues corresponding to positions selected from residue 41, 115, 147, 160, 216, 239, 242, 274, 334, 380 and 438 of SEQ ID NO: 1 (or residue 22, 96, 128, 141, 197, 220, 223, 255, 315, 361 and 419 of SEQ ID NO: 21). In some embodiments, a disulfide bond is present at one or more residues corresponding to positions selected from residue 45, 115, 161, 221 and 241 of SEQ ID NO: 2 (or residue 23, 93, 139, 199 and 219 of SEQ ID NO: 22). The light chain variable region can be aligned with the variable region of the heavy chain, and the light chain constant region can be aligned with the first constant region of the heavy chain. The remaining constant regions of the heavy chains can be aligned with each other.

In some embodiments, an anti-LAG-3 antibody agent can bind to an LAG-3 protein with a K_{D} more than about 1 micromolar. In some embodiments, an anti-LAG-3 antibody agent can bind to an LAG-3 protein with a K_{D} less than or equal to about 1 micromolar (e.g., about 1 μM, 0.9 μM, 0.8 μM, 0.7 μM, 0.6 μM, 0.5 μM, 0.4 μM, 0.3 μM, 0.2 μM, 0.1 μM, 0.05 μM, 0.025 μM, 0.01 μM, 0.001 μM or less). In some embodiments, an anti-LAG-3 antibody agent can bind to an LAG-3 protein with a K_{D} less than or equal to about 100 nanomolar (e.g., about 100 nM, 90 nM, 80 nM, 70 nM, 60 nM, 50 nM, 40 nM, 30 nM, 20 nM, 10 nM, 5 nM, 2.5 nM, 1 nM, 0. 1 nM or less). In some embodiments, an anti-LAG-3 antibody agent can bind to an LAG-3 protein with a K_{D} less than or equal to about 10 nanomolar (e.g., about 10 nM, 9 nM, 8 nM, 7 nM, 6 nM, 5 nM, 4 nM, 3 nM, 2 nM, 1 nM, 0.5 nM, 0.25 nM, 0.1 nM, 0.01 nM or less). In some embodiments, an anti-LAG-3 antibody agent can bind to an LAG-3 protein with a K_{D} less than or equal to about 100 picomolar (e.g., about 100 pM, 90 pM, 80 pM, 70 pM, 60 pM, 50 pM, 40 pM, 30 pM, 20 pM, 10 pM, 5 pM, 2.5 pM, 1 pM, 0.1 pM or less). In some embodiments, an anti-LAG-3 antibody agent can bind to an LAG-3 protein with a K_{D} less than or equal to about 10 picomolar (e.g., about 10 pM, 9 pM, 8 pM, 7 pM, 6 pM, 5 pM, 4 pM, 3 pM, 2 pM, 1 pM, 0.5 pM, 0.25 pM, 0.1 pM, 0.01 pM or less). In some embodiments, an anti-LAG-3 antibody agent can bind to an LAG-3 protein with a K_{D} less than or equal to about 1 nanomolar (e.g., about 1 nM, 0.9 nM, 0.8 nM, 0.7 nM, 0.6 nM, 0.5 nM, 0.4 nM, 0.3 nM, 0.2 nM, 0.1 nM, 0.05 nM, 0.025 nM, 0.01 nM, 0.0 1 nM, or less). In some embodiments, an anti-LAG-3 antibody agent can bind to LAG-3 with a K_{D} less than or equal to 200 pM (e.g., about 200, pM, 190 pM, 175 pM, 150 pM, 125 pM, 110 pM, 100 pM, 90 pM, 80 pM, 75 pM, 60 pM, 50 pM, 40 pM, 30 pM, 25 pM, 20 pM, 15 pM, 10 pM, 5 pM, 1 pM, or less). In some embodiments, an anti-LAG-3 antibody agent can bind to an LAG-3 protein with a K_{D} in a range defined by any two of the foregoing values (e.g., within a range of 1 pM to 1 μM). K_{D} can be measured by any suitable assay. For example, K_{D} can be measured by a radiolabeled antigen binding assay (RIA) (See, e.g., Chen et al., J. Mol. Biol., 293:865-881 (1999); Presta et al., Cancer Res., 57:4593-4599 (1997)). For example, K_{D} can be measured using surface plasmon resonance assays (e.g., using a BIACORE®-2000 or a BIACORE®-3000). Other non-limiting examples include fluorescence activated cell sorting (FACS), separable beads (e.g., magnetic beads), solution phase competition (KINEXA™), antigen panning, and/or ELISA (see, e.g., Janeway et al. (eds.), Immunobiology, 5th ed., Garland Publishing, New York, N.Y., 2001).

In some embodiments, an anti-LAG-3 antibody agent is or comprises a monoclonal anti-LAG-3 antibody or fragment thereof. Examples of antibody fragments include, but are not limited to, (1) a Fab fragment, which is a monovalent fragment consisting of the V_{L}, V_{H}, C_{L}, and C_{H}1 domains, (2) a F(ab′)2 fragment, which is a bivalent fragment comprising two Fab fragments linked by a disulfide bridge at the hinge region, (3) a Fv fragment consisting of the V_{L} and V_{H} domains of a single arm of an antibody (e.g., an scFv), (4) a Fab′ fragment, which results from breaking the disulfide bridge of an F(ab′)2 fragment using mild reducing conditions, (5) a disulfide-stabilized Fv fragment (dsFv), and (6) a single domain antibody (sdAb), which is an antibody single variable region domain (V_{H} or V_{L}) polypeptide that specifically binds antigen.

Additional LAG-3 Agents

Still other LAG-3 agents are also suitable for use in any of the methods (e.g., therapeutic uses and dosing regimens) described herein.

In embodiments, a LAG-3 agent is an antibody, an antibody conjugate, or an antigen-binding fragment thereof. In embodiments, a LAG-3 agent is a small molecule, a nucleic acid, a polypeptide (e.g., an antibody), a carbohydrate, a lipid, a metal, or a toxin. In embodiments, a LAG-3 agent is a small molecule. In embodiments, a LAG-3 agent is a LAG-3 binding agent. In embodiments, a LAG-3 agent is an antibody, an antibody conjugate, or an antigen-binding fragment thereof. In embodiments, a LAG-3 agent is IMP321, relatlimab (BMS-986016), BI 754111, GSK2831781 (IMP-731), Novartis LAG525 (IMP701), REGN3767, MK-4280, MGD-013, GSK-2831781, FS-118, XmAb22841, INCAGN-2385, FS-18, ENUM-006, AVA-017, AM-0003, Avacta PD-L1/LAG-3 bispecific affamer, iOnctura anti-LAG-3 antibody, Arcus anti-LAG-3 antibody, or Sym022, or a LAG-3 inhibitor described in WO 2016/126858, WO 2017/019894, or WO 2015/138920, each of which is hereby incorporated by reference in its entirety.

### Mutation Frequency

Anti-LAG-3 antibody agent of the current disclosure can comprise a heavy chain sequence that is characterized by a mutation frequency of at least about 5%, 6%, 7%, 8%, 9%, 10%, 11%, 12%, 13 %, 14%, 15%, 16%, 17%, 18%, 19%, or 20%, or higher from a germline sequence. The antibody agents of the present disclosure can comprise a CDR3 region that is a light chain sequence with a mutation frequency of at least about 5%, 6%, 7%, 8%, 9%, 10%, 11%, 12%, 13%, 14%, 15%, 16%, 17%, 18%, 19%, or 20%, or higher from a germline sequence. The antibody agents of the current disclosure can comprise a heavy chain and a light chain sequence with a mutation frequency of at least about 5%, 6%, 7%, 8%, 9%, 10%, 11%, 12%, 13%, 14%, 15%, 16%, 17%, 18%, 19%, or 20%, or higher from a germline sequence. The antibody agents of the current disclosure can comprise a V_{H} region from a V_{H} family selected from the group consisting of any one of V_{H} family 4-59.

### Antibody Fragments

In one aspect, an anti-LAG-3 antibody agent according to any of the above embodiments can be an antibody fragment. An antibody fragment comprises a portion of an intact antibody, such as the antigen binding or variable region of the intact antibody. Antibody fragments include, but are not limited to, Fab, Fab′, Fab′-SH, F(ab)2, Fv, diabody, linear antibodies, multispecific formed from antibody fragments antibodies and scFv fragments, and other fragments described below. In some embodiments, the antibody is a full length antibody, e.g., an intact IgG1 antibody or other antibody class or isotype as described herein. (See, e.g., Hudson et al., Nat. Med., 9:129-134 (2003); Pluckthun, The Pharmacology of Monoclonal Antibodies, vol. 113, pp. 269-315 (1994); Hollinger et al., Proc. Natl. Acad. Sci. USA, 90: 6444-6448 (1993); WO93/01161; and U.S. Pat. Nos. 5,571,894, 5,869,046, 6,248,516, and 5,587,458). A full length antibody, intact antibody, or whole antibody is an antibody having a structure substantially similar to a native antibody structure or having heavy chains that contain an Fc region as defined herein. Antibody fragments can be made by various techniques, including but not limited to proteolytic digestion of an intact antibody as well as production by recombinant host cells (e.g., E. coli or phage), as known in the art.

An Fv is the minimum antibody fragment that contains a complete antigen-recognition and antigen-binding site. This fragment contains a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable region (or half of an Fv comprising only three CDRs specific for an antigen) has the ability to recognize and bind antigen, although at a lower affinity than the entire binding site.

A single-chain Fv (sFv or scFv) is an antibody fragment that comprises the V_{H} and V_{L} antibody domains connected into a single polypeptide chain. The sFv polypeptide can further comprise a polypeptide linker between the V_{H} and V_{L} domains that enable the sFv to form the desired structure for antigen binding (See, e.g., Pluckthun, The Pharmacology of Monoclonal Antibodies, vol. 113, Rosenburg and Moore eds., Springer-Verlag, New York, pp. 269-315 (1994); Borrebaeck 1995, infra.

A diabody is a small antibody fragment prepared by constructing an sFv fragment with a short linker (e.g., about 5-10 residues) between the V_{H} and V_{L} domains such that inter-chain but not intra-chain pairing of the V domains is achieved, resulting in a bivalent fragment. Bispecific diabodies are heterodimers of two crossover sFv fragments in which the V_{H} and V_{L} domains of the two antibodies are present on different polypeptide chains (See, e.g., EP 404,097; WO 93/11161; and Hollinger et al, Proc. Natl. Acad. Sci. USA, 90:6444-6448 (1993)).

Domain antibodies (dAbs), which can be produced in fully human form, are the smallest known antigen-binding fragments of antibodies, ranging from about 11 kDa to about 15 kDa. DAbs are the robust variable regions of the heavy and light chains of immunoglobulins (V_{H} and V_{L}, respectively). They are highly expressed in microbial cell culture, show favorable biophysical properties including, for example, but not limited to, solubility and temperature stability, and are well suited to selection and affinity maturation by in vitro selection systems such as, for example, phage display. dAbs are bioactive as monomers and, owing to their small size and inherent stability can be formatted into larger molecules to create drugs with prolonged serum half-lives or other pharmacological activities. (See, e.g., WO9425591 and US20030130496).

Fv and sFv are the only species with intact combining sites that are devoid of constant regions. Thus, they are suitable for reduced nonspecific binding during in vivo use. sFv fusion proteins can be constructed to yield fusion of an effector protein at either the amino or the carboxy terminus of an sFv. The antibody fragment also can be a “linear antibody (See, e.g., U.S. Pat. No. 5,641,870). Such linear antibody fragments can be monospecific or bispecific.

### Chimeric and Humanized Antibodies

In some embodiments, an anti-LAG-3 antibody agent is or comprises a monoclonal antibody, including a chimeric, humanized or human antibody.

In some embodiments, an anti-LAG-3 antibody agent provided herein can be a chimeric antibody (See, e.g., U.S. Pat. No. 4,816,567; and Morrison et al., Proc. Natl. Acad. Sci. USA, 81:6851-6855 (1984)). A chimeric antibody can be an antibody in which a portion of the heavy and/or light chain is derived from a particular source or species, while the remainder of the heavy and/or light chain is derived from a different source or species. In one example, a chimeric antibody can comprise a non-human variable region (e.g., a variable region derived from a mouse, rat, hamster, rabbit, or non-human primate, such as a monkey) and a human constant region. In a further example, a chimeric antibody can be a “class switched” antibody in which the class or subclass has been changed from that of the parent antibody. Chimeric antibodies include antigen-binding fragments thereof.

In some embodiments, a chimeric antibody can be a humanized antibody (See, e.g., Almagro and Fransson, Front. Biosci.,13:1619-1633 (2008); Riechmann et al., Nature, 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005); Padlan, Mol. Immunol., 28:489-498 (1991); Dall'Acqua et al., Methods, 36:43-60 (2005); Osbourn et al., Methods, 36:61-68 (2005); and Klimka et al., Br. J. Cancer, 83:252-260 (2000)). A humanized antibody is a chimeric antibody comprising amino acid residues from non-human hypervariable regions and amino acid residues from human FRs. In certain embodiments, a humanized antibody will comprise substantially all of at least one, and typically two, variable domains, in which all or substantially all of the hypervariable regions (e.g., CDRs) correspond to those of a non-human antibody, and all or substantially all of the FRs correspond to those of a human antibody. A humanized antibody optionally may comprise at least a portion of an antibody constant region derived from a human antibody.

A non-human antibody can be humanized to reduce immunogenicity to humans, while retaining the specificity and affinity of the parental non-human antibody. A humanized antibody can comprise one or more variable domains comprising one or more CDRs, or portions thereof, derived from a non-human antibody. A humanized antibody can comprise one or more variable domains comprising one or more FRs, or portions thereof, derived from human antibody sequences. A humanized antibody can optionally comprise at least a portion of a human constant region. In some embodiments, one or more FR residues in a humanized antibody are substituted with corresponding residues from a non-human antibody (e.g., the antibody from which the CDR residues are derived), to restore or improve antibody specificity or affinity.

Human framework regions that may be used for humanization include but are not limited to: framework regions selected using a “best-fit” method; framework regions derived from the consensus sequence of human antibodies of a particular subgroup of light or heavy chain variable regions; human mature (somatically mutated) framework regions or human germline framework regions; and framework regions derived from screening FR libraries (See, e.g., Sims et al., J. Immunol., 151:2296 (1993); Carter et al., Proc. Natl. Acad. Sci. USA, 89:4285 (1992); Presta et al., J. Immunol., 151:2623 (1993); Baca et al., J. Biol. Chem., 272:10678-10684 (1997); and Rosok et al., J. Biol. Chem., 271:22611-22618 (1996)).

### Human Antibodies

In some embodiments, an anti-LAG-3 antibody agent provided herein is a human antibody. Human antibodies can be produced using various techniques known in the art (See, e.g., van Dijk and van de Winkel, Curr. Opin. Pharmacol., 5: 368-74 (2001); and Lonberg, Curr. Opin. Immunol., 20:450-459 (2008)). A human antibody can be one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies may be prepared by administering an immunogen (e.g., an LAG-3 protein) to a transgenic animal that has been modified to produce intact human antibodies or intact antibodies with human variable regions in response to antigenic challenge (See, e.g., Lonberg, Nat. Biotech., 23:1117-1125 (2005); U.S. Pat. Nos. 6,075,181, 6,150,584, 5,770,429, and 7,041,870; and U.S. Pat. App. Pub. No. US 2007/0061900). Human variable regions from intact antibodies generated by such animals may be further modified, e.g., by combining with a different human constant region.

Human antibodies can also be made by hybridoma-based methods. For example, human antibodies can be produced from human myeloma and mouse-human heteromyeloma cell lines, using human B-cell hybridoma technology, and other methods (See, e.g., Kozbor, J. Immunol., 133: 3001 (1984); Brodeur et al., Monoclonal Antibody Production Techniques and Applications, pp. 51-63 (1987); Boerner et al., J. Immunol., 147: 86 (1991); Li et al., Proc. Natl. Acad. Sci. USA, 103:3557-3562 (2006); U.S. Pat. No. 7,189,826; Ni, Xiandai Mianyixue, 26(4): 265-268 (2006); Vollmers and Brandlein, Histology and Histopathology, 20(3): 927-937 (2005); and Vollmers and Brandlein, Methods and Findings in Experimental and Clinical Pharmacology, 27(3): 185-91 (2005)). Human antibodies may also be generated by isolating Fv clone variable domain sequences selected from human-derived phage display libraries. Such variable domain sequences may then be combined with a desired human constant region.

### Library Derivation

In some embodiments, an anti-LAG-3 antibody agent provided herein may be isolated by screening combinatorial libraries for antibodies with the desired activity or activities (See, e.g., in Hoogenboom et al., Methods in Molecular Biology 178: 1-37 (2001); McCafferty et al., Nature, 348:552-554; Clackson et al., Nature, 352: 624-628 (1991); Marks et al., J. Mol. Biol., 222: 581-597 (1992); Marks and Bradbury, Methods in Molecular Biology, 248: 161-175 (2003); Sidhu et al., J. Mol. Biol., 338(2): 299-310 (2004); Lee et al., J. Mol. Biol., 340(5): 1073-1093 (2004); Fellouse, Proc. Natl. Acad. Sci. USA, 101(34): 12467-12472 (2004); and Lee et al., J. Immunol. Methods, 284(1-2): 119-132 (2004)). Repertoires of V_{H} and V_{L} genes can be cloned separately (e.g., by PCR) and recombined randomly in libraries (e.g., phage libraries), and screened (See, e.g., Winter et al., Ann. Rev. Immunol., 12: 433-455 (1994)). Alternatively, the naive repertoire can be cloned (e.g., from human) to provide a single source of antibodies to a wide range of non-self and also self-antigens without any immunization (See, e.g., Griffiths et al., EMBO J., 12: 725-734 (1993)). Alternatively, naive libraries can be synthetically made by cloning unrearranged V-gene segments from stem cells, and encoding the CDR3 regions using random primers or to rearrange the V-gene segments in vitro (See, e.g., Hoogenboom and Winter, J. Mol. Biol., 227: 381-388 (1992); U.S. Pat. No. 5,750,373, and U.S. Pat. Pub. Nos. US 2005/0079574, US 2005/0119455, US 2005/0266000, US 2007/0117126, US 2007/0160598, US 2007/0237764, US 2007/0292936, and US 2009/0002360). Antibodies or antibody fragments isolated from human antibody libraries can be considered human antibodies or human antibody fragments herein.

### Amino Acid Sequence Variants

In some embodiments, amino acid sequence variants of anti-LAG-3 antibody agents provided herein are contemplated. A variant typically can differ from a polypeptide specifically disclosed herein in one or more substitutions, deletions, additions and/or insertions. Such variants can be naturally occurring or can be synthetically generated, for example, by modifying one or more of the above polypeptide sequences of the disclosure and evaluating one or more biological activities of the polypeptide as described herein and/or using any of a number of techniques well-known in the art. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, antibody variants having one or more amino acid substitutions are provided. Sites of interest for mutagenesis by substitution include the CDRs and FRs. Amino acid substitutions may be introduced into an antibody of interest and the products screened for a desired activity, e.g., retained/improved antigen binding, decreased immunogenicity, or improved ADCC or CDC.

For example, one or more amino acids can be deleted from or inserted into the aforementioned heavy and light chain variable regions. Any number of any suitable amino acids can be deleted from or inserted into the amino acid sequence of the heavy and light chain variable regions. In this respect, at least one amino acid (e.g., 2 or more, 5 or more, or 10 or more amino acids), but not more than 20 amino acids (e.g., 18 or less, 15 or less, or 12 or less amino acids), can be deleted from or inserted into the amino acid sequence of the heavy and light chain variable regions of a polypeptide described herein (e.g., any anti-LAG-3, any anti-PD-1, or any anti-TIM-3 antibody agent described herein). In some embodiments, 1-10 amino acids (e.g., 1, 2, 3, 4, 5, 6, 7, 8, 9, or 10 amino acids) are deleted from or inserted into the amino acid sequence of heavy chain variable regions and/or light chain variable regions. The amino acid(s) can be deleted from or inserted into any one of the aforementioned heavy chain variable regions and/or light chain variable regions in any suitable location. For instance, the amino acid(s) can be deleted from or inserted into a CDR (e.g., CDR1, CDR2, or CDR3) of the heavy chain variable regions and/or light chain variable regions.

In some embodiments, substitutions, insertions, or deletions may occur within one or more CDRs, wherein the substitutions, insertions, or deletions do not substantially reduce antibody binding to antigen. For example, conservative substitutions that do not substantially reduce binding affinity may be made in CDRs. Such alterations may be outside of CDR “hotspots” or SDRs. In some embodiments of the variant V_{H} and V_{L} sequences, each CDR either is unaltered, or contains no more than one, two or three amino acid substitutions.

Alterations (e.g., substitutions) may be made in CDRs, e.g., to improve antibody affinity. Such alterations may be made in CDR encoding codons with a high mutation rate during somatic maturation (See, e.g., Chowdhury, Methods Mol. Biol., 207:179-196 (2008)), and the resulting variant can be tested for binding affinity. Affinity maturation (e.g., using error-prone PCR, chain shuffling, randomization of CDRs, or oligonucleotide-directed mutagenesis) can be used to improve antibody affinity (See, e.g., Hoogenboom et al., Methods in Molecular Biology, 178:1-37 (2001)). CDR residues involved in antigen binding may be specifically identified, e.g., using alanine scanning mutagenesis or modeling (See, e.g., Cunningham and Wells, Science, 244: 1081-1085 (1989)). CDR-H3 and CDR-L3 can often be targeted. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for antibody directed enzyme prodrug therapy) or a polypeptide which increases the serum half-life of the antibody. Examples of intrasequence insertion variants of the antibody molecules include an insertion of 3 amino acids in the light chain. Examples of terminal deletions include an antibody with a deletion of 7 or less amino acids at an end of the light chain.

### Fc Region Variants

In some embodiments, one or more amino acid modifications may be introduced into the Fc region of an antibody agent provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, the present disclosure can contemplate an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. Non-limiting examples of in vitro assays to assess ADCC activity of a molecule of interest is described in U.S. Pat. Nos. 5,500,362 and 5,821,337. Alternatively, non-radioactive assays may be employed (e.g., ACTI™ and CytoTox 96® non-radioactive cytotoxicity assays). Useful effector cells for such assays can include peripheral blood mononuclear cells (PBMC) and Natural Killer (NK) cells. Alternatively, or additionally, ADCC activity of the molecule of interest may be assessed in vivo, e.g., in an animal model (See, e.g., Clynes et al., Proc. Nat'l Acad. Sci. USA, 95:652-656 (1998)). C1q binding assays may also be carried out to confirm that the antibody is able or unable bind C1q and hence contains or lacks CDC activity (See, e.g., WO06/029879, WO99/51642, and WO05/100402; U.S. Pat. No. 6,194,551; and Idusogie et al. J. Immunol. 164: 4178-4184 (2000)). To assess complement activation, a CDC assay may be performed (See, e.g., Gazzano-Santoro et al., J. Immunol. Methods, 202:163 (1996); Cragg, M. S. et al., Blood, 101:1045-1052 (2003); and Cragg et al., Blood, 103:2738-2743 (2004)). FcRn binding and in vivo clearance/half-life determinations can also be performed using methods known in the art (See, e.g., Petkova, S. B. et al., Int'l. Immunol., 18(12):1759-1769 (2006)). Antibodies with reduced effector function can include those with substitution of one or more of Fc region residues 238, 265, 269, 270, 297, 327 and 329; or two or more of amino acid positions 265, 269, 270, 297 and 327, such as an Fc mutant with substitution of residues 265 and 297 to alanine (See, e.g., U.S. Pat. Nos. 6,737,056 and 7,332,581). Antibody variants with improved or diminished binding to FcRs can also be included (See, e.g., U.S. Pat. No. 6,737,056; WO04/056312, and Shields et al., J. Biol. Chem., 9(2): 6591-6604 (2001)). In some embodiments, an antibody variant can comprise an Fc region with one or more amino acid substitutions which improve ADCC, e.g., substitutions at positions 298, 333, and/or 334 of the Fc region.

Antibodies can have increased half-lives and improved binding to the neonatal Fc receptor (FcRn) (See, e.g., US 2005/0014934). Such antibodies can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 (See, e.g., U.S. Pat. No. 7,371,826). Other examples of Fc region variants are also contemplated (See, e.g., Duncan & Winter, Nature, 322:738-40 (1988); U.S. Pat. Nos. 5,648,260 and5,624,821; and WO94/29351).

### Glycosylation Variants

The present disclosure also provides glycosylated antibody variants. In some embodiments, a provided heavy chain, light chain and/or antibody can be glycosylated on one or more sites. In some embodiments, a glycan can be N-linked to an Fc region. In some embodiments, an anti-LAG-3 antibody is glycosylated at Asn297 (Kabat numbering).

In some embodiments, the present disclosure provides a composition comprising one or more glycoforms of a heavy chain, light chain, and/or antibody agent as described herein. In some embodiments, a provided composition comprises plurality of glycoforms, present in specified absolute and/or relative amounts. In some embodiments, the present disclosure provides compositions that may be substantially free of one or more particular glycoforms of a heavy chain, light chain, and/or antibody as described herein. In some embodiments, an amount of a glycoform can be expressed as a “percent”. For any given parameter, “percent” refers to the number of moles of a particular glycan (glycan X) relative to total moles of glycans of a preparation. In some embodiments, “percent” refers to the number of moles of PNGase F-released Fc glycan X relative to total moles of PNGase F-released Fc glycans detected.

In some embodiments, the antibodies are altered to increase or decrease their glycosylation (e.g., by altering the amino acid sequence such that one or more glycosylation sites are created or removed). A carbohydrate attached to an Fc region of an antibody may be altered. Native antibodies from mammalian cells typically comprise a branched, biantennary oligosaccharide attached by an N-linkage to Asn297 of the C_{H}2 domain of the Fc region (See, e.g., Wright et al., TIBTECH,15:26-32 (1997)). The oligosaccharide can be various carbohydrates, e.g., mannose, N-acetyl glucosamine (GlcNAc), galactose, sialic acid, fucose attached to a GlcNAc in the stem of the biantennary oligosaccharide structure. Modifications of the oligosaccharide in an antibody can be made, for example, to create antibody variants with certain improved properties. Antibody glycosylation variants can have improved ADCC and/or CDC function.

In some embodiments, antibody variants provided herein can have a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose can be determined by calculating the average amount of fucose within the sugar chain at Asn297, relative to the sum of all glycostructures attached to Asn297 (See, e.g., WO 08/077546). Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. In some embodiments, the equivalent residue of Asn297 may also be located about ±7 amino acids upstream or downstream of position 297. Such fucosylation variants can have improved ADCC function (See, e.g., Pat. Pub. Nos. US 2003/0157108; US 2004/0093621; US 2003/0157108; WO00/61739; WO01/29246; US 2003/0115614; US 2002/0164328; 2004/0093621; US 2004/0132140; US 2004/0110704; US 2004/0110282; US 2004/0109865; WO03/085119; WO03/084570; WO05/035586; WO05/035778; WO05/053742; WO02/031140; Okazaki et al., J. Mol. Biol., 336:1239-1249 (2004); and Yamane-Ohnuki et al., Biotech. Bioeng., 87: 614 (2004)). Cell lines, (e.g., knockout cell lines) can be used to produce defucosylated antibodies, e.g., Lec13 CHO cells deficient in protein fucosylation and alpha-1,6-fucosyltransferase gene (FUT8) knockout CHO cells (See, e.g., Ripka et al., Arch. Biochem. Biophys., 249:533-545 (1986); Yamane-Ohnuki et al., Biotech. Bioeng., 87: 614 (2004); Kanda, Y. et al., Biotechnol. Bioeng., 94(4):680-688 (2006); WO03/085107; EP 1176195A1, WO04/056312; WO04/057002; WO03/084570; WO03/085119; WO03/05691;4 WO04/024927; and U.S. Pat. Pub. Nos. US 2003/0157108; US 2003/0115614, US 2004/093621, US 2004/110282, US 2004/110704, and US 2004/132140). Other antibody glycosylation variants can also be included (See, e.g., U.S. Pat. No. 6,602,684; Pat. Pub. No. US 2005/0123546; WO03/011878; WO97/30087; WO98/58964; and WO99/22764).

Accordingly, in some embodiments, anti-LAG-3 antibody agents of the present disclosure can be produced by a host cell with one or more of exogenous and/or high endogenous glycosyltransferase activities. Genes with glycosyltransferase activity include β(1,4)-N-acetylglucosaminyltransferase III (GnTII), α-mannosidase II (ManII), β(1,4)-galactosyltransferase (GalT), β(1,2)-N-acetylglucosaminyltransferase I (GnTI), and β(1,2)-N-acetylglucosaminyltransferase II (GnTII). The glycotranferases can comprise a fusion comprising a Golgi localization domain (See, e.g., Lifely et al., Glycobiology, 318:813-22 (1995); Schachter, Biochem. Cell Biol., 64:163-81 (1986); U.S. Prov. Pat. App. Nos. 60/495,142 and 60/441,307; Pat. Pub. Nos. US 2003/0175884 and US 2004/0241817; and WO04/065540). In some embodiments, an anti-LAG-3 antibody agent can be expressed in a host cell comprising a disrupted or deactivated glycosyltransferase gene. Accordingly, in some embodiments, the present disclosure can be directed to a host cell comprising (a) an isolated nucleic acid comprising a sequence encoding a polypeptide having a glycosyltransferase activity; and (b) an isolated polynucleotide encoding an anti-LAG-3 antibody agent of the present disclosure that binds human LAG-3. In some embodiments, the modified anti-LAG-3 antibody agent produced by the host cell has an IgG constant region or a fragment thereof comprising the Fc region. In some embodiments, the anti-LAG-3 antibody agent can be a humanized antibody or a fragment thereof comprising an Fc region. An isolated nucleic acid can be a nucleic acid molecule that has been separated from a component of its natural environment. An isolated nucleic acid can include a nucleic acid molecule contained in cells that ordinarily contain the nucleic acid molecule, but the nucleic acid molecule is present extrachromosomally or at a chromosomal location that is different from its natural chromosomal location.

In one aspect, the present disclosure provides host cell expression systems for the generation of the antibodies of the present disclosure having modified glycosylation patterns. In particular, the present disclosure provides host cell systems for the generation of glycoforms of the antibodies of the present disclosure having an improved therapeutic value. Therefore, the disclosure provides host cell expression systems selected or engineered to express a polypeptide having a glycosyltransferase activity.

Generally, any type of cultured cell line, including the cell lines discussed above, can be used as a background to engineer the host cell lines of the present disclosure. In some embodiments, CHO cells, BHK cells, NS0 cells, SP2/0 cells, YO myeloma cells, P3X63 mouse myeloma cells, PER cells, PER.C6 cells or hybridoma cells, other mammalian cells, yeast cells, insect cells, or plant cells are used as the background cell line to generate the engineered host cells of the disclosure.

The host cells which contain the coding sequence of an antibody agent of the disclosure and which express the biologically active gene products may be identified by at least four general approaches; (a) DNA-DNA or DNA-RNA hybridization; (b) the presence or absence of “marker” gene functions; (c) assessing the level of transcription as measured by the expression of the respective mRNA transcripts in the host cell; and (d) detection of the gene product as measured by immunoassay or by its biological activity.

For example, N-glycan profiling of an anti-LAG-3 antibody agent with an occupied N-glycosylation site can be used to identify glycan species present.

In embodiments, a glycosylation site is on the heavy chain of an anti-LAG-3 antibody agent. In embodiments, a glycosylation site is located at N291 on the heavy chain.

Exemplary oligosaccharide species present in a glycosylated anti-LAG-3 antibody agent include any of G0F, G1F, G2F, Man-5, G0-GN, G0F-GN, G0, G0F+GN, and G1F+GN, as well as other oligosaccharide species (e.g., other oligosaccharide species typically observed on IgGs expressed in mammalian cell culture).

In embodiments, total N-linked oligosaccharides comprise G0F.

In embodiments, total N-linked oligosaccharides comprise G1F.

In embodiments, total N-linked oligosaccharides comprise G2F.

In embodiments, total N-linked oligosaccharides comprise Man-5

In embodiments, total N-linked oligosaccharides comprise G0F and G1F. In embodiments, total N-linked oligosaccharides further comprise G0-GN, G0F-GN, G0, G0F+GN, and/or G1F+GN, or any combination thereof.

In embodiments, total N-linked oligosaccharides comprise G0F and G2F. In embodiments, total N-linked oligosaccharides further comprise G0-GN, G0F-GN, G0, G0F+GN, and/or G1F+GN, or any combination thereof.

In embodiments, total N-linked oligosaccharides comprise G0F and Man-5. In embodiments, total N-linked oligosaccharides further comprise G0-GN, G0F-GN, G0, G0F+GN, and/or G1F+GN, or any combination thereof.

In embodiments, total N-linked oligosaccharides comprise G1F and G2F. In embodiments, total N-linked oligosaccharides further comprise G0-GN, G0F-GN, G0, G0F+GN, and/or G1F+GN, or any combination thereof.

In embodiments, total N-linked oligosaccharides comprise G1F and Man-5. In embodiments, total N-linked oligosaccharides further comprise G0-GN, G0F-GN, G0, G0F+GN, and/or G1F+GN, or any combination thereof.

In embodiments, total N-linked oligosaccharides comprise G2F and Man-5. In embodiments, total N-linked oligosaccharides further comprise G0-GN, G0F-GN, G0, G0F+GN, and/or G1F+GN, or any combination thereof.

In embodiments, total N-linked oligosaccharides comprise G0F, G1F, and G2F. In embodiments, total N-linked oligosaccharides further comprise G0-GN, G0F-GN, G0, G0F+GN, and/or G1F+GN, or any combination thereof.

In embodiments, total N-linked oligosaccharides comprise G0F, G1F, and Man-5. In embodiments, total N-linked oligosaccharides further comprise G0-GN, G0F-GN, G0, G0F+GN, and/or G1F+GN, or any combination thereof.

In embodiments, total N-linked oligosaccharides comprise G0F, G2F, and Man-5. In embodiments, total N-linked oligosaccharides further comprise G0-GN, G0F-GN, G0, G0F+GN, and/or G1F+GN, or any combination thereof.

In embodiments, total N-linked oligosaccharides comprise G1F, G2F, and Man-5. In embodiments, total N-linked oligosaccharides further comprise G0-GN, G0F-GN, G0, G0F+GN, and/or G1F+GN, or any combination thereof.

In embodiments, total N-linked oligosaccharides comprise G0F, G1F, G2F, and Man-5. In embodiments, total N-linked oligosaccharides further comprise G0-GN, G0F-GN, G0, G0F+GN, and/or G1F+GN, or any combination thereof.

### Cysteine Engineered Antibody Variants

In some embodiments, it may be desirable to create cysteine engineered antibodies, e.g., “thioMAbs”, in which one or more residues of an antibody can be substituted with cysteine residues. In some embodiments, the substituted residues can occur at accessible sites of the provided antibodies. Reactive thiol groups can be positioned at sites for conjugation to other moieties, such as drug moieties or linker-drug moieties, to create an immunoconjugate. In some embodiments, any one or more of the following residues may be substituted with cysteine: V205 or equivalent residue (Kabat numbering) of the light chain; A118 or equivalent residue (EU numbering) of the heavy chain; and S400 or equivalent residue (EU numbering) of the heavy chain Fc region. Cysteine engineered antibodies may be generated as described (See, e.g., U.S. Pat. No. 7,521,541).

### Antibody Derivatives

In some embodiments, an antibody agent provided herein may be further modified to contain additional nonproteinaceous moieties that are known in the art and readily available. The moieties suitable for derivatization of the antibody can include but are not limited to water soluble polymers. Non-limiting examples of water soluble polymers can include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water.

The polymer may be of any molecular weight, and may be branched or unbranched. The number of polymers attached to the antibody may vary, and if two or more polymers are attached, they can be the same or different molecules.

In some embodiments, conjugates of an antibody and nonproteinaceous moiety that may be selectively heated by exposure to radiation are provided. In some embodiments, the nonproteinaceous moiety can be a carbon nanotube (See, e.g., Kam et al., Proc. Natl. Acad. Sci. USA, 102: 11600-11605 (2005)). The radiation may be of any wavelength, and can include, but is not limited to, wavelengths that do not harm ordinary cells, but which heat the nonproteinaceous moiety to a temperature at which cells proximal to the antibody-nonproteinaceous moiety are killed.

### Recombinant Methods and Compositions

Antibody agents, antibodies and fragments thereof may be produced using recombinant methods and compositions (See, e.g., U.S. Pat. No. 4,816,567). In some embodiments, an isolated nucleic acid encoding an anti-LAG-3 antibody agent described herein can be provided. Such nucleic acid may encode an amino acid sequence comprising the V_{L} and/or an amino acid sequence comprising the V_{H} of the antibody. In a further embodiment, one or more vectors comprising such nucleic acid can be provided. A vector can be a nucleic acid molecule capable of propagating another nucleic acid to which it is linked. The term can include the vector as a self-replicating nucleic acid structure as well as the vector incorporated into the genome of a host cell into which it has been introduced. Certain vectors can be capable of directing the expression of nucleic acids to which they are operatively linked.

In a further embodiment, a host cell comprising such nucleic acid can be provided. Host cells can be cells into which exogenous nucleic acid has been introduced, including the progeny of such cells. Host cells can include “transformants” and “transformed cells,” which can include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. In one such embodiment, a host cell can comprise (e.g., has been transformed with) a vector comprising a nucleic acid that encodes an amino acid sequence comprising the V_{L} of the antibody and an amino acid sequence comprising the V_{H} of the antibody or a first vector comprising a nucleic acid that encodes an amino acid sequence comprising the V_{L} of the antibody and a second vector comprising a nucleic acid that encodes an amino acid sequence comprising the V_{H} of the antibody. In some embodiments, the host cell can be eukaryotic, e.g., a Chinese Hamster Ovary (CHO) cell or lymphoid cell (e.g., Y0, NS0, Sp20 cell). In some embodiments, a method of making an anti-LAG-3 antibody can be provided, wherein the method can comprise culturing a host cell comprising a nucleic acid encoding the antibody, as provided above, under conditions suitable for expression of the antibody, and optionally recovering the antibody from the host cell or host cell culture medium.

For recombinant production of an anti-LAG-3 antibody agent, an isolated nucleic acid encoding an antibody, e.g., as described above, can be inserted into one or more vectors for further cloning and/or expression in a host cell. Such nucleic acid may be readily isolated and sequenced using conventional procedures.

Suitable host cells for cloning or expression of antibody-encoding vectors can include prokaryotic or eukaryotic cells described herein. For example, antibody agents may be produced in bacteria, e.g., when glycosylation and Fc effector function are not needed (See, e.g., U.S. Pat. Nos. 5,648,237, 5,789,199, and 5,840,523; Charlton, Methods in Molecular Biology, Vol. 248, pp. 245-254 (2003)). After expression, the antibody agent may be isolated from the bacterial cell paste in a soluble fraction and can be further purified.

In addition to prokaryotes, eukaryotic microbes such as filamentous fungi or yeast can be suitable cloning or expression hosts for antibody-encoding vectors (See, e.g., Gerngross, Nat. Biotech., 22:1409-1414 (2004), and Li et al., Nat. Biotech., 24:210-215 (2006)). Suitable host cells for the expression of glycosylated antibody can also be derived from multicellular organisms, including invertebrates and vertebrates. Examples of invertebrates can include plant and insect cells (See, e.g., U.S. Pat. Nos. 5,959,177, 6,040,498, 6,420,548, 7,125,978, and 6,417,429). Examples of vertebrate cells can include mammalian cell lines, monkey kidney CV1 line transformed by SV40 (COS-7); human embryonic kidney line (293 or 293T cells as described, e.g., in Graham et al., J. Gen Virol., 36:59 (1977)); baby hamster kidney cells (BHK); mouse sertoli cells (TM4 cells); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells; MRC 5 cells; FS4 cells; Chinese hamster ovary (CHO) cells, including DHFR-CHO cells; and myeloma cell lines such as Y0, NS0 and Sp2/0. (See, e.g., Yazaki and Wu, Methods in Molecular Biology, Vol. 248, pp. 255-268 (2003)).

### Assays

Anti-LAG-3 antibody agents provided herein may be identified, screened for, or characterized for their physical/chemical properties and/or biological activities by various assays known in the art.

In one aspect, an antibody of the disclosure can be tested for its antigen binding activity, e.g., by ELISA, Western blot, etc. In one aspect, competition assays may be used to identify an antibody that competes with the anti-LAG-3 antibody agents described herein for binding to LAG-3. In some embodiments, such a competing antibody binds to the same epitope (e.g., a linear or a conformational epitope) that is bound by the anti-LAG-3 antibody agents described herein. Exemplary epitope mapping methods are known (See, e.g., Morris “Epitope Mapping Protocols”, Methods in Molecular Biology, vol. 66 (1996)).

In an exemplary competition assay, immobilized LAG-3 can be incubated in a solution comprising a first labeled antibody that binds to LAG-3 and a second unlabeled antibody that is being tested for its ability to compete with the first antibody for binding to LAG-3. The second antibody may be present in a hybridoma supernatant. As a control, immobilized LAG-3 can be incubated in a solution comprising the first labeled antibody but not the second unlabeled antibody. After incubation under conditions permissive for binding of the first antibody to LAG-3, excess unbound antibody can be removed, and the amount of label associated with immobilized LAG-3 can be measured. If the amount of label associated with immobilized LAG-3 is substantially reduced in the test sample relative to the control sample, then it can indicate that the second antibody is competing with the first antibody for binding to lag-3 (See, e.g., Harlow and Lane, Antibodies: A Laboratory Manual, ch. 14 (1996)).

In one aspect, assays can be provided for identifying anti-LAG-3 antibody agents thereof having biological activity. In some embodiments, assays can be provided for identifying anti-LAG-3 antibody agents thereof having neutralization activity for LAG-3. Antibody agents having such biological activity in vivo and/or in vitro can be also provided. In some embodiments, an antibody of the disclosure can be tested for such biological activity.

The “biological activity” of an anti-LAG-3 antibody or fragment can refer to, for example, binding affinity for a particular LAG-3 epitope, neutralization or inhibition of LAG-3 binding to its receptor(s), neutralization or inhibition of LAG-3 activity in vivo (e.g., IC₅₀), pharmacokinetics, and cross-reactivity (e.g., with non-human homologs or orthologs of the LAG-3 protein, or with other proteins or tissues). Other biological properties or characteristics of an antigen-binding agent recognized in the art can include, for example, avidity, selectivity, solubility, folding, immunotoxicity, expression, and formulation. The aforementioned properties or characteristics can be observed, measured, and/or assessed using standard techniques including, but not limited to, ELISA, competitive ELISA, surface plasmon resonance analysis (BIACORE™), or Kinetic Exclusion Assay (KINEXA™), in vitro or in vivo neutralization assays, receptor-ligand binding assays, cytokine or growth factor production and/or secretion assays, and signal transduction and immunohistochemistry assays.

### Immunoconjugates

The present disclosure also provides immunoconjugates comprising an anti-LAG-3 antibody agent provided herein. An immunoconjugate can be an antibody conjugated to one or more heterologous molecule(s). For example, an immunoconjugate can comprise an anti-LAG-3 antibody conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In some embodiments, an immunoconjugate can be an antibody-drug conjugate (ADC) in which an antibody is conjugated to one or more drugs, including but not limited to a maytansinoid; an auristatin such as monomethylauristatin drug moieties DE and DF (MMAE and MMAF); a dolastatin; a calicheamicin or derivative thereof; an anthracycline such as daunomycin or doxorubicin; methotrexate; vindesine; a taxane such as docetaxel, paclitaxel, larotaxel, tesetaxel, and ortataxel; a trichothecene; and CC1065 (See, e.g., U.S. Pat. Nos. 5,208,020, 5,416,064, 5,635,483, 5,780,588, 7,498,298, 5,712,374, 5,714,586, 5,739,116, 5,767,285, 5,770,701, 5,770,710, 5,773,001, 6,630,579, and 5,877,296; EP0425235B1; Hinman et al., Cancer Res., 53:3336-3342 (1993); Lode et al., Cancer Res., 58:2925-2928 (1998); Kratz et al., Current Med. Chem., 13:477-523 (2006); Jeffrey et al., Bioorganic & Med. Chem. Letters, 16:358-362 (2006); Torgov et al., Bioconj. Chem., 16:717-721 (2005); Nagy et al., Proc. Natl. Acad. Sci. USA, 97:829-834 (2000); Dubowchik et al., Bioorg. & Med. Chem. Letters, 12:1529-1532 (2002); and King et al., J. Med. Chem., 45:4336-4343 (2002)).

In some embodiments, an immunoconjugate can comprise an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In some embodiments, an immunoconjugate can comprise an antibody as described herein conjugated to a radioactive atom to form a radioconjugate. Exemplary radioactive isotopes available for the production of radioconjugates can include At211, I131, I125, Y90, Re186, Re188, Sm153, Bi212, P32, Pb212 and radioactive isotopes of Lu. A radioconjugate can comprise a radioactive atom for scintigraphic detection (e.g., tc99m or I123, or a spin label for nuclear magnetic resonance (NMR) imaging, such as iodine-123 again, iodine-131, indium-111, fluorine-19, carbon-13, nitrogen-15, oxygen-17, gadolinium, manganese or iron).

Conjugates of an antibody and cytotoxic agent can be made using bifunctional protein coupling agents, such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (e.g., disuccinimidyl suberate), aldehydes (e.g., glutaraldehyde), bis-azido compounds (e.g., bis(p-azidobenzoyl) hexanediamine), bis-diazonium derivatives (e.g., bis-(p-diazoniumbenzoyl)-ethylenediamine), diisocyanates (such as toluene 2,6-diisocyanate), and bis-active fluorine compounds (e.g., 1,5-difluoro-2,4-dinitrobenzene). For example, a ricin immunotoxin can be prepared (See, e.g., Vitetta et al., Science, 238:1098 (1987)). Carbon-14-labeled 1-isothiocyanatobenzyl-3-methyldiethylene triaminepentaacetic acid (MX-DTPA) is an exemplary chelating agent for conjugation of radionucleotide to the antibody (See, e.g., WO94/11026). The linker may be cleavable, facilitating release of a cytotoxic drug in the cell. Exemplary cleavable linkers can include an acid-labile linker, peptidase-sensitive linker, photolabile linker, dimethyl linker and disulfide-containing linker (See, e.g., Chari et al., Cancer Res., 52:127-131 (1992); U.S. Pat. No. 5,208,020).

Immunoconjugates or ADCs herein expressly contemplate conjugates prepared with cross-linker reagents. Exemplary cross-linker reagents can include BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate).

### Methods and Compositions for Diagnostics and Detection

In some embodiments, any of the anti-LAG-3 antibody agents provided herein can be useful for detecting the presence of LAG-3 in a biological sample. Detecting can encompass quantitative or qualitative detection.

The antibody agents and compositions disclosed herein can be used for a variety of purposes, such as for monitoring the LAG-3 protein level in a subject being tested for a disease or disorder that is responsive to LAG-3 inhibition. These methods can include contacting a sample from the subject diagnosed with such disease or disorder with an antibody described herein, and detecting binding of the antibody to the sample. In some embodiments, the methods can further comprise contacting a second antibody that binds LAG-3 with the sample, and detecting binding of the second antibody. In some embodiments, the methods can further comprise contacting a second antibody agent that specifically recognizes the anti-LAG-3 antibody agent with the sample and detecting binding of the second antibody agent.

According to another embodiment, the present disclosure provides diagnostic methods. Diagnostic methods generally involve contacting a biological sample obtained from a patient, such as, for example, blood, serum, saliva, urine, sputum, a cell swab sample, or a tissue biopsy, with an anti-LAG-3 antibody agent and determining whether the antibody agent preferentially binds to the sample as compared to a control sample or predetermined cut-off value, thereby indicating the presence of the LAG-3. In this respect, the anti-LAG-3 antibody agent can be used in a method to diagnose a disorder or disease in which the improper expression (e.g., overexpression) or increased activity of LAG-3 causes or contributes to the pathological effects of the disease or disorder. In a similar manner, the anti-LAG-3 antibody agent can be used in an assay to monitor LAG-3 protein levels in a subject being tested for a disease or disorder that is responsive to LAG-3 inhibition. Research applications include, for example, methods that utilize the LAG-3-binding agent and a label to detect an LAG-3 protein in a sample, e.g., blood, serum, saliva, urine, sputum, a cell swab sample, or a tissue biopsy. The anti-LAG-3 antibody agent can be used with or without modification, such as covalent or non-covalent labeling with a detectable moiety. For example, the detectable moiety can be a radioisotope (e.g., ³H, ¹⁴C, ³²P, ³⁵s, ¹²⁵I, or ¹³¹I), a fluorescent or chemiluminescent compound (e.g., fluorescein isothiocyanate, rhodamine, or luciferin), an enzyme (e.g., alkaline phosphatase, beta-galactosidase, or horseradish peroxidase), or prosthetic groups. Any method known in the art for separately conjugating an antigen-binding agent (e.g., an antibody) to a detectable moiety may be employed in the context of the present disclosure (see, e.g., Hunter et al., Nature, 194: 495-496 (1962); David et al, Biochemistry, 13: 10144021 (1974); Pain et al, J Immunol. Metk, 40: 219-230 (1981); and Nygren, J, Histochem. and Cytochem., 30: 407-412 (1982)).

LAG-3 protein levels can be measured using the disclosed anti-LAG-3 antibody agents by any suitable methods known in the art. Such methods can include, for example, radioimmunoassay (RIA), and FACS. Normal or standard expression values of LAG-3 can be established using any suitable technique, e.g., by combining a sample comprising, or suspected of comprising, LAG-3 with a LAG-3-specific antibody agent under conditions suitable to form an antigen-antibody agent complex. The antibody agent can be directly or indirectly labeled with a detectable substance to facilitate detection of the bound or unbound antibody. Suitable detectable substances can include various enzymes, prosthetic groups, fluorescent materials, luminescent materials, and radioactive materials (see, e.g., Zola. Monoclonal Antibodies: A Manual of Techniques, CRC Press, inc. (1987)). The amount of LAG-3 polypeptide expressed in a sample is then compared with a standard value.

The anti-LAG-3 antibody agents can be provided in a kit, i.e., a packaged combination of reagents in predetermined amounts with instructions for performing a diagnostic assay. If an anti-LAG-3 antibody agent is labeled with an enzyme, the kit desirably can include substrates and cofactors required by the enzyme (e.g., a substrate precursor which provides a detectable chromophore or fluorophore). In addition, other additives may be included in the kit, such as stabilizers, buffers (e.g., a blocking buffer or lysis buffer), and the like. The relative amounts of the various reagents can be varied to provide for concentrations in solution of the reagents which substantially optimize the sensitivity of the assay. The reagents may be provided as dry powders (typically lyophilized), including excipients which on dissolution can provide a reagent solution having the appropriate concentration.

### Pharmaceutical Formulations

The present invention also provides pharmaceutical formulations (e.g., a pharmaceutically acceptable composition) comprising one or more provided anti-LAG-3 antibody agents as described herein.

In embodiments the present invention comprises any agent described herein. Such pharmaceutical compositions may optionally comprise and/or be administered in combination with one or more additional therapeutically active substances (e.g., a checkpoint inhibitor or an anticancer agent such as niraparib). In some embodiments, provided pharmaceutical compositions are useful in medicine. In some embodiments, provided pharmaceutical compositions are useful as prophylactic agents (i.e., vaccines) in the treatment or prevention of diseases and disorders such as those described herein. In some embodiments, provided pharmaceutical compositions are useful in therapeutic applications, for example in individuals suffering from or susceptible to s diseases and disorders such as those described herein.

In some embodiments, pharmaceutical compositions are formulated for administration to humans. In some embodiments, pharmaceutical compositions are formulated for administration to non-human mammals (e.g., are suitable for veterinary uses).

Pharmaceutical formulations of an anti-LAG-3 antibody agent as described herein can be prepared by mixing such antibody having the desired degree of purity with one or more optional pharmaceutically acceptable carriers (See, e.g., Remington's Pharmaceutical Sciences 16th edition, Osol, A. Ed. (1980)), in the form of lyophilized formulations or aqueous solutions.

Pharmaceutically acceptable carriers are generally nontoxic to recipients at the dosages and concentrations employed. Exemplary pharmaceutical acceptable carriers can include buffers (e.g., phosphate, citrate, and other organic acids); antioxidants (e.g., ascorbic acid and methionine); preservatives (e.g., octadecyldimethylbenzyl ammonium chloride); hexamethonium chloride; benzalkonium chloride; benzethonium chloride; phenol, butyl or benzyl alcohol; alkyl parabens (e.g., methyl or propyl paraben); catechol; resorcinol; cyclohexanol; 3-pentanol; and m-cresol; low molecular weight (less than about 10 residues) polypeptides; proteins, (e.g., serum albumin, gelatin, or immunoglobulins); hydrophilic polymers (e.g., polyvinylpyrrolidone); amino acids (e.g., glycine, glutamine, asparagine, histidine, arginine, or lysine); monosaccharides, disaccharides, and other carbohydrates (e.g., glucose, mannose, or dextrins); chelating agents (e.g., EDTA); sugars (e.g., sucrose, mannitol, trehalose or sorbitol); salt-forming counter-ions (e.g., sodium); metal complexes (e.g., Zn-protein complexes); and/or non-ionic surfactants (e.g., polyethylene glycol (PEG)). Exemplary pharmaceutically acceptable carriers herein can further include interstitial drug dispersion agents (e.g., soluble neutral-active hyaluronidase glycoproteins (sHASEGP)) (See, e.g., U.S. Pat. Pub. Nos. US 2005/0260186 and US 2006/0104968). In one aspect, a sHASEGP can be combined with one or more additional glycosaminoglycanases (e.g., chondroitinases).

In some embodiments, provided pharmaceutical compositions comprise one or more pharmaceutically acceptable excipients (e.g., preservative, inert diluent, dispersing agent, surface active agent and/or emulsifier, buffering agent, etc.). In some embodiments, pharmaceutical compositions comprise one or more preservatives. In some embodiments, pharmaceutical compositions comprise no preservative. Remington's The Science and Practice of Pharmacy, 21st Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, pharmaceutical compositions are provided in a form that can be refrigerated and/or frozen. In some embodiments, pharmaceutical compositions are provided in a form that cannot be refrigerated and/or frozen. In some embodiments, an antibody agent formulation can be lyophilized (See, e.g., U.S. Pat. No. 6,267,958). Antibody agent formulations can be aqueous (See, e.g., U.S. Pat. No. 6,171,586 and WO06/044908). In some embodiments, reconstituted solutions and/or liquid dosage forms may be stored for a certain period of time after reconstitution (e.g., 2 hours, 12 hours, 24 hours, 2 days, 5 days, 7 days, 10 days, 2 weeks, a month, two months, or longer). In some embodiments, storage of antibody compositions for longer than the specified time results in molecular degradation.

Liquid dosage forms and/or reconstituted solutions may comprise particulate matter and/or discoloration prior to administration. In some embodiments, a solution should not be used if discolored or cloudy and/or if particulate matter remains after filtration.

The formulation herein can also contain more than one active ingredient as necessary for the particular indication being treated (e.g., cancer).

Active ingredients can be entrapped in microcapsules (e.g., hydroxymethylcellulose or gelatin-microcapsules and poly-(methylmethacylate). Active ingredients can be entrapped in microcapsules in colloidal drug delivery systems (e.g., liposomes, albumin microspheres, microemulsions, nano-particles and nanocapsules) or in macroemulsions. Sustained-release preparations can be prepared. Suitable examples of sustained-release preparations can include semipermeable matrices of solid hydrophobic polymers containing the antibody, which matrices are in the form of shaped articles (e.g., films or microcapsules). The formulations to be used for in vivo administration can generally be sterile (e.g., by filtration through sterile filtration membranes).

For example, pharmaceutical compositions provided here may be provided in a sterile injectable form (e.g., a form that is suitable for subcutaneous injection or intravenous infusion). For example, in some embodiments, pharmaceutical compositions are provided in a liquid dosage form that is suitable for injection. In some embodiments, pharmaceutical compositions are provided as powders (e.g., lyophilized and/or sterilized), optionally under vacuum, which are reconstituted with an aqueous diluent (e.g., water, buffer, salt solution, etc.) prior to injection. In some embodiments, pharmaceutical compositions are diluted and/or reconstituted in water, sodium chloride solution, sodium acetate solution, benzyl alcohol solution, phosphate buffered saline, etc. In some embodiments, powder should be mixed gently with the aqueous diluent (e.g., not shaken).

Compositions of the pharmaceutical compositions described herein may be prepared by any method known or hereafter developed in the art of pharmacology. In some embodiments, such preparatory methods include the step of bringing active ingredient into association with one or more excipients and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to a dose which would be administered to a subject and/or a convenient fraction of such a dose such as, for example, one-half or one-third of such a dose.

Relative amounts of active ingredient, pharmaceutically acceptable excipient, and/or any additional ingredients in a pharmaceutical composition in accordance with the invention may vary, depending upon the identity, size, and/or condition of the subject treated and/or depending upon the route by which the composition is to be administered. By way of example, the composition may comprise between 0.1% and 100% (w/w) active ingredient.

The pharmaceutical composition can be administered to a mammal using standard administration techniques, including oral, intravenous, intraperitoneal, subcutaneous, pulmonary, transdermal, intramuscular, intranasal, buccal, sublingual, or suppository administration. The composition may be suitable for parenteral administration. The term “parenteral,” as used herein, includes intravenous, intramuscular, subcutaneous, rectal, vaginal, and intraperitoneal administration. In embodiments, the composition is administered to a mammal using peripheral systemic delivery by intravenous, intraperitoneal, or subcutaneous injection.

### Therapeutic Methods

The present disclosure also provides methods and compositions of using a LAG-3 agent (e.g., an agent that is capable of inhibiting LAG-3 signaling such as the disclosed antibody agents) to treat a disease or disorder. The present disclosure provides a composition comprising an effective amount of a LAG-3 agent (e.g., an agent that is capable of inhibiting LAG-3 signaling). In embodiments, a LAG-3 agent is the disclosed immunoglobulin heavy chain polypeptide, the disclosed immunoglobulin light chain polypeptide, the disclosed anti-LAG-3 antibody agents, the disclosed nucleic acid sequence encoding any of the foregoing, or the disclosed vector comprising the disclosed nucleic acid sequence.

As described herein, the composition can be a pharmaceutically acceptable (e.g., physiologically acceptable) composition, which comprises a carrier, e.g., a pharmaceutically acceptable (e.g., physiologically acceptable) carrier, and the disclosed amino acid sequences, antigen-binding agent, or vector. Any suitable carrier can be used within the context of the present disclosure, and such carriers are well-known in the art. The choice of carrier can be determined, in part, by the particular site to which the composition may be administered and the particular method used to administer the composition. The composition optionally can be sterile. The composition can be frozen or lyophilized for storage and reconstituted in a suitable sterile carrier prior to use. The compositions can be generated in accordance with conventional techniques described in, e.g., Remington; The Science and Practice of Pharmacy. 21st Edition, Lippincott Williams & Wilkins, Philadelphia, Pa. (2001).

In embodiments, administration of an agent described herein results in a therapeutic effect (e.g., a desired pharmacologic and/or physiologic effect). A therapeutic effect can encompass partially or completely curing a disease, relieving one or more adverse symptoms attributable to the disease, and/or delaying progression of the disease. To this end, the inventive method comprises administering a therapeutically effective amount of an anti-LAG-3 binding agent. A therapeutically effective amount can be an amount effective, at dosages and for periods of time necessary, to achieve a desired therapeutic result. A therapeutically effective amount may vary according to factors such as the disease state, age, sex, and weight of the individual, and the ability of the binding agent to elicit a desired response in the individual. For example, a therapeutically effective amount of a binding agent of the invention is an amount which decreases LAG-3 bioactivity in a human.

Alternatively, the pharmacologic and/or physiologic effect may be prophylactic, i.e., the effect of completely or partially prevents a disease or symptom thereof (e.g., delaying onset or slowing progression of a disease or symptom thereof). In this respect, the inventive method comprises administering a “prophylactically effective amount” of the binding agent. A “prophylactically effective amount” refers to an amount effective, at dosages and for periods of time necessary, to achieve a desired prophylactic result.

Thus, the present disclosure further provides a method of treating a disorder in a mammal that is responsive to LAG-3 inhibition. The method can comprise administering the aforementioned composition to a mammal having a disorder that is responsive to LAG-3 inhibition, whereupon the disorder is treated in the mammal. A disorder that is “responsive to LAG-3 inhibition” can refer to any disease or disorder in which a decrease in LAG-3 levels or activity has a therapeutic benefit in mammals, e.g., humans, or the improper expression (e.g. overexpression) or increased activity of LAG-3 causes or contributes to the pathological effects of the disease or disorder.

In an embodiment, the invention provides a method of enhancing an immune response in a mammal, or treating or preventing a disease or disorder in a mammal that is responsive to LAG-3 inhibition or neutralization, which method comprises administering to a mammal in need thereof an anti-LAG-3 binding agent or pharmaceutical composition described herein, whereupon an immune response in the mammal is enhanced, or the disease or disorder is treated in the mammal. The immune response is augmented for example by augmenting antigen specific T effector function. The antigen can be a viral (e.g., HIV), bacterial, parasitic or tumor antigen (e.g., any antigen described herein). In embodiments, an immune response is a natural immune response. By natural immune response is meant an immune response that is a result of an infection. In embodiments, an infection is a chronic infection. In embodiments, an infection is an acute infection.

Increasing or enhancing an immune response to an antigen can be measured by a number of methods known in the art. For example, an immune response can be measured by measuring any one of the following: T cell activity, T cell proliferation, T cell activation, production of effector cytokines, and T cell transcriptional profile. In embodiments, an immune response is a response induced due to a vaccination. Accordingly, in another aspect the invention provides a method of increasing vaccine efficiency by administering to the subject a monoclonal antibody or scFv antibody of the invention and a vaccine. The antibody and the vaccine are administered sequentially or concurrently. The vaccine is a tumor vaccine a bacterial vaccine or a viral vaccine.

In embodiments, methods described herein are useful for increasing T cell activation or T cell effector function in a subject.

In embodiments, methods described herein are useful for inducing an immune response in a subject.

In embodiments, methods described herein are useful for enhancing an immune response or increasing the activity of an immune cell in a subject.

In embodiments, methods described herein are useful for treating T-cell dysfunctional disorders (e.g., cancer).

In embodiments, methods described herein are useful for reducing tumors or inhibiting the growth of tumor cells in a subject.

Thus, the inventive method can be used to treat any type of infectious disease (i.e., a disease or disorder caused by a bacterium, a virus, a fungus, or a parasite). Examples of infectious diseases that can be treated by the inventive method include, but are not limited to, diseases caused by a human immunodeficiency virus (HIV), a respiratory syncytial virus (RSV), an influenza virus, a dengue virus, a hepatitis B virus (HBV, or a hepatitis C virus (HCV)). When an inventive method treats an infectious disease, an antibody agent can be administered in combination with at least one anti-bacterial agent or at least one anti-viral agent. In this respect, the anti-bacterial agent can be any suitable antibiotic known in the art. The anti-viral agent can be any vaccine of any suitable type that specifically targets a particular virus (e.g., live-attenuated vaccines, subunit vaccines, recombinant vector vaccines, and small molecule anti-viral therapies (e.g., viral replication inhibitors and nucleoside analogs).

In embodiments, the inventive methods can be used to treat any type of autoimmune disease (i.e., as disease or disorder caused by immune system over-activity in which the body attacks and damages its own tissues), such as those described in, for example, MacKay I. R. and Rose N. R., eds., The Autoimmune Diseases, Fifth Edition, Academic Press, Waltham, Mass. (2014). Examples of autoimmune diseases that can be treated by the inventive method include, but are not limited to, multiple sclerosis, type 1 diabetes mellitus, rheumatoid arthritis, scleroderma, Crohn's disease, psoriasis, systemic lupus erythematosus (SLE), and ulcerative colitis. When the inventive method treats an autoimmune disease, an antibody agent described herein can be used in combination with an anti-inflammatory agent including, for example, corticosteroids (e.g., prednisone and fluticasone) and non-steroidal anti-inflammatory drugs (NSAIDs) (e.g., aspirin, ibuprofen, and naproxen).

Other exemplary disorders that are responsive to LAG-3 inhibition can include, for example, cancer.

Accordingly, in one aspect, the invention provides methods for preventing, treating or alleviating a cell proliferative disease or disorder or a symptom of said disease or disorder in a subject (e.g., a subject having a cancer or a cell proliferative disease or disorder or a subject at risk of a cancer or a cell proliferative disease or disorder). Subjects at risk for cell proliferation-related diseases or disorders include patients who have a family history of cancer or a subject exposed to a known or suspected cancer-causing agent. Administration of a prophylactic agent can occur prior to the manifestation of the disease or disorder such that it is prevented or, alternatively, delayed in its progression.

The inventive method can be used to treat any type of cancer known in the art.

In embodiments, a cancer is an advanced cancer. In some embodiments, a cancer is a stage II, stage III or stage IV cancer. In some embodiments, a cancer is a stage II cancer. In some embodiments, a cancer is a stage III cancer. In some embodiments, a cancer is a stage IV cancer.

In embodiments, a cancer is a metastatic cancer.

In embodiments, methods described herein are useful for reducing tumors or inhibiting the growth of tumor cells in a subject.

In embodiments, a cancer is a recurrent cancer.

Cancers that can be treated with methods described herein also include cancers associated with a high tumor mutation burden (TMB), cancers that microsatellite stable (MSS), cancers that are characterized by microsatellite instability, cancers that have a high microsatellite instability status (MSI-H), cancers that have low microsatellite instability status (MSI-L), cancers associated with high TMB and MSI-H, cancers associated with high TMB and MSI-L or MSS), cancers having a defective DNA mismatch repair system, cancers having a defect in a DNA mismatch repair gene, hypermutated cancers, cancers having homologous recombination repair deficiency/homologous repair deficiency (“HRD”) or characterized by a homologous recombination repair (HRR) gene mutation or deletion, cancers comprising a mutation in polymerase delta (POLD), and cancers comprising a mutation in polymerase epsilon (POLE). In embodiments, a cancer is a cancer is characterized by a homologous recombination repair (HRR) gene mutation or deletion, a mutation in the DNA damage repair (DDR) pathway, BRCA deficiency, isocitrate dehydrogenase (IDH) mutation, and/or a chromosomal translocation. In embodiments, a cancer is a hypermutant cancer, a MSI-H cancer, a MSI-L cancer, or a MSS cancer. In embodiments, a cancer is characterized by one or more of these characteristics.

In some embodiments, a tumor to be treated is characterized by microsatellite instability. In some embodiments, a tumor is characterized by microsatellite instability high status (MSI-H). Microsatellite instability (“MSI”) is or comprises a change that in the DNA of certain cells (such as tumor cells) in which the number of repeats of microsatellites (short, repeated sequences of DNA) is different than the number of repeats that was contained in the DNA from which it was inherited. About 15% of sporadic colorectal cancers (CRC) harbor widespread alterations in the length of microsatellite (MS) sequences, known as microsatellite instability (MSI) (Boland and Goel, 2010). Sporadic MSI CRC tumors display unique clinicopathological features including near-diploid karyotype, higher frequency in older populations and in females, and a better prognosis (de la Chapelle and Hampel, 2010; Popat et al., 2005). MSI is also present in other tumors, such as in endometrial cancer (EC) of the uterus, the most common gynecological malignancy (Duggan et al., 1994). The same reference Bethesda panel originally developed to screen an inherited genetic disorder (Lynch syndrome) (Umar et al., 2004) is currently applied to test MSI for CRCs and ECs. However, the genes frequently targeted by MSI in CRC genomes rarely harbor DNA slippage events in EC genomes (Gunn et al., 1999).

Microsatellite instability arises from a failure to repair replication-associated errors due to a defective DNA mismatch repair (MMR) system. This failure allows persistence of mismatch mutations all over the genome, but especially in regions of repetitive DNA known as microsatellites, leading to increased mutational load. It has been demonstrated that at least some tumors characterized by MSI-H have improved responses to certain PD-1 agents (Le et al., (2015) N. Engl. J. Med. 372(26):2509-2520; Westdorp et al., (2016) Cancer Immunol. Immunother. 65(10):1249-1259). In some embodiments, a cancer has a microsatellite instability of high microsatellite instability (e.g., MSI-H status). In some embodiments, a cancer has a microsatellite instability status of low microsatellite instability (e.g., MSI-Low). In some embodiments, a cancer has a microsatellite instability status of microsatellite stable (e.g., MSS status). In some embodiments microsatellite instability status is assessed by a next generation sequencing (NGS)-based assay, an immunohistochemistry (IHC)-based assay, and/or a PCR-based assay. In some embodiments, microsatellite instability is detected by NGS. In some embodiments, microsatellite instability is detected by IHC. In some embodiments, microsatellite instability is detected by PCR.

In embodiments, a patient has a MSI-L cancer.

In embodiments, a patient has a MSI-H cancer. In some embodiments, a patient has a MSI-H solid tumor. In embodiments, a MSI-H cancer is MSI-H endometrial cancer. In embodiments, a MSI-H cancer is a solid tumor. In embodiments, a MSI-H cancer is a metastatic tumor. In embodiments, a MSI-H cancer is endometrial cancer. In embodiments, a MSI-H cancer is a non-endometrial cancer. In embodiments, a MSI-H cancer is colorectal cancer.

In embodiments, a patient has a MSS cancer. In embodiments, a MSS cancer is MSS endometrial cancer.

In embodiments, a cancer is associated with a POLE (DNA polymerase epsilon) mutation (i.e., a cancer is a POLE-mutant cancer). In embodiments, a POLE mutation is a mutation in the exonuclease domain. In embodiments, a POLE mutation is a germline mutation. In embodiments, a POLE mutation is a sporadic mutation. In embodiments, a MSI cancer also is associated with a POLE mutation. In embodiments, a MSS cancer also is associated with a POLE mutation. In embodiments, a POLE mutation is identified using sequencing. In embodiments, a POLE-mutant cancer is endometrial cancer. In embodiments, a POLE-mutant cancer is colon cancer. In embodiments, a POLE-mutant cancer is pancreatic cancer, ovarian cancer, or cancer of the small intestine.

In embodiments, a cancer is associated with a POLD (DNA polymerase delta) mutation (i.e., a cancer is a POLD-mutant cancer). In embodiments, a POLD mutation is a mutation in the exonuclease domain. In embodiments, a POLD mutation is a somatic mutation. In embodiments, a POLD mutation is a germline mutation. In embodiments, a POLD-mutant cancer is identified using sequencing. In embodiments, a POLD-mutant cancer is endometrial cancer. In embodiments, a POLD-mutant cancer is colorectal cancer. In embodiments, a POLD-mutant cancer is brain cancer.

In embodiments, a cancer has a defective DNA mismatch repair system (e.g., is a a mismatch repair deficient (MMRd) cancer). In embodiments, a cancer has a defect in a DNA mismatch repair gene. In some embodiments, a patient has a mismatch repair deficient cancer.

In embodiments, a MMRd cancer is colorectal cancer.

In embodiments, a cancer is a hypermutated cancer.

In embodiments, a cancer has homologous recombination repair deficiency/homologous repair deficiency (“HRD”) or is characterized by a homologous recombination repair (HRR) gene mutation or deletion.

In embodiments, a cancer (e.g., a MMRd cancer) is characterized by a high tumor mutation burden (i.e., a cancer is a high TMB cancer). In some embodiments, the cancer is associated with high TMB and MSI-H. In some embodiments, the cancer is associated with high TMB and MSI-L or MSS. In some embodiments, the cancer is endometrial cancer associated with high TMB. In some related embodiments, the endometrial cancer is associated with high TMB and MSI-H. In some related embodiments, the endometrial cancer is associated with high TMB and MSI-L or MSS. In embodiments, a high TMB cancer is colorectal cancer. In embodiments, a high TMB cancer is lung cancer (e.g., small cell lung cancer (SCLC) or non-small cell lung cancer (NSCLC) such as squamous NSCLC or non-squamous NSCLC). In embodiments, a high TMB cancer is melanoma. In embodiments, a high TMB cancer is urothelial cancer.

In embodiments, a patient has a cancer with elevated expression of tumor-infiltrating lymphocytes (TILs), i.e., a patient has a high-TIL cancer. In embodiments, a high-TIL cancer is breast cancer (e.g., triple negative breast cancer (TNBC) or HER2-positive breast cancer). In embodiments, a high-TIL cancer is a metastatic cancer (e.g., a metastatic breast cancer).

Non-limiting examples of cancers to be treated by the methods of the present disclosure can include melanoma (e.g., metastatic malignant melanoma), renal cancer (e.g. clear cell carcinoma), prostate cancer (e.g. hormone refractory prostate adenocarcinoma), pancreatic adenocarcinoma, breast cancer, colon cancer, lung cancer (e.g. non-small cell lung cancer), esophageal cancer, head and neck cancer, squamous cell carcinoma, liver cancer, ovarian cancer, cervical cancer, thyroid cancer, glioblastoma, glioma, leukemia, lymphoma, mesothelioma, sarcoma and other neoplastic malignancies. Additionally, the invention includes refractory or recurrent malignancies whose growth may be inhibited using the methods of the invention. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, head and neck, tongue, larynx, and gullet), and adenocarcinoma (for example, prostate, small intestine, endometrium, cervical canal, large intestine, lung, pancreas, gullet, intestinum rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer is a melanoma, renal cell carcinoma., lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma (see, e.g., Bhatia et al, Curr. Oncol. Rep., 13(6): 488-497 (2011)).

In embodiments, a cancer is acute myeloid leukemia (“AML”), acute lymphoblastic leukemia (“ALL”), adenocarcinoma, adenocarcinoma of the lung, adrenocortical carcinoma, anal cancer (e.g,. squamous cell carcinoma of the anus), appendiceal cancer, B-cell derived leukemia, B-cell derived lymphoma, bladder cancer, brain cancer, breast cancer (e.g., triple negative breast cancer (TNBC) or non-triple negative breast cancer), cancer of the fallopian tube(s), cancer of the testes, cerebral cancer, cervical cancer (e.g., squamous cell carcinoma of the cervix), cholangiocarcinoma, choriocarcinoma, chronic myelogenous leukemia, a CNS tumor, colon adenocarcinoma, colon cancer or colorectal cancer (e.g., colon adenocarcinoma), diffuse intrinsic pontine glioma (DIPG), diffuse large B cell lymphoma (“DLBCL”), embryonal rhabdomyosarcoma (ERMS), endometrial cancer, epithelial cancer, esophageal cancer (e.g., squamous cell carcinoma of the esophagus), Ewing's sarcoma, eye cancer (e.g., uveal melanoma), follicular lymphoma (“FL”), gall bladder cancer, gastric cancer, gastrointestinal cancer, glioblastoma multiforme, glioma (e.g., lower grade glioma), head and neck cancer (e.g., squamous cell carcinoma of the head and neck (SCHNC)), a hematological cancer, hepatocellular cancer, Hodgkin's lymphoma (HL)/primary mediastinal B-cell lymphoma, kidney cancer (e.g., kidney clear cell cancer, kidney papillary cancer, or kidney chromophobe cancer), large B-cell lymphoma, laryngeal cancer, leukemia, liver cancer, lung cancer (e.g., non-small cell lung cancer (NSCLC), small cell lung cancer, lung adenocarcinoma, or squamous cell carcinoma of the lung), lymphoma, melanoma, Merkel cell carcinoma, mesothelioma, monocytic leukemia, multiple myeloma, myeloma, a neuroblastic-derived CNS tumor (e.g., neuroblastoma (NB)), non-Hodgkin's lymphoma (NHL), non-small cell lung cancer (NSCLC), oral cancer, osteosarcoma, ovarian cancer, ovarian carcinoma, pancreatic cancer, peritoneal cancer, pheocromocytoma, primary peritoneal cancer, prostate cancer, relapsed or refractory classic Hodgkin's Lymphoma (cHL), renal cancer (e.g., renal cell carcinoma), rectal cancer (rectum carcinoma), salivary gland cancer (e.g., a salivary gland tumor), sarcoma, skin cancer, small cell lung cancer, small intestine cancer, squamous cell carcinoma of the penis, soft tissue sarcoma, squamous cell carcinoma of the esophagus, squamous cell carcinoma of the head and neck (SCHNC), squamous cell carcinoma of the lung, stomach cancer, T-cell derived leukemia, T-cell derived lymphoma, testicular tumor, thymic cancer, a thymoma, thyroid cancer (thyroid carcinoma), uveal melanoma, urothelial cell carcinoma, uterine cancer (e.g., uterine endometrial cancer or uterine sarcoma such as uterine carcinosarcoma), vaginal cancer (e.g., squamous cell carcinoma of the vagina), vulvar cancer (e.g., squamous cell carcinoma of the vulva), or Wilms tumor.

In embodiments, a cancer is adenocarcinoma, endometrial cancer, breast cancer, ovarian cancer, cervical cancer, fallopian tube cancer, testicular cancer, primary peritoneal cancer, colon cancer, colorectal cancer, stomach cancer, small intestine cancer, squamous cell carcinoma of the anus, squamous cell carcinoma of the penis, squamous cell carcinoma of the cervix, squamous cell carcinoma of the vagina, squamous cell carcinoma of the vulva, soft tissue sarcoma, melanoma, renal cell carcinoma, lung cancer, non-small cell lung cancer, adenocarcinoma of the lung, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, a hematological cancer, multiple myeloma, B-cell lymphoma, T-cell lymphoma, Hodgkin's lymphoma/primary mediastinal B-cell lymphoma, chronic myelogenous leukemia, acute myeloid leukemia, acute lymphoblastic leukemia, non-Hodgkin's lymphoma, neuroblastoma, a CNS tumor, diffuse intrinsic pontine glioma (DIPG), Ewing's sarcoma, embryonal rhabdomyosarcoma, osteosarcoma, or Wilms tumor. In embodiments, the cancer is MSS or MSI-L, is characterized by microsatellite instability, is MSI-H, has high TMB, has high TMB and is MSS or MSI-L, has high TMB and is MSI-H, has a defective DNA mismatch repair system, has a defect in a DNA mismatch repair gene, is a hypermutated cancer, is an HRD or HRR cancer, comprises a mutation in polymerase delta (POLD), or comprises a mutation in polymerase epsilon (POLE).

In embodiments, a cancer is large B-cell lymphoma, thymoma, acute myeloid leukemia, testicular tumor, lung adenocarcinoma, non-small cell lung cancer, kidney clear cell cancer, breast cancer, triple negative breast cancer (TNBC), non-triple negative breast cancer (non-TNBC), gastric cancer, lung squamous cell cancer, mesothelioma, pancreatic cancer, cervical cancer, head and neck cancer, melanoma, hepatocellular carcinoma, nasopharyngeal cancer, esophageal cancer, colon adenocarcinoma, colorectal cancer, rectum carcinoma, cholangiocarcinoma, uterine endometrial cancer, sarcoma, bladder cancer, thyroid carcinoma, kidney papillary cancer, glioblastoma multiforme, liver cancer, uterine carcinosarcoma, pheocromocytoma, lower grade glioma, kidney chromophobe, adrenocortical cancer, or uveal melanoma.

In other embodiments, a cancer is a head and neck cancer, a lung cancer (e.g., a non-small cell lung cancer (NSCLC)), a renal cancer, a bladder cancer, a melanoma, Merkel cell carcinoma, a cervical cancer, a vaginal cancer, a vulvar cancer, a uterine cancer, a endometrial cancer, an ovarian cancer, a fallopian tube cancer, a breast cancer, a prostate cancer, a salivary gland tumor, a thymoma, a adrenocortical carcinoma, a esophageal cancer, a gastric cancer, a colorectal cancer, an appendiceal cancer, a urothelial cell carcinoma, or a squamous cell carcinoma (e.g., of the lung; of the anogenital region including anus, penis, cervix, vagina, or vulva; or of the esophagus).

In some embodiments, a cancer for treatment in the context of the present disclosure is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma.

In embodiments a cancer is a lymphoma such as Hodgkin's disease, non-Hodgkin's Lymphoma, Multiple myeloma, Waldenstrom's macroglobulinemia, Heavy chain disease and Polycythemia vera.

In embodiments, a cancer is a squamous cell carcinoma. In embodiments, a cancer is squamous cell carcinoma of the lung. In embodiments, a cancer is squamous cell carcinoma of the esophagus. In embodiments, a cancer is squamous cell carcinoma of the anogenital region (e.g., of the anus, penis, cervix, vagina, or vulva). In embodiments, a cancer is head and neck squamous cell carcinoma (HNSCC).

In embodiments, a cancer is bladder cancer, breast cancer (e.g., triple negative breast cancer (TNBC)), cancer of the fallopian tube(s), cholagiocarcinoma, colon adenocarcinoma, endometrial cancer, esophageal cancer, Ewing's sarcoma, gastric cancer, kidney clear cell cancer, lung cancer (e.g., lung adenocarcinoma or lung squamous cell cancer), mesothelioma, ovarian cancer, pancreatic cancer, peritoneal cancer, prostate cancer, uterine endometrial cancer, or uveal melanoma. In embodiments, a cancer is ovarian cancer, cancer of the fallopian tube(s), or peritoneal cancer. In embodiments, a cancer is breast cancer (e.g., TNBC). In embodiments, a cancer is lung cancer (e.g., non-small cell lung cancer). In embodiments, a cancer is prostate cancer.

In embodiments, a cancer is a CNS or brain cancer such as neuroblastoma (NB), glioma, diffuse intrinsic pontine glioma (DIPG), pilocytic astrocytoma, astrocytoma, anaplastic astrocytoma, glioblastoma multiforme, medulloblastoma, craniopharyngioma, ependymoma, pinealoma, hemangioblastoma, acoustic neuroma, oligodendroglioma, meningioma, vestibular schwannoma, adenoma, metastatic brain tumor, meningioma, spinal tumor, or medulloblastoma. In embodiments, a cancer is a CNS tumor.

In other embodiments, a cancer is melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma (see, e.g., Bhatia et al., Curr. Oncol. Rep., 13(6): 488-497 (2011)).

In some embodiments, a patient or population of patients have a hematological cancer. In some embodiments, the patient has a hematological cancer such as diffuse large B cell lymphoma (“DLBCL”), Hodgkin's lymphoma (“HL”), Non-Hodgkin's lymphoma (“NHL”), follicular lymphoma (“FL”), acute myeloid leukemia (“AML”), acute lymphoblastic leukemia (“ALL”), or multiple myeloma (“MM”). In embodiments, a cancer is a blood-borne cancer such as acute lymphoblastic leukemia(“ALL”), acute lymphoblastic B-cell leukemia, acute lymphoblastic T-cell leukemia, acute myeloblastic leukemia (“AML”), acute lymphoblastic leukemia (“ALL”), acute promyelocytic leukemia(“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia(“CML”), chronic lymphocytic leukemia(“CLL”), hairy cell leukemia and multiple myeloma; acute and chronic leukemias such as lymphoblastic, myelogenous, lymphocytic, and myelocytic leukemias. In embodiments, a hematological cancer is a lymphoma (e.g., Hodgkin's lymphoma (e.g., relapsed or refractory classic Hodgkin's Lymphoma (cHL), non-Hodgkin's lymphoma, diffuse large B-cell lymphoma, or precursor T-lymphoblastic lymphoma), lymphoepithelial carcinoma, or malignant histiocytosis.

In some embodiments, a patient or population of patients have a solid tumor. In embodiments, a cancer is a solid tumor such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, osteosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, uterine cancer, testicular cancer, non small cell lung cancer (NSCLC), small cell lung carcinoma, bladder carcinoma, lung cancer, epithelial carcinoma, skin cancer, melanoma, neuroblastoma (NB), or retinoblastoma. In some embodiments, the tumor is an advanced stage solid tumor. In some embodiments, the tumor is a metastatic solid tumor. In some embodiments, the patient has a MSI-H solid tumor. In embodiments, a solid tumor is a MSS solid tumor. In embodiments, a solid tumor is a POLE-mutant solid tumor. In embodiments, a solid tumor is a MSS solid tumor. In embodiments, a solid tumor is a POLD-mutant solid tumor.

In some embodiments, a patient or population of patients to be treated by the methods of the present invention have or are susceptible to cancer, such as a head and neck cancer, a lung cancer (e.g., a non-small cell lung cancer (NSCLC)), a renal cancer, a bladder cancer, a melanoma, Merkel cell carcinoma, a cervical cancer, a vaginal cancer, a vulvar cancer, a uterine cancer, a endometrial cancer, an ovarian cancer, a fallopian tube cancer, a breast cancer, a prostate cancer, a salivary gland tumor, a thymoma, a adrenocortical carcinoma, a esophageal cancer, a gastric cancer, a colorectal cancer, an appendiceal cancer, a urothelial cell carcinoma, or a squamous cell carcinoma (e.g., of the lung; of the anogenital region including anus, penis, cervix, vagina, or vulva; or of the esophagus). In some embodiments, a patient or population of patients to be treated by the methods of the present invention have or are susceptible to lung cancer (e.g., NSCLC), renal cancer, melanoma, cervical cancer, colorectal cancer, or endometrial cancer (e.g., MSS endometrial cancer or MSI-H endometrial cancer).

In some embodiments, a patient or population of patients to be treated by the methods of the present invention have or are susceptible to non-small cell lung cancer (NSCLC), a hepatocellular cancer, a renal cancer, a melanoma, a cervical cancer, a colorectal cancer, a squamous cell carcinoma of the anogenital region (e.g., squamous cell carcinoma of the anus, penis, cervix, vagina, or vulva), a head and neck cancer, a triple negative breast cancer, an ovarian cancer or a endometrial cancer. In some embodiments, a patient has an advanced stage solid tumor, such as a non-small cell lung cancer (NSCLC), a hepatocellular cancer, a renal cancer, a melanoma, a cervical cancer, a colorectal cancer, a squamous cell carcinoma of the anogenital region (e.g., squamous cell carcinoma of the anus, penis, cervix, vagina, or vulva), a head and neck cancer, a triple negative breast cancer, an ovarian cancer or a endometrial cancer. In some embodiments, a patient has an advanced stage solid tumor with microsatellite instability.

In some embodiments, a cancer is a gynecologic cancer (i.e., a cancer of the female reproductive system such as ovarian cancer, fallopian tube cancer, cervical cancer, vaginal cancer, vulvar cancer, uterine cancer, or primary peritoneal cancer, or breast cancer). In some embodiments, cancers of the female reproductive system include, but are not limited to, ovarian cancer, cancer of the fallopian tube(s), peritoneal cancer, and breast cancer.

In embodiments, a cancer is ovarian cancer (e.g., serous or clear cell ovarian cancer). In embodiments, a cancer is fallopian tube cancer (e.g., serous or clear cell fallopian tube cancer). In embodiments, a cancer is primary peritoneal cancer (e.g., serous or clear cell primary peritoneal cancer).

In some embodiments, an ovarian cancer is an epithelial carcinoma. Epithelial carcinomas make up 85% to 90% of ovarian cancers. While historically considered to start on the surface of the ovary, new evidence suggests at least some ovarian cancer begins in special cells in a part of the fallopian tube. The fallopian tubes are small ducts that link a woman's ovaries to her uterus that are a part of a woman's reproductive system. In a normal female reproductive system, there are two fallopian tubes, one located on each side of the uterus. Cancer cells that begin in the fallopian tube may go to the surface of the ovary early on. The term ‘ovarian cancer’ is often used to describe epithelial cancers that begin in the ovary, in the fallopian tube, and from the lining of the abdominal cavity, call the peritoneum. In some embodiments, the cancer is or comprises a germ cell tumor. Germ cell tumors are a type of ovarian cancer develops in the egg-producing cells of the ovaries. In some embodiments, a cancer is or comprises a stromal tumor. Stromal tumors develop in the connective tissue cells that hold the ovaries together, which sometimes is the tissue that makes female hormones called estrogen. In some embodiments, a cancer is or comprises a granulosa cell tumor. Granulosa cell tumors may secrete estrogen resulting in unusual vaginal bleeding at the time of diagnosis. In some embodiments, a gynecologic cancer is associated with homologous recombination repair deficiency/homologous repair deficiency (“HRD”), a homologous recombination repair (HRR) gene mutation or deletion, and/or BRCA1/2 mutation(s). In some embodiments, a gynecologic cancer is platinum-sensitive. In some embodiments, a gynecologic cancer has responded to a platinum-based therapy. In some embodiments, a gynecologic cancer has developed resistance to a platinum-based therapy. In some embodiments, a gynecologic cancer has at one time shown a partial or complete response to platinum-based therapy (e.g., a partial or complete response to the last platinum-based therapy or to the penultimate platinum-based therapy). In some embodiments, a gynecologic cancer is now resistant to platinum-based therapy.

In embodiments, a cancer is a breast cancer. Usually breast cancer either begins in the cells of the milk producing glands, known as the lobules, or in the ducts. Less commonly breast cancer can begin in the stromal tissues. These include the fatty and fibrous connective tissues of the breast. Over time the breast cancer cells can invade nearby tissues such the underarm lymph nodes or the lungs in a process known as metastasis. The stage of a breast cancer, the size of the tumor and its rate of growth are all factors which determine the type of treatment that is offered. Treatment options include surgery to remove the tumor, drug treatment which includes chemotherapy and hormonal therapy, radiation therapy and immunotherapy. The prognosis and survival rate varies widely; the five year relative survival rates vary from 98% to 23% depending on the type of breast cancer that occurs. Breast cancer is the second most common cancer in the world with approximately 1.7 million new cases in 2012 and the fifth most common cause of death from cancer, with approximately 521,000 deaths. Of these cases, approximately 15% are triple-negative, which do not express the estrogen receptor, progesterone receptor (PR) or HER2. In some embodiments, triple negative breast cancer (TNBC) is characterized as breast cancer cells that are estrogen receptor expression negative (<1% of cells), progesterone receptor expression negative (<1% of cells), and HER2-negative.

In embodiments, a cancer is ER-positive breast cancer, ER-negative breast cancer, PR-positive breast cancer, PR-negative breast cancer, HER2-positive breast cancer, HER2-negative breast cancer, BRCA1/2-positive breast cancer, BRCA1/2-negative cancer, or triple negative breast cancer (TNBC). In embodiments, a cancer is triple negative breast cancer (TNBC). In some embodiments, a breast cancer is a metastatic breast cancer. In some embodiments, a breast cancer is an advanced breast cancer. In some embodiments, a cancer is a stage II, stage III or stage IV breast cancer. In some embodiments, a cancer is a stage IV breast cancer. In some embodiments, a breast cancer is a triple negative breast cancer. In embodiments, a breast cancer is a metastatic breast cancer. In embodiments, a breast cancer is a MSI-H breast cancer. In embodiments, a breast cancer is a MSS breast cancer. In embodiments, a breast cancer is a POLE-mutant breast cancer. In embodiments, a breast cancer is a POLD-mutant breast cancer. In embodiments, a breast cancer is a high TMB breast cancer. In embodiments, a breast cancer is associated with homologous recombination repair deficiency/homologous repair deficiency (“HRD”) or is characterized by a homologous recombination repair (HRR) gene mutation or deletion.

In some embodiments, a patient or a population of patients to be treated by the methods of the present disclosure have or are susceptible to endometrial cancer (“EC”). Endometrial carcinoma is the most common cancer of the female genital, tract accounting for 10-20 per 100,000 person-years. The annual number of new cases of endometrial cancer (EC) is estimated at about 325 thousand worldwide. Further, EC is the most commonly occurring cancer in post-menopausal women. About 53% of endometrial cancer cases occur in developed countries. In 2015, approximately 55,000 cases of EC were diagnosed in the U.S. and no targeted therapies are currently approved for use in EC. There is a need for agents and regimens that improve survival for advanced and recurrent EC in 1L and 2L settings. Approximately 10,170 people are predicted to die from EC in the U.S. in 2016. The most common histologic form is endometrioid adenocarcinoma, representing about 75-80% of diagnosed cases. Other histologic forms include uterine papillary serous (less than 10%), clear cell 4%, mucinous 1%, squamous less than 1% and mixed about 10%.

From the pathogenetic point of view, EC falls into two different types, so-called types I and II. Type I tumors are low-grade and estrogen-related endometrioid carcinomas (EEC) while type II are non-endometrioid (NEEC) (mainly serous and clear cell) carcinomas. The World Health Organization has recently updated the pathologic classification of EC, recognizing nine different subtypes of EC, but EEC and serous carcinoma (SC) account for the vast majority of cases. EECs are estrogen-related carcinomas, which occur in perimenopausal patients, and are preceded by precursor lesions (endometrial hyperplasia/endometrioid intraepithelial neoplasia). Microscopically, lowgrade EEC (EEC 1-2) contains tubular glands, somewhat resembling the proliferative endometrium,with architectural complexity with fusion of the glands and cribriform pattern. High-grade EEC shows solid pattern of growth. In contrast, SC occurs in postmenopausal patients in absence of hyperestrogenism. At the microscope, SC shows thick, fibrotic or edematous papillae with prominent stratification of tumor cells, cellular budding, and anaplastic cells with large, eosinophilic cytoplasms. The vast majority of EEC are low grade tumors (grades 1 and 2), and are associated with good prognosis when they are restricted to the uterus. Grade 3 EEC (EEC3) is an aggressive tumor, with increased frequency of lymph node metastasis. SCs are very aggressive, unrelated to estrogen stimulation, mainly occurring in older women. EEC 3 and SC are considered high-grade tumors. SC and EEC3 have been compared using the surveillance, epidemiology and End Results (SEER) program data from 1988 to 2001. They represented 10% and 15% of EC respectively, but accounted for 39% and 27% of cancer death respectively. Endometrial cancers can also be classified into four molecular subgroups: (1) ultramutated/POLE-mutant; (2) hypermutated MSI+ (e.g., MSI-H or MSI-L); (3) copy number low/microsatellite stable (MSS); and (4) copy number high/serous-like. Approximately 28% of cases are MSI-high. (Murali, Lancet Oncol. (2014). In some embodiments, a patient has a mismatch repair deficient subset of 2L endometrial cancer. In embodiments, an endometrial cancer is metastatic endometrial cancer. In embodiments, a patient has a MSS endometrial cancer. In embodiments, a patient has a MSI-H endometrial cancer. In embodiments, an endometrial cancer is a MSI-L endometrial cancer. In embodiments, an endometrial cancer is a MSS endometrial cancer. In embodiments, an endometrial cancer is a POLE-mutant endometrial cancer (e.g., a MSI-H endometrial cancer comprising a POLE mutation). In embodiments, an endometrial cancer is a POLD-mutant endometrial cancer (e.g., MSI-H endometrial cancer comprising a POLD mutation). In embodiments, an endometrial cancer is a high TMB endometrial cancer. In embodiments, an endometrial cancer is associated with homologous recombination repair deficiency/homologous repair deficiency (“HRD”) or is characterized by a homologous recombination repair (HRR) gene mutation or deletion.

In embodiments a cancer is a gonadal tumor.

In embodiments, a cancer is a non-endometrial cancer (e.g., a non-endometrial solid tumor). In embodiments, a non-endometrial cancer is an advanced cancer. In embodiments, a non-endometrial cancer is a metastatic cancer. In embodiments, a non-endometrial cancer is a MSI-H cancer. In embodiments, a non-endometrial cancer is a MSI-L endometrial cancer. In embodiments, a non-endometrial cancer is a MSS cancer. In embodiments, a non-endometrial cancer is a POLE-mutant cancer (e.g., a MSI-H non-endometrial cancer comprising a POLE mutation). In embodiments, a non-endometrial cancer is a POLD-mutant cancer (e.g., a MSI-H non-endometrial cancer comprising a POLD mutation). In embodiments, a non-endometrial cancer is a solid tumor (e.g., a MSS solid tumor, a MSI-H solid tumor, a POLD mutant solid tumor, or a POLE-mutant solid tumor). In embodiments, a non-endometrial cancer is a high TMB cancer. In embodiments, a non-endometrial cancer is associated with homologous recombination repair deficiency/homologous repair deficiency (“HRD”) or is characterized by a homologous recombination repair (HRR) gene mutation or deletion.

In embodiments, a cancer is a lung cancer. In embodiments, a lung cancer is a squamous cell carcinoma of the lung. In embodiments, a lung cancer is small cell lung cancer (SCLC). In embodiments, a lung cancer is non-small cell lung cancer (NSCLC) such as squamous NSCLC. In embodiments, a lung cancer is an ALK-translocated lung cancer (e.g., ALK-translocated NSCLC). In embodiments, a cancer is NSCLC with an identified ALK translocation. In embodiments, a lung cancer is an EGFR-mutant lung cancer (e.g., EGFR-mutant NSCLC). In embodiments, a cancer is NSCLC with an identified EGFR mutation.

In embodiments, a cancer is a colorectal (CRC) cancer (e.g., a solid tumor). In embodiments, a colorectal cancer is an advanced colorectal cancer. In embodiments, a colorectal cancer is a metastatic colorectal cancer. In embodiments, a colorectal cancer is a MSI-H colorectal cancer. In embodiments, a colorectal cancer is a MSS colorectal cancer. In embodiments, a colorectal cancer is a POLE-mutant colorectal cancer. In embodiments, a colorectal cancer is a POLD-mutant colorectal cancer. In embodiments, a colorectal cancer is a high TMB colorectal cancer.

In embodiments, a cancer is a melanoma. In embodiments, a melanoma is an advanced melanoma. In embodiments, a melanoma is a metastatic melanoma. In embodiments, a melanoma is a MSI-H melanoma. In embodiments, a melanoma is a MSS melanoma. In embodiments, a melanoma is a POLE-mutant melanoma. In embodiments, a melanoma is a POLD-mutant melanoma. In embodiments, a melanoma is a high TMB melanoma.

In embodiments, a cancer is a recurrent cancer (e.g., a recurrent gynecological cancer such as recurrent epithelial ovarian cancer, recurrent fallopian tube cancer, recurrent primary peritoneal cancer, or recurrent endometrial cancer).

In embodiments, immune-related gene expression signatures can be predictive of a response to an anti-PD-1 therapy for cancer as described herein. For example, a gene panel that includes genes associated with IFN-γ signaling can be useful in identifying cancer patients who would benefit from anti-PD-1 therapy. Exemplary gene panels are described in Ayers et al., J. Clin. Invest., 127(8):2930-2940, 2017. In embodiments, a cancer patient has a cancer that is breast cancer (e.g., TNBC) or ovarian cancer. In embodiments, a cancer patient has a cancer that is bladder cancer, gastric cancer, bilary cancer, esophageal cancer, or head and neck squamous cell carcinoma (HNSCC). In embodiments, a cancer patient has a cancer that is anal cancer or colorectal cancer.

In some embodiments, a patient has a tumor that expresses PD-L1. In some embodiments, PD-L1 status is evaluated in a patient or patient population. In some embodiments, mutational load and baseline gene expression profiles in archival or fresh pre-treatment biopsies are evaluated before, during and/or after treatment with an anti-PD-1 antibody agent. In some embodiments, the status and/or expression of TIM-3 and/or LAG-3 are evaluated in patients.

In some embodiments, a patient has previously been treated with one or more different cancer treatment modalities. In some embodiments, at least some of the patients in the cancer patient population have previously been treated with one or more of surgery, radiotherapy, chemotherapy or immunotherapy. In some embodiments, at least some of the patients in the cancer patient population have previously been treated with chemotherapy (e.g., platinum-based chemotherapy). For example, a patient who has received two lines of cancer treatment can be identified as a 2L cancer patient (e.g., a 2L NSCLC patient). In embodiments, a patient has received two lines or more lines of cancer treatment (e.g., a 2L+ cancer patient such as a 2L+ endometrial cancer patient). In embodiments, a patient has not been previously treated with an anti-PD-1 therapy. In embodiments, a patient previously received at least one line of cancer treatment (e.g., a patient previously received at least one line or at least two lines of cancer treatment). In embodiments, a patient previously received at least one line of treatment for metastatic cancer (e.g., a patient previously received one or two lines of treatment for metastatic cancer).

In embodiments, a patient has not been previously treated with an immunotherapy (e.g., a patient has not been previously treated with an anti-PD-1, anti-PD-L1, anti-CTLA-4, anti-TIM-3, and/or anti-LAG-3 therapy). In embodiments, a patient has not been previously treated with an anti-PD-1 immunotherapy. In embodiments, a patient has not been previously treated with an anti-PD-L1 immunotherapy. In embodiments, a patient has not been previously treated with an anti-CTLA-4 immunotherapy. In embodiments, a patient has not been previously treated with an anti-TIM-3 immunotherapy. In embodiments, a patient has not been previously treated with an anti-LAG-3 immunotherapy. In embodiments, a patient who has not been previously treated with an immunotherapy has received at least one other line of treatment (LOT) as described herein. In embodiments, a patient who has not been previously treated with an immunotherapy has received one, two, three, four, or five prior LOT (e.g., any LOT as described herein).

In embodiments, a patient has been previously treated with at least one immunotherapy (e.g., a patient has been previously treated with an anti-PD-1, anti-PD-L1, anti-CTLA-4, anti-TIM-3, and/or anti-LAG-3 therapy). In embodiments, a patient has been previously treated with an anti-PD-1 immunotherapy. In embodiments, a patient has been previously treated with an anti-PD-L1 immunotherapy. In embodiments, a patient has been previously treated with an anti-CTLA-4 immunotherapy. In embodiments, a patient has been previously treated with an anti-TIM-3 immunotherapy. In embodiments, a patient has been previously treated with an anti-LAG-3 immunotherapy. In embodiments, a patient who has been previously treated with an immunotherapy has received at least one other line of treatment (LOT) as described herein. In embodiments, a patient who has not been previously treated with an immunotherapy has received one, two, three, four, or five other LOT (e.g., any LOT as described herein).

In embodiments, a subject is resistant to treatment with an agent that inhibits PD-1. In embodiments, a subject is refractory to treatment with an agent that inhibits PD-1. In embodiments, a method described herein sensitizes the subject to treatment with an agent that inhibits PD-1.

In some embodiments, a disorder to be treated by the methods of the present disclosure is an infectious disease. In some embodiments, the infectious disease is caused by a virus or a bacterium. In some embodiments, the virus is human immunodeficiency virus (HIV), respiratory syncytial virus (RSV), influenza virus, dengue virus, Epstein-Barr virus (EBV) human papillomavirus (HPV), hepatitis B virus (HBV), or hepatitis C virus (HCV), optionally wherein the cancer is virally infected head and neck cancer, cervical cancer, hepatocellular carcinoma, or nasopharyngeal cancer. When the inventive method treats an infectious disease, an anti-LAG-3 antibody agent can be administered in combination with at least one anti-bacterial agent or at least one anti-viral agent. In this respect, the anti-bacterial agent can be any suitable antibiotic known in the art. The anti-viral agent can be any vaccine of any suitable type that specifically targets a particular virus (e.g., live-attenuated vaccines, subunit vaccines, recombinant vector vaccines, and small molecule anti-viral therapies (e.g., viral replication inhibitors and nucleoside analogs).

The disclosed methods can be used to treat any suitable type of autoimmune disease (i.e., as disease or disorder caused by immune system overactivity in which the body attacks and damages its own tissues), such as those described in, for example, MacKay I. R. and Rose N. R., eds., The Autoimmune Diseases, Fifth Edition, Academic Press, Waltham, Mass. (2014). Examples of autoimmune diseases that can be treated by the disclosed methods include, but are not limited to, multiple sclerosis, type 1 diabetes mellitus, rheumatoid arthritis, scleroderma, Crohn's disease, psoriasis, systemic lupus erythematosus (SLE), and ulcerative colitis. When the inventive method treats an autoimmune disease, an anti-LAG-3 antibody agent can be used in combination with an anti-inflammatory agent including, for example, corticosteroids (e.g., prednisone and fluticasone) and non-steroidal anti-inflammatory drugs (NSAIDs) (e.g., aspirin, ibuprofen, and naproxen).

Administration of a composition comprising the disclosed immunoglobulin heavy chain polypeptide, the disclosed immunoglobulin light chain polypeptide, the disclosed anti-LAG-3 antibody agents, the disclosed nucleic acid sequence encoding any of the foregoing, or the disclosed vector comprising the disclosed nucleic acid sequence induces an immune response against a cancer or infectious disease in a mammal. An “immune response” can entail, for example, antibody production and/or the activation of immune effector cells (e.g., T-cells).

Exemplary Dosages and Dosage Regiments for LAG-3 Agents

As used herein, the terms “treatment,” “treating,” and the like can refer to obtaining a desired pharmacologic and/or physiologic effect. In some embodiments, the effect can be therapeutic, i.e., the effect partially or completely cures a disease and/or adverse symptom attributable to the disease. To this end, the disclosed method can comprise administering a “therapeutically effective amount” of a LAG-3 agent. A “therapeutically effective amount” can refer to an amount effective, at dosages and for periods of time necessary, to achieve a desired therapeutic result. The therapeutically effective amount may vary according to factors such as the disease state, age, sex, and weight of the individual, and the ability of an anti-LAG-3 antibody agent to elicit a desired response in the individual. For example, a therapeutically effective amount of a LAG-3 agent can be an amount which decreases LAG-3 bioactivity in a human.

Alternatively, the pharmacologic and/or physiologic effect may be prophylactic, i.e., the effect completely or partially prevents a disease or symptom thereof. In this respect, the disclosed method can comprise administering a “prophylactically effective amount” of a LAG-3 agent. A “prophylactically effective amount” can refer to an amount effective, at dosages and for periods of time necessary, to achieve a desired prophylactic result (e.g., prevention of disease onset).

A typical dose can be, for example, in the range of 1 pg/kg to 20 mg/kg of animal or human body weight; however, doses below or above this exemplary range can be within the scope of the disclosure. The daily parenteral dose can be about 0.00001 μg/kg to about 20 mg/kg of total body weight (e.g., about 0.001 μg /kg, about 0.1 μg/kg , about 1 μg /kg, about 5 μg /kg, about 10 μg/kg, about 100 μg/kg, about 500 μg/kg, about 1 mg/kg, about 5 mg/kg, about 10 mg/kg, or a range defined by any two of the foregoing values), from about 0.1 μg/kg to about 10 mg/kg of total body weight (e.g., about 0.5 μg/kg, about 1 μg/kg, about 50 μg/kg, about 150 μg/kg, about 300 μg/kg, about 750 μg/kg, about 1.5 mg/kg, about 5 mg/kg, or a range defined by any two of the foregoing values), from about 1 μg/kg to 5 mg/kg of total body weight (e.g., about 3 μg/kg, about 15 μg/kg, about 75 μg/kg, about 300 μg/kg, about 900 μg/kg, about 2 mg/kg, about 4 mg/kg, or a range defined by any two of the foregoing values), or from about 0.5 to 15 mg/kg body weight per day (e.g., about 1 mg/kg, about 2.5 mg/kg, about 3 mg/kg, about 6 mg/kg, about 9 mg/kg, about 11 mg/kg, about 13 mg/kg, or a range defined by any two of the foregoing values). Therapeutic or prophylactic efficacy can be monitored by periodic assessment of treated patients. For repeated administrations over several days or longer, depending on the condition, the treatment can be repeated until a desired suppression of disease symptoms occurs, or alternatively, the treatment can be continued for the lifetime of the patient. However, other dosage regimens may be useful and can be within the scope of the disclosure. The desired dosage can be delivered by a single bolus administration of the composition, by multiple bolus administrations of the composition, or by continuous infusion administration of the composition.

In some embodiments, a LAG-3 agent is administered to a subject as a monotherapy to induce an immune response. In some embodiments, an anti-LAG-3 antibody agent is administered as a monotherapy to a patient that has a cancer. The patient can have any kind of cancer. In some embodiments, the cancer includes any one or more of the following: epithelial ovarian cancer (EOC), triple-negative breast cancer (TNBC), post-anti-PD-1/PD-L1 urothelial carcinoma (UC), and anti-PD-1/L1 naïve UC.

In some embodiments, the patient is administered a dose of a LAG-3 agent. In some embodiments, suitable doses include 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 mg/patient. In some embodiments, the dosage is selected from 20, 80, 240 and 720 mg/patient. In embodiments, a suitable dose in within a range of about 240 mg/patient to about 720 mg/patient. In embodiments, a suitable dose is about 240 mg/patient, about 320 mg/patient, about 400 mg/patient about 480 mg/patient, about 500 mg, about 560 mg/patient, about 640 mg/patient, or about 720/mg patient. In embodiments, a suitable dose is about 200 mg/patient, about 300 mg/patient, about 400 mg/patient, about 500 mg/patient, about 600 mg/patient, or about 700 mg/patient. In other embodiments, a suitable dose is about 250 mg/patient, about 300 mg/patient, about 350 mg/patient, about 400 mg/patient, about 450 mg/patient, about 500 mg/patient, about 550 mg/patient, about 600 mg/patient, about 650 mg/patient, or about 700 mg/patient.

In some embodiments, the method includes administering the LAG-3 agent at a dose of 20 mg/patient. In some embodiments, the method includes administering the LAG-3 agent at a dose of 80 mg/patient. In some embodiments, the method includes administering the LAG-3 agent at a dose of 240 mg/patient. In some embodiments, the method includes administering the LAG-3 agent at a dose of 720 mg/patient.

In some embodiments, the method includes administering the LAG-3 agent at a dose of up to about 3000 mg or up to about 2500 mg.

In embodiments, the methods of the present disclosure include administering the LAG-3 agent at a dose of about 20 mg, about 80 mg, about 240 mg, about 500 mg, about 720 mg, about 900 mg, about 1000 mg, about 1200 mg, about 1500 mg, about 1800 mg, about 2100 mg, about 2200 mg, or about 2500 mg. In embodiments, the methods of the present disclosure include administering the LAG-3 agent at a dose of about 500 mg, about 900 mg, about 1000 mg, about 1200 mg, about 1500 mg, about 1800 mg, about 2100 mg, about 2200 mg, or about 2500 mg. In embodiments, the methods of the present disclosure include administering the LAG-3 agent at a dose of about 500 mg. In some embodiments, the methods of the present disclosure include administering the LAG-3 agent at a dose of about 900 mg. In some embodiments, the methods of the present disclosure include administering the LAG-3 agent at a dose of about 1000 mg. In some embodiments, the methods of the present disclosure include administering the LAG-3 agent at a dose of about 1200 mg. In some embodiments, the methods of the present disclosure include administering the LAG-3 agent at a dose of about 1500 mg. In some embodiments, the methods of the present disclosure include administering the LAG-3 agent at a dose of about 1800 mg. In some embodiments, the methods of the present disclosure include administering the LAG-3 agent at a dose of about 2100 mg. In some embodiments, the methods of the present disclosure include administering the LAG-3 agent at a dose of about 2200 mg. In some embodiments, the methods of the present disclosure include administering the LAG-3 agent at a dose of about 2500 mg.

In some embodiments, the methods of the present disclosure include administering the LAG-3 agent at a dose of about 1 mg/kg, about 3 mg/kg, about 10 mg/kg, about 12 mg/kg, about 15 mg/kg, about 20 mg/kg, or about 25 mg/kg. In some embodiments, the methods of the present disclosure include administering the LAG-3 agent at a dose of about 1 mg/kg. In some embodiments, the methods of the present disclosure include administering the LAG-3 agent at a dose of about 3 mg/kg. In some embodiments, the methods of the present disclosure include administering the LAG-3 agent at a dose of about 10 mg/kg. In some embodiments, the methods of the present disclosure include administering the LAG-3 agent at a dose of about 12 mg/kg. In some embodiments, the methods of the present disclosure include administering the LAG-3 agent at a dose of about 15 mg/kg. In some embodiments, the methods of the present disclosure include administering the LAG-3 agent at a dose of about 20 mg/kg. In some embodiments, the methods of the present disclosure include administering the LAG-3 agent at a dose of about 25 mg/kg.

The administration interval for the administration of the anti-LAG-3 antibody can be any interval. For example, in some embodiments, the administration interval is once every week (Q1W), once every two weeks (Q2W), once every three weeks (Q3W), once every four weeks (Q4W), once every five weeks (Q5W), once every six weeks (Q6W), once every seven weeks (Q7W), once every eight weeks (Q8W), once every nine weeks (Q9W), or once every ten weeks (Q10W). In some embodiments, the administration interval is once every two weeks (Q2W).

For example, in some embodiments, the administration interval is once every two weeks (Q2W).

In some embodiments, a dose of about 240 mg of the LAG-3 agent is administered once every two weeks (Q2W).

In some embodiments, a dose of about 500 mg of the LAG-3 agent is administered once every two weeks (Q2W).

In some embodiments, a dose of about 720 mg of the LAG-3 agent is administered once every two weeks (Q2W).

In some embodiments, a dose of about 900 mg of the LAG-3 agent is administered once every two weeks (Q2W).

In some embodiments, a dose of about 1000 mg of the LAG-3 agent is administered once every two weeks (Q2W).

In some embodiments, a dose of about 1500 mg of the LAG-3 agent is administered once every two weeks (Q2W).

In some embodiments, a dose of about 3 mg/kg of the LAG-3 agent is administered once every two weeks (Q2W).

In some embodiments, a dose of about 10 mg/kg of the LAG-3 agent is administered once every two weeks (Q2W).

In some embodiments, a dose of about 12 mg/kg of the LAG-3 agent is administered once every two weeks (Q2W).

In some embodiments, a dose of about 15 mg/kg of the LAG-3 agent is administered once every two weeks (Q2W).

For example, in some embodiments, the administration interval is once every three weeks (Q3W).

In some embodiments, a dose of about 500 mg of the LAG-3 agent is administered once every three weeks (Q3W).

In some embodiments, a dose of about 720 mg of the LAG-3 agent is administered once every three weeks (Q3W).

In some embodiments, a dose of about 900 mg of the LAG-3 agent is administered once every three weeks (Q3W).

In some embodiments, a dose of about 1000 mg of the LAG-3 agent is administered once every three weeks (Q3W).

In some embodiments, a dose of about 1500 mg of the LAG-3 agent is administered once every three weeks (Q3W).

In some embodiments, a dose of about 1800 mg of the LAG-3 agent is administered once every three weeks (Q3W).

In some embodiments, a dose of about 2100 mg of the LAG-3 agent is administered once every three weeks (Q3W).

In some embodiments, a dose of about 2200 mg of the LAG-3 agent is administered once every three weeks (Q3W).

In some embodiments, a dose of about 2500 mg of the LAG-3 agent is administered once every three weeks (Q3W).

In some embodiments, a dose of about 10 mg/kg of the LAG-3 agent is administered once every three weeks (Q3W).

In some embodiments, a dose of about 12 mg/kg of the LAG-3 agent is administered once every three weeks (Q3W).

In some embodiments, a dose of about 15 mg/kg of the LAG-3 agent is administered once every three weeks (Q3W).

In some embodiments, a dose of about 20 mg/kg of the LAG-3 agent is administered once every three weeks (Q3W).

In some embodiments, a dose of about 25 mg/kg of the LAG-3 agent is administered once every three weeks (Q3W).

In embodiments, a LAG-3 agent is a polypeptide comprising a CDR-H1 defined by SEQ ID NO: 5; a CDR-H2 defined by SEQ ID NO: 6; a CDR-H3 defined by SEQ ID NO: 7; a CDR-L1 defined by SEQ ID NO: 8; a CDR-L2 defined by SEQ ID NO: 9; and a CDR-L3 defined by SEQ ID NO: 10. In embodiments, a LAG-3 agent is a polypeptide comprising a heavy chain variable region amino acid sequence having at least 80%, 85%, 90%, 95%, or 98% sequence identity to SEQ ID NO: 3; and a light chain variable region amino acid sequence having at least 80%, 85%, 90%, 95%, or 98% sequence identity to SEQ ID NO: 4. In embodiments, a LAG-3 agent is a polypeptide comprising a heavy chain polypeptide sequence having at least 80%, 85%, 90%, 95%, or 98% sequence identity to SEQ ID NO: 1 or SEQ ID NO: 21; and a light chain polypeptide sequence having at least 80%, 85%, 90%, 95%, or 98% sequence identity to SEQ ID NO: 2 or SEQ ID NO: 22. In embodiments, a LAG-3 agent is TSR-033.

In some embodiments, the LAG-3 agent is administered to a subject as a combination therapy as further described herein (e.g., with anti-PD-1 antibody to induce an immune response). In some embodiments, the anti-LAG-3 antibody agent is administered to a patient having cancer. The patient can have any kind of cancer. In some embodiments, the cancer includes any one or more of the following: epithelial ovarian cancer (EOC), triple-negative breast cancer (TNBC), post-anti-PD-1/PD-L1 urothelial carcinoma (UC), and anti-PD-1/L1 naïve UC. In some embodiments, the patient that receives the anti-LAG-3 antibody agent and the anti-PD-1 antibody agent, first receives an infusion of the anti-LAG-3 antibody agent, followed by an infusion of the anti-PD-1 antibody agent. In some embodiments, the patient that receives the anti-LAG-3 antibody agent and the anti-PD-1 antibody agent, first receives an infusion of the anti-PD-1 antibody agent, followed by an infusion of the anti-LAG-3 antibody agent. In some embodiments, the patient receives an anti-LAG-3 antibody infusion of 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 mg/patient, followed by an anti-PD-1 antibody infusion of 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 mg/patient. In some embodiments, the patient receives an anti-LAG-3 antibody infusion of 20, 80, 240, or 720 mg/patient, followed by an anti-PD-1 antibody infusion of 500 mg/patient. In some embodiments, the patient receives an anti-PD-1 antibody infusion of 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 mg/patient, followed by an anti-LAG-3 antibody infusion of 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 mg/patient. In some embodiments, the patient receives an anti-PD-1 infusion of 500 mg/patient, followed by a 20, 80, 240 or 720 mg/patient. In some embodiments, the administration interval for the combination therapy is once every week (Q1W), once every two weeks (Q2W), once every three weeks (Q3W), once every four weeks (Q4W), once every five weeks (Q5W), once every six weeks (Q6W), once every seven weeks (Q7W), once every eight weeks (Q8W), once every nine weeks (Q9W), or once every ten weeks (Q10W). In some embodiments, the administration interval for the combination therapy is once every three weeks (Q3W).

In some embodiments, the patient first receives a monotherapy treatment regimen as described above, followed by a combination therapy. For example, in some embodiments, the patient can receive a monotherapy of anti-LAG-3 antibody of 20, 80, 240 or 720 mg/patient at intervals of once every week (Q1W), once every two weeks (Q2W), once every three weeks (Q3W), once every four weeks (Q4W), once every five weeks (Q5W), once every six weeks (Q6W), once every seven weeks (Q7W), once every eight weeks (Q8W), once every nine weeks (Q9W), or once every ten weeks (Q10W), followed by the a combination therapy of anti-LAG-3 antibody and anti-PD-1 antibody as described above.

In some embodiments, during combination therapy a patient is administered a dose of anti-LAG-3 antibody followed by a PD-1 inhibitor. In some embodiments, the PD-1 inhibitor is administered at a first dose of about 500 mg once every 3 weeks for multiple cycles, for example, for 3, 4, or 5 cycles followed by a second dose of about 1000 mg once every 6 weeks. In some embodiments, the PD-1 inhibitor is administered at a first dose of about 500 mg once every 3 weeks for 3 cycles followed by a second dose of about 1000 mg once every 6 weeks or more. In some embodiments, the PD-1 inhibitor is administered at a first dose of about 500 mg once every 3 weeks for 5 cycles followed by a second dose of about 1000 mg once every 6 weeks or more. In some embodiments, the second PD-1 inhibitor dose is administered once every 6 weeks.

The composition comprising an effective amount of a disclosed immunoglobulin heavy chain polypeptide, a disclosed immunoglobulin light chain polypeptide, a disclosed anti-LAG-3 antibody agent, a disclosed nucleic acid sequence encoding any of the foregoing, or a disclosed vector comprising a disclosed nucleic acid sequence can be administered to a mammal using standard administration techniques, including oral, intravenous, intraperitoneal, subcutaneous, pulmonary, transdermal, intramuscular, intranasal, buccal, sublingual, or suppository administration. The composition can be suitable for parenteral administration. The term “parenteral,” as used herein, can include intravenous, intramuscular, subcutaneous, rectal, vaginal, and intraperitoneal administration. The composition can be administered to a mammal using peripheral systemic delivery by intravenous, intraperitoneal, or subcutaneous injection.

Once administered to a mammal (e.g., a human), the biological activity of an anti-LAG-3 antibody agent can be measured by any suitable method known in the art. For example, the biological activity can be assessed by determining the stability of a particular anti-LAG-3 antibody agent. In one embodiment, an anti-LAG-3 antibody agent has an in vivo half-life between about 30 minutes and 45 days (e.g., about 30 minutes, about 45 minutes, about 1 hour, about 2 hours, about 4 hours, about 6 hours, about 10 hours, about 12 hours, about 1 day, about 5 days, about 10 days, about 15 days, about 25 days, about 35 days, about 40 days, about 45 days, or a range defined by any two of the foregoing values). In some embodiments, an anti-LAG-3 antibody agent has an in vivo half-life between about 2 hours and 20 days (e.g., about 5 hours, about 10 hours, about 15 hours, about 20 hours, about 2 days, about 3 days, about 7 days, about 12 days, about 14 days, about 17 days, about 19 days, or a range defined by any two of the foregoing values). In some embodiments, an anti-LAG-3 antibody agent has an in vivo half-life between about 10 days and about 40 days (e.g., about 10 days, about 13 days, about 16 days, about 18 days, about 20 days, about 23 days, about 26 days, about 29 days, about 30 days, about 33 days, about 37 days, about 38 days, about 39 days, about 40 days, or a range defined by any two of the foregoing values).

The stability of an anti-LAG-3 antibody agent can be measured using any other suitable assay known in the art, such as, for example, measuring serum half-life, differential scanning calorimetry (DSC), thermal shift assays, and pulse-chase assays. Other methods of measuring protein stability in vivo and in vitro that can be used in the context of the disclosure are described in, for example, Protein Stability and Folding, B. A. Shirley (ed.), Human Press, Totowa, N.J. (1995); “Protein Structure, Stability, and Interactions”, Methods in Molecular Biology, Shiver J. W. (ed.), Humana Press, New York, N.Y. (2010); and Ignatova, Microb. Cell Fact., 4: 23 (2005).

The stability of an anti-LAG-3 antibody agent can be measured in terms of the transition mid-point value (Tₘ), which is the temperature where 50% of the amino acid sequence is in its native confirmation, and the other 50% is denatured. In general, the higher the Tₘ, the more stable the protein. In one embodiment, a disclosed anti-LAG-3 antibody can comprise a transition mid-point value (Tₘ) in vitro of about 60-100° C. For example, an anti-LAG-3 antibody can comprise a Tₘ in vitro of about 65-80° C. (e.g., 66° C., 68° C., 70° C., 71° C., 75° C., or 79° C.), about 80-90° C. (e.g., about 81° C., 85° C., or 89° C.), or about 90-100° C. (e.g., about 91° C., about 95° C., or about 99° C.).

Measuring Tumor Response

In embodiments, methods described herein can provide a clinical benefit to a subject.

In some embodiments, a clinical benefit is a complete response (“CR”), a partial response (“PR”) or a stable disease (“SD”). In some embodiments, a clinical benefit corresponds to at least SD. In some embodiments, a clinical benefit corresponds to at least a PR. In some embodiments, a clinical benefit corresponds to a CR. In some embodiments, at least 1%, 2%, 3%, 4%, 5%, 6%, 7%, 8%, 9%, 10%, 15%, 20%, 25%, 30%, 35%, 40%, 50%, 55%, 60%, 65%, 70%, 75%, 80%, 85%, 90% or 95% of patients achieve a clinical benefit. In some embodiments, at least 5% of patients achieve a clinical benefit. In some embodiments, at least 5% of patients achieve SD. In some embodiments, at least 5% of patients achieve at least a PR. In some embodiments, at least 5% of patients achieve CR. In some embodiments, at least 20% of patients achieve a clinical benefit. In some embodiments, at least 20% of patients achieve SD.

In some embodiments, the clinical benefit (e.g., SD, PR and/or CR) is determined in accordance with Response Evaluation Criteria in Solid Tumors (RECIST). In some embodiments, the clinical benefit (e.g., SD, PR and/or CR) is determined in accordance RECIST guidelines.

In some embodiments, tumor response can be measured by, for example, the RECIST v 1.1 guidelines. The guidelines are provided by E.A. Eisenhauer, et al., “New response evaluation criteria in solid tumors: Revised RECIST guideline (version 1.1.),” Eur. J. of Cancer, 45: 228-247 (2009), which is incorporated by reference in its entirety. The guidelines require, first, estimation of the overall tumor burden at baseline, which is used as a comparator for subsequent measurements. Tumors can be measured via use of any imaging system known in the art, for example, by a CT scan, or an X-ray. Measurable disease is defined by the presence of at least one measurable lesion. In studies where the primary endpoint is tumor progression (either time to progression or proportion with progression at a fixed date), the protocol must specify if entry is restricted to those with measurable disease or whether patients having non-measurable disease only are also eligible.

When more than one measurable lesion is present at baseline, all lesions up to a maximum of five lesions total (and a maximum of two lesions per organ) representative of all involved organs should be identified as target lesions and will be recorded and measured at baseline (this means in instances where patients have only one or two organ sites involved a maximum of two and four lesions respectively will be recorded).

Target lesions should be selected on the basis of their size (lesions with the longest diameter), be representative of all involved organs, but in addition should be those that lend themselves to reproducible repeated measurements.

Lymph nodes merit special mention since they are normal anatomical structures which may be visible by imaging even if not involved by tumor. Pathological nodes which are defined as measurable and may be identified as target lesions must meet the criterion of a short axis of P15 mm by CT scan. Only the short axis of these nodes will contribute to the baseline sum. The short axis of the node is the diameter normally used by radiologists to judge if a node is involved by solid tumour. Nodal size is normally reported as two dimensions in the plane in which the image is obtained (for CT scan this is almost always the axial plane; for MRI the plane of acquisition may be axial, saggital or coronal). The smaller of these measures is the short axis.

For example, an abdominal node which is reported as being 20 mm·30 mm has a short axis of 20 mm and qualifies as a malignant, measurable node. In this example, 20 mm should be recorded as the node measurement. All other pathological nodes (those with short axis P10 mm but <15 mm) should be considered non-target lesions. Nodes that have a short axis <10 mm are considered non-pathological and should not be recorded or followed.

A sum of the diameters (longest for non-nodal lesions, short axis for nodal lesions) for all target lesions will be calculated and reported as the baseline sum diameters. If lymph nodes are to be included in the sum, then as noted above, only the short axis is added into the sum. The baseline sum diameters will be used as reference to further characterize any objective tumor regression in the measurable dimension of the disease.

All other lesions (or sites of disease) including pathological lymph nodes should be identified as non-target lesions and should also be recorded at baseline. Measurements are not required and these lesions should be followed as ‘present’, ‘absent’, or in rare cases ‘unequivocal progression.’ In addition, it is possible to record multiple nontarget lesions involving the same organ as a single item on the case record form (e.g., ‘multiple enlarged pelvic lymph nodes’ or ‘multiple liver metastases’).

In some embodiments, tumor response can be measured by, for example, the immune-related RECIST (irRECIST) guidelines, which include immune related Response Criteria (irRC). In irRC, measurable lesions are measured that have at least one dimension with a minimum size of 10 mm (in the longest diameter by CT or MRI scan) for nonnodal lesions and greater than or equal to 15 mm for nodal lesions, or at least 20 mm by chest X-ray.

In some embodiments, Immune Related Response Criteria include CR (complete disappearance of all lesions (measurable or not, and no new lesions)); PR (decrease in tumor burden by 50% or more relative to baseline); SD (not meeting criteria for CR or PR in the absence of PD); or PD (an increase in tumor burden of at 25% or more relative to nadir). Detailed description of irRECIST can be found at Bohnsack et al., (2014) ESMO, ABSTRACT 4958 and Nishino et al., (2013) Clin. Cancer Res. 19(14): 3936-43.

In some embodiments, tumor response can be assessed by either irRECIST or RECIST version 1.1. In some embodiments, tumor response can be assessed by both irRECIST and RECIST version 1.1.

### Combination Therapies

Provided herein are methods that comprise administering a LAG-3 agent (e.g., an anti-LAG-3 antibody agent) in combination with one or more additional therapeutic agents.

For example, a LAG-3 agent (e.g., an anti-LAG-3 antibody agent) can be administered in combination with other agents for the treatment or prevention of the diseases disclosed herein, such as agents that are cytotoxic to cancer cells, modulate the immunogenicity of cancer cells, or promote immune responses to cancer cells. In this respect, for example, an anti-LAG-3 antibody agent can be used in combination with at least one other anticancer agent including, for example, any chemotherapeutic agent known in the art, ionization radiation, small molecule anticancer agents, cancer vaccines, biological therapies (e.g., other monoclonal antibodies, cancer-killing viruses, gene therapy, and adoptive T-cell transfer), and/or surgery. When the disclosed method treats an infectious disease, a LAG-3 agent (e.g., an anti-LAG-3 antibody agent) can be administered in combination with at least one anti-bacterial agent or at least one anti-viral agent. In this respect, the anti-bacterial agent can be any suitable antibiotic known in the art. The anti-viral agent can be any vaccine of any suitable type that specifically targets a particular virus (e.g., live-attenuated vaccines, subunit vaccines, recombinant vector vaccines, and small molecule anti-viral therapies (e.g., viral replication inhibitors and nucleoside analogs). When the disclosed method treats an autoimmune disease, an anti-LAG-3 antibody can be used in combination with an anti-inflammatory agent including, for example, corticosteroids (e.g., prednisone and fluticasone) and non-steroidal anti-inflammatory drugs (NSAIDs) (e.g., aspirin, ibuprofen, and naproxen).

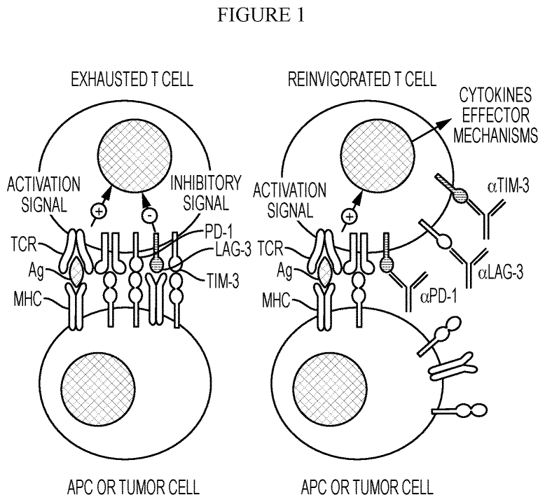

In some embodiments, when a LAG-3 agent (e.g., an anti-LAG-3 antibody agent) is used to treat cancer or an infectious disease, the anti-LAG-3 antibody can be administered in combination with other agents that inhibit immune checkpoint pathways. See, e.g., FIG. 1.

Checkpoint Inhibitors

The LAG-3 agent (e.g., an anti-LAG-3 antibody agent) can be administered in combination with other agents that inhibit immune checkpoint pathways. Combination treatments that simultaneously target two or more of these immune checkpoint pathways have demonstrated improved and potentially synergistic antitumor activity (see, e.g., Sakuishi et al., J. Exp. Med., 207: 2187-2194 (2010); Ngiow et al., Cancer Res., 71: 3540-3551 (2011); and Woo et al., Cancer Res., 72: 917-927 (2012)).

In embodiments, a checkpoint inhibitor is an agent capable of inhibiting any of the following: PD-1 (e.g., inhibition via anti-PD-1, anti-PD-L1, or anti-PD-L2 therapies), CTLA-4, TIM-3, TIGIT, LAGs (e.g., LAG-3), CEACAM (e.g., CEACAM-1, -3 and/or -5), VISTA, BTLA, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, TGFR (e.g., TGFR beta), B7-H1, B7-H4 (VTCN1), OX-40, CD137, CD40, IDO, or CSF-1R. In embodiments, a checkpoint inhibitor is a small molecule, a nucleic acid, a polypeptide (e.g., an antibody), a carbohydrate, a lipid, a metal, or a toxin. In embodiments, a checkpoint inhibitor is an antibody, an antibody conjugate, or an antigen-binding fragment thereof.

In embodiments, an immune checkpoint inhibitor is an agent that inhibits programmed death-1 protein (PD-1) signaling, cytotoxic T-lymphocyte-associated protein 4 (CTLA-4), T-cell immunoglobulin domain and mucin domain 3 protein (TIM-3) T cell immunoglobulin, lymphocyte activation gene-3 (LAG-3), and ITIM domain (TIGIT), indoleamine 2,3-dioxygenase (IDO), or colony stimulating factor 1 receptor (CSF1R). In some embodiments, methods are provided for treating or preventing cancer, infection diseases, or autoimmune disease in a mammal, comprising administering (i) an antibody agent that binds to a LAG-3 protein and (ii) an agent that inhibits PD-1 signaling and/or an agent that inhibits T-cell immunoglobulin and mucin-domain-containing 3 (TIM-3).

Agents that Inhibit CTLA-4

In embodiments, an immune checkpoint inhibitor is a CTLA-4 inhibitor (e.g., an antibody, an antibody conjugate, or an antigen-binding fragment thereof). In embodiments, a CTLA-4 inhibitor is a small molecule, a nucleic acid, a polypeptide (e.g., an antibody), a carbohydrate, a lipid, a metal, or a toxin. In embodiments, a CTLA-4 inhibitor is a small molecule. In embodiments, a CTLA-4 inhibitor is a CTLA-4 binding agent. In embodiments, a CTLA-4 inhibitor is an antibody, an antibody conjugate, or an antigen-binding fragment thereof. In embodiments, a CTLA-4 inhibitor is ipilimumab (Yervoy), AGEN1884, or tremelimumab.

Additional Agents that Inhibit LAG-3

In embodiments, an immune checkpoint inhibitor is a further LAG-3 inhibitor (e.g., an antibody, an antibody conjugate, or an antigen-binding fragment thereof). In embodiments, a LAG-3 inhibitor is a small molecule, a nucleic acid, a polypeptide (e.g., an antibody), a carbohydrate, a lipid, a metal, or a toxin. In embodiments, a LAG-3 inhibitor is a small molecule. In embodiments, a LAG-3 inhibitor is a LAG-3 binding agent. In embodiments, a LAG-3 inhibitor is an antibody, an antibody conjugate, or an antigen-binding fragment thereof. In embodiments, a LAG-3 inhibitor is IMP321, relatlimab (BMS-986016), BI 754111, GSK2831781 (IMP-731), Novartis LAG525 (IMP701), REGN3767, MK-4280, MGD-013, GSK-2831781, FS-118, XmAb22841, INCAGN-2385, FS-18, ENUM-006, AVA-017, AM-0003, Avacta PD-L1/LAG-3 bispecific affamer, iOnctura anti-LAG-3 antibody, Arcus anti-LAG-3 antibody, or Sym022, or a LAG-3 inhibitor described in WO 2016/126858, WO 2017/019894, or WO 2015/138920, each of which is hereby incorporated by reference in its entirety.

Agents that Inhibit TIGIT

In embodiments, an immune checkpoint inhibitor is a TIGIT inhibitor (e.g., an antibody, an antibody conjugate, or an antigen-binding fragment thereof). In embodiments, a TIGIT inhibitor is a small molecule, a nucleic acid, a polypeptide (e.g., an antibody), a carbohydrate, a lipid, a metal, or a toxin. In embodiments, a TIGIT inhibitor is small molecule. In embodiments, a TIGIT inhibitor is a TIGIT binding agent. In embodiments, a TIGIT inhibitor is an antibody, an antibody conjugate, or an antigen-binding fragment thereof. In embodiments, a TIGIT inhibitor is MTIG7192A, BMS-986207, or OMP-31M32.

Agents that Inhibit IDO

In embodiments, an immune checkpoint inhibitor is an IDO inhibitor. In embodiments, an IDO inhibitor is a small molecule, a nucleic acid, a polypeptide (e.g., an antibody), a carbohydrate, a lipid, a metal, or a toxin. In embodiments, an IDO inhibitor is small molecule. In embodiments, an IDO inhibitor is an IDO binding agent. In embodiments, an IDO inhibitor is an antibody, an antibody conjugate, or an antigen-binding fragment thereof.

Agents that Inhibit CSF1R

In embodiments, an immune checkpoint inhibitor is a CSF1R inhibitor. In embodiments, a CSF1R inhibitor is a small molecule, a nucleic acid, a polypeptide (e.g., an antibody), a carbohydrate, a lipid, a metal, or a toxin. In embodiments, a CSF1R inhibitor is small molecule. In embodiments, a CSF1R inhibitor is a CSF1R binding agent. In embodiments, a CSF1R inhibitor is an antibody, an antibody conjugate, or an antigen-binding fragment thereof.

Agents that Inhibit PD-1 Signaling

In one embodiment, a disclosed anti-LAG-3 antibody can be administered in combination with an antibody that binds to LAG-3 and/or an antibody that binds to PD-1. In this respect, a method of treating a disorder that is responsive to LAG-3 inhibition (e.g., cancer or an infectious disease) in a mammal can further comprise administering to the mammal a composition comprising (i) an antibody that binds to a LAG-3 protein and (ii) a pharmaceutically acceptable carrier or a composition comprising (i) an antibody that binds to a PD-1 protein and (ii) a pharmaceutically acceptable carrier.

Programmed Death 1 (PD-1) (also known as Programmed Cell Death 1) (encoded by the gene Pdcd1) is a type I transmembrane protein of 268 amino acids originally identified by subtractive hybridization of a mouse T cell line undergoing apoptosis (Ishida et al., Embo J., 11: 3887-95 (1992)). The normal function of PD-1, expressed on the cell surface of activated T cells under healthy conditions, is to down-modulate unwanted or excessive immune responses, including autoimmune reactions.

PD-1 is a member of the CD28/CTLA-4 family of T-cell regulators, and is expressed on activated T-cells, B-cells, and myeloid lineage cells (Greenwald et al., Annu. Rev. Immunol., 23: 515-548 (2005); and Sharpe et al., Nat. Immunol., 8: 239-245 (2007)). PD-1 is an inhibitory member of the CD28 family of receptors that also includes CD28, CTLA-4, ICOS and BTLA. PD-1 is expressed on activated B cells, T cells, and myeloid cells (Agata et al., supra; Okazaki et al. (2002) Curr. Opin. Immunol 14:391779-82; Bennett et al. (2003) J. Immunol. 170:711-8).

Two ligands for PD-1 have been identified, PD ligand 1 (PD-L1) and PD ligand 2 (PD-L2), both of which belong to the B7 protein superfamily (Greenwald et al, supra). PD-1 has been shown to negatively regulate antigen receptor signaling upon engagement of its ligands (PD-L1 and/or PD-L2).

Favorable response rates have been observed with certain PD-1/L1 checkpoint inhibitors in the clinic, however, there remains a considerable unmet need for alternative treatments in patients who exhibit primary resistance or suffer a relapse due to acquired or adaptive immune resistance. (Sharma et al., Cell, 2017; 168(4):707-723)

In some embodiments, an anti-LAG-3 antibody agent is administered to a subject who is receiving, has received or will receive treatment with an agent that inhibits PD-1 signaling. In some embodiments, an agent that inhibits PD-1 signaling is administered to a subject who is receiving, has received or will receive treatment with an anti-LAG-3 antibody agent.

Agents that inhibit PD-1 signaling for use in combination therapies of the present disclosure include those that bind to and block PD-1 receptors on T cells without triggering inhibitory signal transduction, agents that bind to PD-1 ligands to prevent their binding to PD-1, agents that do both, and agents that prevent expression of genes that encode either PD-1 or natural ligands of PD-1. Compounds that bind to natural ligands of PD-1 include PD-1 itself, as well as active fragments of PD-1, and in the case of the B7-H1 ligand, B7.1 proteins and fragments. Such antagonists include proteins, antibodies, anti-sense molecules and small organics.

In some embodiments, an agent that inhibits PD-1 signaling binds to human PD-1. In some embodiments, an agent that inhibits PD-1 signaling binds to human PD-L1.

In some embodiments, an agent that inhibits PD-1 signaling for use in combination therapies of the present disclosure is an antibody agent. In some embodiments, a PD-1 antibody agent binds an epitope of PD-1 which blocks the binding of PD-1 to any one or more of its putative ligands. In some embodiments, a PD-1 antibody agent binds an epitope of PD-1 which blocks the binding of PD-1 to two or more of its putative ligands. In a preferred embodiment, a PD-1 antibody agent binds an epitope of a PD-1 protein which blocks the binding of PD-1 to PD-L1 and/or PD-L2. PD-1 antibody agents of the present disclosure may comprise a heavy chain constant region (F_{c}) of any suitable class. In some embodiments, a PD-1 antibody agent comprises a heavy chain constant region that is based upon wild-type IgG1, IgG2, or IgG4 antibodies, or variants thereof.

In some embodiments, an agent that inhibits PD-1 signaling is a monoclonal antibody, or a fragment thereof. In some embodiments, an antibody agent that inhibits PD-1 signaling is a PD-1 antibody or fragment thereof. Monoclonal antibodies that target PD-1 that have been tested in clinical studies and/or received marketing approval in the United. Examples of antibody agents that target PD-1 signaling include, for example, any of the antibody agents listed in the following Table 1:

TABLE 1

| Antibody Agent | |
|---|---|
| Target (Format) | Developer |
| Opdivo Nivolumab | Bristol-Myers Squibb |
| PD-1 (Human IgG4) | ONO |
| Keytruda Pembrolizumab | Merck |
| PD-1 (Humanized IgG4) | |
| Tecentriq | Roche |
| Atezolizumab | |
| PD-L1 (Human IgG1) | |
| Imfinzi | Astra Zeneca |
| Durvalumab | |
| PD-L1 (Human IgG1) | |
| Bavencio | Merck KGaA/Pfizer |
| Avelumab | |
| PD-L1 (Human IgG1) | |
| PDR001 | Novartis |
| PD-1 (Humanized IgG4) | |
| REGN2810 (SAR-439684) | Sanofi, Regeneron |
| PD-1 (fully human IgG4) | |
| BGB-A317 | BeiGene |
| PD-1 (Humanized IgG4) | |
| engineered to not bind FcγRI | |
| LY3300054 | Eli Lilly |
| PD-L1 | |
| BI 754091 | Boehringer Ingelheim |
| (anti-PD-1) | |
| IBI308 | Innovent Biologics |
| (anti-PD-1) | (Eli Lilly) |
| INCSHR-1210 | Incyte |
| (anti-PD-1) | |
| JNJ-63723283 | Janssen Research & |
| (anti-PD-1) | Development, LLC |
| JS-001 | Shanghai Junshi Bioscience |
| (anti-PD-1) | Co., Ltd. |
| MEDI0680 (AMP-514) | MedImmune Inc |
| anti-PD-1 (Humanized IgG4) | |
| MGA-012 | MacroGenics |
| (anti-PD-1) | |
| PF-06801591 | Pfizer |
| (anti-PD-1) | |
| REGN-2810 | Regeneron |
| (anti-PD-1) | |
| TSR-042 | TESARO |
| anti-PD-1 (Humanized IgG4) | |
| CX-072 | CytomX Therapeutics |
| anti-PD-L1 | |
| FAZ053 | Novartis |
| anti-PD-L1 | |
| PD-L1 millamolecule | Bristol-Myers Squibb |

In some embodiments, an antibody agent that inhibits PD-1 signaling is atezolizumab, avelumab, BGB-A317, BI 754091, CX-072, durvalumab, FAZ053, IBI308, INCSHR-1210, JNJ-63723283, JS-001, MEDI-0680, MGA-012, nivolumab, PDR001, pembrolizumab, PF-06801591, REGN-2810, TSR-042, any of the antibodies disclosed in WO2014/179664, or derivates thereof. In some embodiments, an antibody agent that inhibits PD-1 signaling is a PD-1 antibody selected from the group consisting of BGB-A317, BI 754091, CX-072, FAZ053, IBI308, INCSHR-1210, JNJ-63723283, JS-001, LY3300054, MEDI-0680, MGA-012, nivolumab, PD-L1 millamolecule, PDR001, pembrolizumab, PF-06801591, REGN-2810, and TSR-042. In some embodiments, an antibody agent that inhibits PD-1 signaling is a PD-1 antibody selected from the group consisting of nivolumab, pembrolizumab, and TSR-042.

In some embodiments, a PD-1 binding agent is TSR-042, nivolumab, pembrolizumab, atezolizumab, durvalumab, avelumab, PDR-001, tislelizumab (BGB-A317), cemiplimab (REGN2810), LY-3300054, JNJ-63723283, MGA012, BI-754091, IBI-308, camrelizumab (HR-301210), BCD-100, JS-001, CX-072, BGB-A333, AMP-514 (MEDI-0680), AGEN-2034, CS1001, Sym-021, SHR-1316, PF-06801591, LZMO09, KN-035, AB122, genolimzumab (CBT-501), FAZ-053, CK-301, AK 104, or GLS-010, or any of the PD-1 antibodies disclosed in WO2014/179664.In embodiments, an immune checkpoint inhibitor is a PD-1 inhibitor. In embodiments, a PD-1 inhibitor is a PD-1 binding agent (e.g., an antibody, an antibody conjugate, or an antigen-binding fragment thereof). In embodiments, a PD-1 inhibitor is a PD-L1 or PD-L2 binding agent is durvalumab, atezolizumab, avelumab, BGB-A333, SHR-1316, FAZ-053, CK-301, or, PD-L1 millamolecule, or derivatives thereof.

In some embodiments, a PD-1 antibody agent is as disclosed in International Patent Application Publication WO2014/179664, the entirety of which is incorporated herein. In embodiments, a PD-1 antibody agent is as disclosed in International Patent Application No. PCT/US18/13029, the entirety of which is incorporated herein. In embodiments, a PD-1 antibody agent is as disclosed in International Patent Application No. PCT/US17/59618, the entirety of which is incorporated herein.

In some embodiments, a PD-1 antibody agent comprises a heavy chain variable domain that is 90%, 95%, 97%, 98%, 99% or 100% identical to SEQ ID NO:23. In some embodiments, a PD-1 antibody agent comprises a light chain variable domain that is 90%, 95%, 97%, 98%, 99% or 100% identical to SEQ ID NO:24. In some embodiments, a PD-1 antibody agent comprises a heavy chain variable domain that is 90%, 95%, 97%, 98%, 99% or 100% identical to SEQ ID NO:23 and a light chain variable domain that is 90%, 95%, 97%, 98%, 99% or 100% identical to SEQ ID NO:24.

In some embodiments, a PD-1 antibody agent comprises one or more CDR sequences as disclosed in International Patent Application Publication WO2014/179664, the entirety of which is incorporated herein. In some embodiments, a PD-1 antibody agent comprises one or more CDR sequences that is 90%, 95%, 97%, 98%, 99% or 100% identical to SEQ ID NOs: 25-30.

In some embodiments, a PD-1 antibody agent comprises one, two or three heavy chain CDR sequences that is 90%, 95%, 97%, 98%, 99% or 100% identical to CDR sequences of SEQ ID NOs: 25-27. In some embodiments, a PD-1 antibody agent comprises one, two or three light chain CDR sequences that is 90%, 95%, 97%, 98%, 99% or 100% identical to CDR sequences of SEQ ID NOs: 28-30. In some embodiments, a PD-1 antibody agent comprises one, two or three heavy chain CDR sequences that is 90%, 95%, 97%, 98%, 99% or 100% identical to CDR sequences of SEQ ID NOs: 25-27 and one, two or three light chain CDR sequences that is 90%, 95%, 97%, 98%, 99% or 100% identical to CDR sequences of SEQ ID NOs: 28-30. In some embodiments, a PD-1 antibody agent comprises six CDR sequences of SEQ ID NOs: 25-30.

In embodiments, a PD-1 inhibitor is TSR-042. SEQ ID NOs: 39 and 40 describe an exemplary humanized monoclonal anti-PD-1 antibody (TSR-042) utilizing a human IGHG4*01 heavy chain gene, and a human IGKC*01 kappa light chain gene, as scaffolds. There is a single Ser to Pro point mutation in the hinge region of the IgG4 heavy chain. This mutation is at the canonical S228 position. Without wishing to be bound by theory, it is envisioned that this point mutation serves to stabilize the hinge of the antibody heavy chain.

An anti-PD-1 antibody TSR-042 heavy chain
polypeptide
(CDR sequences)
SEQ ID NO: 39
EVQLLESGGGLVQPGGSLRLSCAASGFTFSSYDMSWVRQAPGKGLEWVST
ISGGGSYTYYQDSVKGRFTISRDNSKNTLYLQMNSLRAEDTAVYYCASPY
YAMDYWGQGTTVTVSSASTKGPSVFPLAPCSRSTSESTAALGCLVKDYFP
EPVTVSWNSGALTSGVHTFPAVLQSSGLYSLSSVVTVPSSSLGTKTYTCN
VDHKPSNTKVDKRVESKYGPPCPPCPAPEFLGGPSVFLFPPKPKDTLMIS
RTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK
An anti-PD-1 antibody TSR-042 light chain
polypeptide
(CDR sequences)
SEQ ID NO: 40
DIQLTQSPSFLSAYVGDRVTITCKASQDVGTAVAWYQQKPGKAPKWYWAS
TLHTGVPSRFSGSGSGTEFTLTISSLQPEDFATYYCQHYSSYPWTFGQGT
KLEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLS
SPVTKSFNRGEC

Table 2 shows the expected residues involved in disulfide linkages of an exemplary anti-PD-1 antibody agent heavy chain having an amino acid sequence as set forth in SEQ ID NO: 39. Table 3 shows the expected residues involved in disulfide linkages of an exemplary anti-PD-1 antibody agent light chain having an amino acid sequence as set forth in SEQ ID NO: 40.

TABLE 2

| Cysteine residue | anti-PD-1 mAb HC |
|---|---|
| ID after | Residue (position in |
| Edelman^{a} | SEQ ID NO: 39) |
| I | 22 |
| II | 96 |
| III | 130 |
| IV | 143 |
| V | 199 |
| VI | 222 |
| VII | 225 |
| VIII | 257 |
| IX | 317 |
| X | 363 |
| XI | 421 |

TABLE 3

| Cysteine residue | anti-PD-1 mAb LC |
|---|---|
| ID after | Residue (position in |
| Edelman^{a} | SEQ ID NO: 40) |
| I | 23 |
| II | 88 |
| III | 134 |
| IV | 194 |
| V | 214 |

This exemplary anti-PD-1 antibody exhibits an occupied N-glycosylation site at asparagine residue 293 in the CH2 domain of each heavy chain in the mature protein sequence (SEQ ID NO:39). The expressed N-glycosylation at this site is a mixture of oligosaccharide species typically observed on IgGs expressed in mammalian cell culture, for example, shown below is the relative abundance of glycan species from a preparation of this exemplary anti-PD-1 antibody cultured in Chinese Hamster Ovary (CHO) cells (Table 4).

TABLE 4
Glycan Analysis of an anti-PD-1 antibody binding agent TSR-042

| | Abundance (% of total | |
|---|---|---|
| Species | oligosaccharide) | Description of Glycan |
| G0 | <0.1% | Nonfucosylated agalactobiantennary |
| | | complex-type oligosaccharide |
| G0F | 19.5% | Core fucosylated agalactobiantennary |
| | | complex type oligosaccharide |
| G1 | 0.1% | Nonfucosylated monogalactosylated |
| | | biantennary complex type oligosaccharide |
| G1F | 45.6% | Core fucosylated monogalactosylated |
| | | biantennary complex type oligosaccharide |
| G2F | 27.4% | Core fucosylated galactosylated biantennary |
| | | complex type oligosaccharide |
| M5 | 0.5% | Oligomannosidic N-glycan, Man₅GlcNAc₂ |

In some embodiments, a PD-1-binding agent (e.g., an anti-PD-1 antibody such as TSR-042) is administered at a dose of about 1, 3 or 10 mg/kg. In some embodiments, a PD-1-binding agent (e.g., an anti-PD-1 antibody such as TSR-042) is administered according to a regimen that includes a dose of about 1, 3 or 10 mg/kg every two weeks. In some embodiments, a PD-1-binding agent (e.g., an anti-PD-1 antibody such as TSR-042) is administered according to a regimen that includes a dose of about 1, 3 or 10 mg/kg every three weeks. In some embodiments, a PD-1-binding agent (e.g., an anti-PD-1 antibody) is administered according to a regimen that includes a dose of about 1, 3 or 10 mg/kg every four weeks. In some embodiments, a PD-1-binding agent (e.g., an anti-PD-1 antibody such as TSR-042) at a dose of about 500 mg. In some embodiments, a PD-1-binding agent (e.g., an anti-PD-1 antibody such as TSR-042) is administered according to a regimen that includes a dose of about 500 mg every two weeks. In some embodiments, a PD-1-binding agent (e.g., an anti-PD-1 antibody such as TSR-042) is administered according to a regimen that includes a dose of about 500 mg every three weeks. In some embodiments, a PD-1-binding agent (e.g., an anti-PD-1 antibody such as TSR-042) is administered according to a regimen that includes a dose of about 500 mg every four weeks. In some embodiments, a PD-1-binding agent (e.g., an anti-PD-1 antibody such as TSR-042) is administered according to a regimen that includes a dose of about 1000 mg every six weeks. In some embodiments, a PD-1-binding agent (e.g., an anti-PD-1 antibody such as TSR-042) is administered according to a regimen that includes a first dose of about 500 mg every three weeks (Q3W) for the first 2-6 (e.g., the first 2, 3, 4, 5, or 6) dosage cycles and a second dose of about 1000 mg every six weeks (Q6W) until treatment is discontinued (e.g., due to disease progression, adverse effects, or as determined by a physician). In some embodiments, a PD-1-binding agent (e.g., an anti-PD-1 antibody such as TSR-042) is administered according to a regimen that includes a first dose of about 500 mg every three weeks (Q3W) for the first four dosage cycles and a second dose of about 1000 mg every six weeks (Q6W) until treatment is discontinued (e.g., due to disease progression, adverse effects, or as determined by a physician). In embodiments, a PD-1 binding agent is an anti-PD-1 antibody. In embodiments, a PD-1 binding agent is TSR-042.

In certain methods, an anti-PD-1 antibody agent can be administered prior to (e.g., 5 minutes, 15 minutes, 30 minutes, 45 minutes, 1 hour, 2 hours, 4 hours, 6 hours, 12 hours, 24 hours, 48, hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a LAG-3 binding agent to a subject in need thereof.

Agents that Inhibit TIM-3 Signaling

TIM-3 has been proposed to play a role in T-cell exhaustion and limiting the antitumor immune response and is targeted to treat cancer, infectious disease, or autoimmune disease.

TIM-3 is a 60 kDa type 1 transmembrane protein comprised of three domains: an N-terminal Ig variable (IgV)-like domain, a central Ser/Thr-rich mucin domain, and a transmembrane domain with a short intracellular tail (see, e.g., Kane, L. P., Journal of Immunology, 184(6): 2743-2749 (2010)). TIM-3 was initially identified on terminally differentiated Th1 cells, and negatively regulates the T-cell response by inducing T-cell apoptosis (see, e.g., Hastings et al., Eur. J. Immunol., 39(9): 2492-2501 (2009)). TIM-3 also is expressed on activated Th17 and Tc1 cells, and dysregulation of Tim-3 expression on CD4+ T-cells and CD8+ T-cells is associated with several autoimmune diseases, viral infections, and cancer (see, e.g., Liberal et al., Hepatology, 56(2): 677-686 (2012); Wu et al., Eur. J. Immunol., 42(5): 1180-1191 (2012); Anderson, A. C., Curr. Opin. Immunol., 24(2): 213-216 (2012); and Han et al., Frontiers in Immunology, 4: 449 (2013)).

Putative ligands for TIM-3 include phosphatidylserine (Nakayama et al., Blood, 113: 3821-3830 (2009)), galectin-9 (Zhu et al., Nat. Immunol., 6: 1245-1252 (2005)), high-mobility group protein 1 (HMGB1) (Chiba et al., Nature Immunology, 13: 832-842 (2012)), and carcinoembryonic antigen cell adhesion molecule 1 (CEACAM1) (Huang et al., Nature, 517(7534): 386-90 (2015)).

TIM-3 functions to regulate various aspects of the immune response. The interaction of TIM-3 and galectin-9 (Gal-9) induces cell death and in vivo blockade of this interaction exacerbates autoimmunity and abrogates tolerance in experimental models, strongly suggesting that TIM-3 is a negative regulatory molecule. In contrast to its effect on T-cells, the TIM-3-Gal-9 interaction exhibits antimicrobial effects by promoting macrophage clearance of intracellular pathogens (see, e.g., Sakuishi et al., Trends in Immunology, 32(8): 345-349 (2011)). In vivo, suppression of TIM-3 has been shown to enhance the pathological severity of experimental autoimmune encephalomyelitis (Monney et al., supra; and Anderson, A. C. and Anderson, D. E., Curr. Opin. Immunol., 18: 665-669 (2006)). Studies also suggest that dysregulation of the TIM-3-galectin-9 pathway could play a role in chronic autoimmune diseases, such as multiple sclerosis (Anderson and Anderson, supra). TIM-3 promotes clearance of apoptotic cells by binding phosphatidyl serine through its unique binding cleft (see, e.g., DeKruyff et al., J. Immunol., 184(4):1918-1930 (2010)).

Inhibition of TIM-3 activity, such as through use of monoclonal antibodies, is currently under investigation as an immunotherapy for tumors based on preclinical studies (see, e.g., Ngiow et al., Cancer Res., 71(21): 1-5 (2011); Guo et al., Journal of Translational Medicine, 11: 215 (2013); and Ngiow et al., Cancer Res., 71(21): 6567-6571 (2011)).

In some embodiments, an anti-LAG-3 antibody agent is administered to a subject who is receiving, has received or will receive treatment with an agent that inhibits TIM-3 signaling. In some embodiments, an agent that inhibits TIM-3 signaling is administered to a subject who is receiving, has received or will receive treatment with an anti-LAG-3 antibody agent. In some related embodiments, the subject is receiving, has received or will receive treatment with an agent that inhibits PD-1 signaling.

In some embodiments, an agent that inhibits TIM-3 signaling for use in combination therapies of the present disclosure is an antibody agent. In some embodiments, an anti-TIM-3 antibody agent binds an epitope of TIM-3 which blocks the binding of TIM-3 to any one or more of its putative ligands. TIM-3 antibody agents of the present disclosure may comprise a heavy chain constant region (Fₑ) of any suitable class. In some embodiments, a TIM-3 antibody agent comprises a heavy chain constant region that is based upon wild-type IgG1, IgG2, or IgG4 antibodies, or variants thereof.

In some embodiments, an agent that inhibits TIM-3 signaling is a monoclonal antibody, or a fragment thereof. In some embodiments, an antibody agent that inhibits TIM-3 signaling is a TIM-3 antibody or fragment thereof. Monoclonal antibodies that target TIM-3 that have been tested in clinical studies and/or received marketing approval in the United States.

In some embodiments, a TIM-3 antibody agent is MBG453, LY3321367, Sym023, or a derivative thereof. In some embodiments, a TIM-3 antibody agent is as disclosed in International Patent Application Publication WO2016/161270, the entirety of which is incorporated herein. In some embodiments, a TIM-3 antibody agent comprises a heavy chain variable domain that is 90%, 95%, 97%, 98%, 99% or 100% identical to a variable domain of SEQ ID NO:31. In some embodiments, a TIM-3 antibody agent comprises a light chain variable domain that is 90%, 95%, 97%, 98%, 99% or 100% identical to a variable domain of SEQ ID NO:32. In some embodiments, a TIM-3 antibody agent comprises a heavy chain variable domain that is 90%, 95%, 97%, 98%, 99% or 100% identical to a variable domain of SEQ ID NO:31 and a light chain variable domain that is 90%, 95%, 97%, 98%, 99% or 100% identical to a variable domain of SEQ ID NO:32.

In some embodiments, a TIM-3 antibody agent comprises a heavy chain that is or comprises a sequence that is 90%, 95%, 97%, 98%, 99% or 100% identical to SEQ ID NO:31. In some embodiments, a TIM-3 antibody agent comprises a light chain that is or comprises a sequence that is 90%, 95%, 97%, 98%, 99% or 100% identical to SEQ ID NO:32. In some embodiments, a TIM-3 antibody agent comprises a heavy chain that is or comprises a sequence that is 90%, 95%, 97%, 98%, 99% or 100% identical to SEQ ID NO:31 and a light chain that is or comprises a sequence that is 90%, 95%, 97%, 98%, 99% or 100% identical to SEQ ID NO:32.

In some embodiments, a TIM-3 antibody agent comprises one, two or three heavy chain CDR sequences that is 90%, 95%, 97%, 98%, 99% or 100% identical to CDR sequences of SEQ ID NOs: 33-35. In some embodiments, a TIM-3 antibody agent comprises one, two or three light chain CDR sequences that is 90%, 95%, 97%, 98%, 99% or 100% identical to CDR sequences of SEQ ID NOs: 36-38. In some embodiments, a TIM-3 antibody agent comprises one, two or three heavy chain CDR sequences that is 90%, 95%, 97%, 98%, 99% or 100% identical to CDR sequences of SEQ ID NOs: 33-35 and one, two or three light chain CDR sequences that is 90%, 95%, 97%, 98%, 99% or 100% identical to CDR sequences of SEQ ID NOs: 36-38. In some embodiments, a TIM-3 antibody agent comprises six CDR sequences of SEQ ID NOs: 33-38.

In embodiments, a TIM-3 antibody agent is as described in WO 2016/161270, which is hereby incorporated by reference in its entirety. In embodiments, a TIM-3 antibody agent is as described in PCT/US17/59619, which is hereby incorporated by reference in its entirety. In embodiments, a TIM-3 antibody agent is as described in PCT/US18/13021, which is hereby incorporated by reference in its entirety.

In embodiments, a TIM-3 inhibitor is TSR-022. TSR-022 comprises a humanized monoclonal anti-TIM-3 antibody comprising a heavy chain whose amino acid sequence comprises SEQ ID NO: 31 and a light chain whose amino acid sequence comprises SEQ ID NO:32. This anti-TIM-3 antibody utilizes a human IGHG4*01 heavy chain gene, and a human IGKC*01 kappa light chain gene, as scaffolds. Further, there is a single Ser to Pro point mutation in the hinge region of the IgG4 heavy chain at the canonical 5228 position. Without wishing to be bound by theory, it is envisioned that this point mutation serves to stabilize the hinge of the antibody heavy chain.

Additional biophysical and biochemical characterization of this exemplary humanized monoclonal anti-TIM-3 antibody is also provided regarding observed disulfide linkages and glycosylation. Lys-C and trypsin digested peptides were well separated and detected by on-line LC-MS analysis. The disulfide bond linkages were confirmed by comparison of total ion chromatograms in the non-reduced (NR) condition with the reduced condition. Disulfide linkages are consistent with the expected disulfide linkage pattern for an IgG4 molecule. The residues involved in the expected inter- and intrachain disulfide linkages are tabulated below (Tables 5, 6, and 7).

TABLE 5
Expected residues involved in disulfide linkages of an exemplary
anti-TIM-3 antibody agent heavy chain having an amino acid
sequence as set forth in SEQ ID NO: 31.

| | anti-TIM-3 mAb HC |
|---|---|
| Cysteine residue | Residue (position in |
| ID | SEQ ID NO: 31) |
| I | 22 |
| II | 96 |
| III | 127 |
| IV | 140 |
| V | 196 |
| VI | 219 |
| VII | 222 |
| VIII | 254 |
| IX | 314 |
| X | 360 |
| XI | 418 |

TABLE 6
Expected residues involved in disulfide linkages
of an exemplary anti-TIM-3 antibody agent light chain having
an amino acid sequence as set forth in SEQ ID NO: 32.

| Cysteine residue | anti-TIM-3 mAb LC Residue |
|---|---|
| ID | (position in SEQ ID NO: 32) |
| I | 23 |
| II | 88 |
| III | 134 |
| IV | 194 |
| V | 214 |

TABLE 7
Exemplary disulfide bond assignments for an anti-TIM-3
antibody TSR-022

| | | Linkage site on | Linkage site on |
|---|---|---|---|
| Disulfide | | HC (position in | LC (position in |
| bond NO. | Disulfide-containing peptides | SEQ ID NO: 31) | SEQ ID NO: 32) |
| DS1 | VTITCR=FSGSGSGTDFTLTISSLQPEDF | | 23 |
| | AVYYCQQSHSAPLTFGGGTK | | 88 |
| DS2 | SGTASVVCLLNNFYPR=VYACEVTHQGL | | 134 |
| | SSPVTK | | 194 |
| DS3 | SFNRGEC=GPSVFPLAPCSR | 127 | 214 |
| | GEC=GPSVFPLAPCSR | | |
| DS4 | LSCAAASGFTFSSYDMSWVR=AEDTA | 22 | |
| | VYYCASMDYWGQGTTVTVSSASTK | 97 | |
| DS5 | STSESTAALGCLVK=TYTCNVDHK | 140 | |
| | STSESTAALGCLVK=TYTCNVDHKPSNTK | 196 | |
| DS6 | YGPPCPPCPAPEFLGGPSVFLFPPK=YGPP | 219 | |
| | CPPCPAPEFLGGPSVFLFPPK | | |
| | YGPPCPPCPAPEFLGGPSVFLFPPK=YGPP | 222 | |
| | CPPCPAPEFLGGPSVFLFPPKPK | | |
| DS7 | TPEVTCVVVDVSQEDPEVQFNWYVDGV | 254 | |
| | EVHNAK=CK | 314 | |
| DS8 | NQVSLTCLVK=WQEGNVFSCSVMHEAL | 360 | |
| | HNHYTQK | 418 | |

LC: light chain;
HC: heavy chain

This exemplary anti-TIM-3 antibody exhibits an occupied N-glycosylation site at asparagine residue 290 in the CH2 domain of each heavy chain in the mature protein sequence (SEQ ID NO:31). The expressed N-glycosylation at this site is a mixture of oligosaccharide species typically observed on IgGs expressed in mammalian cell culture, for example, shown below is the relative abundance of glycan species from a preparation of this exemplary anti-TIM-3 antibody cultured in Chinese Hamster Ovary (CHO) cells (Table 8).

TABLE 8
Glycan Analysis of an anti-TIM-3 antibody binding agent

| | Abundance (% of total | |
|---|---|---|
| Species | oligosaccharide) | Description of Glycan |
| G0F | 20.1% | Core fucosylated agalactobiantennary |
| | | complex-type oligosaccharide |
| G1F | 41.9% | Core fucosylated monogalactosylated |
| | | biantennary complex type oligosaccharide |
| G2F | 29.0% | Core-fucosylated galactosylated biantennary |
| | | complex type oligosaccharide |
| G2FS1 | 3.2% | Monosialylated core fucosylated |
| | | galactosylated biantennary complex type |
| | | oligosaccharide |
| G2FS2 | 1.2% | Disialylated core fucosylated galactosylated |
| | | biantennary complex type oligosaccharide |
| M5 | 0.4% | Oligomannosidic N-linked oligosaccharide, |
| | | Man₅GlcNAc₂ |

For example, a TIM-3 inhibitor (e.g., TSR-022) can be administered in a dose of about 1, 3 or 10 mg/kg (e.g., about 1 mg/kg; about 3 mg/kg; or about 10 mg/kg) or a flat dose between about 100-1500 mg (e.g., a flat dose about 100 mg; a flat dose about 200 mg; a flat dose about 300 mg; a flat dose about 400 mg; a flat dose about 500 mg; a flat dose about 600 mg; a flat dose about 700 mg; a flat dose about 800 mg; a flat dose about 900 mg; a flat dose about 1000 mg; a flat dose about 1100 mg; a flat dose about 1200 mg; a flat dose about 1300 mg; a flat dose about 1400 mg; or a flat dose about 1500 mg).

In some embodiments, an anti-TIM-3 antibody agent (e.g,. an anti-TIM-3 antibody) is administered at a dose of 0.1, 1, 3 or 10 mg/kg. In some embodiments, an anti-TIM-3 antibody agent is administered according to a regimen that includes a dose of 0.1, 1, 3 or 10 mg/kg every two weeks. In some embodiments, an anti-TIM-3 antibody agent is administered according to a regimen that includes a dose of 1, 3 or 10 mg/kg every three weeks.

In some embodiments, an anti-TIM-3 antibody agent is administered according to a regimen that includes a dose of 1, 3 or 10 mg/kg every four weeks. In some embodiments, an anti-TIM-3 antibody agent at a fixed dose within a range of 200 mg to 1,500 mg. In some embodiments, an anti-TIM-3 antibody agent at a fixed dose within a range of 300 mg to 1,000 mg. In some embodiments, an anti-TIM-3 antibody agent is administered according to a regimen that includes a fixed dose every two weeks. In some embodiments, an anti-TIM-3 antibody agent is administered according to a regimen that includes a fixed dose every three weeks. In some embodiments, an anti-TIM-3 antibody agent is administered according to a regimen that includes a fixed dose every four weeks.

In certain methods, an anti-TIM-3 antibody agent can be administered prior to (e.g., 5 minutes, 15 minutes, 30 minutes, 45 minutes, 1 hour, 2 hours, 4 hours, 6 hours, 12 hours, 24 hours, 48, hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a LAG-3 binding agent to a subject in need thereof.

Triple Combination Therapy Using LAG-3, PD-1, and TIM-3 Agents

In embodiments, a combination therapy is a triple blockade therapy comprising administration of a LAG-3 agent, a PD-1 agent, and a TIM-3 agent to a subject.

Despite successes with therapies targeting immune checkpoint molecules, many patients do not benefit from currently approved PD-1 and CTLA-4-directed antibodies. In these patients, other mechanisms of immune suppression may coincide to prevent effective antitumor immunity. Thus, there is a need to develop additional therapies directed against additional immunological targets.

T cell hypo-responsiveness, termed T-cell exhaustion, involves immune inhibitory receptors expressed by T cells, including cytotoxic T-lymphocyte-associated antigen-4 (CTLA-4), programmed death-1 (PD-1), T-cell immunoglobulin and mucin-domain containing-3 (TIM-3) and Lymphocyte activation gene-3 (LAG-3). Further, LAG-3 is widely associated with exhausted or dysfunctional T cells and is frequently co-expressed with both PD-1 and TIM-3.

In another aspect, an agent that inhibits LAG-3 signaling (e.g., an anti-LAG-3 antibody agent) is administered to a subject who is receiving, has received or will receive treatment with an agent that inhibits TIM-3 signaling and/or treatment with an agent that inhibits PD-1 signaling. In some embodiments, an agent that inhibits TIM-3 signaling is administered to a subject who is receiving, has received or will receive treatment with an anti-LAG-3 antibody agent and treatment with an agent that inhibits PD-1 signaling. In some embodiments, an agent that inhibits PD-1 signaling is administered to a subject who is receiving, has received or will receive treatment with an agent that inhibits TIM-3 signaling and treatment with an anti-LAG-3 antibody agent. In embodiments, a patient is further administered one or more additional therapeutic agents in addition to the LAG-3, PD-1, and TIM-3 agents described herein, and/or a patient receives one or more additional treatment modalities. For example, treatment with LAG-3, PD-1, and TIM-3 agents can occur in combination with one or more of surgery, a radiotherapy, a chemotherapy, an immunotherapy, an anti-angiogenic agent, or an anti-inflammatory), or treatment with LAG-3, PD-1, and TIM-3 agents can occur in combination with one or more additional therapeutic agents (e.g., a PARP inhibitor such as niraparib) as described herein.

In particular, certain methods described herein relate to triple combination therapy or triple blockade therapy, wherein a PD-1 agent, a TIM-3 agent, and a LAG-3 agent are administered to a subject. Such triple combination therapy can be more efficacious and provide additional benefit to some patients (e.g., triple combination therapy can be more efficacious or provide additional benefit to a patient when compared to monotherapy using a PD-1 agent, a TIM-3 agent, and a LAG-3 agent or compared to dual therapy using any combination of a PD-1 agent, a TIM-3 agent, and a LAG-3 agent).

Suitable PD-1 agents include any of the agents that inhibit PD-1 signaling as described herein. In embodiments, a PD-1 agent is a small molecule, a nucleic acid, a polypeptide (e.g., an antibody, an antibody conjugate, or an antigen-binding fragment thereof), a carbohydrate, a lipid, a metal, or a toxin. In embodiments, a PD-1 agent is a PD-1 binding agent. In embodiments, a PD-1 agent is a PD-1 binding agent (e.g., an antibody, an antibody conjugate, or an antigen-binding fragment thereof). In embodiments, a PD-1 agent is an antibody, an antibody conjugate, or an antigen-binding fragment thereof. In embodiments, a PD-1 binding agent is TSR-042, nivolumab, pembrolizumab, atezolizumab, durvalumab, avelumab, PDR-001, tislelizumab (BGB-A317), cemiplimab (REGN2810), LY-3300054, JNJ-63723283, MGA012, BI-754091, IBI-308, camrelizumab (HR-301210), BCD-100, JS-001, CX-072, BGB-A333, AMP-514 (MEDI-0680), AGEN-2034, CS1001, Sym-021, SHR-1316, PF-06801591, LZMO09, KN-035, AB122, genolimzumab (CBT-501), FAZ-053, CK-301, AK 104, or GLS-010, or any of the PD-1 antibodies disclosed in WO2014/179664. In embodiments, a PD-1 agent is TSR-042.

Suitable TIM-3 agents include any of the agents that inhibit TIM-3 signaling as described herein. In embodiments, a TIM-3 agent is a small molecule, a nucleic acid, a polypeptide (e.g., an antibody, an antibody conjugate, or an antigen-binding fragment thereof), a carbohydrate, a lipid, a metal, or a toxin. In embodiments, a TIM-3 agent is a TIM-3 binding agent. In embodiments, a TIM-3 agent is a TIM-3 binding agent (e.g., an antibody, an antibody conjugate, or an antigen-binding fragment thereof). In embodiments, a TIM-3 agent is an antibody, an antibody conjugate, or an antigen-binding fragment thereof. In some embodiments, a TIM-3 agent is MBG453, LY3321367, Sym023, or a derivative thereof. In some embodiments, a TIM-3 agent is as disclosed in International Patent Application Publication WO2016/161270, the entirety of which is incorporated herein. In embodiments, a TIM-3 agent is TSR-022.

Suitable LAG-3 agents include any of the agents that inhibit LAG-3 signaling as described herein. In embodiments, a LAG-3 agent is a small molecule, a nucleic acid, a polypeptide (e.g., an antibody, an antibody conjugate, or an antigen-binding fragment thereof), a carbohydrate, a lipid, a metal, or a toxin. In embodiments, a LAG-3 agent is a LAG-3 binding agent. In embodiments, a LAG-3 agent is a LAG-3 binding agent (e.g., an antibody, an antibody conjugate, or an antigen-binding fragment thereof). In embodiments, a LAG-3 agent is an antibody, an antibody conjugate, or an antigen-binding fragment thereof. In embodiments, a LAG-3 agent is a IMP321, relatlimab (BMS-986016), BI 754111, GSK2831781 (IMP-731), Novartis LAG525 (IMP701), REGN3767, MK-4280, MGD-013, GSK-2831781, FS-118, XmAb22841, INCAGN-2385, FS-18, ENUM-006, AVA-017, AM-0003, Avacta PD-L1/LAG-3 bispecific affamer, iOnctura anti-LAG-3 antibody, Arcus anti-LAG-3 antibody, or Sym022, or a LAG-3 inhibitor described in WO 2016/126858, WO 2017/019894, or WO 2015/138920, each of which is hereby incorporated by reference in its entirety. In embodiments, a LAG-3 agent is a polypeptide comprising a CDR-H1 defined by SEQ ID NO: 5; a CDR-H2 defined by SEQ ID NO: 6; a CDR-H3 defined by SEQ ID NO: 7; a CDR-L1 defined by SEQ ID NO: 8; a CDR-L2 defined by SEQ ID NO: 9; and a CDR-L3 defined by SEQ ID NO: 10. In embodiments, a LAG-3 agent is a polypeptide comprising a heavy chain variable region amino acid sequence having at least 80%, 85%, 90%, 95%, or 98% sequence identity to SEQ ID NO: 3; and a light chain variable region amino acid sequence having at least 80%, 85%, 90%, 95%, or 98% sequence identity to SEQ ID NO: 4. In embodiments, a LAG-3 agent is a polypeptide comprising a heavy chain polypeptide sequence having at least 80%, 85%, 90%, 95%, or 98% sequence identity to SEQ ID NO: 1 or SEQ ID NO: 21; and a light chain polypeptide sequence having at least 80%, 85%, 90%, 95%, or 98% sequence identity to SEQ ID NO: 2 or SEQ ID NO: 22. In embodiments, a LAG-3 agent is TSR-033.

In embodiments, a method comprises administration of α-PD-1 agent that is TSR-042 and α-TIM-3 agent that is TSR-022. In embodiments, a method further comprises administration of a LAG-3 agent that is a polypeptide comprising a CDR-H1 defined by SEQ ID NO: 5; a CDR-H2 defined by SEQ ID NO: 6; a CDR-H3 defined by SEQ ID NO: 7; a CDR-L1 defined by SEQ ID NO: 8; a CDR-L2 defined by SEQ ID NO: 9; and a CDR-L3 defined by SEQ ID NO: 10. In embodiments, a method further comprises administration of a LAG-3 agent that is a polypeptide comprising a heavy chain variable region amino acid sequence having at least 80%, 85%, 90%, 95%, or 98% sequence identity to SEQ ID NO: 3; and a light chain variable region amino acid sequence having at least 80%, 85%, 90%, 95%, or 98% sequence identity to SEQ ID NO: 4. In embodiments, a method further comprises administration of a LAG-3 agent that is a polypeptide comprising a heavy chain polypeptide sequence having at least 80%, 85%, 90%, 95%, or 98% sequence identity to SEQ ID NO: 1 or SEQ ID NO: 21; and a light chain polypeptide sequence having at least 80%, 85%, 90%, 95%, or 98% sequence identity to SEQ ID NO: 2 or SEQ ID NO: 22. In embodiments, a method further comprises administration of a LAG-3 agent that is TSR-033.

The dosage of a PD-1 agent, a TIM-3 agent, and a LAG-3 agent can be independently administered according to any dosage regimen described herein.

In embodiments, a PD-1 agent (e.g., a PD-1 binding agent such as TSR-042) is administered as a flat dose of about 500 mg. In embodiments, a PD-1 agent (e.g., a PD-1 binding agent such as TSR-042) is administered as a flat dose of about 1000 mg. In embodiments, a PD-1 agent (e.g., a PD-1 binding agent such as TSR-042) is administered to a subject once every week (Q1W), once every two weeks (Q2W), once every three weeks (Q3W), once every four weeks (Q4W), once every five weeks (Q5W), once every six week (Q6W), once every seven weeks (Q7W), or once every eight weeks (Q8W). In embodiments, a PD-1 agent (e.g., a PD-1 binding agent such as TSR-042) is administered to a subject once every three weeks (Q3W). In embodiments, a PD-1 agent (e.g., a PD-1 binding agent such as TSR-042) is administered to a subject once every six weeks (Q6W).

In embodiments, a TIM-3 agent (e.g., a TIM-3 binding agent such as TSR-022) is administered as a flat dose of no more than about 1200 mg or no more than about 900 mg. In embodiments, a TIM-3 agent (e.g., a TIM-3 binding agent such as TSR-022) is administered as a flat dose of about 300 mg. In embodiments, a TIM-3 agent (e.g., a TIM-3 binding agent such as TSR-022) is administered as a flat dose of about 100 mg. In embodiments, a TIM-3 agent (e.g., a TIM-3 binding agent such as TSR-022) is administered as a flat dose of about 900 mg. In embodiments, a TIM-3 agent (e.g., a TIM-3 binding agent such as TSR-022) is administered as a flat dose of about 1200 mg. In embodiments, a TIM-3 agent (e.g., a TIM-3 binding agent such as TSR-022) is administered to a subject once every week (Q1W), once every two weeks (Q2W), once every three weeks (Q3W), once every four weeks (Q4W), once every five weeks (Q5W), once every six week (Q6W), once every seven weeks (Q7W), or once every eight weeks (Q8W). In embodiments, a TIM-3 agent (e.g., a TIM-3 binding agent such as TSR-022) is administered to a subject once every three weeks (Q3W).

A LAG-3 agent can be administered in any dose or dosing regimen described herein.

In embodiments, a LAG-3 agent is administered as a flat dose of no more than about 2500 mg, about 2000 mg, or about 1500 mg. In embodiments, the methods of the present disclosure include administering the LAG-3 agent at a dose of about 20 mg, about 80 mg, about 240 mg, about 500 mg, about 720 mg, about 900 mg, about 1000 mg, about 1200 mg, about 1500 mg, about 1800 mg, about 2100 mg, about 2200 mg, or about 2500 mg. In some embodiments, the methods of the present disclosure include administering the LAG-3 agent at a dose of about 1 mg/kg, about 3 mg/kg, about 10 mg/kg, about 12 mg/kg, about 15 mg/kg, about 20 mg/kg, or about 25 mg/kg.

In embodiments, a LAG-3 agent is administered as a flat dose of no more than about 1000 mg, about 1200 mg, about 1500 mg, about 1800 mg, about 2100 mg, about 2200 mg, or about 2500 mg. In some embodiments, a LAG-3 agent is administered as a flat dose of no more than about 1000 mg, about 1200 mg, about 1500 mg. In embodiments, a LAG-3 agent is administered as a flat dose of about 20 mg, a flat dose of about 80 mg, a flat dose of about 240 mg, about 720 mg, about 900 mg, about 1000 mg, about 1500 mg, about 1800 mg, about 2100 mg, about 2200 mg, or about 2500 mg. In embodiments, a LAG-3 agent is administered as a flat dose of about 20 mg. In embodiments, a LAG-3 agent is administered as a flat dose of about 80 mg. In embodiments, a LAG-3 agent is administered as a flat dose of about 240 mg. In embodiments, a LAG-3 agent is administered as a flat dose of about 720 mg. In embodiments, a LAG-3 agent is administered as a flat dose of about 900 mg. In embodiments, a LAG-3 agent is administered as a flat dose of about 1000 mg. In embodiments, a LAG-3 agent is administered as a flat dose of about 1500 mg. In embodiments, a LAG-3 agent is administered as a flat dose of about 1800 mg. In embodiments, a LAG-3 agent is administered as a flat dose of about 2100 mg. In embodiments, a LAG-3 agent is administered as a flat dose of about 2200 mg. In embodiments, a LAG-3 agent is administered as a flat dose of about 2500 mg. In embodiments, a LAG-3 agent is administered at about 3 mg/kg, about 10 mg/kg, about 12 mg/kg, about 15 mg/kg, about 20 mg/kg, or about 25 mg/kg. In embodiments, a LAG-3 agent is administered to a subject once every week (Q1W), once every two weeks (Q2W), once every three weeks (Q3W), once every four weeks (Q4W), once every five weeks (Q5W), once every six week (Q6W), once every seven weeks (Q7W), or once every eight weeks (Q8W). In embodiments, a LAG-3 agent is administered to a subject once every two weeks (Q2W). In embodiments, a LAG-3 agent is administered as a flat dose of about 240 mg, about 720 mg, about 900 mg, about 1000 mg, or about 1500 mg to a subject once every two weeks (Q2W). In embodiments, a LAG-3 agent is administered at about 3 mg/kg, about 10 mg/kg, about 12 mg/kg, about 15 mg/kg to a subject once every two weeks (Q2W). In embodiments, a LAG-3 agent is administered to a subject once every three weeks (Q3W). In some embodiments, a LAG-3 agent is administered as a flat dose of about 720 mg, about 900 mg, about 1000 mg, about 1500 mg, about 1800 mg, about 2100 mg, about 2200 mg, or about 2500 mg to a subject once every three weeks (Q3W). In embodiments, a LAG-3 agent is administered at about 10 mg/kg, about 12 mg/kg, about 15 mg/kg, about 20 mg/kg, or about 25 mg/kg to a subject once every three weeks (Q3W). In embodiments, an-anti LAG-3 agent is a polypeptide comprising a CDR-H1 defined by SEQ ID NO: 5; a CDR-H2 defined by SEQ ID NO: 6; a CDR-H3 defined by SEQ ID NO: 7; a CDR-L1 defined by SEQ ID NO: 8; a CDR-L2 defined by SEQ ID NO: 9; and a CDR-L3 defined by SEQ ID NO: 10. In embodiments, an-anti LAG-3 agent is a polypeptide comprising a heavy chain variable region amino acid sequence having at least 80%, 85%, 90%, 95%, or 98% sequence identity to SEQ ID NO: 3; and a light chain variable region amino acid sequence having at least 80%, 85%, 90%, 95%, or 98% sequence identity to SEQ ID NO: 4. In embodiments, an-anti LAG-3 agent is a polypeptide comprising a heavy chain polypeptide sequence having at least 80%, 85%, 90%, 95%, or 98% sequence identity to SEQ ID NO: 1 or SEQ ID NO: 21; and a light chain polypeptide sequence having at least 80%, 85%, 90%, 95%, or 98% sequence identity to SEQ ID NO: 2 or SEQ ID NO: 22. In embodiments, an-anti LAG-3 agent is TSR-033.

In embodiments, a PD-1 agent (e.g., a PD-1 binding agent such as TSR-042) is administered as a flat dose of about 500 mg and a TIM-3 agent (e.g., a TIM-3 binding agent such as TSR-022) is administered as a flat dose of about 300 mg.

In embodiments, a method comprises administration of: a PD-1 agent (e.g., a PD-1 binding agent such as TSR-042) at an initial flat dose of about 500 mg; a TIM-3 agent (e.g., a TIM-3 binding agent such as TSR-022) is administered at an initial flat dose of about 300 mg; and a LAG-3 agent is administered as a flat dose of about 20 mg, about 80 mg, about 240 mg, about 500 mg, about 720 mg, about 900 mg, about 1000 mg, about 1200 mg, about 1500 mg, about 1800 mg, about 2100 mg, about 2200 mg, or about 2500 mg. In embodiments, a LAG-3 agent is administered as a flat dose of about 20 mg, a flat dose of about 80 mg, a flat dose of about 240 mg, or a flat dose of about 720 mg. In embodiments, a LAG-3 agent is administered as a flat dose of about 240 mg, about 500 mg, about 720 mg, about 900 mg, or about 1000 mg. In embodiments, administration of each of the PD-1 agent, TIM-3 agent, and LAG-3 agent occurs once every three weeks (Q3W). In embodiments, an-anti LAG-3 agent is a polypeptide comprising a CDR-H1 defined by SEQ ID NO: 5; a CDR-H2 defined by SEQ ID NO: 6; a CDR-H3 defined by SEQ ID NO: 7; a CDR-L1 defined by SEQ ID NO: 8; a CDR-L2 defined by SEQ ID NO: 9; and a CDR-L3 defined by SEQ ID NO: 10. In embodiments, an-anti LAG-3 agent is a polypeptide comprising a heavy chain variable region amino acid sequence having at least 80%, 85%, 90%, 95%, or 98% sequence identity to SEQ ID NO: 3; and a light chain variable region amino acid sequence having at least 80%, 85%, 90%, 95%, or 98% sequence identity to SEQ ID NO: 4. In embodiments, an-anti LAG-3 agent is a polypeptide comprising a heavy chain polypeptide sequence having at least 80%, 85%, 90%, 95%, or 98% sequence identity to SEQ ID NO: 1 or SEQ ID NO: 21; and a light chain polypeptide sequence having at least 80%, 85%, 90%, 95%, or 98% sequence identity to SEQ ID NO: 2 or SEQ ID NO: 22. In embodiments, an-anti LAG-3 agent is TSR-033.

Such triple combination therapies can be used to treat any of the disorders that are responsive to LAG-3 inhibition (e.g., as described herein). For example, these triple combination therapies can be used to treat patients having a cancer such as large B-cell lymphoma, thymoma, acute myeloid leukemia, testicular tumor, lung adenocarcinoma, non-small cell lung cancer, kidney clear cell cancer, breast cancer, triple negative breast cancer (TNBC), non-triple negative breast cancer (non-TNBC), gastric cancer, lung squamous cell cancer, mesothelioma, pancreatic cancer, cervical cancer, head and neck cancer, melanoma, hepatocellular carcinoma, nasopharyngeal cancer, esophageal cancer, colon adenocarcinoma, colorectal cancer, rectum carcinoma, cholangiocarcinoma, uterine endometrial cancer, sarcoma, bladder cancer, thyroid carcinoma, kidney papillary cancer, glioblastoma multiforme, liver cancer, uterine carcinosarcoma, pheocromocytoma, lower grade glioma, kidney chromophobe, adrenocortical cancer, or uveal melanoma.

PARP Inhibitors

In embodiments, an additional therapy is a poly (ADP-ribose) polymerase (PARP) inhibitor.

In embodiments, a PARP inhibitor inhibits PARP-1 and/or PARP-2. In some embodiments, the agent is a small molecule, a nucleic acid, a polypeptide (e.g., an antibody), a carbohydrate, a lipid, a metal, or a toxin. In related embodiments, the agent is ABT-767, AZD 2461, BGB-290, BGP 15, CEP 8983, CEP 9722, DR 2313, E7016, E7449, fluzoparib (SHR 3162), IMP 4297, INO1001, JPI 289, JPI 547, monoclonal antibody B3-LysPE40 conjugate, MP 124, niraparib (ZEJULA) (MK-4827), NU 1025, NU 1064, NU 1076, NU1085, olaparib (AZD2281), ONO2231, PD 128763, R 503, R554, rucaparib (RUBRACA) (AG-014699, PF-01367338), SBP 101, SC 101914, simmiparib, talazoparib (BMN-673), veliparib (ABT-888), WW 46, 2-(4-(trifluoromethyl)phenyl)-7,8-dihydro-5H-thiopyrano[4,3-dlpyrimidin-4-ol, and salts or derivatives thereof. In some related embodiments, an agent is niraparib, olaparib, rucaparib, talazoparib, veliparib, or salts or derivatives thereof. In certain embodiments, an agent is niraparib or a salt or derivative thereof. In certain embodiments, an agent is olaparib or a salt or derivative thereof. In certain embodiments, an agent is rucaparib or a salt or derivative thereof. In certain embodiments, an agent is talazoparib or a salt or derivative thereof. In certain embodiments, an agent is veliparib or a salt or derivative thereof.

Niraparib, (3S)-3-[4-{7-(aminocarbonyl)-2H-indazol-2-yl}phenyl]piperidine, is an orally available, potent, poly (adenosine diphosphate [ADP]-ribose) polymerase (PARP)-1 and -2 inhibitor. See WO 2008/084261 (published on Jul. 17, 2008), WO 2009/087381 (published Jul. 16, 2009), and PCT/US17/40039 (filed Jun. 29, 2017), the entirety of each of which is hereby incorporated by reference. Niraparib can be prepared according to Scheme 1 of WO 2008/084261.

In some embodiments, niraparib can be prepared as a pharmaceutically acceptable salt. One of skill in the art will appreciate that such salt forms can exist as solvated or hydrated polymorphic forms. In some embodiments, niraparib is prepared in the form of a hydrate.

In certain embodiments, niraparib is prepared in the form of a tosylate salt. In some embodiments, niraparib is prepared in the form of a tosylate monohydrate. The molecular structure of the tosylate monohydrate salt of niraparib is shown below:

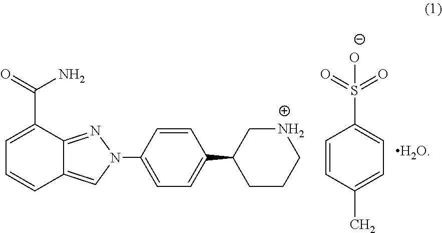

Niraparib is a potent and selective PARP-1 and PARP-2 inhibitor with inhibitory concentration at 50% of control (IC₅₀)=3.8 and 2.1 nM, respectively, and is at least 100-fold selective over other PARP-family members. Niraparib inhibits PARP activity, stimulated as a result of DNA damage caused by addition of hydrogen peroxide, in various cell lines with an IC₅₀ and an inhibitory concentration at 90% of control (IC₉₀) of about 4 and 50 nM, respectively.

In embodiments, niraparib is administered at a dose equivalent to about 100 mg of niraparib free base (e.g., a pharmaceutically acceptable salt of niraparib such as niraparib tosylate monohydrate is administered at a dose equivalent to about 100 mg of niraparib free base). In embodiments, niraparib is administered at a dose equivalent to about 200 mg of niraparib free base (e.g., a pharmaceutically acceptable salt of niraparib such as niraparib tosylate monohydrate is administered at a dose equivalent to about 200 mg of niraparib free base In embodiments, niraparib is administered at a dose equivalent to about 300 mg of niraparib free base (e.g., a pharmaceutically acceptable salt of niraparib such as niraparib tosylate monohydrate is administered at a dose equivalent to about 300 mg of niraparib free base).

### Articles of Manufacture

In one aspect of the present disclosure, an article of manufacture containing materials useful for the treatment, prevention and/or diagnosis of the disorders described above is provided. The article of manufacture can comprise a container and a label or package insert on or associated with the container. Suitable containers can include, for example, bottles, vials, syringes, IV solution bags, etc. The containers may be formed from a variety of materials such as glass or plastic. The container can hold a composition which is by itself or combined with another composition effective for treating, preventing and/or diagnosing the condition and may have a sterile access port (for example, the container may be an intravenous solution bag or a vial having a stopper pierceable by a hypodermic injection needle). At least one active agent in the composition can be an antibody of the present disclosure. The label or package insert can indicate that the composition is used for treating the condition of choice. Moreover, the article of manufacture may comprise (a) a first container with a composition contained therein, wherein the composition comprises an antibody of the disclosure; and (b) a second container with a composition contained therein, wherein the composition comprises a further cytotoxic or otherwise therapeutic agent. The article of manufacture in this embodiment of the disclosure may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The present disclosure also provides isolated nucleic acid sequences encoding the polypeptides for anti-LAG-3 antibody agents and components thereof. In some embodiments, a nucleic acid encodes an anti-LAG-3 antibody agent or component thereof. In some embodiments, a nucleic acid encodes a heavy chain and/or light chain of an anti-LAG-3 antibody agent. In some embodiments, a nucleic acid encodes a heavy chain polypeptide of SEQ ID NO:1 or 21. In some embodiments, a nucleic acid encodes a light chain polypeptide of SEQ ID NO:2 or 22. In some embodiments, a nucleic acid encodes a heavy chain variable domain of SEQ ID NO:3. In some embodiments, a nucleic acid encodes a light chain variable domain of SEQ ID NO:4. In some embodiments, a nucleic acid encodes a heavy chain variable domain that includes 1, 2, or 3 CDR sequences selected from SEQ ID NOs: 5-7. In some embodiments, a nucleic acid encodes a heavy chain variable domain that includes 1, 2, or 3 CDR sequences selected from SEQ ID NOs: 8-10.

| SEQUENCE LISTING | | |
|---|---|---|
| Heavy chain full length amino acid sequence with a signal | | |
| sequence. The underlined, non-bolded sequence identifies the | | |
| signal sequence, the italicized sequence identifies the IgG | | |
| HC γ4 constant domain, with the Serine to Proline stabilizing | | |
| mutation is shown in bold with no underline, the shaded | | |
| sequence identifies the hinge region, and the glycosylation | | |
| site (N291) is shown in bold and underline. | | |
| SEQ ID NO: 1 | | |
| MDWTWRILFLVAAATGAHSEVQLVQSGAEVKKPGATVKISCKASGFSIKDDYIHWVQQ | | |
| APGKGLEWMGWIDAMNDDSQYSSKFQGRVTITVDTSTNTAYMKLSSLRSEDTAVYYCT | | |
| YAFGGYWGQGTTVTVSSASTKGPSVFPLAPCSRSTSESTAALGCLVKDYFPEPVTVSWNSGA | | |
| LTSGVHTFPAVLQSSGLYSLSSVVTVPSSSLGTKTYTCNVDHKPSNTKVDKRVESKYGPPCPPC | | |
| PAPEFLGGPSVFLFPPKPKDTLMISRTPEVTCVVVDVSQEDPEVQFNWYVDGVEVHNAKTKP | | |
| REEQFNSTYRVVSVLTVLHQDWLNGKEYKCKVSNKGLPSSIEKTISKAKGQPREPQVYTLPPS | | |
| QEEMTKNQVSLTCLVKGFYPSDIAVEWESNGQPENNYKTTPPVLDSDGSFFLYSRLTVDKSRW | | |
| QEGNVFSCSVMHEALHNHYTQKSLSLSLGK | | |
| Light chain full length amino acid sequence with a signal | | |
| sequence. The underlined, non-bolded sequence identifies the | | |
| signal sequence, and the italicized sequence identifies the IgG | | |
| LC constant domain. | | |
| SEQ ID NO: 2 | | |
| MDMRVPAQLLGLLLLWLRGARCDIVMTQTPLSLSVTPGQPASISCRSSQSLVHSDSNTY | | |
| LHWYLQKPGQSPQLLIYLVSNRFSGVPDRFSGSGSGTDFTLKISRVEAEDVGVYFCGQST | | |
| HVPYAFGGGTKVEIKRTVAAPSVFIFPPSDEQLKSGTASVVCLLNNFYPREAKVQWKVDNAL | | |
| QSGNSQESVTEQDSKDSTYSLSSTLTLSKADYEKHKVYACEVTHQGLSSPVTKSFNRGEC | | |
| Heavy chain variable region amino acid sequence | | |
| SEQ ID NO: 3 | | |
| EVQLVQSGAEVKKPGATVKISCKASGFSIKDDYIHWVQQAPGKGLEWMGWIDAMNDD | | |
| SQYSSKFQGRVTITVDTSTNTAYMKLSSLRSEDTAVYYCTYAFGGYWGQGTTVTVSS | | |
| Light chain variable region amino acid sequence | | |
| SEQ ID NO: 4 | | |
| DIVMTQTPLSLSVTPGQPASISCRSSQSLVHSDSNTYLHWYLQKPGQSPQLLIYLVSNRFS | | |
| GVPDRFSGSGSGTDFTLKISRVEAEDVGVYFCGQSTHVPYAFGGGTKVEIK | | |
| SEQ ID NOs: 5-7 - Heavy chain CDR1, CDR2, and CDR3 amino acid | | |
| sequences, respectively | | |
| SEQ ID NO: 5 | | |
| DDYIH | | |
| SEQ ID NO: 6 | | |
| WIDAMNDDSQYSSKFQG | | |
| SEQ ID NO: 7 | | |
| AFGGY | | |
| SEQ ID NOs: 8-10 - Light chain CDR1, CDR2, and CDR3 amino acid | | |
| sequences, respectively | | |
| SEQ ID NO: 8 | | |
| RSSQSLVHSDSNTYLH | | |
| SEQ ID NO: 9 | | |
| LVSNRFS | | |
| SEQ ID NO: 10 | | |
| GQSTHVPYA | | |
| Heavy chain full length coding sequence (5′ to 3′) with | | |
| a signal sequence | | |
| SEQ ID NO: 11 | | |
| ATGGACTGGACCTGGAGGATCCTCTTCTTGGTGGCAGCAGCCACAGGTGCCCACTCC | | |
| GAGGTGCAGCTGGTGCAGTCCGGCGCTGAGGTGAAGAAGCCTGGCGCCACCGTGAA | | |
| GATCTCCTGCAAGGCCTCCGGCTTCAGCATCAAGGACGACTACATCCACTGGGTGCA | | |
| GCAGGCCCCCGGAAAAGGCCTGGAGTGGATGGGCTGGATCGACGCCATGAACGACG | | |
| ACTCCCAGTACTCCAGCAAGTTCCAGGGCAGGGTGACAATCACCGTGGACACCTCCA | | |
| CCAACACCGCCTACATGAAGCTGTCCTCCCTGCGGTCCGAGGATACCGCCGTGTACT | | |
| ACTGCACCTACGCCTTCGGCGGATACTGGGGCCAGGGCACCACAGTGACCGTGTCCT | | |
| CCGCTAGCACCAAGGGCCCATCCGTCTTCCCCCTGGCGCCCTGCTCCAGGAGCACCT | | |
| CCGAGAGCACAGCCGCCCTGGGCTGCCTGGTCAAGGACTACTTCCCCGAACCGGTG | | |
| ACGGTGTCGTGGAACTCAGGCGCCCTGACCAGCGGCGTGCACACCTTCCCGGCTGTC | | |
| CTACAGTCCTCAGGACTCTACTCCCTCAGCAGCGTGGTGACCGTGCCCTCCAGCAGC | | |
| TTGGGCACGAAGACCTACACCTGCAACGTAGATCACAAGCCCAGCAACACCAAGGT | | |
| GGACAAGAGAGTTGAGTCCAAATATGGTCCCCCATGCCCACCATGCCCAGCACCTG | | |
| AGTTCCTGGGGGGACCATCAGTCTTCCTGTTCCCCCCAAAACCCAAGGACACTCTCA | | |
| TGATCTCCCGGACCCCTGAGGTCACGTGCGTGGTGGTGGACGTGAGCCAGGAAGAC | | |
| CCCGAGGTCCAGTTCAACTGGTACGTGGATGGCGTGGAGGTGCATAATGCCAAGAC | | |
| AAAGCCGCGGGAGGAGCAGTTCAACAGCACGTACCGTGTGGTCAGCGTCCTCACCG | | |
| TCCTGCACCAGGACTGGCTGAACGGCAAGGAGTACAAGTGCAAGGTCTCCAACAAA | | |
| GGCCTCCCGTCCTCCATCGAGAAAACCATCTCCAAAGCCAAAGGGCAGCCCCGAGA | | |
| GCCACAGGTGTACACCCTGCCCCCATCCCAGGAGGAGATGACCAAGAACCAGGTCA | | |
| GCCTGACCTGCCTGGTCAAAGGCTTCTACCCCAGCGACATCGCCGTGGAGTGGGAGA | | |
| GCAATGGGCAGCCGGAGAACAACTACAAGACCACGCCTCCCGTGCTGGACTCCGAC | | |
| GGCTCCTTCTTCCTCTACAGCAGGCTAACCGTGGACAAGAGCAGGTGGCAGGAGGG | | |
| GAATGTCTTCTCATGCTCCGTGATGCATGAGGCTCTGCACAACCACTACACACAGAA | | |
| GAGCCTCTCCCTGTCTCTGGGTAAATGA | | |
| Light chain full length coding sequence (5′ to 3′) | | |
| with a signal sequence | | |
| SEQ ID NO: 12 | | |
| ATGGACATGAGGGTCCCCGCTCAGCTCCTGGGGCTCCTGCTACTCTGGCTCCGAGGT | | |
| GCCAGATGTGACATCGTGATGACCCAGACACCCCTGTCCCTGTCCGTGACACCTGGA | | |
| CAGCCCGCCTCCATCTCCTGCAGGTCCTCCCAGTCCCTGGTGCACTCCGACTCCAAC | | |
| ACCTACCTCCACTGGTACCTGCAGAAGCCTGGCCAGTCCCCCCAGCTGCTGATCTAC | | |
| CTGGTGTCCAACCGGTTCAGCGGCGTGCCTGACAGGTTCAGCGGAAGCGGCTCCGGC | | |
| ACCGACTTCACCCTGAAGATCTCCAGGGTGGAGGCCGAGGATGTGGGCGTGTACTTC | | |
| TGCGGCCAGTCCACCCACGTGCCCTATGCTTTCGGCGGCGGCACCAAGGTGGAGATC | | |
| AAGCGTACGGTGGCTGCACCATCTGTCTTCATCTTCCCGCCATCTGATGAGCAGTTG | | |
| AAATCTGGAACTGCCTCTGTTGTGTGCCTGCTGAATAACTTCTATCCCAGAGAGGCC | | |
| AAAGTACAGTGGAAGGTGGATAACGCCCTCCAATCGGGTAACTCCCAGGAGAGTGT | | |
| CACAGAGCAGGACAGCAAGGACAGCACCTACAGCCTCAGCAGCACCCTGACGCTGA | | |
| GCAAAGCAGACTACGAGAAACACAAAGTCTACGCCTGCGAAGTCACCCATCAGGGC | | |
| CTGAGCTCGCCCGTCACAAAGAGCTTCAACAGGGGAGAGTGTTGA | | |
| Heavy chain variable region coding sequence (5′ to 3′) | | |
| SEQ ID NO: 13 | | |
| GAGGTGCAGCTGGTGCAGTCCGGCGCTGAGGTGAAGAAGCCTGGCGCCACCGTGAA | | |
| GATCTCCTGCAAGGCCTCCGGCTTCAGCATCAAGGACGACTACATCCACTGGGTGCA | | |
| GCAGGCCCCCGGAAAAGGCCTGGAGTGGATGGGCTGGATCGACGCCATGAACGACG | | |
| ACTCCCAGTACTCCAGCAAGTTCCAGGGCAGGGTGACAATCACCGTGGACACCTCCA | | |
| CCAACACCGCCTACATGAAGCTGTCCTCCCTGCGGTCCGAGGATACCGCCGTGTACT | | |
| ACTGCACCTACGCCTTCGGCGGATACTGGGGCCAGGGCACCACAGTGACCGTGTCCT | | |
| CC | | |
| Light chain variable region coding sequence (5′ to 3′) | | |
| SEQ ID NO: 14 | | |
| GACATCGTGATGACCCAGACACCCCTGTCCCTGTCCGTGACACCTGGACAGCCCGCC | | |
| TCCATCTCCTGCAGGTCCTCCCAGTCCCTGGTGCACTCCGACTCCAACACCTACCTCC | | |
| ACTGGTACCTGCAGAAGCCTGGCCAGTCCCCCCAGCTGCTGATCTACCTGGTGTCCA | | |
| ACCGGTTCAGCGGCGTGCCTGACAGGTTCAGCGGAAGCGGCTCCGGCACCGACTTC | | |
| ACCCTGAAGATCTCCAGGGTGGAGGCCGAGGATGTGGGCGTGTACTTCTGCGGCCA | | |
| GTCCACCCACGTGCCCTATGCTTTCGGCGGCGGCACCAAGGTGGAGATCAAG | | |
| SEQ ID NOs: 15-17 - Heavy chain CDR1, CDR2, and CDR3 coding | | |
| sequences (5′ to 3′), respectively | | |
| SEQ ID NO: 15 | | |
| GACGACTACATCCAC | | |
| SEQ ID NO: 16 | | |
| TGGATCGACGCCATGAACGACGACTCCCAGTACTCCAGCAAGTTCCAGGGC | | |
| SEQ ID NO: 17 | | |
| GCCTTCGGCGGATAC | | |
| SEQ ID NOs: 18-20 - Light chain CDR1, CDR2, and CDR3 coding | | |
| sequences (5′ to 3′), respectively | | |
| SEQ ID NO: 18 | | |
| AGGTCCTCCCAGTCCCTGGTGCACTCCGACTCCAACACCTACCTCCAC | | |
| SEQ ID NO: 19 | | |
| CTGGTGTCCAACCGGTTCAGC | | |
| SEQ ID NO: 20 | | |
| GGCCAGTCCACCCACGTGCCCTATGCT | | |
| Heavy chain sequence without signal peptide | | |
| SEQ ID NO: 21 | | |
| EVQLVQSGAEVKKPGATVKISCKASGFSIKDDYIHWVQQAPGKGLEWMGWIDAMNDD | | |
| SQYSSKFQGRVTITVDTSTNTAYMKLSSLRSEDTAVYYCTYAFGGYWGQGTTVTVSSAS | | |
| TKGPSVFPLAPCSRSTSESTAALGCLVKDYFPEPVTVSWNSGALTSGVHTFPAVLQSSGL | | |
| YSLSSVVTVPSSSLGTKTYTCNVDHKPSNTKVDKRVESKYGPPCPPCPAPEFLGGPSVFL | | |
| FPPKPKDTLMISRTPEVTCVVVDVSQEDPEVQFNWYVDGVEVHNAKTKPREEQFNSTYR | | |
| VVSVLTVLHQDWLNGKEYKCKVSNKGLPSSIEKTISKAKGQPREPQVYTLPPSQEEMTK | | |
| NQVSLTCLVKGFYPSDIAVEWESNGQPENNYKTTPPVLDSDGSFFLYSRLTVDKSRWQE | | |
| GNVFSCSVMHEALHNHYTQKSLSLSLGK | | |
| Light chain sequence without signal peptide | | |
| SEQ ID NO: 22 | | |
| DIVMTQTPLSLSVTPGQPASISCRSSQSLVHSDSNTYLHWYLQKPGQSPQLLIYLVSNRFS | | |
| GVPDRFSGSGSGTDFTLKISRVEAEDVGVYFCGQSTHVPYAFGGGTKVEIKRTVAAPSVF | | |
| IFPPSDEQLKSGTASVVCLLNNFYPREAKVQWKVDNALQSGNSQESVTEQDSKDSTYSL | | |
| SSTLTLSKADYEKHKVYACEVTHQGLSSPVTKSFNRGEC | | |
| anti-PD-1 antibody agent heavy chain variable domain | | |
| SEQ ID NO: 23 | | |
| EVQLLESGGGLVQPGGSLRLSCAASGFTFSSYDMSWVRQAPGKGLEWVSTISGGGSYTY | | |
| YQDSVKGRFTISRDNSKNTLYLQMNSLRAEDTAVYYCASPYYAMDYWGQGTTVTVSS | | |
| A | | |
| anti-PD-1 antibody agent light chain variable domain | | |
| SEQ ID NO: 24 | | |
| DIQLTQSPSFLSAYVGDRVTITCKASQDVGTAVAWYQQKPGKAPKLLIYWASTLHTGVP | | |
| SRFSGSGSGTEFTLTISSLQPEDFATYYCQHYSSYPWTFGQGTKLEIKR | | |
| anti-PD-1 antibody agent CDR sequences | | |
| HC - CDR1 | GFTFSSYDMS | SEQ ID NO: 25 |
| HC - CDR2 | TISGGGSYTY | SEQ ID NO: 26 |
| HC - CDR3 | PYYAMDY | SEQ ID NO: 27 |
| LC - CDR1 | KASQDVGTAVA | SEQ ID NO: 28 |
| LC - CDR2 | WASTLHT | SEQ ID NO: 29 |
| LC - CDR3 | QHYSSYPWT | SEQ ID NO: 30 |
| An anti-TIM-3 antibody heavy chain polypeptide | | |
| (SEQ ID NO: 31) | | |
| EVQLLESGGGLVQPGGSLRLSCAAASGFTFSSYDMSWVRQAPGKGLDWVSTISGGGTY | | |
| TYYQDSVKGRFTISRDNSKNTLYLQMNSLRAEDTAVYYCASMDYWGQGTTVTVSSAST | | |
| KGPSVFPLAPCSRSTSESTAALGCLVKDYFPEPVTVSWNSGALTSGVHTFPAVLQSSGLY | | |
| SLSSVVTVPSSSLGTKTYTCNVDHKPSNTKVDKRVESKYGPPCPPCPAPEFLGGPSVFLFP | | |
| PKPKDTLMISRTPEVTCVVVDVSQEDPEVQFNWYVDGVEVHNAKTKPREEQFNSTYRV | | |
| VSVLTVLHQDWLNGKEYKCKVSNKGLPSSIEKTISKAKGQPREPQVYTLPPSQEEMTKN | | |
| QVSLTCLVKGFYPSDIAVEWESNGQPENNYKTTPPVLDSDGSFFLYSRLTVDKSRWQEG | | |
| NVFSCSVMHEALHNHYTQKSLSLSLGK | | |
| An anti-TIM-3 antibody light chain polypeptide | | |
| (SEQ ID NO: 32) | | |
| DIQMQSPSSLSASVGDRVTITCRASQSIRRYLNWYHQKPGKAPKLLIYGASTLQSGVPSR | | |
| FSGSGSGTDFTLTISSLQPEDFAVYYCQQSHSAPLTFGGGTKVEIKRTVAAPSVFIFPPSDE | | |
| QLKSGTASVVCLLNNFYPREAKVQWKVDNALQSGNSQESVTEQDSKDSTYSLSSTLTLS | | |
| KADYEKHKVYACEVTHQGLSSPVTKSFNRGEC | | |
| anti-TIM-3 antibody agent CDR sequences | | |
| HC - CDR1 | GFTFSSYDMS | SEQ ID NO: 33 |
| HC - CDR2 | TISGGGTYTYYQDSVKG | SEQ ID NO: 34 |
| HC - CDR3 | MDY | SEQ ID NO: 35 |
| LC - CDR1 | RASQSIRRYLN | SEQ ID NO: 36 |
| LC - CDR2 | GASTLQS | SEQ ID NO: 37 |
| LC - CDR3 | QQSHSAPLT | SEQ ID NO: 38 |

An anti-PD-1 antibody heavy chain polypeptide (CDR
sequences)
SEQ ID NO: 39
EVQLLESGGGLVQPGGSLRLSCAASGFTFSSYDMSWVRQAPGKGLEWVSTISGGGSYTY
YQDSVKGRFTISRDNSKNTLYLQMNSLRAEDTAVYYCASPYYAMDYWGQGTTVTVSS
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRW
QEGNVFSCSVMHEALHNHYTQKSLSLSLGK
An anti-PD-1 antibody light chain polypeptide (CDR sequences)
SEQ ID NO: 40
DIQLTQSPSFLSAYVGDRVTITCKASQDVGTAVAWYQQKPGKAPKWYWASTLHTGVP
SRFSGSGSGTEFTLTISSLQPEDFATYYCQHYSSYPWTFGQGTKLEIKRTVAAPSVFIFPPS
DEQLKSGTASVVCLLNNFYPREAKVQWKVDNALQSGNSQESVTEQDSKDSTYSLSSTLT
LSKADYEKHKVYACEVTHQGLSSPVTKSFNRGEC

### EXAMPLES

### Example 1

### Disulfide Bond Linkage Analysis of TSR-033

This example describes disulfide bond linkage analysis of an exemplary anti-LAG-3 antibody agent TSR-033 comprising a heavy chain of SEQ ID NO: 21 and a light chain of SEQ ID NO: 22. The amino acid residues mentioned in each example were numbered according to SEQ ID NO: 21 and SEQ ID NO: 22.

Lys-C and trypsin digested peptides were well separated and detected by on-line LC-MS analysis. The disulfide bond linkages were confirmed by comparison of total ion chromatograms in the non-reduced (NR) condition with the reduced condition. Lys-C and trypsin digestion yielded eight disulfide bond (DS1-DS8) containing peptides. All of them were confirmed by accurate masses under non-reduced condition, which was further confirmed by depleting these peptides with the reduction. Due to incomplete digestion, nine peptides, with two forms of DS5 (DS5a and DS5b) were detected and the unique hinge region with two inter-chain disulfide bonds (DS6) was shown in a single peptide. Inter- and intra-disulfide bonds were identified, and exemplary disulfide bond assignments are shown in Table 9.

TABLE 9
Exemplary disulfide bond assignments for an anti-LAG-3 antibody

| | | Linkage Sites | | |
|---|---|---|---|---|
| | | on | | |
| | | HC without | | |
| | | signal sequence | | |
| | | (SEQ ID | Linkage site | Linkage site |
| | | NO: 21) | on HC with | on LC with |
| | | and | signal | signal |
| | | LC without | sequence | sequence |
| Disulfide | Disulfide- | signal sequence | (position in | (position in |
| bond | containing | (SEQ ID | SEQ ID | SEQ ID |
| NO. | peptides | NO: 22) | NO: 1) | NO: 2) |
| DS1 | DIVMTQTPLSLSVTPGQP | LC23-LC93 | | 45 |
| | ASISCR=VEAEDVGVYFC | | | 115 |
| | GQSTHVPYAFGGGTK | | | |
| DS2 | SGTASVVCLLNNFYPR=V | LC139-LC199 | | 161 |
| | YACEVTHQGLSSPVTK | | | 221 |
| DS3 | GEC=GPSVFPLAPCSR | LC219-HC128 | 147 | 241 |
| DS4 | ISCK=SEDTAVYYCTYAF | HC22-HC96 | 41 | |
| | GGYWGQGTTVTVSSAST | | 115 | |
| | K | | | |
| DS5a | STSESTAALGCLVK=TYT | HC141-HC197 | 160 | |
| | CNVDHK | | 216 | |
| DS5b | STSESTAALGCLVK=TYT | HC141-HC197 | | |
| | CNVDHKPSNTK | | | |
| DS6 | YGPPCPPCPPAPEFLGGPS | HC220-HC220 | 239 | |
| | VFLFPPK=YGPPCPPCPAP | and | 242 | |
| | EFLGGPSVFLFPPK | HC223-HC223 | | |
| DS7 | TPEVTCVVVDVSQEDPEV | HC255-HC315 | 274 | |
| | QFNWYVDGVEVHNAK=CK | | 334 | |
| DS8 | NQVSLTCLVK=WQEGNV | HC361-HC419 | 380 | |
| | FSCSVMHEALHNHYTQK | | 438 | |

LC: 1eft chain;
HC: heavy chain

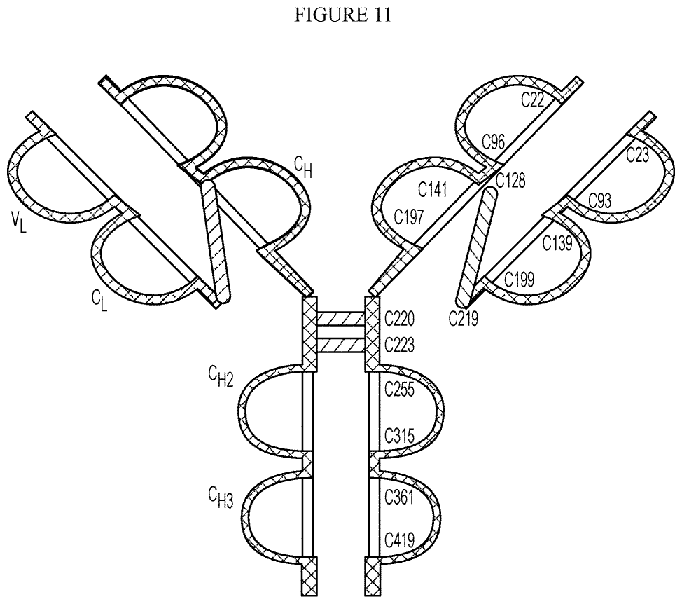

The twelve (12) intrachain disulfide bonds and four (4) interchain disulfide bonds of the exemplary anti-LAG-3 antibody are shown in FIG. 11.

### Example 2

### N-Glycan Profiling of TSR-033

This example describes N-glycan profiling of characterization of an exemplary anti-LAG-3 antibody agent TSR-033 comprising heavy chain of SEQ ID NO: 21 and light chain of SEQ ID NO: 22. The relative abundance of glycan species from a preparation of this exemplary anti-LAG-3 antibody cultured in Chinese Hamster Ovary (CHO) cells was determined. This exemplary anti-LAG-3 antibody exhibits an occupied N-glycosylation site, and the expressed N-glycosylation at this site is a mixture of oligosaccharide species typically observed on IgGs expressed in mammalian cell culture.

For example, N-glycans were released by PNGase F and labeled with 2-AB followed by HILIC separation and fluorescence detection (FLD).

The glycosylation site of the anti-LAG-3 antibody is on the heavy chain N291.

Exemplary N-glycan analyses for two exemplary lots of the anti-LAG-3 antibody agent are shown in Table 10. As shown in Table 3, detected glycans include G0F, G1F, G2F, and Man-5, as well as other oligosaccharide species.

TABLE 10
N-Glycan analysis of exemplary lots of the
anti-LAG-3 antibody agent

| Species | Lot 1* | Lot 2* |
|---|---|---|
| G0F | 62.3% | 70.2% |
| G1F | 21.6% | 14.3% |
| G2F | 4.0% | 2.1% |
| Man-5 | 3.7% | 4.5% |
| other species | 8.4% | 8.9% |

*Amounts refer to percentage of total N-linked oligosaccharides

### Example 3

### Binding Studies Using TSR-033

TSR-033 Binding to Recombinant Soluble or Cell-Surface-Expressed LAG-3

This example describes binding affinity characterization of an exemplary anti-LAG-3 antibody agent TSR-033 comprising heavy chain of SEQ ID NO: 21 and light chain of SEQ ID NO: 22.

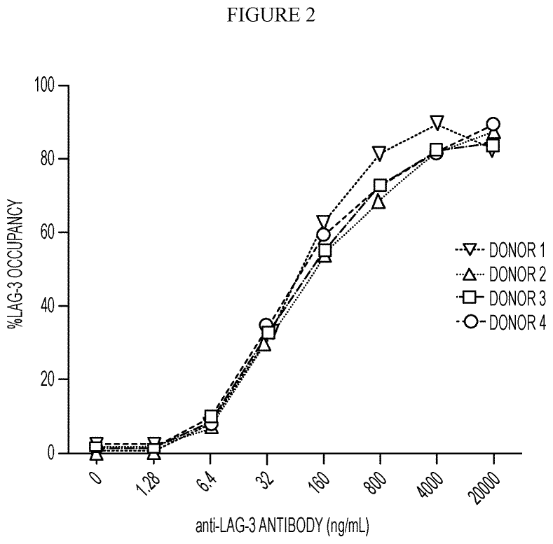

Surface plasmon resonance (SPR) was used to assess the binding of an exemplary anti-LAG-3 antibody agent to either recombinant soluble or cell-surface-expressed human and cynomolgus monkey (cyno) LAG-3 (Table 11) and to SEB-stimulated human donor peripheral blood mononuclear cells (PBMCs) (FIG. 2). Thus, the exemplary anti-LAG-3 antibody agent is able to strongly bind to both soluble LAG-3 and LAG-3 expressed on the cell surface.

TABLE 11
Binding of an Exemplary anti-LAG-3 Antibody to
Recombinant LAG-3

| | Kinetic parameters | | | LAG-3 expressing |
|---|---|---|---|---|
| | (SPR) soluble LAG-3 | | | CHO cells |
| Species | K_{assoc} (ms)⁻¹ | K_{dissoc} (s)⁻¹ | K_{D} (nM) | EC₅₀ (nM) |
| Human LAG-3 | 1.3 × 10⁵ | 1.3 × 10⁻⁴ | 0.95 | 0.8 |
| Cyno LAG-3 | 1.1 × 10⁵ | 2.6 × 10⁻⁴ | 2.0 | 2.6 |

CHO = Chinese hamster ovary;
EC₅₀ = half-maximal effective concentration;
K_{assoc} = association rate constant;
K_{dissoc} = dissociation rate constant;
K_{D} = equilibrium constant;
SPR = surface plasmon resonance.

Ligand Competition Studies

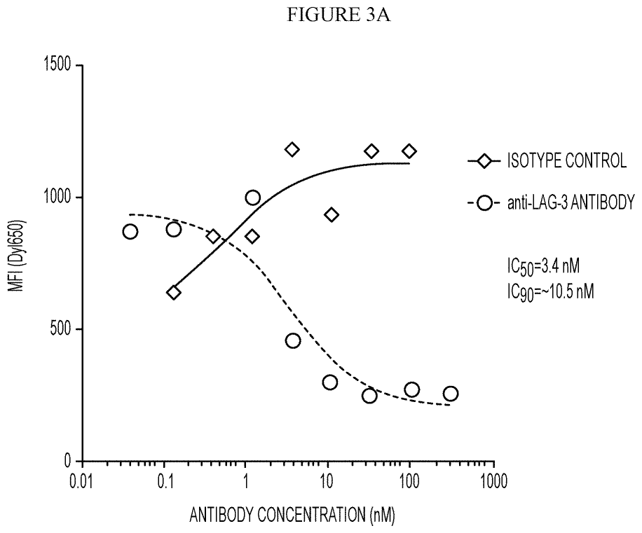

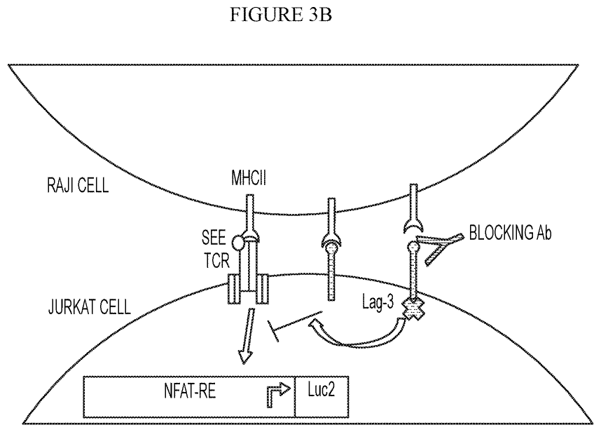

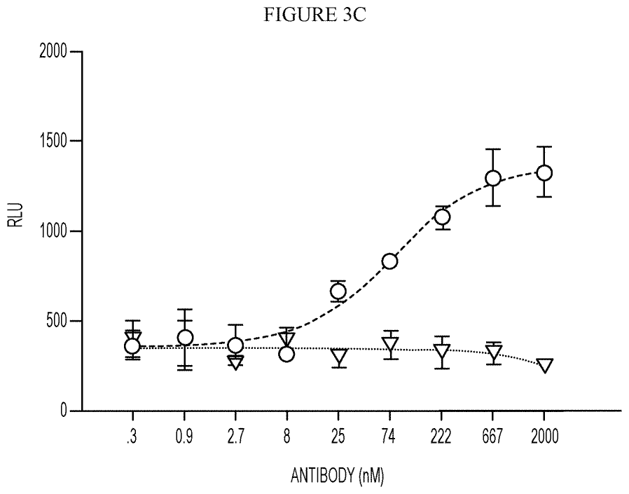

This example describes the ability of an exemplary anti-LAG-3 antibody agent TSR-033 to compete with a receptor for ligand binding. Specifically, this example uses Daudi cells, which express high levels of endogenous MHC class II and have been widely used to characterize LAG-3:MHC class II binding. (Huard et al. Proc Natl Acad Sci. 1997; 94:5744-5749). As shown in FIG. 3A shows that an exemplary anti-LAG-3 antibody agent is a potent antagonist of this interaction as determined by a flow cytometry assay that measured the ability of an exemplary anti-LAG-3 antibody agent to disrupt the binding of a DyLight 650 (DyL650)-labeled LAG-3 fusion protein to these cells.

TSR-033 Blocks LAG-3/MHC-II Binding as Assessed by a LAG-3 Reporter Gene Assay

This example describes the ability of an exemplary anti-LAG-3 antibody agent TSR-033 to block LAG-3/MHC-II binding as assessed by a LAG-3 reporter gene assay (FIG. 3B). Specifically, this example uses Raji cells, which express high levels of endogenous MHC class II. As shown in FIG. 3C and consistent with the above experiment, unlike isotype control (triangles), an exemplary anti-LAG-3 antibody agent is a potent antagonist of this interaction as determined by a reporter gene assay that measured the ability of an exemplary anti-LAG-3 antibody agent (circles) to disrupt the binding of LAG-3 expressed on Jurkat cells to MHC Class II expressing Raji cells using an NFAT reporter gene readout.

### Example 4

### Activation of Primary Human T Cells In Vitro by TSR-033

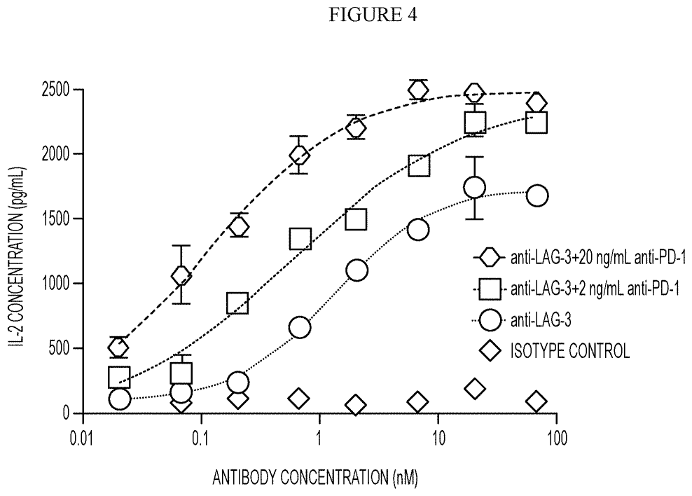

This example describes the ability of an exemplary anti-LAG-3 antibody agent to activate primary human T cells in vitro. Specifically, this example evaluated an exemplary anti-LAG-3 antibody agent TSR-033 in a mixed lymphocyte reaction (MLR) assay. In this MLR assay, primary human CD4+ T cells were mixed with monocyte-derived dendritic cells from a different donor. In these studies, dendritic cells and allogeneic CD4+ T cells were incubated in the presence of an exemplary anti-LAG-3 antibody agent or isotype control for 48 hours, and activation of T cells was determined by measuring the level of interleukin-2 (IL-2) secretion. As shown in FIG. 4, the exemplary anti-LAG-3 antibody agent dose-dependently increased IL-2 production. Moreover, this effect was further enhanced by combination with an anti-PD-1 antibody at a concentration of 2 or 20 ng/mL. These data demonstrate that blocking LAG-3 with an exemplary anti-LAG-3 antibody agent alone or in combination with anti-PD-1 can result in a potent enhancement in T-cell activation (FIG. 4).

### Example 5

### Modulation of Cytokine Release by TSR-033

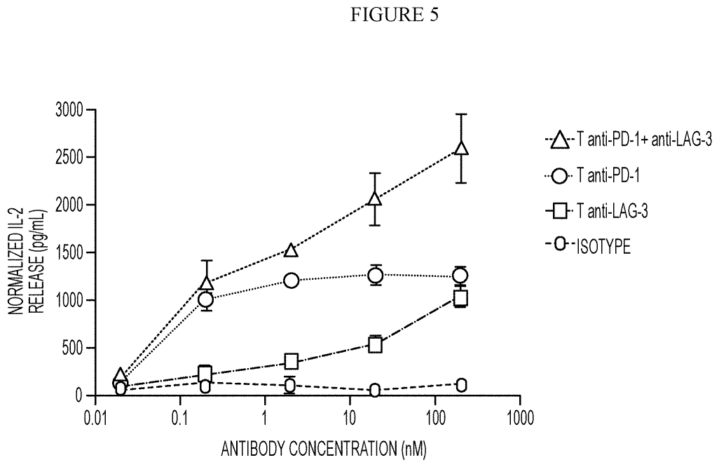

This example describes the ability of an exemplary anti-LAG-3 antibody agent TSR-033 to modulate cytokine release from SEB-activated T cells in vitro. Specifically human PBMCs (from 5 donors) were stimulated with 100 ng/mL SEB for 3 days and then IL-2 induction was assessed. In these studies, it was found that treatment with an exemplary anti-LAG-3 antibody agent dose-dependently increased IL-2 production. Moreover, dual blockade of LAG-3 and PD-1 by treatment with both an exemplary anti-LAG-3 antibody agent and an exemplary anti-PD-1 antibody agent TSR-042 resulted in a greater induction of IL-2 production than either agent alone (FIG. 5).

### Example 6

### Monotherapy Using Anti-LAG-3 Antibodies and Combination Therapy Using Anti-PD-1 and Anti-TIM-3 Antibodies in an In Vivo Xenograft Tumor Model

This example describes characterization of an exemplary, readily availabile anti-LAG-3 antibody agent C9B7W (anti-mouse LAG-3), alone or in combination with an exemplary, readily-available anti-PD-1 antibody agent RMP1-14 (anti-mouse PD-1), in an in vivo xenograft tumor model. Specifically, A20 lymphoma cells (murine DLBCL line; 200,000 cells per mouse) were implanted subcutaneously into Balb/c mice and tumors were grown to 30-50 mm before randomization (n=10 per group) for treatment. Treatment was with isotype control, exemplary anti-LAG-3 antibody agent, exemplary anti-PD-1 antibody agent, or a combination of anti-LAG-3 and anti-PD-1. Dosing for each was 10 mg/kg, twice per week.

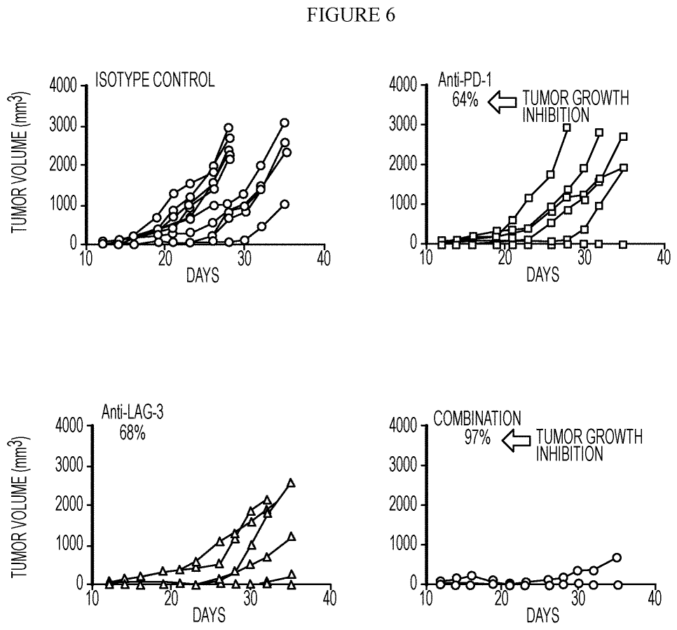

Dual blockade of PD-1 and LAG-3 exhibited further potentiated anti-tumor activity, compared to anti-PD-1 monotherapy. As shown in FIG. 6, LAG-3 blockade synergized strongly with anti-PD-1 to inhibit tumor growth (coefficient of drug interaction, CDI=0.25).

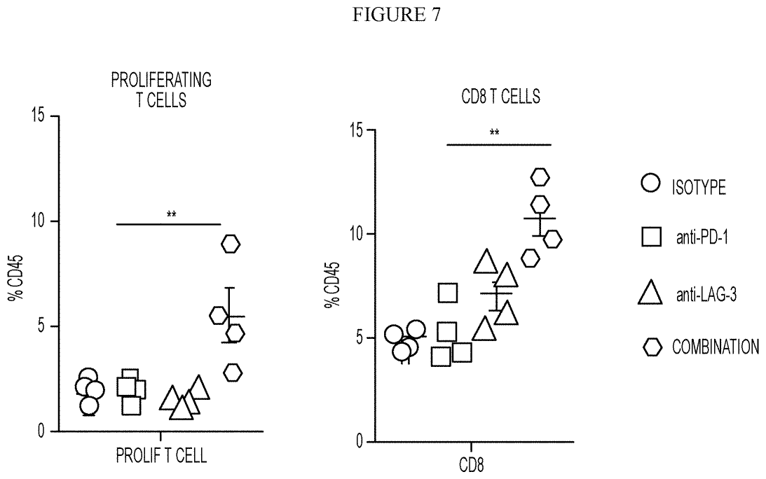

Of these xenograft mice, an n=4 per group were sacrificed on day 36, and pharmacodynamic changes of immune cells in the spleen were assessed. There were significant increases in proliferating T cells in the spleens of animals in the combination group, relative to anti-PD-1, concordant with enhanced immuno-stimulation (**p<0.01; ANOVA). See, FIG. 7.

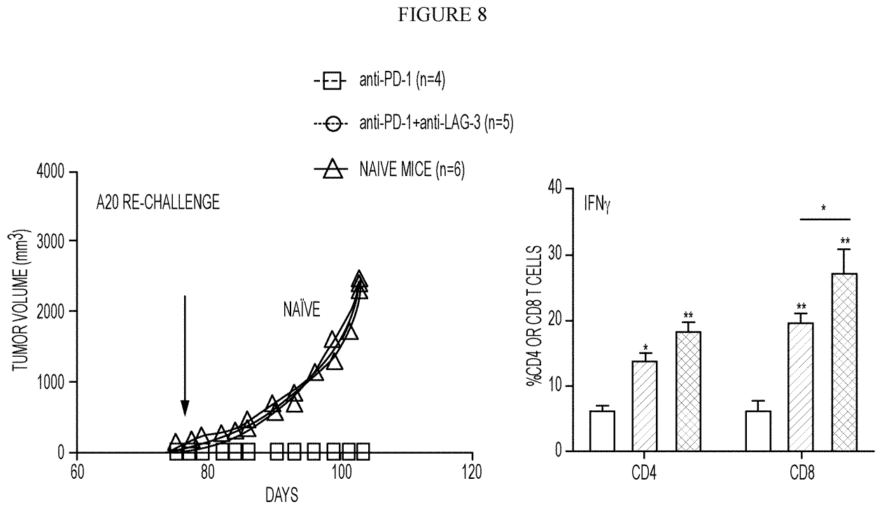

Surviving animals in each group (n=4 for anti-PD-1 and n=6 for anti-PD1+ anti-LAG-3) were monitored for tumor-free survival for 40 days, followed by re-challenge with A20 lymphoma cells (200,000 per mouse). Consistent with the development of immunological memory, tumor regrowth was not observed in either the anti-PD-1 or combination groups (see FIG. 8), though a significantly higher proportion of splenocytic interferon gamma-positive (IFNγ+) CD8 T-cells were observed in the combination arm.

Thus, these experiments demonstrate that anti-LAG-3 treatment in combination with anti-PD-1 treatment, can robustly inhibit tumor growth and induce immunostimulation. Moreover, this combination treatment engenders immunological memory in treated subjects.

### Example 7

### Monotherapy Using TSR-033 and Combination Therapy Using Exemplary Anti-LAG-3 and Anti-PD-1 Antibodies in an In Vitro T-Cell Exhaustion Model

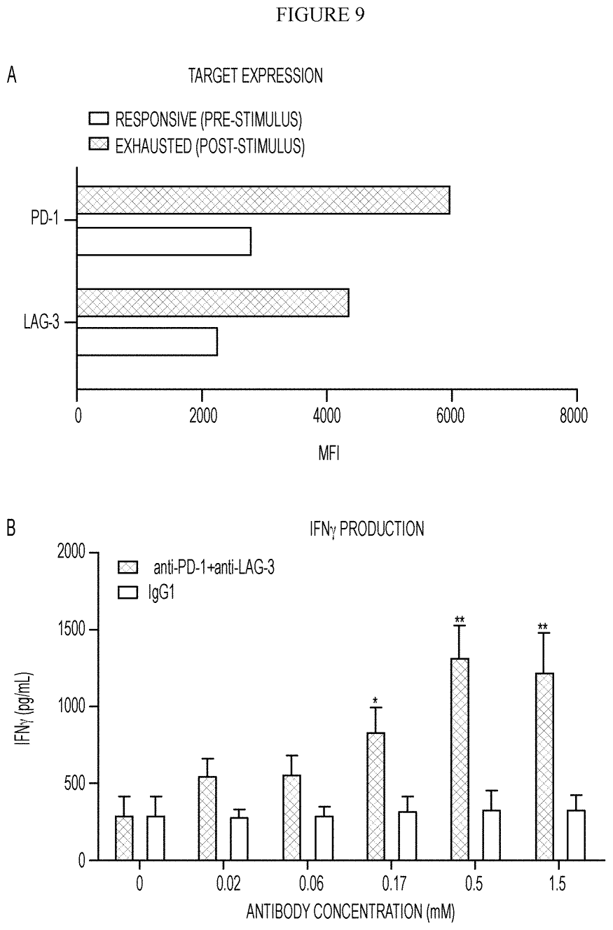

This example describes characterization of an exemplary anti-LAG-3 antibody agent C9B7W (anti-mouse LAG-3), alone or in combination with an exemplary anti-PD-1 antibody agent RMP1-14 (anti-mouse PD-1), in a murine in vitro T-cell exhaustion model. Specifically, CD4 T-cell receptor transgenic T cells were stimulated in vitro with a super-agonist altered-peptide ligand, which leads to an exhausted phenotype. This exhausted phenotype is characterized by increased expression of PD-1 and LAG-3 (FIG. 9A).

Moreover, unlike either agent alone (data not shown) or isotype control, a combination of PD-1 and LAG-3 blockade could significantly enhance IFNγ production in this system (FIG. 9B).

Having thus described at least several aspects and embodiments of this invention, it is to be appreciated that various alterations, modifications, and improvements will readily be apparent to those skilled in the art. Such alterations, modifications, and improvements are intended to be part of this disclosure, and are intended to be within the spirit and scope of the invention. Accordingly, the foregoing description and drawing are by way of example only and the invention is described in further detail by the claims that follow.

### Example 8

### Clinical Studies of Anti-LAG-3 Therapy with TSR-033, Dual Blockade Using TSR-033 and TSR-042, and Triple Blockade Therapy Using TSR-033, TSR-042, and TSR-022

Clinical Studies of TSR-033 Monotherapy and TSR-033/TSR-042 Combination Therapy

A clinical study in human patients was performed using either anti-LAG-3 antibody TSR-033 as a monotherapy, or using a combination of anti-LAG-3 antibody TSR-033 and anti-PD-1 antibody TSR-042.

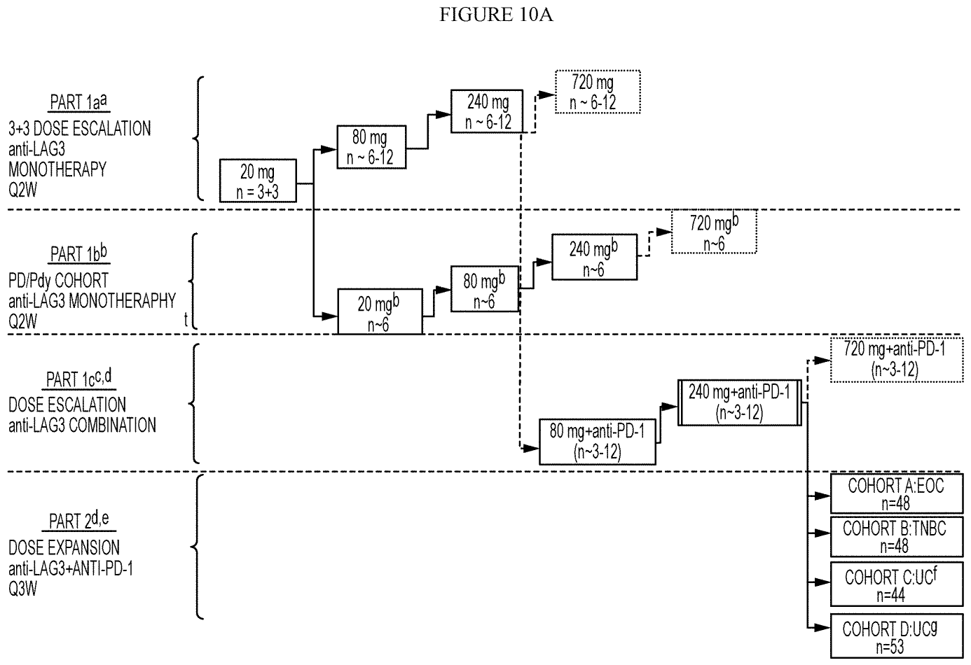

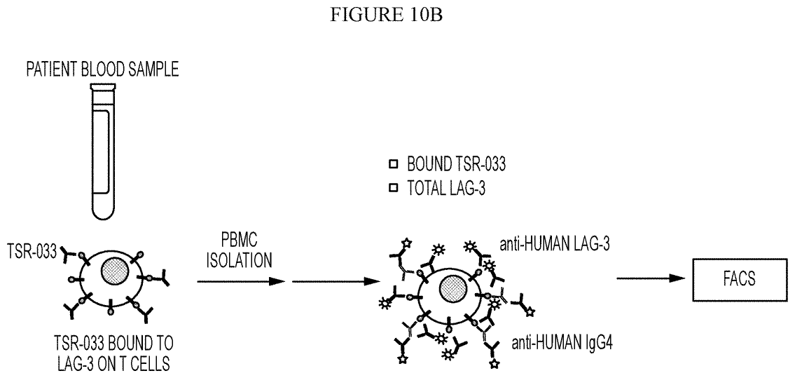

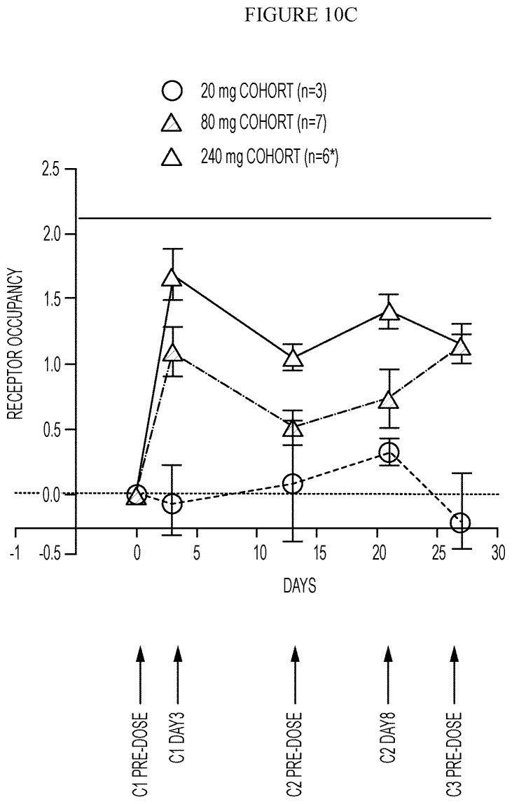

A design schema for these studies is presented in FIG. 10A. Patients with any one or more of the following tumor types were considered for inclusion in the study: solid tumors, advanced solid tumors, epithelial ovarian cancer (EOC), triple-negative breast cancer (TNBC), post-anti-PD-1/PD-L1 urothelial carcinoma (UC), and anti-PD-1/L1 naïve UC.

The primary objectives of this study include: to evaluate anti-tumor activity of anti-LAG-3 as a monotherapy and as a combination therapy with anti-PD-1 in patients with solid tumors, to define the recommended dose and schedule of anti-LAG-3 as a monotherapy and as a combination therapy with anti-PD-1, and to evaluate the safety and tolerability of anti-LAG-3 as a monotherapy and as a combination therapy with anti-PD-1.

Receptor Occupancy Studies of TSR-033

A direct binding assay was used to measure the binding of an exemplary anti-LAG-3 antibody agent TSR-033 in samples from patients. Receptor occupancy (RO) is expressed as a ratio of bound TSR-033 to total LAG-3 and was normalized to baseline (pre-dose) for each patient.

PBMCs were isolated from fresh whole blood samples from patients and treated either with isotype control or TSR-033 at saturating concentrations (both at 50 μg/mL), followed by a cocktail of detection antibodies: (1) anti-human IgG4 secondary antibody (for bound TSR-033) (2) anti-human LAG-3 antibody which does not cross compete with TSR-033 (for total LAG-3 levels) and (3) antibody cocktail for detection of T cells. The ratio of TSR-033 to total LAG-3 on T cells was determined by flow cytometry. The saturation binding aliquot was run in parallel as a control to assess the range of the assay. A schematic is provided in FIG. 10B

FIG. 10C depicts receptor occupany (RO) as measured using patient T cells. From the data, there appears to be increasing target engagement observed with increasing doses. For example, at early time points receptor occupancy approaches saturation at a 240 mg dose (top data set) and approximately 50% at an 80 mg dose (middle data set). Further, there appears to be a significant correlation between serum concentrations of TSR 033 and LAG-3 receptor occupancy in data collected to date.

Dosing Schedules

A series of ascending anti-LAG-3 doses was evaluated in this human clinical study, either alone as a monotherapy or in combination with anti-PD-1. The monotherapy human clinical study includes the following dose escalations: 20 mg/patient, 80 mg/patient, 240 mg/patient, and 720 mg/patient. Dosages between 240 mg/patient and 720 mg/patient will also be evaluated. For example, TSR-033 has been administered to patients at dosages of 20 mg/patient, 80 mg/patient, and 240 mg/patient. Patients receiving anti-LAG-3 monotherapy were administered an anti-LAG-3 dose every 14 days±1 day (Q2W) via 30 (−5 and +15)-minute intravenous (IV) infusion.

For the combination therapies comprising anti-LAG-3 and anti-PD-1, the anti-PD-1 will be dosed at 500 mg/patient in combination with ascending doses of anti-LAG-3 every 21 days±1 day (Q3W). The ascending doses in this study include: 20 mg/patient, 80 mg/patient, 240 mg/patient, 720 mg/patient, and doses between 240-720 mg/patient.

Table 12 also provides exemplary doses of LAG-3 agents (e.g., TSR-033) as administered in exemplary Q2W and Q3W schedules. The exemplary doses of Table 12 are also suitable as doses of a LAG-3 agent (e.g., TSR-033) in combination therapies (e.g., dual or triple blockade therapy).

TABLE 12
Dosing Schedules for an Exemplary LAG-3 Agent TSR-033

| Q2W Administration | | Q3W Administration | |
|---|---|---|---|
| (once every two weeks) | | (once every three weeks) | |
| Flat Dose | Weight-based Dose | Flat Dose | Weight-based Dose |
| about 240 mg | about 3 mg/kg | about 500 mg | about 10 mg/kg |
| about 500 mg | about 10 mg/kg | about 720 mg | about 12 mg/kg |
| about 720 mg | about 12 mg/kg | about 900 mg | about 15 mg/kg |
| about 900 mg | about 15 mg/kg | about 1000 mg | about 20 mg/kg |
| about 1000 mg | — | about 1500 mg | about 25 mg/kg |
| about 1000 mg | — | about 1800 mg | — |
| — | — | about 2100 mg | — |
| — | — | about 2200 mg | — |
| — | — | about 2500 mg | — |

For example, a LAG-3 agent (e.g., TSR-033) can be administered as: a flat dose of about 240 mg once every two weeks (Q2W), a flat dose of about 500 mg once every two weeks (Q2W), a flat dose of about 720 mg once every two weeks (Q2W), a flat dose of about 900 mg once every two weeks (Q2W), a flat dose of about 1000 mg once every two weeks (Q2W), a flat dose of about 1500 mg once every two weeks (Q2W), a weight-based dose of about 3 mg/kg once every two weeks (Q2W), a weight-based dose of about 10 mg/kg once every two weeks (Q2W), a weight-based dose of about 12 mg/kg once every two weeks (Q2W), a weight-based dose of about 15 mg/kg once every two weeks (Q2W), a flat dose of about 500 mg once every three weeks (Q3W), a flat dose of about 720 mg once every three weeks (Q3W), a flat dose of about 900 mg once every three weeks (Q3W), a flat dose of about 1000 mg once every three weeks (Q3W), a flat dose of about 1500 mg once every three weeks (Q3W), a flat dose of about 1800 mg once every three weeks (Q3W), a flat dose of about 2100 mg once every three weeks (Q3W), a flat dose of about 2200 mg once every three weeks (Q3W), a flat dose of about 2500 mg once every three weeks (Q3W), a weight-based dose of about 10 mg/kg once every three weeks (Q3W), a weight-based dose of about 12 mg/kg once every three weeks (Q3W), a weight-based dose of about 15 mg/kg once every three weeks (Q3W), a weight-based dose of about 20 mg/kg once every three weeks (Q3W), or a weight-based dose of about 25 mg/kg once every three weeks (Q3W).

Clinical Studies of Triple Blockade Therapy Using TSR-033, TSR-042, and TSR-022

Triple blockade therapy can be studied by administering to a subject a LAG-3 agent TSR-033, a PD-1 agent TSR-042, and a TIM-3 agent TSR-022. One or more of the agents can be administered on a Q2W or Q3W schedule while the other agent(s) are administered on a Q6W schedule. One or more of the agents can initially be administered at a Q2W or Q3W schedule (at an initial dose) and subsequently administered at a Q3W schedule (at the initial dose, at a lower dose or at a higher dose) after 2, 3, 4, 5, or 6 cycles. For example, a PD-1 agent TSR-042 can be initially administered at a dose of 500 mg/patient on a Q23W schedule and subsequently administered at a dose of 1000 mg/patient on a Q6W schedule after 2, 3, 4, 5, or 6 cycles. The three agents can be administered on a Q2W schedule. The three agents can be administered on a Q3W schedule. Alternatively, the PD-1 agent TSR-042 and TIM-3 agent TSR-022 can be administered on a Q3W schedule, and the LAG-3 agent TSR-033 can be administered on a Q2W schedule. The PD-1 agent TSR can be administered on a Q6W schedule, and the LAG-3 agent TSR-033 and 042 and TIM-3 agent TSR-022 can be administered on a Q2W or Q3W schedule.

TSR-022 and TSR-042 can be administered according to any dosage regimen herein, and TSRO-022 and TSR-042 can be administered on a Q3W or Q6W schedule.

TSR-033 can be administered according to any dosage regimen herein, including the exemplary dosing regimens of Table 12. For example, TSR-033 can be administered as: a flat dose of about 240 mg once every two weeks (Q2W), a flat dose of about 500 mg once every three weeks (Q2W), a flat dose of about 720 mg once every two weeks (Q2W), a flat dose of about 900 mg once every two weeks (Q2W), a flat dose of about 1000 mg once every two weeks (Q2W), a flat dose of about 1500 mg once every two weeks (Q2W), a weight-based dose of about 3 mg/kg once every two weeks (Q2W), a weight-based dose of about 10 mg/kg once every two weeks (Q2W), a weight-based dose of about 12 mg/kg once every two weeks (Q2W), a weight-based dose of about 15 mg/kg once every two weeks (Q2W), a flat dose of about 500 mg once every three weeks (Q3W), a flat dose of about 720 mg once every three weeks (Q3W), a flat dose of about 900 mg once every three weeks (Q3W), a flat dose of about 1000 mg once every three weeks (Q3W), a flat dose of about 1500 mg once every three weeks (Q3W), a flat dose of about 1800 mg once every three weeks (Q3W), a flat dose of about 2100 mg once every three weeks (Q3W), a flat dose of about 2200 mg once every three weeks (Q3W), a flat dose of about 2500 mg once every three weeks (Q3W), a weight-based dose of about 10 mg/kg once every three weeks (Q3W), a weight-based dose of about 12 mg/kg once every three weeks (Q3W), a weight-based dose of about 15 mg/kg once every three weeks (Q3W), a weight-based dose of about 20 mg/kg once every three weeks (Q3W), or a weight-based dose of about 25 mg/kg once every three weeks (Q3W).

TSR-033 also can be administered at a dose of: 20 mg/patient, 80 mg/patient, 240 mg/patient, 720 mg/patient, and intermediate doses between 240-720 mg/patient. TSR-033 also can be administered at a dose of up to about 1000 mg/patient (e.g., a dose of about 20, 80, 240, 500, 720, 900, or 1000 mg/patient). A dose of TSR-033 can be less than a dose used for TSR-033 monotherapy. Such a dose can be administered once every two weeks (Q2W) or once every three weeks (Q3W).

Dose modification (e.g., dose escalation) of TSR-022 also can be studied. For example, TSR-022 can be administered at a dose of 100 mg, 300 mg, 900 mg or 1200 mg.

Dosing of TSR-042 can be fixed at 500 mg or 1000 mg.

### Example 9

### Additional Studies Relating to Mono-, Dual- and Triple-Blockade Therapies

The expression of PD-1, TIM-3, and LAG-3 on patient-derived tumor infiltrating leukocytes (TILs) was studied. Further evaluation of a functional role for dual or triple blockade of PD-1, TIM-3, and LAG-3 in vivo was undertaken using humanized mouse tumor models.

Flow cytometry was used to enumerate immune cell populations in a panel of human tumor samples, including non-small cell lung cancer (NSCLC). NOG-EXL humanized mice were administered test antibodies after tumors reached 80-120 mm³. Antibodies were administered intraperitoneally twice weekly at a 10 mg/kg dose. Murine tumors and spleens were collected at termination, followed by immune-phenotyping for T cells and myeloid cells.

Co-Expression of PD-1, TIM-3, and LAG-3 on Multiple TIL Subsets Across Human Tumors

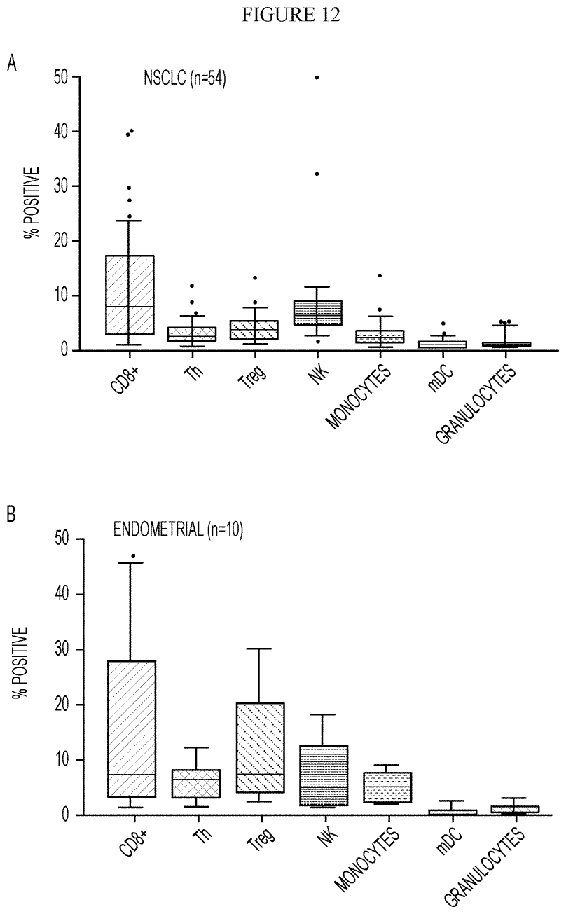

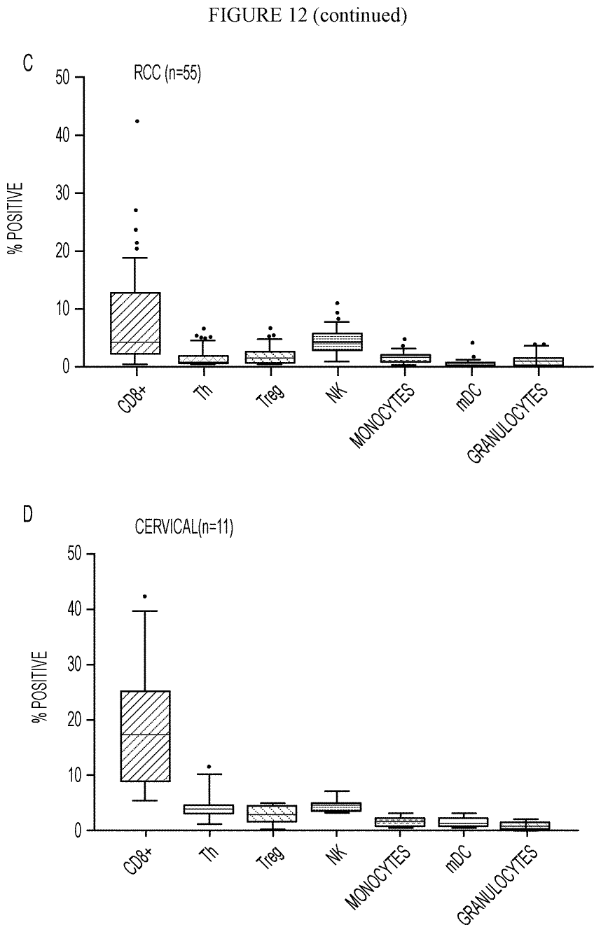

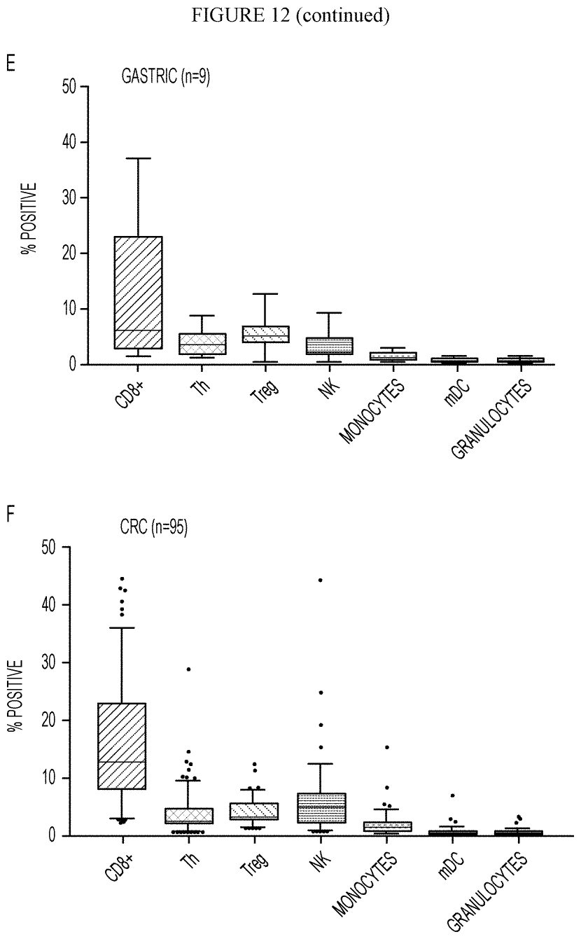

Primary resected tumors from multiple patients with different cancers were collected and dissociated into single cell suspensions using both enzymatic and mechanical disruption. Cells were immediately stained with three antibody panels including lineage markers for T and myeloid cell populations and immune checkpoint receptors. See FIGS. 12A-12F. Appreciable co-expression of PD-1, TIM-3, and LAG-3 was detected on tumor infiltrating cells, particularly CD8+ T cells, in non-small cell lung cancer (NSCLC) (FIG. 12A), endometrial (FIG. 12B), renal (RCC) (FIG. 12C), cervical (FIG. 12D), gastric (FIG. 12E), and colorectal cancer (CRC) (FIG. 12F).

Dual or Triple Checkpoint Expression Mark Dysfunctional CD8+ T Cells

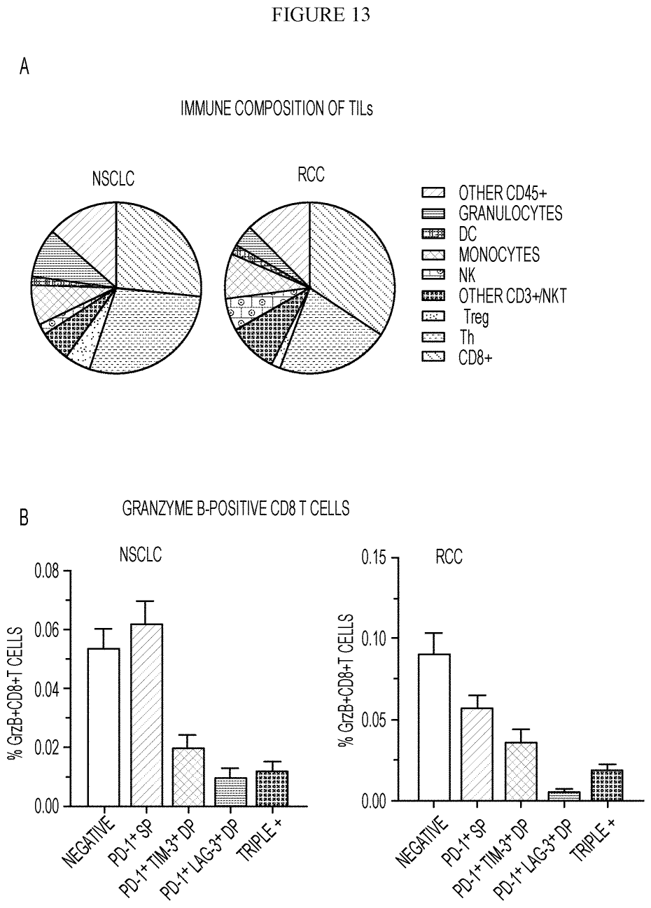

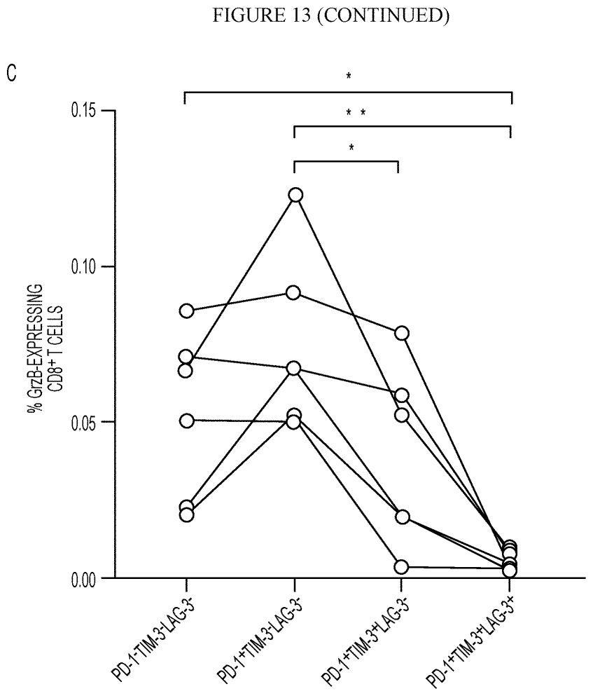

Primary resected tumors were dissociated into single cell suspensions using both enzymatic and mechanical disruption. Cells were immediately stained with three antibody panels including linear markers for T and myeloid cell populations, immune checkpoint receptors. FIG. 13A shows the immune composition of tumor-infiltrating leukocytes as determined by flow cytometry using tumor samples from NSCLC and RCC patients. FIG. 13B depicts studies using granzyme B as a functional marker for T and NK cells.

To understand the functional consequences of triple checkpoint expression, TILs were isolated and analyzed from primary EGFR+ NSCLC and found that PD-1⁺TIM-3⁺LAG-3⁺ cytotoxic T cells were highly dysfunctional, as assessed by Granzyme B status (FIG. 13C). Primary resected tumors from NSCLC patients were dissociated and CD8⁺ T cells were characterized with respect to checkpoint expression and functional status using Granzyme B (GrzB) (N=6). Statistical differences were detected by one-way ANOVA with Holm-Sidak's multiple comparison correction. *p<0.05; **p<0.01; ****p<0.0001. As shown in FIG. 13C, dual or triple checkpoint expression marks dysfunctional CD8+ T cells.

NSCLC Tumors

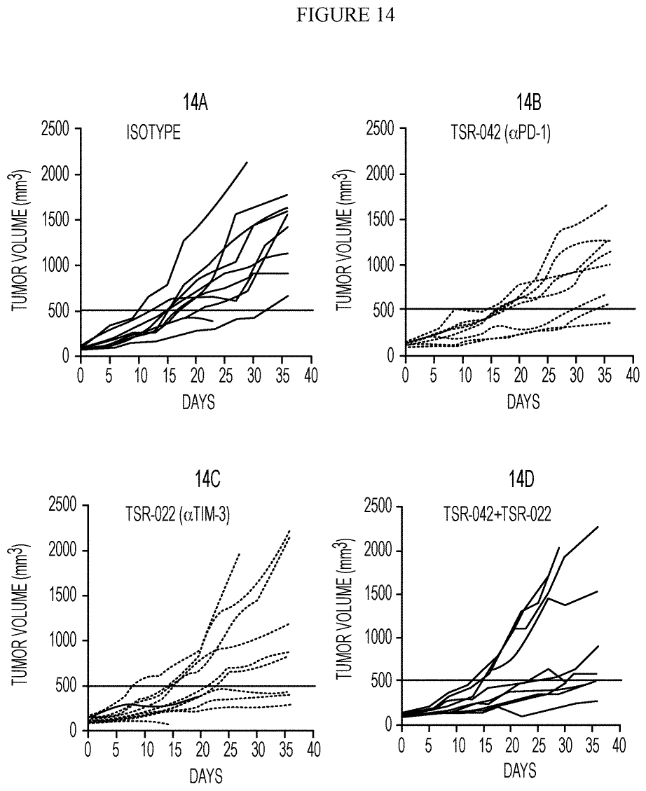

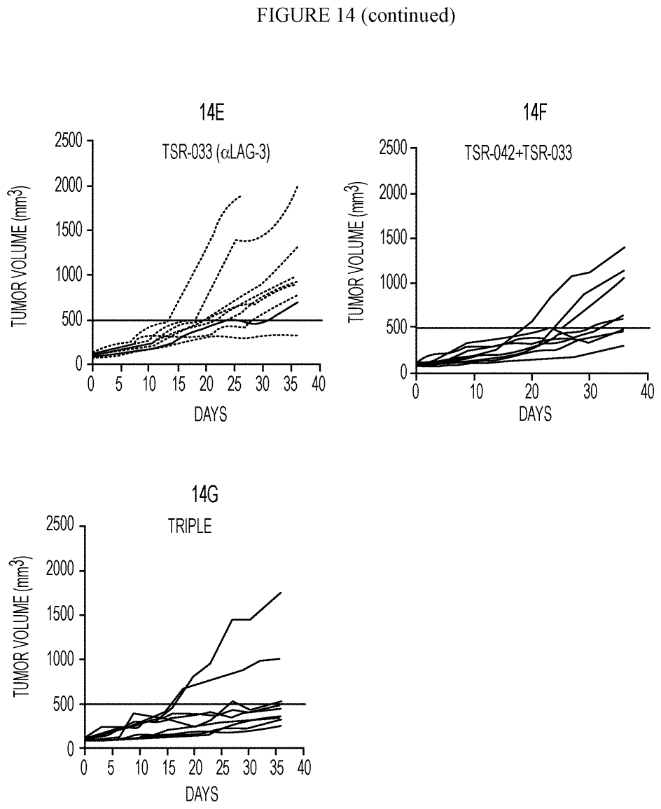

Humanized NOG-EXL mice (Taconic) were inoculated subcutaneously with A549 non-small cell lung cancer (NSCLC) (FIG. 14A-14G) cell lines and monitored for tumor growth. Mice were randomized when tumor volumes reached 80-120 mm³ and treated with the following test antibodies intraperitoneally twice weekly: human IgG4 isotype control or humanized antibodies targeting human PD-1 (TSR-042), TIM-3 (TSR-022), and LAG-3 (TSR-033). The immune checkpoint antibodies were dosed either singly or in combination at 10 mg/kg as depicted (n=5-10 animals per treatment arm). Tumor growth was monitored for 30-35 days. FIG. 14A relates to treatment with a human IgG4 isotype control; FIG. 14B relates to treatment with TSR-042; FIG. 14C relates to treatment with TSR-022; FIG. 14D relates to treatment with a combination of TSR-042 and TSR-022; FIG. 14E relates to treatment with TSR-033; FIG. 14F relates to treatment with a combination of TSR-042 and TSR-033; and FIG. 14G relates to treatment with a triple combination of TSR-042, TSR-022, and TSR-033.

NSCLC tumors were collected from all animals remaining on the study at termination (day 37 post-randomization) and processed immediately. To prepare single cell suspensions, tumor samples were digested, followed by staining and immunophenotyping by flow cytometry using markers for T and myeloid cells. Cells were gated on singlet, live populations.

Increased TILs and Reduced Intratumoral Tregs with Triple Checkpoint Blockade

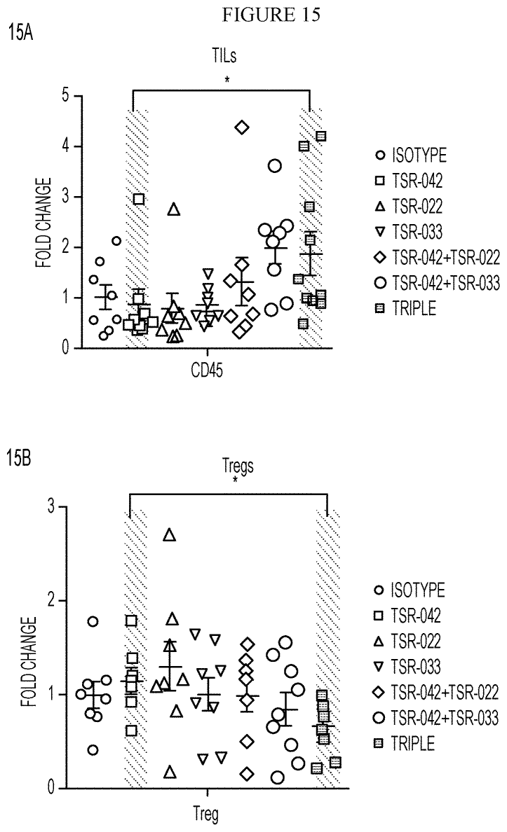

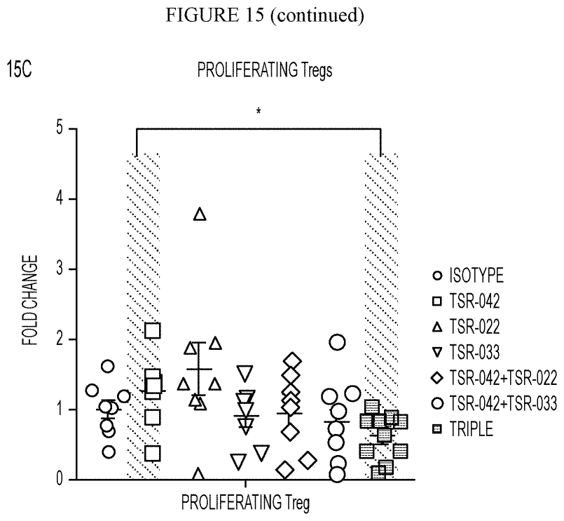

Immune cell populations for each treatment arm have been normalized to isotype control. FIG. 15A shows the fold change in tumor-infiltrating lymphocytes (CD45). FIG. 15B shows the fold change in regulatory T cells (Tregs), with Tregs being identified as CD4+FOXP3+. FIG. 15C shows the fold change in proliferating Tregs, and Ki-67 was used as a marker for proliferating cells. An asterisk is used to identify a p<0.05 in unpaired Student's T-test, comparing α-PD-1 monotherapy to ether dual or triple checkpoint combinations.

Reduction in Tumor Associated Macrophages (TAMs) and Increased M1/M2 Ratios Upon Dual or Triple Checkpoint Blockade

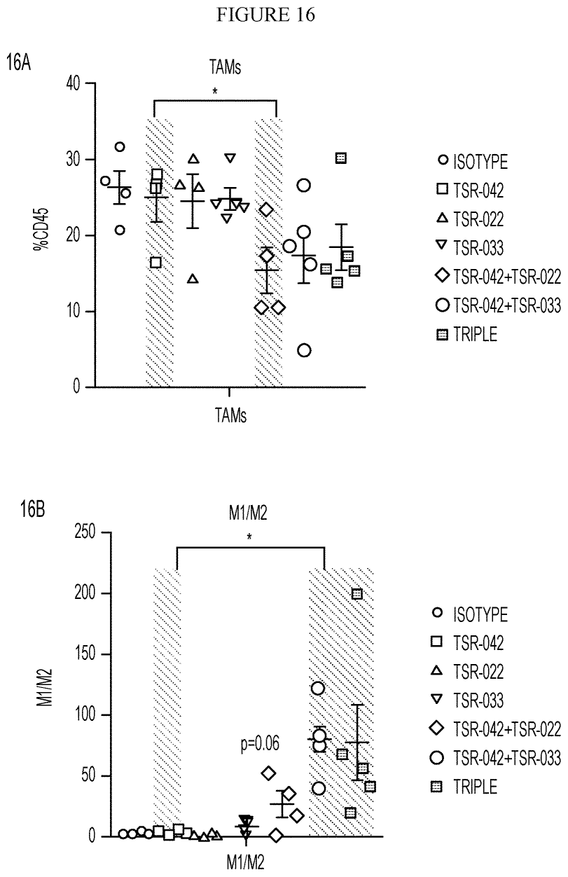

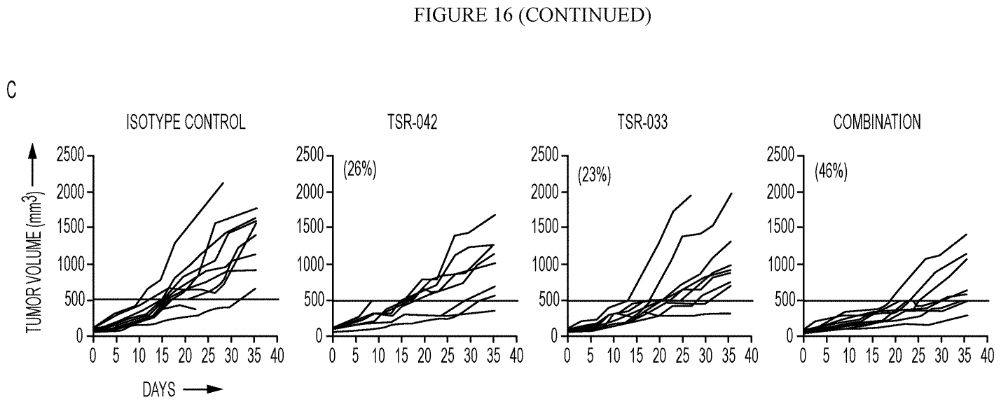

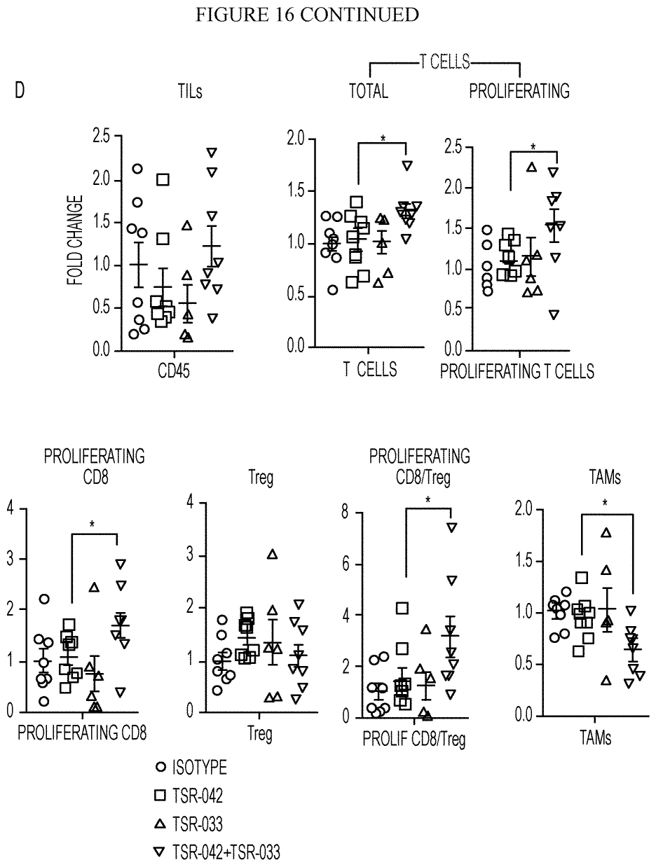

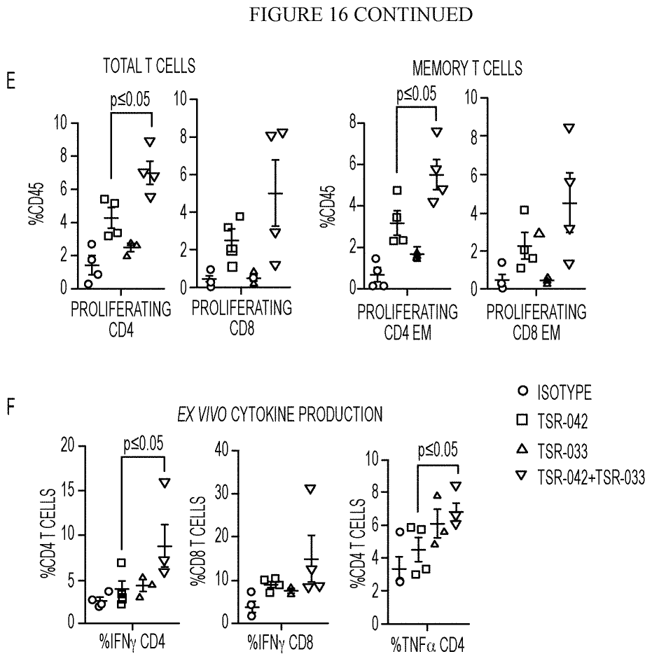

Tumor associated macrophages (TAMs) were identified as CD11b⁺CD6⁸⁺; M2 TAMs as CD11b⁺CD68⁺CD209⁺HLA-DR^{lo/-}; and M1 TAMs as CD11b⁺CD68⁺CD209⁻HLA-DR^{hi}. An unpaired Student's T-test *p<0.05 was used to compare α-PD-1 monotherapy to double or triple treatment arms. See FIGS. 16A (TAMs) and 16B (M1/M2).

Combination of TSR-033 and TSR-042 for In Vivo Anti-Tumor Activity

This example describes the ability of an exemplary anti-LAG-3 antibody agent in combination with an exemplary PD-1 agent to demonstrate anti-tumor activity in vivo.

Dual blockade of LAG-3 and PD-1 improves therapeutic efficacy and immune activation in a humanized NSCLC tumor mouse model. As shown in FIG. 16C, the combination of anti-LAG-3 with anti-PD-1 (both administered 10 mg/kg ip twice per week) has an additive effect (coefficient of drug interaction, CDI=1.001) on restricting tumor growth in HuNOG-EXL mice inoculated with A549 cells.

Mice were randomized at tumor volumes of 80-120 mm³, followed by administration of the immunotherapeutic agents. Tumor growth inhibition at termination for each treatment arm is indicated in parentheses. Relative to anti-PD-1 monotherapy, the combination group had increased tumor infiltrating lymphocytes, intra-tumoral proliferating T cells, CD8/Treg ratios and reduced TAMs (unpaired Student's T-test) (FIG. 16D). Data represent two independent experiments (n=10 per treatment arm) and have been normalized to fold change over isotype control for each treatment arm.

Combination treatment also led to increased splenic T cell proliferation, with significant increases in proliferating CD4 as well as CD4 effector-memory T cells relative to anti-PD-1 monotherapy; proliferating CD8 and CD8 effector-memory T cells were also elevated in the combination group but the trend did not reach significance (FIG. 16E).

Further, ex vivo stimulation of splenocytes with phorbol myristate acetate (PMA)/ionomycin, a common T cell stimulus, yielded higher percentages of IFNγ and TNFα producing CD4 T cells in animals dosed with combination therapy (FIG. 16F), suggesting increased functional invigoration of T cells in the combination group relative to anti-PD-1 alone.

TNBC Tumors

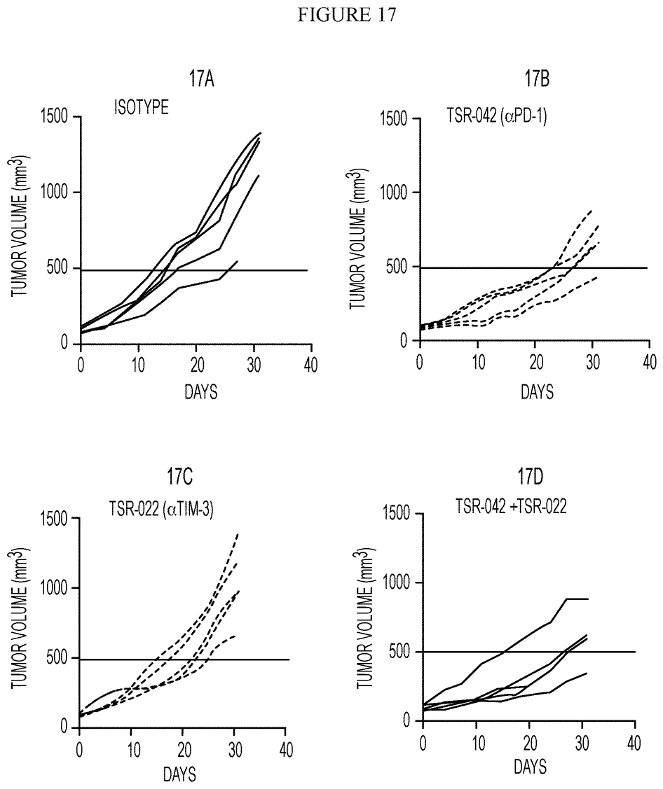

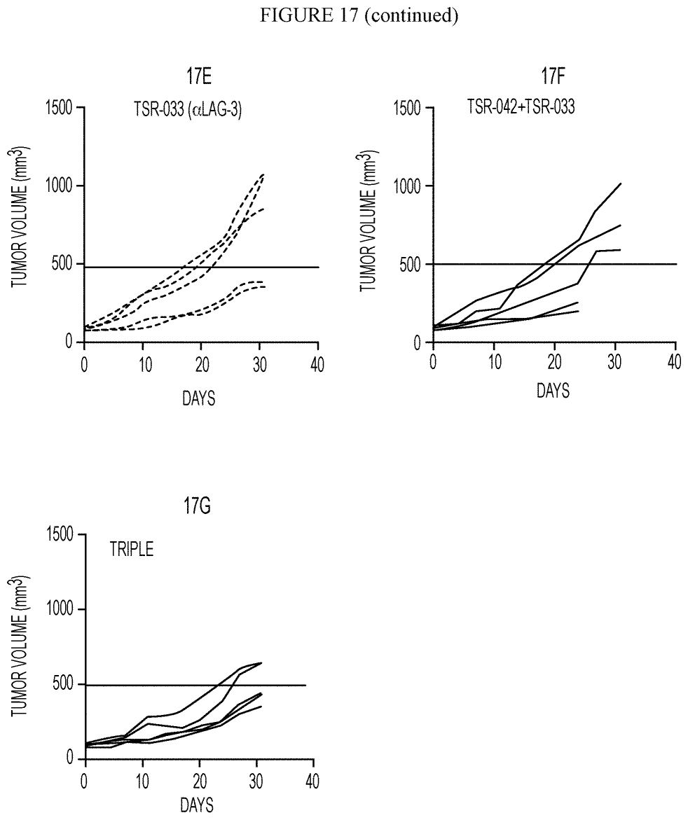

Humanized NOG-EXL mice (Taconic) were inoculated subcutaneously with MDA-MB436 triple negative breast cancer (TNBC) (FIG. 17A-17G) cell lines and monitored for tumor growth. Mice were randomized when tumor volumes reached 80-120 mm³ and treated with the following test antibodies intraperitoneally twice weekly: human IgG4 isotype control or humanized antibodies targeting human PD-1 (TSR-042), TIM-3 (TSR-022), and LAG-3 (TSR-033). The immune checkpoint antibodies were dosed either singly or in combination at 10 mg/kg as depicted (n=5-10 animals per treatment arm). Tumor growth was monitored for 30-35 days. FIG. 17A relates to treatment with a human IgG4 isotype control; FIG. 17B relates to treatment with TSR-042; FIG. 17C relates to treatment with TSR-022; FIG. 17D relates to treatment with a combination of TSR-042 and TSR-022; FIG. 17E relates to treatment with TSR-033; FIG. 17F relates to treatment with a combination of TSR-042 and TSR-033; and FIG. 17G relates to treatment with a triple combination of TSR-042, TSR-022, and TSR-033.

EMT-6 Breast Cancer Line

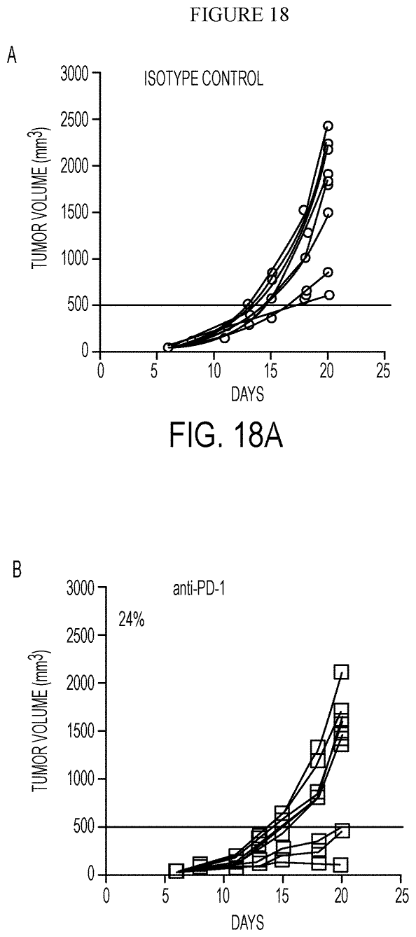

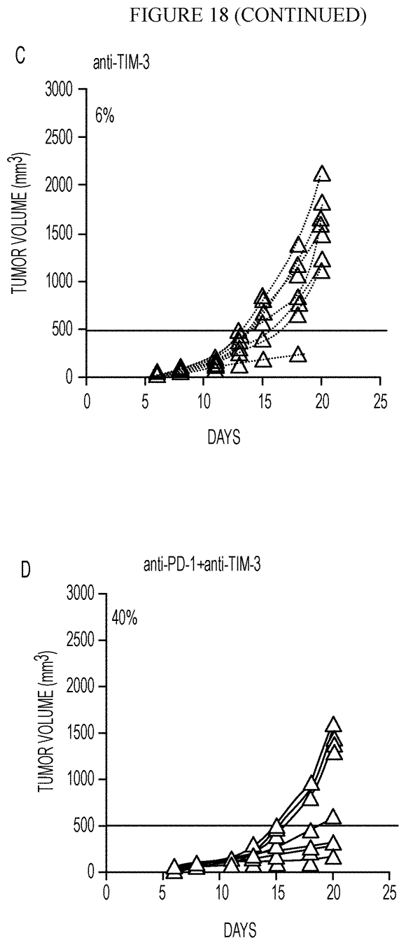

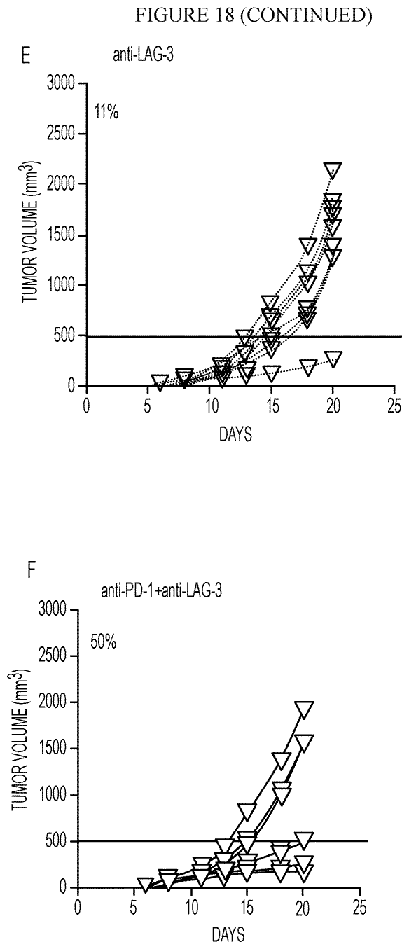

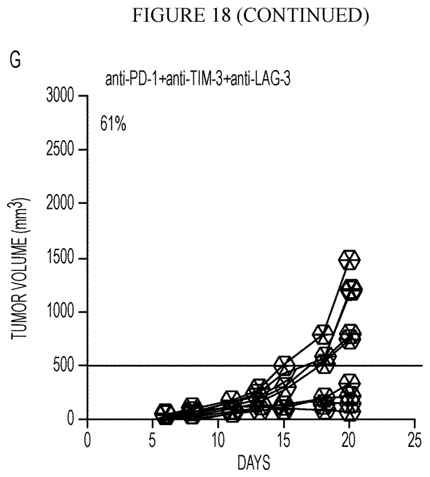

FIGS. 18A through 18G depict a study in a syngeneic tumor mouse model wherein BALB/c mice were first inoculated subcutaneously with EMT-6 breast carcinoma cell lines and then treated with surrogate test antibodies that were administered intraperitoneally twice weekly, with dosing at 10 mg/kg. Tumor volume (mm³) was measured from 0-20 days following treatment with: an isotype control (FIG. 18A); anti-PD-1 antibody (FIG. 18B); anti-TIM-3 antibody (FIG. 18C); a combination of anti-PD-1 and anti-TIM-3 (FIG. 18D); anti-LAG-3 antibody (FIG. 18E); a combination of anti-PD-1 and anti-LAG-3 (FIG. 18F); and a combination of anti-PD-1, anti-TIM-3 antibody, and anti-LAG-3 (FIG. 18G).

New Framework for Identifying Cancers for Treatment with Triple Blockade Therapy

PD-1, TIM-3 and LAG-3 are expressed to varying degrees across cancers. In this example, a framework was developed to identify cancers in which therapeutic benefit may be preferentially derived from triple blockade of PD-1, TIM-3 and LAG-3.

Signatures were derived to define the presence of tumor infiltrating lymphocytes (lymphoid index), tumor infiltrating myeloid cells (myeloid index), tumor interferon (interferon index), tumor cytokines (cytokine index) as well as measures of tumor mutational burden (TMB) and homologous recombination deficiency (HRD or HRR gene mutations). These signatures were then compared in The Cancer Genome Atlas (TCGA) to identify tumor types that may preferentially respond based on the concurrent presence of several of the factors above but also to define tumor types where a subset may preferentially respond based on the marker profile.

To derive the signatures, K-means clustering was performed on the combined 10,000-sample pan-cancer dataset at gene level using a range of different k (k=20, 21, . . . , 200). Fisher's exact test was used to obtain p value of the canonical pathways and Gene Ontology term association for each cluster for any given k. For each k, combined p values for top 20 clusters were calculated. The optimal k of 62 was selected for smallest combined p value among all k. The average expression of all genes in each cluster was used as the index to represent transcriptional status of the cluster.

Based on TCGA's RNA-seq data, it was determined that the mammalian cancer genome can be usefully represented by 62 non-overlapping, functionally relevant groups of genes (transcription clusters) whose intra-group transcript level is coordinately regulated across cancer types. Although the transcription clusters were identified through non-supervised clustering analysis, it was observed that genes with known similar functions clustered together. Transcription clusters were found to be more robust than any single gene, and to be better than conical pathways because they were optimized for transcriptome analysis by non-supervised clustering without prior knowledge. Such clusters may provide additional insights than canonical pathways.

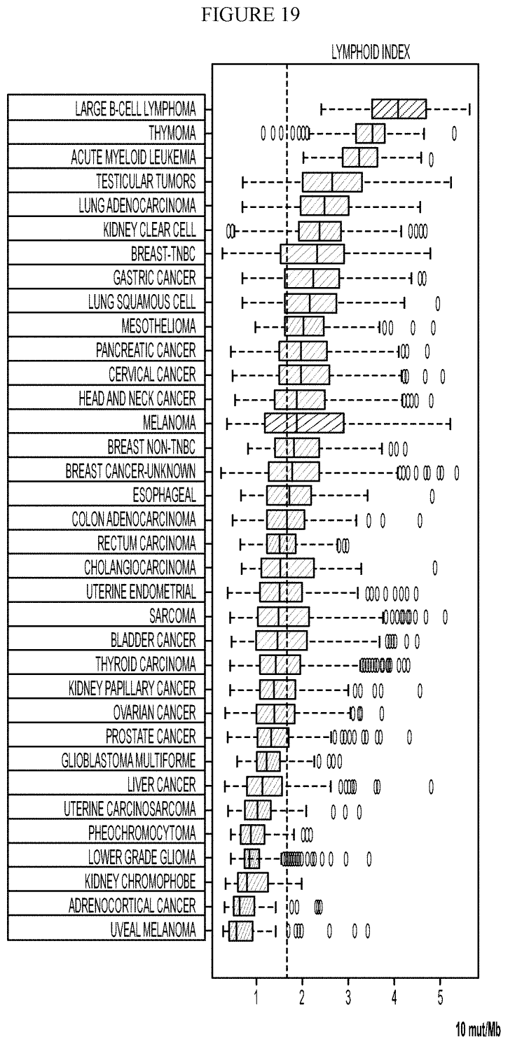

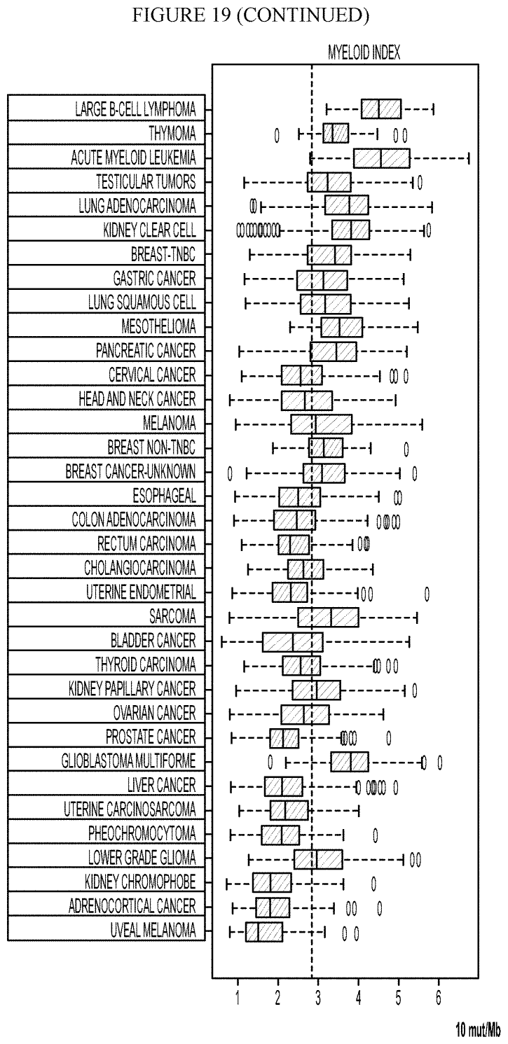

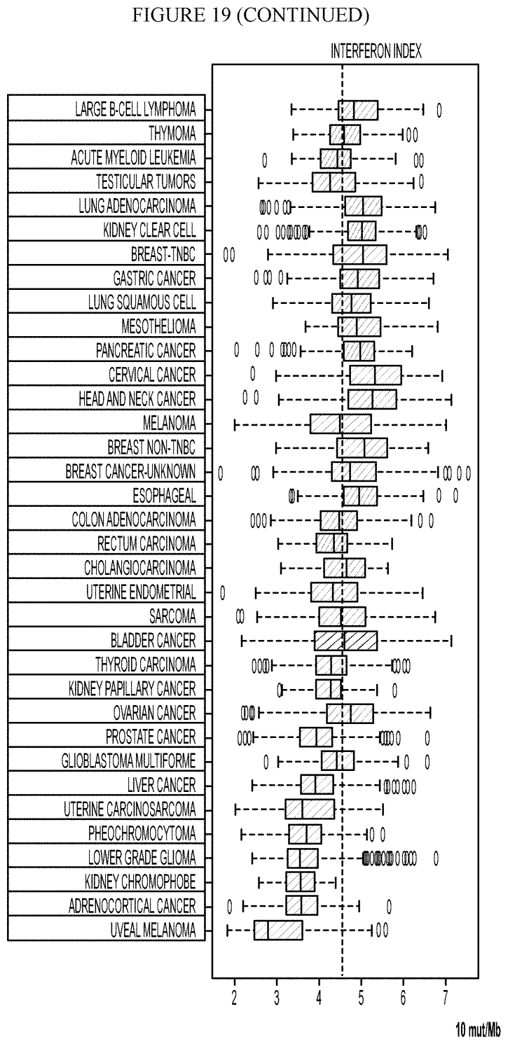

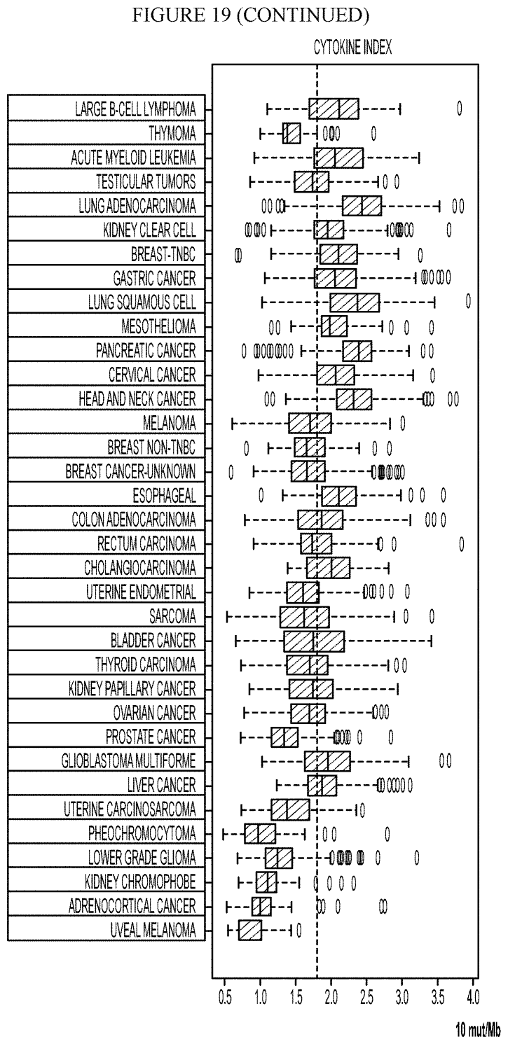

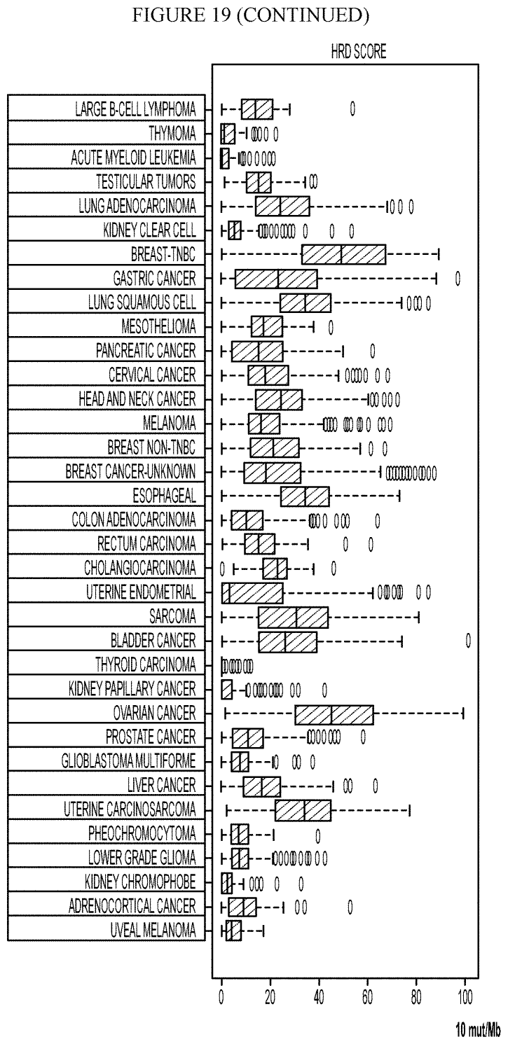

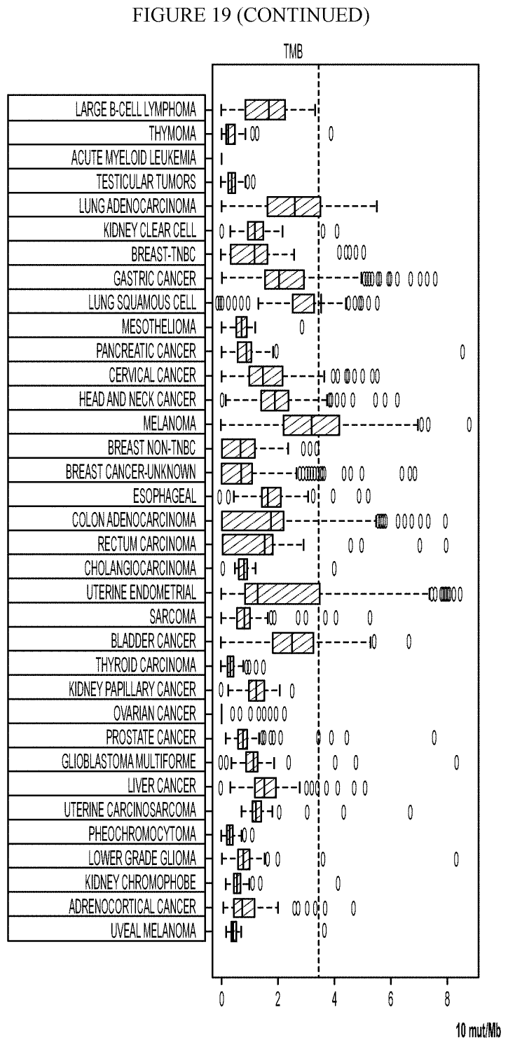

A least four immune clusters were identified, termed lymphoid, myeloid, interferon, and cytokine. Lymphoid cluster is enriched for genes related to T cells, B cells, and NK cells; and myeloid cluster is enriched for genes related to macrophages, neutrophils, monocytes, etc. Both lymphoid index and myeloid index correlate with leucocyte percentage in the TCGA gastric dataset. A summary of the signatures used is shown in FIG. 19.

The following cancers were identified as having the highest lymphoid index: Large B-cell lymphoma, thymoma, acute myeloid leukemia, testicular tumors, lung adenocarcinoma, kidney clear cell, triple negative breast cancer, gastric cancer, lung squamous carcinoma and mesothelioma.

Cancers characterized by high lymphoid index and myeloid index were next identified. The top indications from this analysis were large B-cell lymphoma, acute myeloid leukemia, kidney clear cell, lung adenocarcinoma, thymoma, testicular tumors, breast-TNBC, mesothelioma, pancreatic cancer and lung squamous cell.

In addition, cancers characterized by high lymphoid, myeloid, interferon, cytokine indexes and tumor mutational burden were identified. From this analysis, the top indications were lung adenocarcinoma, large B-cell lymphoma, lung squamous cell, breast-TNBC, kidney clear cell, head and neck cancer, gastric cancer, pancreatic cancer, cervical cancerand mesothelioma

Another patient selection marker for PD-L1-based checkpoint immunotherapy is defects in DNA repair pathways (Teo et al., 2018, J Clin. Oncology). To identify tumor types that displayed the highest levels of lymphoid, myeloid, interferon/cytokine indices overlaid with TMB and defects in DNA repair pathways, measurements of HRD and defects in HRR genes were developed and the TCGA database evaluated. When this analysis was performed, the top indications were lung adenocarcinoma, lung squamous cell, bBreast-TNBC, gastric cancer, head and neck cancer, large B-cell lymphoma, esophageal, pancreatic cancer, cervical cancer, kidney clear cell, mesothelioma, melanoma, bladder cancer, colon adenocarcinoma.

Collectively, these analyses indicate that tumors with high levels of the individual markers or an overlay of multiple markers would be more likely to respond to triple PD-1, LAG-3 and TIM-3 checkpoint blockade. For example, even in cancers that are not generally positive for high lymphoid, myeloid, interferon/cytokine, TMB or defects in DNA repair, one can increase the likelihood of successful treatment by determining which subsets patients are positive for these markers either alone or in combination. Examples would include subsets of endometrial, colorectal, non-small cell lung, gastric cancers and melanoma (FIG. 19).

Another factor important in regulating T-cell exhaustion is the presence of tumor-associated viruses such as HPV, hepatitis B/C, EBV. Overlaying viral infection with the markers above, top tumor types are virally infected head and neck cancer, cervical cancer, hepatocellular carcinoma, and nasopharyngeal cancer.

Another patient selection marker for PD-1 monotherapy is microsatellite instability (MSI-H) or defects in the mismatch repair pathway (dMMR). As shown in FIG. 12B, there are high levels of PD-1, TIM-3 and LAG-3 expression in endometrial cancer, around 20% of which are reported to be MSI-H. These tumors are also expected to have high TMB, as shown in FIG. 19. Given the predisposition of MSI-H tumors, including endometrial to respond to PD-1 monotherapy (pembrolizumab label) and the expression of TIM-3 and LAG-3, triple combinations can be used to treat MSI-H tumors. In addition, given the breadth of PD-1, TIM-3 and LAG-3 expression, non-MSI-H endometrial tumors can also be treated with doublet or triplet PD-1, TIM-3 and LAG-3 blockade.

In sum, in two non-clinical xenograft models, triple blockade of PD-1, TIM-3 and LAG-3 with TSR-042, TSR-022 and TSR-033 was particularly effective, an effect that was associated with increased CD45⁺ TILs in the triple combination treated animals. These studies show that dual blockade of PD-1 with α-TIM-3 or α-LAG-3 resulted in improved efficacy over monotherapy in humanized mouse tumor models. Additionally, a triple combination of all three checkpoint inhibitors is associated with further anti-tumor activity: triple blockade of PD-1, TIM-3, and LAG-3 led to significant pharmacodynamic changes relative to PD-1 monotherapy, increasing TILs, reducing intratumoral Tregs, and impacting TAM populations with the tumor microenvironment. Thus, given the potential role of PD-1, TIM-3 and LAG-3 in T-cell dysfunction, these observations indicate that concurrent blockade of all 3 checkpoints may potentially elicit stronger and more durable anti-tumor immune responses than the single or dual combinations and can be a particularly effective therapeutic strategy.

### Equivalents

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications, websites and other reference materials referenced herein to describe the background of the invention and to provide additional detail regarding its practice are hereby incorporated by reference.

What is claimed is:

1. A polypeptide that is capable of binding Lymphocyte Activation Gene-3 (LAG-3) comprising: (a) an amino acid sequence of SEQ ID NO: 5, (b) an amino acid sequence of SEQ ID NO: 6, and (c) an amino acid sequence of SEQ ID NO: 7.

2.-4. (canceled)

5. A polypeptide that is capable of binding Lymphocyte Activation Gene-3 (LAG-3) comprising: (a) an amino acid sequence of SEQ ID NO: 8, (b) an amino acid sequence of SEQ ID NO: 9, and (c) an amino acid sequence of SEQ ID NO: 10.

6.-7. (canceled)

8. A polypeptide that is capable of binding Lymphocyte Activation Gene-3 (LAG-3), wherein said polypeptide comprises
a heavy chain variable region comprising:
a CDR-H1 comprising an amino acid sequence at least 80% identical to SEQ ID NO: 5;
a CDR-H2 comprising an amino acid sequence at least 90% identical to SEQ ID NO: 6;
a CDR-H3 comprising an amino acid sequence at least 80% identical to SEQ ID NO: 7;
and
a light chain variable region comprising:
a CDR-L1 defined by an amino acid sequence at least 90% identical to SEQ ID NO: 8;
a CDR-L2 defined by an amino acid sequence at least 90% identical to SEQ ID NO: 9; and
a CDR-L3 defined by an amino acid sequence at least 85% identical to SEQ ID NO: 10.

9.-21. (canceled)

22. A polypeptide that is capable of binding Lymphocyte Activation Gene-3 (LAG-3), comprising:
a heavy chain polypeptide sequence according to SEQ ID NO: 1; and
a light chain polypeptide sequence according to SEQ ID NO:2.

23.-54. (canceled)

55. A composition comprising the polypeptide of claim 3 and a pharmaceutically acceptable carrier.

56.-65. (canceled)

66. A method of treating cancer, comprising administering a pharmaceutically effective amount of the composition of claim 55 to a human in need thereof.

67.-68. (canceled)

69. The method of claim 66, wherein the cancer is:
i) a cancer associated with a high tumor mutation burden (TMB);
ii) a cancer that is microsatellite stable (MSS),
iii) a cancer that is characterized by microsatellite instability,
iv) a cancer that has a high microsatellite instability status (MSI-H),
v) a cancer that has a low microsatellite instability status (MSI-L),
vi) a cancer associated with high TMB and MSI-H,
vii) a cancer associated with high TMB and MSI-L or MSS,
viii) a cancer that has a defective DNA mismatch repair system,
ix) a cancer that has a defect in a DNA mismatch repair gene,
x) a hypermutated cancer,
xi) a cancer comprising a mutation in polymerase delta (POLD)
xii) a cancer comprising a mutation in polymerase epsilon (POLE),
xiii) a cancer that has homologous recombination repair deficiency/homologous repair deficiency (“HRD”) or is characterized by a homologous recombination repair (HRR) gene mutation or deletion;
xiv) adenocarcinoma, endometrial cancer, breast cancer, ovarian cancer, cervical cancer, fallopian tube cancer, testicular cancer, primary peritoneal cancer, colon cancer, colorectal cancer, small intestine cancer, squamous cell carcinoma of the anus, squamous cell carcinoma of the penis, squamous cell carcinoma of the cervix, squamous cell carcinoma of the vagina, squamous cell carcinoma of the vulva, soft tissue sarcoma, melanoma, renal cell carcinoma, lung cancer, non-small cell lung cancer, adenocarcinoma of the lung, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, a hematological cancer, multiple myeloma, B-cell lymphoma, T-cell lymphoma, Hodgkin's lymphoma/primary mediastinal B-cell lymphoma, chronic myelogenous leukemia, acute myeloid leukemia, acute lymphoblastic leukemia, non-Hodgkin's lymphoma, neuroblastoma, a CNS tumor, diffuse intrinsic pontine glioma (DIPG), Ewing's sarcoma, embryonal rhabdomyosarcoma, osteosarcoma, or Wilms tumor; or
xv) a cancer of xiv), wherein the cancer is MSS or MSI-L, is characterized by microsatellite instability, is MSI-H, has high TMB, has high TMB and is MSS or MSI-L, has high TMB and is MSI-H, has a defective DNA mismatch repair system, has a defect in a DNA mismatch repair gene, is a hypermutated cancer, is an HRD or HRR cancer, comprises a mutation in polymerase delta (POLD), or comprises a mutation in polymerase epsilon (POLE).

70.-99. (canceled)

100. The method of claim 69, wherein the method further comprises administering another therapeutic agent or treatment.

101. The method of claim 100, wherein the method further comprises administering one or more of surgery, a radiotherapy, a chemotherapy, an immunotherapy, an anti-angiogenic agent, or an anti-inflammatory.

102. The method of claim 69, wherein the human has been further administered or will be administered an immune checkpoint inhibitor.

103. (canceled)

104. The method of claim 102, wherein an immune checkpoint inhibitor is an inhibitor of PD-1, TIM-3, CTLA-4, TIGIT, CEACAM, VISTA, BTLA, LAIR1, CD160, 2B4, CD80, CD86, B7-H3 (CD276), B7-H4 (VTCN1), HVEM, KIR, A2aR, MHC class I, MHC class II, GALS, adenosine, TGFR, B7-H1, B7-H4 (VTCN1), OX-40, CD137, CD40, IDO, or CSF1R.

105.-175. (canceled)

176. The method of claim 69, wherein the method comprises administering the LAG-3 binding agent at a dose of about 1 to about 5000 mg, about 1 mg, about 2 mg, about 2.5 mg, about 3 mg, about 4 mg, about 5 mg, about 6 mg, about 7 mg, about 8 mg, about 9 mg, about 10 mg, about 50 mg, about 100 mg, about 200 mg, about 250 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 2000 mg, about 3000 mg, about 4000 mg, or about 5000 mg.

177.-182. (canceled)

183. The method of claim 69, wherein the method comprises administering the LAG-3 agent every week, every two weeks, every three weeks, every four weeks, every five weeks, every six weeks, every seven weeks, or every eight weeks.

184. (canceled)

185. The method of claim 69, wherein the method comprises administering the LAG-3 agent at a dose of about 20 mg, about 80 mg, about 240 mg, about 500 mg, about 720 mg, about 900 mg, about 1000 mg, or about 1500 mg every two weeks, or about 240-720 mg or about 240-1500 mg every two weeks.

186.-234. (canceled)

235. The method of claim 69, wherein the LAG-3 agent is administered parenterally, intravenously, or subcutaneously.

236.-254. (canceled)

255. A method for treating cancer in a human, the method comprising intravenously administering an anti-LAG-3 antibody comprising a heavy chain at least 90% identical to SEQ ID NO: 1 and a light chain at least 90% identical to SEQ ID NO: 2.

256. The method of claim 255, wherein the cancer is non-small cell lung cancer.

257. A method for treating cancer in a human, the method comprising intravenously administering an anti-LAG-3 antibody comprising:
(i) a heavy chain variable region comprising:
a CDR-H1 comprising an amino acid sequence at least 80% identical to SEQ ID NO: 5;
a CDR-H2 comprising an amino acid sequence at least 90% identical to SEQ ID NO: 6; and
a CDR-H3 comprising an amino acid sequence at least 80% identical to SEQ ID NO: 7; and
(ii) a light chain variable region comprising:
a CDR-L1 comprising an amino acid sequence at least 90% identical to SEQ ID NO: 8;
a CDR-L2 comprising an amino acid sequence at least 90% identical to SEQ ID NO: 9; and
a CDR-L3 comprising an amino acid sequence at least 85% identical to SEQ ID NO: 10.

258. The method of claim 257, wherein the cancer is non-small cell lung cancer.

259. The method of claim 257, wherein the method further comprises administering one or more checkpoint inhibitors.
